SEQUENCE LISTING (9 sequences)

SEQ ID NO: 1 - protein, 19 aa, Artificial sequence
  Antibody to human AdipoR1 raised in rabbits
NLQEFRYGLEGGCTDDTLL

SEQ ID NO: 2 - protein, 13 aa, Artificial sequence
  Antibody to human AdipoR2 raised in rabbits
FMIGGGCSEEDAL

SEQ ID NO: 3 - DNA, 17741 bp, Homo sapiens
atcacgcacttcaggctgcgataacgaggtggagtgtagaatccgacgaacgtcctctaccccggggcgggtggaggttacctggctgcggcggggaggtgggcaggcggcagggaaggcggggcagcagggcggggcccggggcgggaggtgtggccggcgggcgcgggcctgtgattgggactccgcgagagggcctaggattggagggcccaggcagagcccgcgaggaggtgacgcggctgcggaggtgacgcgggaggtcgcgcgccccttccggcgcggggagggcgctgaagatcggggccgctcggccgcaggccgcctccagcgccgcgggatgtagcgcgggggaccgcggcccccagcagagcccgcctgcccggctgtaagtctcccaggggctgcgcggtgggcagggactcggcgggagcgcggggaccccgggggccccgacccaggcctgcggaggagacagctgttgggggaacccgctccgacttgccagggggctcgggccccagccaaggagccgcctggcctccaggcatccagccggtcaggttgtaaacatttccggcccctcccactgcgcgccgcctccgaggcggcctaggtgcggtgggcgctcgtccccggctgtcccccgactcccggcagtcctgtgagtgctgcgcctttagccccacggggtggagatgcatgggttccccagatcgggaccagagagccagagttaagcctggccccgaggccgccgcgcgtggggagggagggctgcgggcctaggtcgcgaaccgcctctcagcggggaacagccgcattgggttgccaggaagccctaaggttcctccaccaacaatctttggcaggtcaagtcctgacagtgccctcactttgatggggtgggaagggaggttggtgatcgctctgcagggttaaggtaatcctgaaggctgaaatgcggggcagaatcgaaggtactttgggggaaaatgaaatttcattaccttaggtatctaggattgatgtattctatcttctgtttgagaattttggtcttcaagccttcgatttttaagaaaatagataagtgttttttttccctcagtttctggtgctctttgggagaatgggaaactgacaaggccaatttcagtttacaccgtttcagatgtcaaaggagcgctaggaggccatggtactttactagattaaccatagtacttccctcaagtgattcagaagacgggctagaaaatacagcggagaatacataggctgccatctacaaaacaaatgattgtaatggctggttcttttctgaaatgaagtaatagtggggtcggttcactgcttttctcttttcctagtagttgggtaggctacgatggtgcaacttcggttcctgaggttgtatactatctgtatactgccaggaatttagggtggtgctaaacagattgcaatgtcagggtaccggcttgcagctactaaatgtatttactccaggaataacttttatgtacccatattgtgatttgcacttgggaaatcagtcataaaaactgatgttacctgtgaaggcccatgttatggtagacctccgcaaaactatacctaattgtagagtagaaactgaaagctttctattctcaatggtcaagactttgcctgcaggtaggagagaatctcagaaaaataacagggagcaacattaatccactgatccccattgctgatttataaagtacaccaccttaagtaggcttttaggctattttcagttttttaagtctggcctcattttacctttccaggactctatggaaaaatgaattgtttactgccctggggactgtgttctttcatggcatgttcacaccatataccctcctggaatgccctgtttccatctctatctatcaatctcctgtcataaaatcttccacaaaactttttctgatcacttcaactaaagttgcacttttcttttgaattcctcctgcacataatggcactaagccttgtagtgatattaggaaagttatttcccccatttagaggccaacaaccatttcaaccctctgactcctctttgccttgtctctgtggccacaacaccatgcgtattgtaagtaatcaatcggtattctcagattaatgaaggttagaaattgcccttctttctaggccagtgtatttgttctctattcccataggaaatttcctttgacttctaatacaaagccaaactgaaaccctttaacactcctgctacattatcctgtcccatctacttattatcacacaggtaagataagcagtcgtctttatatctagacgctttgtgaattatggtgagttttttttcttggaccagtactcatcaacgttgttatgtccatttgtccctaatgtcacctcctggattttgttcctctcaccatgctaaaagttgattcccattttcctgctcagaaatgttgggggcataggccgggcacagtggctcatgcctctaatcccaacactttgggaggctgaggcgagtggatcacctgaggtcaggagttcgagaccaacctggtcaacatggcaaaaccctgtctctactaaaaatacaaaaattatccggatgtggtggcaggtgcctgtaatcccagctacttgggaggctgaggcaagagaattgcttgaacctggaaggcggaggttgcagtgagcagatcatgccactgcactccaccctgggggatagagcaaggcccttgtctccaaaaataaaagtgggggctggcattggctgggcacggtggctcacacctgtaatcccagcactttgggaggctgatgcaggagaattgcttgagtccaggagtataggctgcagtaagctatgatcgtgccacacactctagcctgggcctggaactggctgaacttgggagcaagaccctgactccaaaaagaaaaaatgggctggcattaaaatccttcatatgcctgtatactgttcacttttttttttgtcatgacattagcaaagtttgcctgtctgtcagggctcaaaaacacctcttcagccaggcacagtggctcatacctgtaatcccagcactttgggatgccaaggctgggcaacatagtgagaccttgtctctacaaaaaaataaaattagctgggcatggtggcacatgcctgtagtcccggttactaagacttgggaagctaaggtgggaggatcgcttgagtctggcacattgaggctacagtgagctgagatcacaccactgcactccagcctgaatgacagagcaagacccttttaaaaaaaaaaaaagacttctgttggggaagagctacaaaggaagtcagactgtttttccacttcagagtctgggacgcttggcatcgagtaggagagctgaggccctatatactgtagttcggtgatttcaaacatgtgcctttgctttgattcatatttctccaacaaagctgtgaaattatcctgcctaagagacagtagataggggaaaaatggctgtattctttatctatactgctagactggtcttgctttaggacaagaatcaagctaggtatggatagaggctacccatagactctctaaaggcccaggaactaaactcttagaaactttgtgtctttgcctccaggaaaggtgtgccgtagcctggaaaacttgggaattggttcacaatataaatgtctccaaaaactataagtgaaataactctccagctaactggctggtgattaaaaatcaatcttttccccatgtttggcacaggattatcttatttataggagtacaaacaacctccctgctccacagctttactctctaatatcctaggtttacccagttggaaagcttagtggtaaaggtttattagcttgagttgaagaaatctcatcaaaaatgcactcagtcgagtttggctcaaatataatttgcagtattgggtgtggcaagcttgaggggaaaggcatttcctctaaaatacgtaaggacatagaaagctggcagaaggttggctcacctaggatcgttctaacaccttaggggtttgttctgattttacaccatagactaaaacacacttgtaattctccattgtttgtgagtgatccaccccatagaagtggtattttcagatcctagtattatttgctcttctttcttgccagacgtccgatccctatctctccctttcctgtctctgtgttactacagtttagaagaccaaactggacaatggactttgctcactatgatgacatgatctccattgacctccattgtatgtactttaatgcatgatgatatattttgtatgatgagttgtagctgatggctggtttgtgagttaactggggtctcaggttttggtgactctgggtctagaagatgtagagaggtggtataggagctagtgatagaggagctagagggaatcggaagaactaatagtaaatgatttggactgaaaaaggaaaagactttgatgatagccagaagacttgagggcataaaaacttttcagatcaaatgatgaaattagtaagaatgccaatctggattctgcacatgatctttggaccctaattgcccaacttaaattttctctatattattctcattgcctctagtaaaatagttacatagcactcaaagttactaaatttagtgtgtgaccattctagttgaggggttctctccctccactgccctcctggagcaatcaaattgtatcacttatttttgtgcatatcacaaactacctatgttatctggctctttgaaaacaatactataacttgttcaacttgtattttgcacagtgtcttgtattttgcacagtgtcttagaatgccttagggtgctagtcctaaatacacttgctaaatattcaatgaatgtgttgacttactctggggaaagggataagagaagccaccacaagcacaagaaaatgcacataagcccttatctaagtggctagaagctaagtcatatgctggcaagctggaatctgccccattcattcattcattttgaaatgggttctaactaaaataaagtatatttattattttatgatccatgtattgcatctcagaaccagaattttgttttgttttgttttgtttctgccctggtagcctgatctaagttacttccaacagtaacttagtaagtaacaagctaagtgtgcccctcttttcagatttatctaagatgcccaaaaatatttatgttttttgcttgtgatataaagttgttttttggtttttttttttttgcttgcgatataaagtattttttagtaaagctgctgattcagtagtacttcactttgaggaaatatgttttgaggtcagcctattgagaaagatttccttgtgtccctctcacacgggttgcccatcctcgaggttttcataggcttccagcttcctgttctccttagctcaattgttcactatgactgtgtgctggcatctcgtcagagtcttgcacatttaaaggaagaggaactgtctatgtgttgacaccatataaaggtctgctgtatagcaagttattgtttttgtctctcttggactagattgccggttgctttcaactgtgcttgtgggttgagcatagcatgactcatgactgtttagaagcttgaggattgcccttacctgatgtgttagtaagtgttctccagaaagcaccaataggatacatacatatggatagagatgggatttattcggggaattggctcatgagattatggaggctaagtcccacactatgccatctcaagctagacaatgaggaaagtccataactggttcagtttaaggctgaaggcctgagaacccaaggggctattggtgcacgtcccggaatcccaaggcaggagaacctggagtttctgatgtccaagggcaggaaaagaagggtgtcccagctccaggagggaaggagggggagagacagagagagttagggagtcagagaaagagatttataatccctgagaaggaattgtgattactcttagataaaaaagaactgacacaagatatcaaagatatttgtatatagtgaatagccatggatgatatagtgtctccctcaggagcaaagttatgtttactgcccaatataaaagattggggctccctaagcttaagtttcctctcccggaatgcaacccactccatgtgcagttgttacctggccctccctgcattgcaatgtggttatttgggtttgggtggggcttgggtaaccaatgcaagaaaatgctaatattctggctactgctattgctataactaataaagtcctttgtatgggggtagaggtggggagagaactaataatttacagcatagtggaaatctttggaaactttctccctctttccctctttttttatttgcatttccctgaggcctgtttaaagactactgtgaaaatccaacccaggaaaacctaaagtgtctgagatttttcctagaaaatggaattctgggtttttctgattttgtgtgtgcctgtctctagtttctgaaatactcatttgagtagggcttagattatgggttgtggtggttgaaacaagttattgtggagctggttattaggtatgtgtgtggcaaagggtaaattggggctgcctccttgtcttcaccttcctgcttccaggtaagttcttgcttgcttgcaggacagattgtgatgaaagcttgttatcccagggttgaactcaggctataatcctgtaatagaattagtagacttcgcagagaaattaaccactaggcatctgtcccacaaccaagttccttttgtatgtatgacacatttatcagagcatttgtagataaggtctaactgagttcagttggcatttaattcatttctaagcctggatgatcttgtagtaattgcagccagatgttggctttatatttttattcctcggtctcagagctaggcctttcttgggcagagtgagctttgggagtgatagatgagattcttctcactaaatggttccatacctgtgggagggagttgagggtgggtagctgtttttctatggtgacagtttggtgcagtaaccatagatcatatgaaagcaagcaactaggtgttacattttcagccttacctataagattagtgaggttctgggtaaaggttgacattaaagagggaggacaggaatagatttcatttaagcaattaactggcctttagaattggggacaggatatgagttgattagcttcttattcagtattagagtatagaaatgaaactgtaagaggaataaattgtcaatccttcatgtttgtattttcttttagtgtctaccatcagagggagatctctgccccctggggctgagagaccccaacctttccccaagctgaagctgcagggtattgaggtaccagccagatgtcttcccacaaaggatctgtggtggcacaggggaatggggctcctgccagtaacagggaagctgacacggtggaactggctgaactgggacccctgctagaagagaagggcaaacgggtaatcgccaacccacccaaagtaagtcatcttcccaggagtcatccccttgttcagtttctctccttagaactctgtcattttctcttttttaagagctcaccgtccaaaaggaggaacattgcatcctggaaatgtctctatccaagtccctatgatactgttataaacatcttgttgtctgctttccccattgtaagcttttgggaatgcactcatctttgtaacaccagtgcttagcacaaggcctgcagtccaataaataatcagtaaatatctgaatgaatgaaggaagcagtcaattaggattaggtttgacttcatataacacacaaaaatccttacagttttgacaggacagaagtctctcaagcaaaagaatttcagaagtaaccaatccagggctgatatggaggatccaccactgtcaggcacctgtgttgctctcccatcactcttaatatgtgcccatcatacccacattctaccccccatgaaagggaaacgagaagaaaggccctctctttcccttttatttttattgagataagatctcgccatgttgcctaggctgatcttaaactcctaggctcaagtgatcctcctgcctcagcctcccaaagtgttggaattacaggtatgagccactgtgcccggccaactctttgccttttaaagggactatcagggccgggcacggtggctcacacctgtaatcccaacactttgagaggccgaggcaggtggatcacctgagatcaggagttcgagaccagcctggccaacatagtgaagccccctctctactaaaaacacaaaaattagctgggtgtggtggcagtcgcctgtaatcccagctactcgggaggctgaggcaggagaatcgcttgaatctgggaggcagaggttgcagtgagccgagatcgtgccattgcactccagcctgggcaacgagtgaaaccccctctcagaaaaaacaaaaataaataaagggactatcaggaagtacctcataagacttcggccagaacttacttacataactatattcagctgcagtggaggttagaaagtgcagcctgagtaaaattgcagtaacattgccaccctgaacaaaaccgtgggctttgttacttaggaagagagagaatggatgttagggtagactggctggaagctgtagcacaaaatctaagatggaccatgtgtttactagtgaccatcagcaacaggagagaaatagcccagaaaaggagttgaactgccaggctttctatagccatgattcggagccttgggatagagagagagaaagttctctccctttcacaggcaagtgctctcctgggtgtgttttttttttttttttttggagacagagtctcgctgtcgccctggctggagtgcagtggcacgttctcggctcactgcaggctccgccctccagggttcatgccattctcctgcctcagcctcccgagtagctgggactacaggtgcccgccacctcgcccggctaattttttgtatttttagtagagacagggtttcaccgtgttagccaggatggtctcgatctcctgacctcgtgatccgcccgcctcggcctcccaaagtgctgggattacaggcgtgagccaccgcgcccggccctctgggtgcgcttttttaggccctggagtcagtaccgctgttttccatggaaccagttctgaggccctggtactcctgggagctgctgtagatacaaagtttcaatcatgacagcatccctagagtcagtgaacagggaactctgaacagaattattgttatggaactctctgagactctgtgtctttttcgttgttttgaaaacgtaggctgggcatggtggctcagcctgtaatcccagcactttgggaggccgaggcaggaggatcacgtgaggtcaggagattgagaacagcctggccaacatggtgaaaccccatctctactaaaaatacaaaaattagtcaggcatggtggtgggcgcctgtaatcccagctacttggaggccgaggcatgagaattgcttgaacccgggaggcggaggttgcagtgaacagagatcgcaccactgtactccagcctgggtgacagagggatattccatttcaaaaaaaaaaaaagaaagaaagaaaatgtaatggcttagaaaagagaactctttgggaaactgaggaaagttaagaaccactatcctaaagaaaaggatgtcattcagtacctagagctggtgtattcccccaatgctagtctctgtgacagtccttcttttacgcactgcatagaatgttcactccagcatttacgaagtattaataaatttctaactagtgttgtaaactcagtgtgtatgcttttggagaagagaccacaagaccctctgtaggaaagaggctgggcagaaggcattaggtttgggtttttctgggtctctctgctctggaatagtacaatttatgagatgactccctggattgttgtgggttttttcccccataggctgaagaagagcaaacatgcccagtgccccaggaagaagaggaggaggtgcgggtactgacacttcccctgcaagcccaccacgccatggagaagatggaagagtttgtgtacaaggtatggcctatgagcttctgtcctggaaggggaagggcagcaggcctgtctgggatgtccccagccagcaagcttgcactgggtcgaagagcttttattcctgggaaggagtggatttgttttttggttaagatgaggtaaatcaatgtgaagactggctaaatttttactgcctactctgtgaggcattgttcaaaatatattcttaaaacggattcttttctatgcaaggggcgcctttggaagtcatcaaaatatttcaatatgacagtaatatgaatgcctttgttaacacattaaatatacatgaatgatgagcactaatgtattttattaaaatttgtaatttctaagtaatgatgaatagaaacagtattttgagatacctgcagaactaatgtgtgagagtatctgatttctattggtgacaaagccacaagttctactaatactgtggtttgttgcttatgcttataattgaggaaaatgctcaatttacttagaggtaaatggaaatacaattttttccatctattccctggatcctgtccatagtgcatggatcctagattaagaacccctactctaaaaagatgtggtaagaacttagaatctaattgggggagggatgagacatgagatagaatctcctggagaggggcaaaggagggatctgtacgttttttaaaacaacgtttataatttttttgtttttattcagtatatcaattttgaaatttcaagtagcctaaaggaggttgtaccatatatatatatatatttttttgagacagagttttgctcttgtctcccagctgcagtggcacgatctcagctcactgcaacctctgcctgccaggttcaagcacttctcctgcctcagcctcccgagtagctgggattacaggcatgtgccaccacgcctggcttatttttgtatttttagtagagacggggtttctccatgttggtcaggctagtctcgaactcccaacctcaggtgatccgcccgcctcagcctcccaaagtgctgggattacaggcgtgaaccactgcgcccagcctggttggaccatattttcttaaaaggttgagaaacacttacctaaaacttctccagataaactaacatgaaacaaagacaaagagattttttgtttgtttgtttttgtttttttgttttttttgaatacagagtcttgctcggtcgcccaggctggagtgcagtggtgcaatctcggctcactgcaacccccgcccgccaggttcaaaccattctcctgcctcagcctcctgagtagctgggattacaggtgcccgccaccatgcccggccaattttttgtgtttttagtagagacagggtttcaccatgttagtcaggcaggtcttgaactcctgacctcaggtgatccacctgcctcagcctcccaaagtgctaagattacaggcatgagccgctacactcagccaggattttttttttttttttaatggtcaaaatatgagtcaggactctgctagccccattcatgctatgtcttggatatcccccagtccaggctggttctccactattacctaggaccattcagagcctgactgacatcttcttagactagttctccatcactgcttgtccttggggaaggtactatctagtgccctacccagagaaagtgtatatgttctaggtctgggagggacgttggagggtcatcccatatgatgtgctccctgactggctaaaggacaacgactatctgctacatggtcatagacctcccatgccctcctttcgggcttgcttcaagagcatcttccgcattcatacagaaactggcaacatctggacccatctgcttggtgagtgatttggatggggaaaggtggaatttgttgtctccctaactgcgaagggattatagagcccaacaaagttgttcagatcgattggcagctgggattctacctttaaactactaatgactttaagctaataaccttggttgacccttgatcactgatctttaagactgagagtaaaaggcctagaaaccaaagaaattgggactagatatttttttgtgatccctggcagaactgttctaggtggagacaattccttgtacttcgtaatttccaactgtaaaagtaaaatgccaacatttcaaaaaatggtcttttttgtctgggtttctgtgaccagtgccactttctttgaaacattggactgcaattacacagctaaacctcatagctttctgattttttttttatttcaatagctcttgggttacaaaggggtgggggggggtttggttacatggatgaattatatactggtgaattctgagattttaatgcacccatcaccttagtagtgtacattgtactcaatatgtagtttttctgtctcacacccctatctccccctcccgcttctaagtctccatagtccattatatcattctatatgcctttgcgtactcatagcttagctcccacttataagtgagaacatatacagtatttgatttttcattcctaagttacttcacttagaatagtggccttcagcttcatccaggttgctgcaaaagacattattgcattcctttttatggctgagtagtattccatggtgtacatacaccacatttcctttattcactcattggtcaatggacacttaggttggttccatatctttagagttgtgaattgtgctacaataaacatacatgtgcatgtgtctttttcatatgactgcttttcctttgggtagacacctggtagtgggattggtggatcaaatagtagctctacttttagttctttaaggaatctctatacccttttccatagaggttatactagtttataatttacattcccacaaggagtgtataagcattccccttttaccatatccatgccaacatctgttgttttttgactttttaataatggcctttcttgcagaagtaaggtggtatttcattgtgcttttaatttgtatttccctgatgattagtgatgttgcctgtagctctctgattcaaaaacatcaaccatcaaagtgtacacattcccttttttcttacaggttttttttattttaccctcctgcaggtttcgtgctgtttctctttttgggaatcttgaccatgctcagaccaaatatgtacttcatggcccctctacaggagaaggtggtttttgggatgttctttttgggtgcagtgctctgcctcagcttctcctggctctttcacaccgtctattgtcattcagagaaagtctctcggactttttccaagtgagttaaaagaacaccataggaaaatactgtgcactgttgtcacatcttgagatcccagtgcttactgggtgataggagctcatatcactcctaaaggtttcttgatatgccagaggaaaggctctctgttctgtggcgatagtggggtgatgaagcttttctcttaaacacttcatgaacagacttactgcatcctcttcaacagatggagagaccaattgtggaatatattttctacctgaaaagcactatcttttgtgaggcttagagtgccagtgcactggctgctttccaagaccttgtacaagcgtaacttactattagcccaagccaaagtagggtgtggtaaatagtctatgcttcattctgctggtgctcctacccagcagtcaagtaaagcttatctttgctgcccatgcattgctggaagctccagatgtccatcatcgtgtgctttctctcttgcccagccgtgagcctgaggttctatttgtttgcttgtttttgttgttattgttggttttggtggcgacagtggtgtattattttgttttttctaacaacaccagtaagaaggtacattttcctcatttaaaaagtcaactaagatagaaaaagctagagtatcccttaatactactcccaatttaagaattttgtttttagaactcattctatcagaataggagaattttcaaagcctttcctgagtgtgatgctgggaaccagctggattgtgatggaatttctaaattcaagtgtctactatgggagatacggctgttcactagtaagggaaagggatagagttctttgaatggggattcttagagatttttccatcgtaaaatagaagtgaccttgtggaattttaatccagaatacaggatagattgtatgggagtctacaatacaggatagattgagggaatggttaaatctgggagcaggtggagaacagaaagagctagttaaatgagccatacaaattcatttcccccactgatttgtttcatttttgtggttctttttagactggactattcagggattgctcttctaattatggggagctttgtcccctggctctattattccttctactgctccccacagccacggctcatctacctctccatcgtctgtgtcctgggcatttctgccatcattgtggcgcagtgggaccggtttgccactcctaagcaccggcagacaagagcaggtaggtcggaagatgtacgtacacctcagaggtatttagaccttaggcctgataggctcaaacctatttgagccttaaggtttgcagcttgagaaccccacccaaaccctgatttgtgtttcagaactttcaaaacagcatcaatttcaaagctcagttctcaacccccagtgtcatactatttcagtagaatgccaataggacagatcattacccagctggtcaattaactgcaagcacagatgtagctactcttatacttggtttgtgacagtttaaaaatgtagcaagcacagtaattttagtgacccgagtcccaagctcagaccaagggattaaaattaaaacactcagaggcattttactaagggaatggaatacctggtttggtgaatgaaaataccttgagcataaacaggtaacaaagtaatagctacaagcataaacatctttgtgttgtctcatctcatctggactcttattttaagcttttattacttgcagcaacaccaccaattaccactagtctttagtccctttacaattaatatctgctatgttttaaccttcagttaaaataattaaatattgtccgggagcagtggctcacgcctgtaatcccagcactttggggggccgaggcaggcggatcacttgaagtcaggagtttgagaccagcctggccgacatggtaaaaccctgtttttatttaaaatataaaaattagtcggcgttgtggcaggtgcctgtaattccagctacttgagaggctgaggcgggagaatcacttgaacctggtcgggggcagaggttgtagtgagccaagatcacgccactgcactccagcctgggcaacaaagcaagactctatctcaaaaaaaaaaaagaaagaaagaaagaaaagaaaaaaataataataattaaatatttatcaagcaccagccacgtgccatgcactgtgcttaaacaaaggtatttacaaagatggagaggtggtccctgcccttcagttatgtgtagtgtaatgatgagacacatacccattcttcccttgctcttgccttacttgtgtgtcccagcagctacaggataactgctaatctgggtatatctgtctactcaggaggtcagagttctggttatgtaagacttttatgatcttaacaggaagcctatcctatgcttctctgacttgggaggcaactgaagccttgacatctaagttctgtacccaagttagccatcagagagtcttgacataccatttttacctaaattccttaacagataatgaagaatatgagatctttgttcttggtgcttaaaaggtagtaagcacttacacagcttcttcccattcaggacatagaagaaatatctgttgagtgttatccatcttgtagctaagtttcctaaacatgtactattcatccaaaaatagctaattatcataaacattgaaattccagagtaagacagcagtgccttccatgcggtggctgtttccttgtttgacagtcttatgtctgttctgttcaggcgtgttcctgggacttggcttgagtggcgtcgtgcccaccatgcactttactatcgctgagggctttgtcaaggccaccacagtgggccagatgggctggttcttcctcatggctgtgatgtacatcactggagctggcctttatgctgctcgaattcctgagcgcttctttcctggaaaatttgacatatgggtaagaaatgaatcttccagcaggtcctgtgatggtgagtgaacagcccaaagttaaagggctgaggaaaaggtggtatgagactgggatcatatagtgtttggaggtacatatatgtgcattgaaggtaaatcatcaatattaacattttggtgtatctctgacaaggactgtgtttttaaaactttctaaatataccactttaatgttattaacaaatttgtttaattaaggataaactagaagaagcaaatgctttctcctacttgtttcttattcctccttgcattatcctaaaggctcttttctttttattcctacagttccagtctcatcagattttccatgtcctggtggtggcagcagcctttgtccacttctatggagtctccaaccttcaggaattccgttacggcctagaaggcggctgtactgatgacacccttctctgagccttcccacctgcggggtggaggaggaacttcccaagtgcttttaaaaataacttctttgctgaagtgagaggaagagtctgagttgtctgtttctagaagaaacctcttagagaattcagtaccaaccaagcttcagcccactttcacacccactgggcaataaactttccatttccattctcctagctggggatggggcatggtcaaacttagccatcccctcctcagcaaggcatctaccggcccctcacagagacagtactttgaaactcatgttgagattttaccctctcctccaaccattttgggaaaattatggactgggactcttcagaaattctgtcttttcttctggaagaaaatgtccctcccttacccccatccttaactttgtatcctggcttataacaggccatccatttttgtagcacacttttcaaaaacaattatataccctggtcccatctttctagggcctggatctgcttatagagcaggaagaataaagccaccaacttttacctagcccggctaatcatggaagtgtgtccaggcttcaagtaacttgagttttaattttttttttttcttggcagagtaatgtaaaatttaaatggggaaagatatttaatatttaatactaagctttaaaaagaaacctgctatcattgctatgtatcttgatgcaaagactatgatgttaataaaagaaagtacagaagacacttggcattcaaa

SEQ ID NO: 4 - DNA, 2190 bp, Homo sapiens
attgggactccgcgagagggcctaggattggagggcccaggcagagcccgcgaggaggtgacgcggctgcggaggtgacgcgggaggtcgcgcgccccttccggcgcggggagggcgctgaagatcggggccgctcggccgcaggccgcctccagcgccgcgggatgtagcgcgggggaccgcggcccccagcagagcccgcctgcccggcttgtctaccatcagagggagatctctgccccctggggctgagagaccccaacctttccccaagctgaagctgcagggtattgaggtaccagccagatgtcttcccacaaaggatctgtggtggcacaggggaatggggctcctgccagtaacagggaagctgacacggtggaactggctgaactgggacccctgctagaagagaagggcaaacgggtaatcgccaacccacccaaagctgaagaagagcaaacatgcccagtgccccaggaagaagaggaggaggtgcgggtactgacacttcccctgcaagcccaccacgccatggagaagatggaagagtttgtgtacaaggtctgggagggacgttggagggtcatcccatatgatgtgctccctgactggctaaaggacaacgactatctgctacatggtcatagacctcccatgccctcctttcgggcttgcttcaagagcatcttccgcattcatacagaaactggcaacatctggacccatctgcttggtttcgtgctgtttctctttttgggaatcttgaccatgctcagaccaaatatgtacttcatggcccctctacaggagaaggtggtttttgggatgttctttttgggtgcagtgctctgcctcagcttctcctggctctttcacaccgtctattgtcattcagagaaagtctctcggactttttccaaactggactattcagggattgctcttctaattatggggagctttgtcccctggctctattattccttctactgctccccacagccacggctcatctacctctccatcgtctgtgtcctgggcatttctgccatcattgtggcgcagtgggaccggtttgccactcctaagcaccggcagacaagagcaggcgtgttcctgggacttggcttgagtggcgtcgtgcccaccatgcactttactatcgctgagggctttgtcaaggccaccacagtgggccagatgggctggttcttcctcatggctgtgatgtacatcactggagctggcctttatgctgctcgaattcctgagcgcttctttcctggaaaatttgacatatggttccagtctcatcagattttccatgtcctggtggtggcagcagcctttgtccacttctatggagtctccaaccttcaggaattccgttacggcctagaaggcggctgtactgatgacacccttctctgagccttcccacctgcggggtggaggaggaacttcccaagtgcttttaaaaataacttctttgctgaagtgagaggaagagtctgagttgtctgtttctagaagaaacctcttagagaattcagtaccaaccaagcttcagcccactttcacacccactgggcaataaactttccatttccattctcctagctggggatggggcatggtcaaacttagccatcccctcctcagcaaggcatctaccggcccctcacagagacagtactttgaaactcatgttgagattttaccctctcctccaaccattttgggaaaattatggactgggactcttcagaaattctgtcttttcttctggaagaaaatgtccctcccttacccccatccttaactttgtatcctggcttataacaggccatccatttttgtagcacacttttcaaaaacaattatataccctggtcccatctttctagggcctggatctgcttatagagcaggaagaataaagccaccaacttttacctagcccggctaatcatggaagtgtgtccaggcttcaagtaacttgagttttaattttttttttttcttggcagagtaatgtaaaatttaaatggggaaagatatttaatatttaatactaagctttaaaaagaaacctgctatcattgctatgtatcttgatgcaaagactatgatgttaataaaagaaagtacagaagacacttggcattcaaaaaaaaaaaaaaaaaa

SEQ ID NO: 5 - DNA, 2143 bp, Homo sapiens
atcacgcacttcaggctgcgataacgaggtggagtgtagaatccgacgaacgtcctctaccccggggcgggtggaggttacctggctgcggcggggagtttagaagaccaaactggacaatggactttgctcactatgatgacatgatctccattgacctccatttgtctaccatcagagggagatctctgccccctggggctgagagaccccaacctttccccaagctgaagctgcagggtattgaggtaccagccagatgtcttcccacaaaggatctgtggtggcacaggggaatggggctcctgccagtaacagggaagctgacacggtggaactggctgaactgggacccctgctagaagagaagggcaaacgggtaatcgccaacccacccaaagctgaagaagagcaaacatgcccagtgccccaggaagaagaggaggaggtgcgggtactgacacttcccctgcaagcccaccacgccatggagaagatggaagagtttgtgtacaaggtctgggagggacgttggagggtcatcccatatgatgtgctccctgactggctaaaggacaacgactatctgctacatggtcatagacctcccatgccctcctttcgggcttgcttcaagagcatcttccgcattcatacagaaactggcaacatctggacccatctgcttggtttcgtgctgtttctctttttgggaatcttgaccatgctcagaccaaatatgtacttcatggcccctctacaggagaaggtggtttttgggatgttctttttgggtgcagtgctctgcctcagcttctcctggctctttcacaccgtctattgtcattcagagaaagtctctcggactttttccaaactggactattcagggattgctcttctaattatggggagctttgtcccctggctctattattccttctactgctccccacagccacggctcatctacctctccatcgtctgtgtcctgggcatttctgccatcattgtggcgcagtgggaccggtttgccactcctaagcaccggcagacaagagcaggcgtgttcctgggacttggcttgagtggcgtcgtgcccaccatgcactttactatcgctgagggctttgtcaaggccaccacagtgggccagatgggctggttcttcctcatggctgtgatgtacatcactggagctggcctttatgctgctcgaattcctgagcgcttctttcctggaaaatttgacatatggttccagtctcatcagattttccatgtcctggtggtggcagcagcctttgtccacttctatggagtctccaaccttcaggaattccgttacggcctagaaggcggctgtactgatgacacccttctctgagccttcccacctgcggggtggaggaggaacttcccaagtgcttttaaaaataacttctttgctgaagtgagaggaagagtctgagttgtctgtttctagaagaaacctcttagagaattcagtaccaaccaagcttcagcccactttcacacccactgggcaataaactttccatttccattctcctagctggggatggggcatggtcaaacttagccatcccctcctcagcaaggcatctaccggcccctcacagagacagtactttgaaactcatgttgagattttaccctctcctccaaccattttgggaaaattatggactgggactcttcagaaattctgtcttttcttctggaagaaaatgtccctcccttacccccatccttaactttgtatcctggcttataacaggccatccatttttgtagcacacttttcaaaaacaattatataccctggtcccatctttctagggcctggatctgcttatagagcaggaagaataaagccaccaacttttacctagcccggctaatcatggaagtgtgtccaggcttcaagtaacttgagttttaattttttttttttcttggcagagtaatgtaaaatttaaatggggaaagatatttaatatttaatactaagctttaaaaagaaacctgctatcattgctatgtatcttgatgcaaagactatgatgttaataaaagaaagtacagaagacacttggcattcaaaaaaaaaaaaaaaaaa

SEQ ID NO: 6 - protein, 375 aa, Homo sapiens
MSSHKGSVVAQGNGAPASNREADTVELAELGPLLEEKGKRVIANPPKAEEEQTCPVPQEEEEEVRVLTLPLQAHHAMEKMEEFVYKVWEGRWRVIPYDVLPDWLKDNDYLLHGHRPPMPSFRACFKSIFRIHTETGNIWTHLLGFVLFLFLGILTMLRPNMYFMAPLQEKVVFGMFFLGAVLCLSFSWLFHTVYCHSEKVSRTFSKLDYSGIALLIMGSFVPWLYYSFYCSPQPRLIYLSIVCVLGISAIIVAQWDRFATPKHRQTRAGVFLGLGLSGVVPTMHFTIAEGFVKATTVGQMGWFFLMAVMYITGAGLYAARIPERFFPGKFDIWFQSHQIFHVLVVAAAFVHFYGVSNLQEFRYGLEGGCTDDTLL

SEQ ID NO: 7 - DNA, 126910 bp, Homo sapiens
ttttttttccctagacagagtcttgctctgtcacccaggctggagtgcagtggcgtgatcttggctcactgcaacctctgcctcccaggttcaagcaattcttctgcctcagcctcccgagtagctgggactacaggtgtgtaccaccacgcctggctgatttttgtatttttagtagagatggggtttcaccatattggccaggctggtctcaaactcctgacctaaaatatccacccggctcggcctcccaaagtgctgggattacaggcgtgagccacggcacccggccgccaaatctctattttctgtttctcattttatgctgtcttctgggtaaattcaaccagctgatttggctgtgtcgaatctgccgtttaacttattcactaagttttatttataatttctggaagtttgattgttttgcatttctacctgttcttatttcattcactcttgtttattcttgcatttcttcttttacacattttaaatgtgtaaaaatacacatacacattaaaaatactaactttatcgtatatcctctatcatattgttctaaaagtaaaccttaagctcacataaggcacaggctcatggttattaattcctcagggaaggtttttttttttccccttgtcccctaccttctagagcaaatgaagcttgaattcacaggtactgtagtggtcttggttccctttgtgtttcaggcacctagttatttaactttctttcttgagtgttcagctttgtatttattttagtttttggagacagaatctcattctgtcacccaggctggagtgtagtggcatatccatagctcactgcaacctcaagctactgggttcaagagatcctcccacctaagtctcccaaggagctaggcctacgggtacatgccaccatgcttgttttgtagagatgggggtcttgctatgttgtctaggttggtctcgaactcctggcttcaagtgatcctcctgccttgggctcccaaagtgtgctgggattacaggcaggagctactgcacccaacctggcttttttttccccctgagatggagtctcactctgtcgcccaggctggagtgcagtggtgcgatctcggctcactgcaaccccccgctcccgggttcaagcgtttctcttgcctcagcctcccaagtagctgggattacaggtgcccaccaccacacctggctaatttttgtatttttagtagagatggggtttcaccatgttggccaggctagtcttaaactcctgacctcgtgatctgcccgccttggcctcccaaagtgctgggattacaggtgtgcaccaccccgcccggccctggctttgtatttaaaagaagccttattgagtttactcagcagttgtaggtgtttgtagcaggataattttcagattatctggtctgcccatttgttaaacttgagatctattaattaactctatgttttcattaagccattttttattatggtaaaatacacataccataaaaattattaattaacatttaataggatgagtaaaattgcagacagagagcccatttcagtcgtgccactgcactccagcatgggcaacagagcgagactctctctcaaaaaaaaaaaagagcccattttagttgctgtgtgaggaaggacttcgtaataggctagtaggaagcaaaggatttccaaggaaggagagagaggcagaaagtgaagaatatattttgggaatggtaaatagttggtttggcaggagcttggtacgtggtacatgtaataataatgtgagtgaatttctgaaatattaagttagggccagattgtggagagccttggatttcagcttaaaggaattcggactttgagagcttttgaaagtttttgaacttaactcattttgaaagtgggtcttggcagcagtagatggagtggattggaggatgttgagattgacgacagggagaccaattaggaacatattggagtagtccagggaggcatgaaggcacagaatagggataaggtctgacaaaggctcagagctgttgaaatgaccaaagcataattgtagttattaaccaaattggggacaagaggagatgccaatacagacatatgacagcattctctagagtaagtcacttcccagcggaatagcattatgaactcttagagacttgctggtgtcttctaatgaagtcacatgctgtttataaaatgtcggctaattggcctatggagccatccagagccctcctttgatctgaacccagacttgaaaaagcatgcgtcagtgcaatttgaaaaacagtattaaaatagagaaagtagagtaaaacagatagatctgtagctctacagagtataaacaaccgtttttagcatcagctcaaagagttttaagtggtctcccgttatttgcttctacccttaataagatcttctaaaatcattgctctggactttcaatcttcaaatttcttagcaggacaatttaatcatcacctatttctattgggttatatatctttttttttttttttttttttttgagacagagtcttggtctgttgcccaggctggagtgcagtggcgccatctcggctcaccgcaacctccgcctcccgggttgaaacaattctcctgcctcagcctcctgagtaactgggattacaggcatgcaccacctcgcccagctactttttgtatttttagtagagaccgggtttcaccatggtggtcaggctggtctcaaactccttaccttgtgatctgcctgccttggcctcccaaagtgctgggattataggcatgagccaccgcacccagcctgggttttatatctagttagcatggtgacaacccagataacttggtttgcatttgttttcccattgctctagtatgttccaaggtcgttttcatatatatcctgtatgtgaattcccctttcccaaattgttgtggattgtatcataacttgtcaattagctaaatgtaaatgagcatcagctttctagagatttaatttttttttagcgtttaattgctgaatggaaagcaatagcactatgaagccaagaagttatttggagaagaacttgaattgcttgattctgaggatgggtaatcaaaatgtgattgtgaagaaatggagttattgtatatgttatatataatttattatatatgttatatataatttattacatatatatttaaaacttgaatgatatattgaaattacattgagcgtactaataaaaagcattcagatacacttggctccctttttcccctttccttccttctattaacaattattgagcactcagtgtgggccaggcactgtgttatatgctggaagttatagtggtttattcttcaattttgaaataatttggcagttgataaaaatctttaaagtttgttttttttaaaatagtcagttctggccgggcgcggtgactcactcctgtaatcccagcactttgggaggccaaggtgggtggatcacctgaggtcgagagttcgagattagcctggctaacatggtgaaaccctgtctctactaaaaatgcaaaattagctgggcatggtggtgcacgcctgtaatcccagctactcgggaggctgaggcaggagaattgcttgaacgggggaggcggaggttgcagtgagctggaattgtgccactgcactctagcctgggcgacagagtaagactccgtctcaaaaaaagtcagttcttggattttgatgacatggtactatgtagggcttacagggttattagtgccctgagtccacacttcacagagttattagatttaaattttcaaattttattttagagacagggtctttttctgttggccagactggagtgcagtggtgcaatcatagctgactgaagcctggaactcctgggctcaagcaatgctcccacttcagcctcctgagtaggtcggactacaggtatgcaccactacacctggctagttaaaaaaaatttttttttgtagagacaagggccttaccatttgcccaggctggtactgaactcctaggttcaggcaatcttcccacctcgtcttcccaaagtgttgggattacaggcatgagccaccttgcccggcctcatcattagacttaaagatctttttgtctttggtagttgttttacaattttaaacaactagtggctgtagtaggcattcttaaccaggaataaagtcataaatgtaaatgtatggtctcggaggagcaggggcagtcctctgtgaatctgtaaatatttggtgtatatagttatttttctgagggtatagatgctcaaaggagcctataacccaaaaaaggttcagagcaactgtttgccttgcttcattcagcttccagaatagtggcctttgttacgtagactagtgactatgtgcttgggcattacagtcacactccctgtatttgaatcctggctctgctatgtaatagctgtgtgaccttaaacaagtcatttaaccccctgtgcctcagtttcctcatctgtaaaatggcgataacaatagtatttatgtcatggcgttgtggtgaggattacatgagttaacgtatataaattactcaaaaaatgctcagcacatagtaaatattctatacatatatcagctattgttattataataattacatgggtatggcattgatggtaatgatggtgaaaaccatgatgacttctgccttcctgaagccacttaagagtgagtaacaacagccaaagttacttaggaataaaagattaaaaaaaaaaaaaggagcatgacaatggacggccacaataaatgttacccagctggtcattctaggaaaagctgataccgcccttcccttcctgttctagtttatggccagccaggctctgaaggttcgacagtctgactcaaccactgagaggaatatattccagttgaagcacagtggcaagagcaatttgttttttagatttagctgaaatatattctgattctattttttagccttggatggaactggctgtttgccacggtttgttttttccccatggaatgtgtgttgttgatgctctaccatgatagacagcaagtggggaaatgggaacttttattagctgacctaaagctcgtctctcctagttctgccacaaaacactctggcagtcttaagagacatgtcctgtatctccatctccacctactgacctccccagctgaaagggttaggcaaagtaaatcagcttagaatttattccaaaggtctttgactttttctagcttttaatgaccaaagttttcagataattgagtttcagggctatctgatcacctttgttgggtcaattttgcctctcctcaaatgcctgagaatgatgtttttttcatgtattatttttggggtctctactttttttctttatcccttccttgcacaaataactgccagttcctgaagccactgcccccaggaatttgcttcctgtaacaacaggtacttctctgtctgggctctccctctggaaaaagctcattcaaggatcccctgattccaaagtgccaagtagaaattgtataaattgaattaattcagatgctgtgtatcaggcttaaaatccaatagatatgtaggtaattgtctgaatattttccgcagtaaaagactgaagtctaaggtctaactcccattgcagtacataaattgcagtgcccagtgcaaagggaaaatgaagggtctcttattcaaaagttattaaaaatttcaagacagtaacaacagagcatcaaaccaagaataggggccaggcgcggtggctcatgcctgtaatcccagcactttgggaggccgaggtgggcagatcacctgaggtcaggagttcaagaccagcctggccaacatggtgaaaccccatctctactaagaatacaaaaaattagctgggtgtggtggtgcatacttgtaataccagctacttgggaggctgaggcaggagaatcgcttgaacctcagaggcagaggttgcagtgaggtgagatcgcaccattgcatgacagcctgggcaagaagagcgaaatgtggtctcaaaaaaaaaacacaaaaaacaaaacccaagcatagggcactttcgagtacagggccccgagcaactaactgcataggtcatccacctaggaagccaggcctgcatggagcccacgaatcagaatctttggaatctgactggaaaaaaaaaatctgacttttggggcctaccagaatctctgggataatatctgggaattgccttttttttttttttttttttttttttaaatacaggatcttgctctgttgctcaggctggagtgcagtggcttgatcatggctcactgcagcctcaatctcctgggctcaagcaaacctccaacctcagccttccaagtagctgggactacaggtgtgctcacactggttatttttaaaagggtctcactatgttgctcaggctggtctcgaactcctggactcaagtgatcctcctgcctcagcctcccaaagtgctgggattacaggtgtgagccaccgtgcccagctggaattaacattcttaataagctctctagatgttgacttttgagaatctcacacctattttcttcatttgcttgggaagaaggaagagcaatgttgggtttttgtttgtttgttgtttgtttgtttttgagatggagtctcgctctgttgcccaggctagagagcaatggcgcaatctcggctgactgcaacttctgcctcccaggttcaagcaattcccctgcctcagcctccggagtagctgggactacaggtgcaccaccatgcctggctaattttttgtattgtttagtagagacaaggtttcaccatgttggccaggatggtcttcatctcctgatcttgtgatctgtccaccttggcctcccaaagtgctgggattacatgcatgagccactgcgcctggctcatttttttttttaagagacagagtcaccctctgtcacccaggctggagtgcagtggcatgatcatagctcaatgtagtatcgatcttctgggctcaagtgatcctcccacctcagcctcctgagtagctgggactacaggtgtgtgccagccaccatgcctggctttttattttaatttctgtagagatggagtctcactatgctgcccaggctggtcttgaactcctgggctcaagtgatcctcctgccttggccttccaaagtgctggaatgacaggtgtgagccaccatgcctggccaattatcttttcttttcttttcttttttctgagacagagtctggttctgttgcccaggctggagtacagtggtgggaactcaactcactgcagccttagcctcctggtttcaagcaattcttgtgcctcagccacggagtagctgggattataggtgtgtgccaccatgcttggctaatttttgtatttttattagagacagggtttcgacatgttggccaggctggtctcaaactcctgaccttaagtgatccgcccgcctctgcctctcaaagtgagccactgtgcctggccccaattctcttttaaaagtgttcggcttgctttcctagatgggcaactgacagcacttatgttgcatctctagcagcatttctttgagctcttttactctttttctgttttcctctttctcccctatcctcttttgttttacgcttgtcttgggcctgcccgcaatacctcaggtgtggctagacttggctgacgacatccaaggcatatgagattattagagaaagtttgaggggaaggagtaagacttttgatagtctggatttttcattaagaattttatttttgctatagcctgcttttcctacccagttttgcttttgcttctaatctaaaccaaaagaaaaggaaaaaaaaattttgagaaacaaagttatgagaacaatctcaacccctacctcagtgattatttagtcacaatcattggaattcaattccagatcagttcttcttcctccaccgtaaatgcctgccagtccttgcaacttatttctgcaaattgtcacttcaccagattgtgacctcaagggctcagggattcatcctgtataacttgatgaaagtatttatatcaaactatcctgactaaacctggtcctgagttctgagtttagctatacatcattgagatattgcaaggttaataaacctctttagattatggatttaaaattttagcacaacaaataaaatgctctttttttggcttacagttctgtgtgtgtgggtgagcgcataagcagtcactgagtagaacagatgctgtcttgaattttttcttaataaaaagatgacagatttttaattactgggtgcttctaaattacttaagaaatgacatttttctttattgttttcacctgtatttcctgttgttccgtagttgtagcttctaatacgcatctttattactctctatttttaaggtaatgaaaactttgtgtttctattttcattctcacagcaaaggagagattaaatggaaacaaccctttataactttgaaatattatgtaatcctggcattcttaaaagaattttttgcttgccttttactttggcaaaatacttgtatctaaagatatataaagaacactgatggccaggcatggtgactcacgtctgtaatccaagcactttgggaggccgaggcaggcagatcgcttgaggtcagaagttcgagaccagcatggtcaacatggtgaaaccctgtctctactaaaaatacaaaaattagcctggtgtggtggcacacacctgcgattccagctatttgggaggctgaggtataagaattgcttgaacccaggaagcggaggttgcggtgaggagagattgcaccactgcactccagcttgggtgaaaaagtgagactctgtctcaaaaaaaaaaaaaaaaaaaagaacacttatggctcaataataagagggaaaacagcccattttaaaaaataggtaaaaagtttgaacagacacatcacaaaagaaaatataagaaaggccaataaacacatgaaaagtctcaacatcattaacaatcagaaaaaggcaaattaaaaccacaatgagataacactgacatacccactagaatagctaaaataaaaaatttgacaatactaagggttagcaaggatgtggaacaactggaaatctcatatattgctggtgagggtgtaaaatggggaaacaatttggcagtttctcataaagttaaacataaatttatatgattttgctaattcacccccttatcaaaagtaaatgaaaacacatgtcctctaaagatttgtacataaatgttcacaatacctttatttataacaatcagggactagacgttgggcacagtggctcacatttgtaatcccagcactttcggaggccaacatgggaggatcgcttgaggccaagagtttgaggacagcctgggcaatttagcaagaccctgtctttacaaaaaataataaaattagcgaggcatggtggcacatgcctgtagtccctgctacttgggaggctgatataggaggattgtttgtgctcaggagatcaaggctgcagtgaggtacgactgcatcactgcgctccatcctgggtgacagagtgagaccctgtctcacaaaacaacaacaacaaaaacaagaaattacccaatcattcatcaataggtgaatggataaacaaatggaatactacctagcaacggaaagtgacgaactatgatgaatgcaatagcatgaatgaaactcaaaaaccccatgctgagagaaagtcgacagagcaaaagagtacatatcgcacgattccattaatgggaaattctagaagaggcaaaagcattctatagtggcagaaagaatatcagtggttgcttggagccaggcttgggagaggaaatactgactccaaataggcacagggggattgcggggggtgttggagatgttgtatattttgactgaggttatgcttccacgggagtatacatttttcaaacctcatttgaactgtacacttaaaaagagggtgcattttatataaaatatacttcaataaagttgtttttctcaactactaaaaaagttcagagaagcctctaatatatgtccatgtacataccacctaggattcataaatgttaaatgttttgttacttgcttcatctttttcatatagaagaaatgaaatattacagataaagatgatctctccatagctcccaggcttattcttcaccccttcccaccggcaagtacattcctaaagttgtatttatcctttctgtccatctaaaacatgtattcaccgacatcttcattttattgatgaaggtatcaaagcttaacaagagctaatgcataagccatagataaacaatgcctttgtcagcagcacagccagtaatgatgttctatcttaagttcaatctgcaggactatgttatctaatctgcgtttgaggagctgcgaatgtaatttatgtaattattggagtgtttatactggtctttctcttcagtttttttttttgttttgttttgttttttttttttttttttggagacggagtctcactctgtcgcccaggctggagtgcagtggcgccatctctgctcactgcaagctccgcctcccgggttcacaccattctcctgcctcagcctcccaagtagctgggactacaggcgcctgccaccacgcctggctaatttttttgtatttttagtagagatggggtttcactgtgttagccaggatggtctcgatctcctgacctcatgatccgcccgcctcggcctcccaaagtgctgggattacaggtgtgagccatggcgcccggccctgattaatgtagttttaaacaacataacaatttgatatcagtgattcgtttaattgcaattaagatggaatacagatatttatgtatttccctctaattttctcaagtagattactagactatttgcaaagcatctaacaccactgaatcctaaatagctggctggatttcactgttctatggacactatatgagacaaccagagggactgatgtgacgacattccttgccctttcttttgatttatcttttttatggtgtatcttctatgttgtccagatttccccctcagttctaatgtttatattttgaaagctgataaatattggagctgaaaagaagagtttaggctagaattaagtattttgtaataacgaagctgttaatttccctttgtaattttttttttttaaactcacagacacaggtctctgaaagaacactctaattctggttttcactggcgaagtaatttgtcttttttccttcttaaaaatagatacgattctgataagaaaaatgatagcttgtcttttctctataagagaagcaatataaaccagtttcaaactcaagtttaaatttgcactgggatctagggtattttttctttcagaatttgaaagttggctcggtacaatggctcacacctataatcccagcactttgggaggtaggaggattgcttgagcccaggagtttgagaccagcctgggcaacatagtgagactctctttttatgaaaaaaaaaaaaaatttaaagctgaacgttcatttgcttttagctttagatacatattcttcttcttctttttttttttttttttagagatggggtctcactctgttgcccaggctggagtgcagtggtgccatcatagctcactgcagcctggaactcctgagcccagtgatcctccagccttggcctctggagtagctgggactacaggtgcgcactaccatgccttagatacatatgcgaatctcattggctattgccttggatatcacaatggagtttaactctgtccatcatgtcagcaatatactatagtgtcactggagccacaactggattatggatgtgactcacctagtgatgcgccatccttttcaataacatctgataattgcagagatgactacatcagactagttgagccacagggtactgttgggtcactggtgactggcatgtgggataggggtggaagggaatctttttttggtgctgcagaagataaagggctctaccctcctgtggagtagtgtgataccccaagaatggagagaacagaaattctaagatacgcagctgatgctggaggttagagaattttactgagtttttcttgttttttggacagctaatcagaagacaatatataaagtttagtaactgaagtatggtcagggagagggcctgaaaattctgctttggaatatttaatggtgacatttagataagggttaagggtgaagccagccttatggatgtggggatcaggggaatgttcctcaggtgctcaggtcagggacagtggcaacatcctcaatagttaaggaggtgtgggtattttgaggaaatgatgtggtttgtttcatcactatataagcatcaggaactggcagtagtgatatgtgaatgggatcaaggtaaccgagaacaagatgtgaaactttagggaaatggtaagtctgtctttcatctggttcagcacacggtgaactgttccagaggtagcttcttaaagcccttcgttttgtcaaaccagaatcttttttttttttttttttgagacagagtcttgctctgtcacacaggctggagtgcagtggcacggtctcagctcactgcaacctctgcctctcaggttcaagtgattctcctgcctcagcctccagagtacctgggattacaggcacgcgcgaccatgcttggctaattttttgtatttttgtagagacggggtttcgccatgttggccaggctggttttgaactcctgacctcaagtgatctgcccatctcggcctcccaaagtgctaggattacaggtgtgagccaccacgcttggcctggaaaccagtctttaagagaaagggatgcaggaaagattggagatctttgaagaatctctttttaagaaaattatctttttcgctctagttctgtcacctttagtagactctttattaagcctaagaatcttatacttcttatctctgttctatgtgagttggtataacgggtcaaattcacaatccagaaatgcctgtattgtttctgatgtgctgaggaggtgtacaattgtcttccttgatttcaacctcaaaagctttacatctttacctgggaacatagaagtcattattattatgtctttcacaaattatgtaaacttaaaaaaattatattttcaatgtaaaccactccttggaggaaattagttcctttaacatttatttcctaattttaatttctactagttttatctctttcaagcttaaatgagtgggccttagttctgtattattgccatcttaatcatgttctttgtgattttagtagctttactttttaaaacttacatttccataatgaagacttctgtatttttgtttttttcgtcatatatccgactccagctccctcaagacctgtcagcttttccctgcaccctttgttatctcttgctgtggtccaaaggtaaggacagtaagccgtgggcacagactttccctaaatcgtatggatttcctttagtcaataacaagaattttgatagttaaggcatttacgccttaagaatgtgataaattccccctcctactgaatgtaaagaactatttttctacgcagactaactcttatattcacttttcttatagaataagtacgtaaccaaatctgttcttggcttctaagaagttagaactcaacgtaaagatgacttgtggaaaattacttactcattctgggtaaatggacgcaggtatttagtgcttaagtaggttctgatataccattacattaacatcattttaaaaatcgctctgaattatatgtattttaagagtgactgggttttaaaaaaaatagaagtggtagagcataaatcataatctttaataaggtaataaaaggcaaacactattgctctgggcaaaactgggctaatgcttttggatttctttccgcagtcttcacagacgacgctcagcatcctatcagcccttggcagagacacacgatccagcctaaatcctcaaattctggctcttgcccaaagtgtgtagccagtatccatagagactgcgaaaagcatccgcgagccggggcggccccctcctcctcagctccaaattgcttcctttttcggtgggagaaaaaactccgaggagttgtgtgagcgcctaaggagcgcacgctcctccggccctcaacacccagtatagttttcgctaagttcctggccgaaccacagctcccacgatgcacccaggttttagagcggtcagaggggcggcttccggtttaggacagcgcggctgcgcacggcgtgtggtcttattggctcgaaccgcctcgtgcgatgcgcgtcacggcgacgtgcggtcgtgggggcggggagacgcggcggcggcggtcgctgtcgcggcggcgagagcgcgattccagaagcggcatcgcggcggcggcagcggcggcggctacaccgggcttggccccctccctcctccgttcccccctcctcccccctcccctcagcggtggctcccaagaagtccgagacacgcggtgaggcggcgacgggctccgggtgagggtctctggagtgtgggcgccgtttcctgggcgcagagggagagcttggggtcggggggaggtgagagctgaggggcgaatgtccctggcgcgggcgggagagcgcgctctcggcccggcccgcaacttgattcctaactctgacctgccgcccggcgcgccttggcctgagggtcggccgtgtggggacctggagccgccctccttgcgaccgctgtcggaggagtccggcgcccgctgttcccctcgcctcgcctttcgcggcggagggtcggagcctgctgccctctgctcaccttcgggtgcagttgctccagctccctgcccgcggagaggatctgcccgcccctcttcgtctcttgtctgtttttatcacttctaggaatttgtttggtggaggcttgggatgggctctctcagcgctactttcacagtgactggggtggggaagagttccctcacctttttcaggtggcgcgtcaggcagcctgcttggtacctggagtcctgttttcccaagcccccgtccgtcagtgttcgcttcttctaaaattaccgatgacagggttataaatcagtttttagggggctcggagagagtttgggttggaaactgttagctcttttttcctcctggcttttgatcttgatttaagcgtttgcctctgaaattttgacaacttcataagatggtccaaaagtgaattttggtagaaagatttaaatgcacagttgggattttatgggggtacggtgacttcattgcaggttgttgactttttttttaaaataagatgttcttttaattttttgactctgggtttctgagccttggttattgtcccccatttctgttccaaaagcagactttttttttttttatcaccccttcccctcacccagagaatccctttaaacgccaagaaagctgtcataacatttagcattatttgatcacagaacttttccaaatgaaatgctaaaaattccttcagaaaaaagcatccatctacccatagcattattttttggggggaggctatacgtcttgtcagttctgaaagtttgaagtgacctcttatcaaaggccacccttatgtctccttaaacgactcttaggtaattcctagatgttttcatccgttctgataaatttgttgtcagagtgttgattgtgatgatctgcaactaaaattatttcatcagtaaagatttccacacatgtggcagctgcttgttggtgaattatgaaatttgaaaaaattgcaaattaaatgtgaaaggcatatttcttttaacttctgattgtaactgtctgtcctccagtatacgttcctatctagactttggtctgcttagcagatttcctcttttcacttgacactaggaatgggagtaggatttccagagcctaaaagagagtttgttttttttttctatccctaggctccttgtatgaatttataaataaatggttttttctatcaatagtatcaatttagctggtgcactttttttttttagtataaatcacattttgtttcctaaaatgaaaataattttgttttattctttatttcagggattgacagattttaaagcctgcaggcccagtcctgccagttgcctctttttgtaaataaagatttgattaggcctggtgcgatcgctcacgctggtaatcccagcactttgggaggtcgaggcgggcagataacctgagatcaggagtttgagaccagcttggccaacatggtgaaaccccgtccctattaaaactacaaaaattagccgggtgtggtggcgcactcttgttatcccagctgcccgggaggctgaggcaggagaatcgtttaaacccgggaggtagaggttgcagtgagccaagattgcaccattgcactccagtctggacaacaagagcgaaactccgtcttaaaaaaaagaggaagaaaaaagtttttattagaacatagccatatgcagtcgtttaggtattgtctgtagtttcttttggggtacagcagcagagttgagtagttgtgccagagagccatgtggcctgtaacaccttatatttatttactatgtggaccttaatataaaaagtttgccgagctccattaaaaatgatacatagtcaatgttgaaaatttaaatgagtcagaaaattgtaaaggagaaagtaaaagttatttacaagtacaccacttggggctttttttctgtgattatagaatttgttggtgattattttttatataaatgaactcaaattgataaatgttggtttgtaatctgcttttttttttttttgttcattttgttttttgagacggagtttcgttcttgttgcccaggctggagtgcaatggcacgatcttggctcactgcaacctctgcctcctgggttcaagcaattctcctgcctcagcctccctagtagctgggattacaggtgtgcgccaccaagcccggccaattttttttttgtatttttagtagagacggggtttcaccatgttggccaggctggtctcgaactcctcatgtgatccacctgcctcacccctcaaagtgctgggattacaggtgtgagcaaccgtgcccagccctgtaatctgcttttttaagaagtatttcattatttgtatttggattttgattgcttttgtattttctgcattggaggtctttctagaaaggggcttatgaaccgggttttcatgattagtcttaccagtcaagatttaaaaagaaaatccaggaataataaatgcttggtaatgaagatgctggttaacatttattgagcacttactatgtactagacaatttccactactttacgtgtatgaaattatttaattgctgtggcagctctgtgagtgtatactatgatttctgttttacaggtgaggaaactaaggcaaggagaaaaattaagtaactgttggaagtttatcagctagcaaatggtggagctaggatttgatttcatgcagtctgatttgagaaacccaaattcttaacaatatgctacagagcctctacttacattattgctcagtgtagaaagtttagacagtccagagatatactgtcatcccttggtgtctgtagggactgcttctaggacctttggtgaatatcaagatgcacagatgctcaaatctctgatataaatggcatagtatttgcatataacctgtgcatatcctcccaaatactttatatcatttctagattacttataatacctaatagaatataaatgctatgtaaatagttgttatactgtattgtttggggaataaagacaagaaaaaaaagtctgcatgtttcaaatacagatgctatattaaactttttggatccaagcttggttgaatccacagacataagagcccatggatatggagggccaactgtataatgtaaaaaaattccttctcatgcgctagtttccagttttcatcctttgtagataaccaggggcaacagtttgatataggtcttttcacctctctttgtgtgcatatacaagtaattatttgcagatttgtctgcatattcagaatttaaaaattacacttttttttagcagctttagatgtacagaaatttgagccgatagcacagaaagttcccatattcccttcccttccctcgtttctcctattgcaaacatcttgcattaatttggcacatttattgcatttgatgagcccatatcaatatgttgttatttattggtctatagtttacatagggcttattctttgtgttgcagttttttttttttttttgttttgtttttttaagatctcactctgtatctcaggctggagtgcactggtgccgtcatggctctgcagcctcgacctcccgggctcaagcgatcctcccaccttagcctcccaaagtgttagggttacagacatgagccactgcacttggccgagagttattttttacaaaaatgggatcaagcccagtggtggcttgcgcctgtagttccagctacttgggaggttgaggtgggaggattgcttgaggctaggagtttgagaccagtctgggcaacacagcatgactctgaccagtctgggcaacacagcatgactctgtctcttaaaaaaaaaaaaaagtgggctgagggtggtggctaacgtcggtaatccttgcactttgggaggccaaggcaggtggatcacctgaggtcaggagttcgggaccagcctggccaacatgaatgaaaccccctctctaccaaaaatatattaaaaaaaaaattatctgggtatggtggcgcgcgcctgtaatcccaactactcaggaggctgaggcaggagaatcgcttgaaactgggaggtggaggttgcagtgaaccgagattgcaccactgcgctctagcctgggcaacaggagcaaaactccatctcaaaaaaaaaaaaaaaaaatgacgaaacccgtcattcagcagtcattcaccttttttggtggttttcaggatatagtgcttttggactttctaatgtaaacattcttgatttactaattttcagattaatgacaatttattttgggaatggaaggctggcttggatgttactttttctctctttttatttttctatttttttttttaagtgtatctcatagtggcagcagcttttccccaaaggaatgcttattttttttgacgggacattctggctatacctaatcatcaaagttcttcttttttctctttatctctggatttggaaaatgattcattttggttttcttcctcataaattctttttggtctgtccagccattgcagttgctagtcatgtctccttgtcagactttggctttttatgactgtcatcaatgttagaaaaaattacttgctccttatcactatttcaatacatttcctcttgggctttccctgggggcagcactgaattttttccttagttgagccatgcagattttctagaagtcatcgtttagtttggcttttcactcctccatgggtgctgttcagcttctaggagatttcttgcagtttctacttgtcaacagattcaccagcagcaccctcctcctcactgagactggtgcttccccacccatctttatcattttcagccttctctttttctttctcttttcttttttccttcttttttttttttttggttacagcgtcttactctgttgcccagactggagtgcagtggtgcaatcgtagcttactgcaacctccgcttcatgggctcaagtgatcctcccacttcagcttcccaagtagccgggattacaggcatgcaccaccatgcctggctaatgtttgtattttttgtagaaacagggttttgccatgttgtccaggctggtcttgaactcctggcctcgagcgatcctcctccctcagcctcttgaatagctggatttaacaggcaggagccattgtccctggccatattcttgcactgttgattttactggggcctctgtcagcttaccctggaattctgcatcatctgaggatttggaagaactgaatcacagttatagtagacagcatcatattcttatccttgtgtgttttatactgggacaggtcttgaggaagttcttctgtcatggctaggggacattgagctattacctcttatagcactgatttggactgtcatgtcttccctaaagttcttgtccataacataatggatggtcacagtcatgagcaataactgagtgatctctgggactaacaggtgatgaaatgcatgtttagcaaacagtgatttctgttacttctctgtggtgcagcagcagaaacttttgcccaaaggcatagaagttagagggcttatgaatttctgccaatctggagataagatttatgagcatcatgttaactcaaacctctgcttagagatttctagctgtgtgcttcaggagtgttttggtaaaactttgaggatcatgaataaagctaatagcttaaaagttaaacactgtgagttttttcttcttttgtttcctgggcacttttcataaccttttcagtgttttgtgtttggagccactcttcccagtgacatactgcacttgtaggtctcttgtcactttatcctcagattctgagtctgtcctatttgtttctcttcttcatcttatcctaggaagaatgtcaaggtggcaaccaatatctcaaaacatgcagttgtgatgacattgacagttttggcatcagtccggatgtttcttgtagagttcagtcatcacatccaaagatatcttggctgcagttacattgctgtctcttaacacgatgtatatgaaattctgcagtactatatccactttattcttgtgttttacagttatattcttgctatcagtcacaacatgagtatgtaaagtctcaaaagcttattatggcaacttggaagttcaaaaaagagctctagcaaacttgatggatggatatgatacttctcagcaagatcaaagctttgtaaaatgttattagatgattttgattcaagacagtacagtataattgtaggaaagtagatcttttagctcttgaggaaagttacttagatgttctgggtaacagtgaccaatctgtgccataaatatcaccagctctgtcaattctttgccaggtttattggttttaatttgagaatctccatattggatttgtagtgtatatagtactgctgtagtaaccctttaatgtcagccagtggctccactttattagattctataactgctgcttgttactgggcagctagtttctgcaggacatgttggctgtagactggatccacagtgagtagttctcagagaacccgtactgctgacatttgcaaacccatttcagctgtggtgtggagctgtggcgttgtacttctggatgtgaccagaaacagtgtttgggagctcagcaggtggtgctgtcttggctgattgtgtgtgtgtgtgtgtgtgtgtgtgtgtgtgtattttatttaatttctgagacagggtcttgctctgtcacccaggctggagtgccatggcacaatcacggctcactgccctctcgaactcaaacagtcctcccaactgagactcccacgtagctgggactatagatgcacgccaggctaatttttgcatttttaatggagacgaggttttgccatgttgcccaggccggccttgaactcctggactcaagtgatcctctttgccttagcttctcaaagtgctgggattataggcttgagccaccatgacaggccctgtgtgtgtgtttttaaaattaatatttgtggcttactttacatctgcatctactctcttctttttagtagttctctagtgtagtattattaaatggatatgctataacttaatactgctcttactgatggatatttagattatataaatggattgtaaattgcccatacattttatgtatatttattgcaagctaattcccctttagtaattttgtaatagttgaagaaaacacatttttcattttgatgtgtgttatcacattgccacccagaaaggtcacagcaatgtaaactctcaccagaaatgtgggtggagtctagcttctctgtgtcattacaaattaaaaaaaaaaatctttgtttatctgataggaggttaagcatttgcagtatattctatctaactgggagatatgctttgtttcaatagcattcttttaaagttgatttacgattacttcaggtggagcatgcattctgttcccgggcctcccttaggtggagctgcttcacctatttacattctctgcttggctcttgaaagcatatggttttgttatcactgatcaaaaagaatctgggtgcctgcctaatagactatatatttcttgagggtaaggaacgtgtaactttcttgataatctactggcagttccggtatatagcaggtggtaagtaaatggtcttttaaaaatctaaattggttcatgaaccagtaactgttggggttactgctgagtgaagtgctttaattttgttaaaaataatattttcaaattgtgaaatacaaacttgaaaaactgcagtgcatgctgggtgcggtcgctcaggcctgtaatcccagcactttgggacgccaaggtgggcggatcacctgaggttgggagtttgagaccagcctggccaacatggcgaaacccccgtctctactgaaaatactaaaacaaaacaaaacaaaaaaaaccaaaaacaaaaattagccaggcatcatggccactgcctgtaatccctgctacttgggagaccgaggcaggataatcacttgaacccgggaggcagaggttgcggtgagccgagatagcaccattgcactccagcctgggcaacaagagcgaaactccgtcttaaaaagaaaagaaaaacttcattataaaattgtcactttggcatatgttgtctctcatctttcaactctaagataagttaaataggacgaactgatatcttaggacctgttaggaaataattagcctattttataagtacatttggtatttagaaattggaagttgaggaatttattaaaaaggggatttgtttaacattacttatggttttgagttttctggattggagtataccatgtgttagaaggggatatatgttggccaatgatattatagattggcttaattagacaagtgtccagacagaaggaaaagggaagctaaattctgagaattgccagattatcttcaggcatgggaaaaataactggaaaaggacacttaagttttttaatggaagactacaacaaaatgatattttctttataactgatcatattacacattcttactggatttattgaatataagaatattcttatttccacaatgtctgtgtactagagaacaaaacacaaagcatatggtcttaaaaggggcaaaattagttagcttattacactttggccatttaggcctttcttttgtatgtttgttgttagtatatcagctgattatttttcattgacagcatattcactttaatcttgtatgttttgaaattaagtttagtgaacatgtatgcctagttgtatccatgtatccattcctttggttaaggaggatgtctttgtggtaggggttacattatgaaaacttcagcagaatggcacagaagattgactttattttcttgaaatttattttatcctatttcttccagaaaggatatctggtaacttaaaataaaagatctggaagtgtaataaaatatgtgtaaaaagaagaaatcagggaaaggacaaagcatatattcttttcccaagtgacagcataattcaatgactaagaataaaaattgggtttgagtttgcttggcagcgagggtaaaaatggaagtatggtaaaatacatagcactaaactaaaaggaggaacaagaccaatttttcaggggtgaaatcctctcttcacagatttctaagaaactatttaaactggacattatataaaagctgttttgttgttgttagtggccctagatttaaaaaaaaaaaaaaggtgagggctaagcattaagatttcaagccaatgaaatacattttaggggttgatttgaatttaggctgcagatgtatgttttatttggccaatatatgttttttttccttaaaatatttcaaacatacatgagactacagagaccaatataattaacattcatatattaaccatacagctgtagcatatcttagcatgttcccatatttctcatttttttgaggaaaaaaaatcatgtacagttgaagcaacttcttggattacagtttactttcttcccagaagttaactattattctgagtatggtgttgatctttttttttttttttttttggaaacagggtctcactctgtcacccaggctagagtacaggggtatgatcatggctcactgcagcccatctcctttgcttgagcagtcctcccacttcagccttccaagtagctgggaactcaggtttacaccactgtgcccagctaatatttgtatattttgtagagattgggtttcgcctgttgcccaggctggtctcctgtgctcaagcagtccatccacctcggcctcccaaaagtgctgggattacaggtgtgagccaccactcctggcctgatcatttttaatgcatgtttttatgtaattactacatgggtatatattcacaaatcatagtattgctttgcatattttcagatttgatgtaaatgatatattctgcttattctataatttgtctttcatgaaaatttttttgatttatatgaaactgctgatacgttaccattttgacctacaaaaatgacagtttcatatggcccaccctaatagtcatacttttatctgctgtaaaattattttctattatatgaatatagccagataaaatttatttattctcatgttgataggcatttagattgtttccaggttttcatgtattaaaaataatgttacattgaacattccttattaggaatcattgtgcttacatttaagatttttggcaagttttaaaaactgcatgttgtggctgggcactgtggcttgagcctgtaatatcagcactttgggaggctgaggtgggcggatcacctgaggtcaggagtttgagaccagcctgaccaacatggtgaaactccatctctcctaaaaatacaaacattagctgggcggggtggcgcgtgcctgtaatcccagctactcaggaggctgaggcaggagaatcgcttgaacctgggaggcagaggtttcagtgagccgagatcacgccattgcactccagcttgggtgatgagcgaaactccatcttaaaaaacaaatgaacaaacagaaaaaaccccacatgtcgcgactccctaatggattgtgaaatcaattcaggaattagaaaaatatcaggttgtattgcaaacaataaggataaaatgttttgtgaaatttttatttctgtgtgtatgtatgtggttattaagtttgaaatgaagccctctatggtgaattacccagaagtgcaattattgggctgaagattttggaggttatgggaatttttttagctgtattggatattgtcaaatttgctgtcactttgcatattttagcactagagtatgagaatttgccatcttccgattgttatgattaatggcattgtcagattttttacatgtttacaaatcagatgagtgtgaaatgttaccacattgtcttaatttttatttttgattatggggttgagtatgttttcaaatgttagatgactattcaagtgtttgtttcttgaattggctgttcttttccttccacagccatttgggtggtctttctaaattgattttagaaagttttaagttcattacatacttcgattatgtttgttaagtgaattgcaactgtcttttctcagtctgtgtggcttatctcttaactttatatggtttgttttgttaaatagaaatgattattataaaatatatcactattttcttgtttgtgttttatttaagaaacctggggtgatcactctgtggttctcctcatgcacattaacaagtttgttacttcatttcttcagttgatctcccttttgtgaggtggtttttaagtgaatcattaaagagcaaaggagaagttttcccgtggctcccacgctgctattcaaaacttcctgggttagtaagccttcttgtgaattgtctttaaaatgtttattaaaaactcttaggaactctcatataaagtcttaaatttgtgtacaaagatgctctctgacattgcttgactagtgaagaaagttaaaacaatctaaatgttaaaaatatattatatgtatccagacttcagaatttttttgtagacattaagagaataagctagagctatacgttgtgacatgaaaatgcctactatatactagttaagttttttaaaaagtattttgcagagaaaatatgcacagtataatcttacagtgcaaaatttttgcaggcacatatgtatatgatatatgcaatctatatatgcatatagaagggtctgaaaatatataatagttgccactggagtggtatgtgtgtttgtgttgctttaatttttttcagtacacatttaaacagaagtaaaaaacaatttaaaagaaatttggctgggtgtggtggctcatggctgtaatctcagtactttgggaagctgagataggaagtttgcttgagcccaagggttcgagaccaacctgggcaacagagtgagacctgtctctacaaaaaacttaaaaaattagccaagtgtggtagtgtgtgccgggactgaggtgggaggatagctcgaacctgggaggtcaaggctgcagtgagctgtgattgagccactgcactccagtctgggtgacagagtgaaactctgtctcacgaataaataaataaataaataaataaatgggagaatcttaataaccttggagtatggaatagatttgtttgtttttttgtttgcgacagggctcaagtaatcccaccaccccagcctctgtagtagctaagactagaggcatgtgccaccacacatgattaatttaatttagttttgtgaggacaggatctcactatgttgcccaggctggtctcaaactcctggcctgactgatcctcctgcctcagcctcccaaagtgctgggattacagatgtgaaccgctatgcccccaccagtttttttttttttttttttgggtcttggttctacctggaattttttttttttttttttttttggtgtgtgatgtgaggcagggatcaaatttaattttattatatgtatacccaataaccatattactgttaacagagtgttttgaatttttgagttaataatttttaaaagcagctcatggtttcttgttaagcacaattgcagtctttttgtccttcaccaggctgcttcatatattcaagtttgtgatctctgtgaccagagtgatatgattggtggatgacttgatccagaaatagaacctagaagagtagatacagagtatattttgtaggtctcactcacatgtcatttctcttactatttaaatggaaattagattgcactttctttcttctctaataaacttgctttcacttaaaaaagaaattggattgcagacaatttgaggatatacattaatgaggaaacagattgctggttttttgcattgctggctacagaccttggaaactagagaagcacatggttgattgattgatatcttgttctaaaacttgagcatacttaaatgtatttggtcatccagctattatcacatatactaacgtttgagacttttaattttgactttagggtacatgtatcaatacagttgtcttaactttctaagatgtttgatataaaacactgtaaatcacttagtttaagtatgaagaccatttttgtttcctcttaagagggtttttattacacttttagtctaagaagtgttgctgctgaggccttcagatgtatattctaaatttttcttttttctttttattccttttctttctcatactggtggtcagaagaaggccttatgatttagtagaattgagcaatattgataaagattgtagatacaagtttatttttagatttcttagacttgtaaaattgagaaagataattaaccacttccttgaattttccatttgcagcattataaagcaaaacaaacaaacagacaaaacagacttgtgctctggagggtaatatgtgagatgagatagagccccgaatgattgttatttaataatgggagaaaggtgctttatcacttcaaagtagggttttgattttgcctgaaaggttaactgtacaggccacatgttttatctgaacagattttatttttgaagtagtttaaacagtgggctatgtggtattatagtacagatgatttatggaaggaagtctttacatatttgatatattttggcacaaaatatataatcctagatttttcaggatttccctgactttaatatttagaactgttgttccttgtattagcatttttatgtacagattgagcatctctaatccaaaaatctgaaatctaaaatgctcccaaacctgaaactattcgacgccacaagtggaaaattccatacctgacttccagtgacaagttgcagtcagaactttgtttcatgcacacaattacaaaacattgtataaaattaccttcaggctatgaaacaaatatattttgtgtttagacttgggttatatccccaagatattttattatgtatatacaaatattccagaatctgaaaaaattccaaatccaaaacacttctgggtactaatctttgtttcaaaaacaatagtcattattatagatcttgggaaatgaggataaccgttgtaatatggctgtaaaagagttgtagaaagacttttctcctttaaagacattgtttttgtagctttgaccactaccagtggctataatgcattgtttgtggctttattgaagtcctataagatggtgataaaagcatggactttgaagtcagatagataaagatttgaattcagggtcttccacttaagtttgtgttttgatgaattgcttgatctccgtgaacctcagtttcttcatctataaaatgggaccccacttcataaggattgttggagatgataggtgaagggtcaactactgggccttgttcaaagttgtcttcagtagtaaagtaacaattcaccttctcctacagagagataacatcatgatgtgggaagaagggaactacttacttttgtgggaggcagtgttgtataataggccatttgttgcttctctgttttgtacgttgtgtaaccttagggagattatccagaaagtttaccctcagtcttctcattggtaagtagagataatgttacctatgtaacagcagagttttaaagctaaatgagaatatttataaaagctcactttatctggcacatggtaacatggtaacttctattaataaaattttatggtaataagcaatattatgcttctgaagtaggtcacaagaagggcttttggttggagaaaccagaattgttatgaattagtccgtgtttactttgagggagtaaagactagggagtattcattgaaggaagactgtttaggaggctactattacttggctaaaatacaaataatgtgactcaatagtttctcaaaattttgtgtaatctaggcttatgtaaataaactttactgttttattaggatttaaaatcaactttattgaaatataatttaaacaatgaaatatactcattttaggggtgcagttagatgacttttgataatgtacatacccacccagataaccatcaccctaatcaaggtaatggaatttctgttatctccaaaaccttcctcgtgtccctcacagtcagtgcaccacacccctcccctcctctggttcaaggaaaccactgatttgcatacattcattttgtctcttagaatttcatgtaagtgggctgggcaaggtggctcatgcccatatttccatcactttgagaggctgaggtgggaggatctcttgagccaggagttcaaggctgcagtgggccaggatcacaccactgcagtccagcttagtgacagtgtgagacccggtctcaagaaaaaaactttgtgtgtgtaaatggaatcatactgtacatattctttaatgcctggctgctttcactccgcatgtttttgagagtcctccatgttgctttgtgttccagtagtttgttgccttttattgctgagtagtattcactgtggtatggctataccacaatttgtttcttcattcacctgatatttgggtttccagtttttggctattatgattaaagtggcaatgaacattgatgtacaagtctttttgtggacttaagtttttatttctcttgggcaaataggagtagaatagctgagtcatatggtgtatattttgcagtttcttaataagttaaacagtttttcaaactgattgtgccattttatcttcctgtaatctcatttacattatatgctgtgtttttaattttaacttttttttttttagtgtgaattgatatcttattgtggtttttatttacatttttctttttaaaagttttgttttttgttgtgagacagggtcttgtctgttgtccaggttgatgtgatcctggctcactgcagtcttgacttcccaggctcaaggaatctttccaccccagccttccaagtagctgggactacaggtgcatgccactacatccagctaattttattttgtaaaaatttttatgtagagatgagatcttgctgtgtgcccaggctggtctttaaatctggggctcaagtgatcttcccgcctcagtctcccaaagtgctgggattataggtgtcagccaccatgccttgccttatttgcatttttctgatagcaaatgatgttgaacacttcttcatgtgttcattggccatttgtatatccttttattagatgaggtgtctaattcttaattcatttttaaaaaagcttttaaaaattactgagttacaagttctttatatgttgtggatacaagtactttgtcagctgtgtgaattttgaatatacagtcagcgctccttgtctgagggttccacatccacagatttaacgaatctaagatggaaaatgtttggtaaaaaaataacaatatgcgaataaaaaataatacaaataaaaaatacagtataacagctatttatatagcatttaatcatattaggtattataaataatctagagattgatttaaagtataaggaagatgtgcatatgcaaatatgggttttttttttttttaaataagggacttgagcatcctcgaattttggtgttcagttttgggtggattaggagggtagagaggggttcttgaaaccagtccaccagggataccgagagatgactgtattcttttgttctgtggcttgcctttcattttcttaacagtctttggaagagcagatgtttcttaattttgttgagtccagtttatcactttttacttatggttagtgctttttgtgtcttaagaagtatttagctacaccaaggttgtaaagattttcacctgtgtttattttggaagccttacagttttagacattacatttaagcctatgatctatttcaagttaatttttgtgtaggatgtgaggtaaaagttcaggttctcctctcccatatgaatatccagttgttcaagcaccatttggtaaaaagactatcctttcctcatttaattactttgacctttgttgaaaatcagttgtccataaatgagtgggtctttttattattgcattgatttgtatacctatacttttttttttttttgagacagagtcttgttctgtcatccaggctggagtgcagtggcacagtcttgggtcactgcaacatccgcctcccaggttcaagtgattctcctgcctcagcctcccgagtagctgggattacaggtgtgcaccaccacgcccggctaatttttgtatttttagtagagatggggtttcaccatgttggccaggctggtctcgaactcttgacttcaggtgatccacctgcctatacctcccaaagtgctggattacatgtgtgagccactgtgcctggccctgcatatctatacttatgccagtactacacttgattactgtagctttatggtaaattttggttactctagattctttgcatttccataaattttggtattaatagtgtcagtttctatcaaaaagcctgctggaatttcagtttgtgttgtattaaatatacagatcattagggagagaattgtcgtttaaaaaatattgagtcttctggtccttgaaaatggtgtatcttattattttttaaggttctttagtttctctcagcagtgttttgtagttttcagtgtaggaagtgttggacatcttttattggatttattcctaattattttaagtgcttacatgggaatttttttaaagtttcattttttccaatttggtgctagtatgtagattacaattgattggtggtctgtctagcatgtagaaattccttaataagtagtcagatggaatttaatttgttctcatctaactttctggagaaaataatcatctaaaagaaaaaagattatctctgaatttgaatgggttttaaatttgtatgactgtggcttattttaatttctctgaatttgtaacgtagaagtacatttggattttatggtaaaatatacgatattggatataagtggagacatgtagagatcattcaacagatgagaggatatagctatagaaaagcagagttgaaaatttttagatggtaaaatattcaattttatattaaatataatattttaaagtagaaagtgcttgagtagtatgattaaaaagaggaaacttgttttcatttgaatacattttggcctacaagaatgtctgttactggcttaagtatttttgtttgtgtataccgtcagatgatttaccatttcctagccaaacctgatttgttggcccagtacaactctcttctctcttttaagctgtgtctttgatagtatttctgttcatcctgcctagaagattgtgttctcacacagttttctgttgttggttctaaaacatggtgtaaatgagaggattgtaaaatgcgccaatcagcactctgtaaaaatgcaccagtcagtgctctgtggctagctagaggtttgtaaaatggacccctcagcacactgtaaaatggaccaatcagtgctttgtaaaatggaccaatcagcaggatatgggcggggataaataagggaataaaagctgggcaccccagccatcagctgcaacccagtcaggtccccttccatgctgtggaagcttttttctttcgctcttcacaataaatcttgctgctgctcactctttgggtccgtgccacctttaagagctgtaacactcaccgtgaaggtctgcggcttcattcttgaagttagcgagaccacgagcccactggaaggaaccaactctggacacatcttggtgacccagacgggaccattggaccccttttgcttgctattctgtcctatttttccttacaattcaggggctaaacaccagggcacctgtcggccagttaaaagcaactagcgcggccaccggactaaagacacgggtgttaggctttctgggaaagggctctctaacaacccccgactcttcagagttgggagcgttggtttgcctggaaccagcttcctctttgcctgtacttctgggctgagccaagggttgacaagaggggaaagccattcagctgcggggtcccaacaaaaagttggttgaccctgtagccatgaggggaactctgtaggtcagccaaaggttcagtgggtcggtccaggggtccttggtagaagttgttagttgaactcatttggggttccatttgtaagaccatctatagcttgatggcatcgatcctggaggaaacaaatttgacaaggagtttaaaaatacgggcctgaaggcgagtaatagcaagatggctgttacgggacctagaaaggggagaagccatgttgcccagctccagaggttggtataagagtttgaaaggcgttgtctgatttcagaagccttttcctgtaaacgccaggtggcgtctcatactatccctcactggttagtgtaaaagcaacattcttcccctaagaaggtgcatagtcctcctttctcagcagtgaggaagtctaggccttggcggttttggagagtcactgctgccaaagagtctatctgggattgtagaggaaggatagattttgttatttcttgcaagctgtctgagaaatcctttgagagtgtgtggtagtaggataatgaagtagataaatctgctattccaattcctgtagcagtggccattcctaaccctataagtaggggtattagttgtatggccctgtgctgatggacttgagctttgaaggggcactgatagagtctgatttcctggggcaatgttaatgttgggacttaggaagactaaggtgtaggtgcctgtccagttggtggggaggcagatacaggttgaagttccacttaagaagaatatgccttggctgggtagacagaaatttaccctggcttttaaaggaatagggtacactgttttttctttactacttctatctctgtgtctctctttgtctcttcctctctctctctctctctctctctctctttctctctttctgacttcttttctgtctcttcctctctctgtttctctctttctgactccctctttgtctgtctcttcctctctctctctttcctctctgctggtctttccctgcctctgccagctgcttatgctgctgttctcccctctcctttcagcagtgtaagactgccacctccttgggtttttgcactgtgtgcaataactccgtgatttccttatggtatttaatggggttcacccagaggttaggaactccctttctttccatattgcatcatgggcacgaaggattagataagcatacttgttatctatacacatttattctttttccctttcccagttctaaggcttgggtaagtgccactagttctgctatctggccgctggtccctgggggaggaggcttactttcaagtactgttacatcactaactatggcataacctgccctttgtatcccattcttcacagatgaactgccatcagtgtataggttaaggtcaggattagctaaggggacttctaagagatcctcttgggtggcataagtctggactataatttgttggcagtcatgctcgattggttccccatcctctgggagaaaagtggcagggttgagagccgcacagatgcatatttgaagcatcggtccctcaaggagtagcacctggtatccaagcaggcggttgtctgatagccataaacttcctttggcacctagtgtgccatttacatcatgagtagtccagacagtgagatcctttcctagtattattttgatagcctctgatataagacggccactgctgcaactacacgtaaacagtgaggctagccttttgccactggttgtagggttgtcccgtgagtctgagtaaggacttcaagagctattcctgctctctctctgacgtataaagagaagttttgtcttgtgggaaggcttaaggctggagcttgtactagggcctgctttaaggatttgaaggctgtttctgcctctggttcccattctactagatgagtatttgccctctgggtctcctttattagagtatagagtggcctggccatcttgctgtatccagggatctgtagtcggcaaaagctggtgattccaaggaacccccgcaactgttttaatgtcttagggcgaggataagccagtataggctgtattcgtttcttgctgagggccctggttcctctggctaagattaggcctagatatttgacttgttgtaggcagagctgggcctttgatttagatgccttgtacccttgattagctagaaagttcaagagatctcgagtagcttgctggcatgaggcttccgaactggcagccaaaagtaaatcatccacaaactgaaggaccagagtgcctggacttgagaagtggcctagatcttggaccagtgcctgaccaaacaggtgagggctatccctgaacctttggggcaagactgtccacgtaagttgggacgtgtgatctgtaggatcctcaaaggcaaagagaaactgggagtcagagtgcaggggaatacagaagaaggcatccttgaggtccagaactgtgaacgattctgcttcctttggtatttgagagaacagggtatagggcttgggtacaactggatatagaggaattactgcttcattgatgagtctaagatcttgcactagtctccactgactggtttttgtactcctagaattggggtgttgcagggactgctgcattttcttactaggccttgagcttttaaatgtctaacaatatcctgtaatcctttatgagcttcaggccttaagggatattgcctttgataagaaaaagtggtgggatcttttagccttatttggagtgggtgggcatattttgcccttctgaattgtccttccaatgcccaggcttcagggttgattccctcctcaagcaggggacaacaaattggtaacttgttccccatattcatgtagataatagctccagctttggctaatatgttcctccctaataagggtatgggactttcaggcataacaagaaaggcatgtgaaaagagcaaagtctcccaattaccactgagaaggtgggagaaatacctggttacagactgtcccaggattcctcggatggtaatggaccttgaggacagctgtccgggacaggagattaacactgagcctcggatggtaacggaccttgaggacagccgtctgggacaggagattaacactgataaagctgtaccggtgtccaggaggaagtcaatttcctggccctcaatggttaaacatacccggggttcagtgagggtgatgacatgagctggcgcttgccccgggcaccctcagtcctgttggatcatctggttgggggcttctggcccagagaacctttgtcctttggggcagtgcaccttccagtgattgccttggcatagtggacatgggcgagggggcagcttgtttctcgttggacaatcttttttaaagtgtccttgcaaaccaaattgataacaagcctaaccgggtgattggcctgctccattttctgtcctctctgaaccaccaaggtctgtttgtctgagggccatgactaaggctgtagcctttctcttatctcacttttccttttcagcctgttcttctcgatccctactgtagaacacggaggttgccaggtttaataatgcgtccaaattttgttcaggggccagggctagcttttggagctttatcctgatatctgcagctgactgggtaataaacttatcttttaggatcaattgaccctcgagtgagtcgggtaacagggaagtatattttcttaaggcctcctgtagccgctcgaggaaggcggtaggattttcttcctttccctgagttatggtggatgtcattgaataattcatgggctttttcctaattctgcttagtccttctaggacacaggttaacagatgttcacgactccagtccccatgatctgagtcgaggacccggtggggatccacactgagaatggcttgctgaccagtagggaatttgtccctttcttcgactgtcattctgtcatttacgtgactgagatgccaggtatctccaaactcttgggctgcaactaaagctgcattcttttcattaaaggccagggtttgatctaacaatagcatgacatctctccaagtgaggttgaaggtttgccctagaccctataggacatctatatacctatcaggatcatctgaaaacttccccaggtctactgtcatctgctttaaatcagagagagagagaaggggacatgtacctgggttgggccaaattcccctccccctacagcttgaaggggacataaccgatagcctgggagtttttgtggtcctttggagatttctttgcttgtttccttctgggtggtggagattagaggaggcttatcattaataggaaggggagctgtaggaaggctaggatatgggggtaagctgagaggtcctcctccccaatattgcaagctttgcatagttgtggattatccttcagtgaaaagaaagcttggacataaggtatttcactccgtttgtcttccctcttacagaaaaggtcaagctgcaggatagtattgtaatttatatttccctcaggtggccatttttgaccatcagagagagaatattggggccaggccatagtgcagaaaaaaatgagccgcctctttttcagggtttgcaggtcaaattggtcccagtggcttaggatgcatttcaagggtgagcctgttgatgcctgtgtgttccccatctgaaagaaaaaacctcctgtggttttggtttgttccccgctaccctctcaaccccgagttatcgtggctttaaaaaccggaccattgtcgctttggctggcctatagaattcccctctggaggacactaccactgcagggccccttctttacccctatccagcaggaagtagctagagtgggcatcgcccaattcctagcagcagttggggtgtcctgtttagaggggggattgagaggtgaagccagctggacttctgggttgggtggggacttggagaactttgatatcttacaagaggattgtaaaatgcgccagtcagcgctctgtaaaaatgcaccagtcagtgctctgtggctagctagaggtttgtaaaatggaccagtcagcaccctgtaaaatggaccaatcagtgccctgtaaaatggaccaatcagcaggacatggagggggacaaataagggaataaaagctggccaccccagccagcagcggcaacctggtcgggtctccttccacaccgtagaagctttgttctttcgctcttcacagtaaatcttgctgctgctcactctttgggtccgtgccacctttaagagctgtaacactcaccacgaagttctgcggcttcattcttgaagtcagcgagtccatgaacccactggaaggaaccaactccgcacacataaagatgaggaaaattgttcttttggttttgacgactttactatgtgcagttagcattgctgaatgtggtggtctctgagagcagtgtaattctgatgatacatacaggcatttgtctattaaaaatgggatgaaattgaagatggtttattgccttttatatcatcttttggatttgtgataaagacaaacctttgaaaatgtttgtagttattaatatgaccagaaactgaaactgtttcagtttggcacaggagagattagtagtagtattgctgttagtcattatgtaagagatgataatgtttggatttaaccttcgattgtttgtaatatgatattcctaacattcctgactaaaaggctagtgaataaaaaataatgcatttacaactatattaaaactgcattgcagaaagcatatgaaatttcaagtttaggaaatttcctgctttgaaacttttttcttcacacttcacgctttcagcacttatgtgtgatgcatctagaagatgcatctggttttgccaaatacagtactgcacctgaattacattttcggtatttattcagtatttattttcagtctttatttcctaaaggttaattttctttaaaaccttagcaatttttcatccagcccttttcatgttataatcatttgcttgtgtgattttaccacacactgtatccaactaggtagcattactggaaaacttatttgattttttgccaaatgggagatggctggaatattttttgtagtagagaatatgtggagaaaatgctttaattgatccctgaccaaatacagcaagaaaaacttaagtaaaccttaataagcaggcaatagaagttaaggtgataatattctgactttggatataaacactcaaacatgagctctgctactggaaagtaaaaaataaaattagtgttaagttgagatgagtcaacttgttgatcatctgcctagcctactgtgataggaatggttgtaagacttggaagtttttccttcctaagttgataagcatttgagtggaaagaaagtcacttttctcttgatgttttttcactttggaattttcagactgtttaaagtcttttaaaattttctgagtcacttctttattttctaggctgctcatattaagagaagtattgcctttttatcctttaaaatgtaataccctcatctttagtggttgtccctgcttaggttatattgcttagaacataacgtttatgtttggttaaaatttcactgagccaccgggcacggaggctcacacctgtaatcccagcactttgggaggcaaggcaggtggatcacctgaggtcgggagttcgagaccagcctgatgaacatggagaaaccctgtctctgctaaaaatacaaaattagccaggcgtggtggcacatgcctgtaatcccagctacttgggaggctgaggcaggagaattgcttgaacctgggaggcggaggttgtggtgagctgagatcgtgccattgtactccaacctgggcaatgagaatgaaattccatcttaaggaaaaaaaaaaaattcagcttctgaaaagcactagggactaagcaagttgactatgtaggctttattattagatttgtgtgtttgtattataaggaaaacaaatttttagtaaaatataatgtattttgttcattgtttagttcattgctggattgctggtctctagaacttttcctggtacagaataaacatataataagtattttttaaatgatgtacttaattttattaccctaccatgcttgaaactgtgactttatattatagtttcctttagcaaaaggtgaaactatagtcaatatatagattataagtaaatttttaaaaatcttcatttaccgtaatagcatcattttacatgtatacctgcttttcagatttgtaactcatttggtttaagtttggggcatttggatgtttctgatgttgaatgataaaatgtaggcttaaaaaaattatccttcgagttttatgctgctgtattgtgtatagcctctctttcataatgatacttggactgtgatggtataaagtgactcttattttaatctgaatgcattcgagatatttcaagttcttgaaagactgttacacttcttgggatggtcatcatcatatttttgaagcagagatcctaaaacttaaaaacctcagagaaagtccagcatctattttcaactccttgttttttaaagctttagaacttgactttgtactctttggtgaggtgacaaaacaaattcagatttttttttttaactgaggacttcaaaaaatttttgtgatgtttatagtattttgtctctttttagaaagtcaaatttgttgaggtatagtttacactcagtgaaattcacccattttagatgtatggttctgtgaattttgacaaacattactgaaatggctaaagtcgactgctatctagcagacagtgctaaaatagcaacttagcagttaaaatgtcatctttttatcactttgggaagattctcattggtatctttccatgggtacaataagagaccagcagtgtggcaggctttcactgtctttttccctcaactctacctcacataaaggtgagattttcctattttcttaggaaagtcagctagtggggtagggtgataagctaggacgggttccttgtgattttatttaaaggttggtctacttaatgctgggggaaaaaagaagtgtagtgatacaacatgttgcctacctttacattttcaaaataattttaaggaaataaactgctgtttgattcgaaatgattctttgtgttatggcagttaagattttattaacaccagggctcgagaatgcctgttaaaaatctaggtgttgctccttctcagctaccaactatatctttttttaagggccatattagtaacatatgttgggaacttttgccatgttaccaattatgctttttcagttttttttttgagacttttctactcaaaaaggttctgagtatttagaaagtactccaattgtaatcattctttttattgcttctggaatgtagcagggctccatgttcttccccatttcttgacagtttcttaatatcttggaacattgttgtagagtataatgtcctatctgtgttgaaatacttggctttggcatttaagtgctatcatgcatttatttgagaacagtttatggctcattcagacattgtggataagagcattgacttaggtgtcaggcagaataaatttaatctgactctgccatttagtaactatatgaacatggataagttaattataactgctctagctgttttgtcatctgccaaatggggaaaataatcatatgtatcttccaggattgttgtaaggattaaagagataatccttgggaaatattttattgcatgtagtaaacactcagagcatgttagctgctcctgtcatcctcactcagcagaagtttttttttgttttcatcttttttttttttttggagatagtcttgctctgtccccaggctggagtgcagtggtgcagtctcagctcactgcaacctccgcctcctgggttcaagcagttctcctgcgacagcctgtggagtagctgggattacaggtatgtaccaccatgcccagctaattgttatagttttttagcagagatggggttttaccatgttggccaggctggtctcgacctcctgacctcaagtgatctgtctgccccggcctcccaaagtgttggaattacaggcttgagccaccgtgtctgtcctcttttcatcccagaccatgcttggtgttgtggatacaaagtgaattctaaagaaggcctccccatcttaagaagtttcatagttcaagaagggaagacaggcatgtaaacagataaatgcaataagttgtgacatatgctatatcagaaacaaatgaaaagaataaaggagttagtatcataagcctggggaggtcagggaaagacatttctatccctgacttatttttgatgtgtgaatagtagtttcctgttgacttatttgacagatatttactgaatacactttacttttgccaggcactattttaggtgctgggaataccactggtttcatggaagacaatttttccatggactggtgggcagggggtggaggggatggttttgggatgatccaagcacattacatttattgtgcactttatttctattattactacattgtaatatataatgaaataattatataactcaccataatgtggaatcagtgggagccctgagcttgttctttcacagctagactgtcccatatgggagtgatgggagacagtggcagatcatcaggcattagattctcataaggagcgtgcaacctagatcacaatagggttcaggcttctatgagaatctaatgccaccgctgattttacaggaggcggagctcaggtggtaatgcaagcagtggggagcagctgtaaacacagatgaagcttttgagcccctcctgctgtgaggccaggttcctaacaggctatggaattggcttgtagcctaggggttggggacccctgctgtataggagttgtgaaaggaaattaaatcttggggccctcaatccctaaactaaaggaaaaagtcaagtggggaactgcttagggctaacctgccttccattctattcaaaatcatccctctgctcactgagatagatacatatctgattgcctcccttggaaaggctaatcagaaatgcagccattggtctctcacctaactttaacctggaagctccctccctgatttgagtcttcttgcctttgctttaagttgtcctgcctttccagaccaaaccaatgtacttcttacatatattgatcggtgtctcaagtctccccgaaatgtataaaactaaactttgccccggccaccttgagcacatgttgtcaggacttcctgaggctgtgtcatgggagcgtcctcaacctaggcaaaataaaataaacttttttttttttttttttttttttttttttgaggtggagtctcactgtcatccaggctggagtgcagtggcacgatctcggctcactgcaacctccacctcccgggttcaagtgattctcttccctcagcctcctgagtagctgggattacaggcacatgccaccacacctggctaattcttgtatttttagtacagatggggtttcaccatgttggccaggctggtcttgaattcctgacctcaactgatccgcttgccttggcctgccacagtgctgggattacaggcgtgagccactgtgcccagcctgcaaaataaactttctaaattaactgagacctgtctcaaattttcagggttcacagagtaaagaagacagataatgtctctactctcaggagctttcagtctagtgggaaaagatcagtagtaaacaaaaatatcagagacaagtgctatgaaagagactaaaaggtgatgtaataaggagagactgaggggttgctttacattgcattgtcagggaaggatcctttgagtaggtaacctttgagatcttcctacaaaagagtcagccatggcaaagattagagggatgagcattctaccatagggaaccatgttacacaaagccccttgagctcttggagtatttaaggagcagacaggtcagtgtggctggaatgtaatgggcaaggggtagagtggtgcaaaatgagatcagagatagtcagggtctaaatcatttaaggctttgtaagccagagtaaggagtttctaggtgtaaacgaaagccagtggagggctgtagcagggggaacgccttttgaaaaagattactgtcgctgccgtgtgaagactggattctgaggataagggtaacaagggtggaatttaggagacctctggaggctgctgtattagtataggtgagaggtggtggtggcttgggccagagagtaaattagtggagaaaaataggttgatttgggctgtgttttgaagattgaatcaacaggacttgctgactgcatgtgatcaaggcaaatcctaggacttgagcacttagaagattggtggtgcagtttactgaaatggataagactggaggggagagcaagtttggtgggacaatcatgagttctgttttgttaagtttgaggtgccaattagacatctatgtagagatttcatacttggtggtaggtaatagtagtactgtggaattcagtgagagtttagggccagggataaatatttaggagtaactgatgtatgtctgctagatttggtaagtgaccttgacaagtgtggtcttagtgggaatggaaactagattgtagagaagtatcaggtaaaggatggaatgagaatatgaagttggggaatggagacagtgacaatagactcctaagaaattttcctgtgaaaagggaagcagaaaaatggttgtgaccacaagtaaggcccagattcgaatcttacttttatacttgcctggatacagttattatttgagtgcattctaaagtttaagatattttatgtaccctacgcagtcctctctacaaatattgaggtagatgatacttttccttctgtgacatgagagtccttagtttagggaagttaagtgacagtccaggtagaacctgtatttggactcactcactttgactgtagatgcagagtatttttcattataccaaaatgctccttaaacttttattatcttggtcaaatccaatctaccttagagtttttggaaggattacaagatgagagatgtgaaaattctttgcaagctctaaattgttacacaaatatagaaattcctttctttaaaatgccccattatgtcttctttgtgcatgactttttaggatatagtaggtagtggccttgaagttcattttggatcatagcctatctcattagagcccagagaatgtaagcagattgttaaagacatagaacctgtcaaagctatctatggcatatgaatcgaacatggatcatgtttaattgaacatttttaccctggtttaatgatcttgtctccttcgcatgttccatgaggtctcataatcctccccaaaggagctcttcccaaaaataattccttctaaaaatgtggaagggctgggcacagtggctcatgcctgtaatcccagcactttgggatgctgaggcaggtggatcacctgaggtcaggaattggagcctggccaacatagtgaaaccccgtctctactgaaaaaaaaaaaaaaaaaaaaaaaattagccaggcgtggtggtgggtgcctgtgcctgtactcccagttactcaggagactgtcggaagaattgcttgaagcgagaggtggaggttgcagtgagccaagatcgtgtcactgcattccagtctgggtgacagagcaagactccatctcaaaaacaaaacaaaacaaaacaaaacaacaacaacaaaagtggaagtaattttaaaaaaactgcaaaaaagccaatcctgaaaggttacaaactatatcattccaaaagccgtggaaaacagtaacgtgagtcctcaaagaattaaaaatagaattaccctgtgatccagcaattctacttctgggtatatactcaaaagaactgaaagcagcagggtcttgaaggggtatttctacacccatgctcatagcagcattattgacaaatgctggaagtagtccagctgtccccaaatggaggaacagataagcaaaatgttactggtatacatacaaaatggaatctttttttttttttgggagacagagtcttgcactgttgcccgggctgaagtgcaatggtgtgatcttggctcaccgcaacctctgcctcccgggttcaagcgattctcctgcctcagcctccctagtggctgggattacaggtgcccgccacgacgcccagctaattttttgtatttttagtagagacggggtttcactgtgttggccaggctggtcttgaactcctgacctcgtgatccacctgccttggcctcccaaagtgctgggattacagatgtgagccaccgcacccggccacaaagtggaatcttatttcacctttcaaaggagagaaattctgacatgctacaacctggatgaatcttgagaacaatacactaagtgaaactggccagtgaaaccagtcacaaaaagacaaatactgtatgattccactttaatgaggtacttagagtagtacaaatcacagagacagaaagaatggtaattgccaggggttgaggagaagagggaatggagagttactgtttaacgggtatagtttcagttttgcaagttgtagagttctagagctagatggttgtaagggtcacacaacaatataaatgtactcagtaacattaactgtgcacttaaagataatttagatggtaaattttatgttatgtgtattttactatgccatcatgcctggctaatttttttgttttgttttgattttctggtgacagggtctcactctgtcgcccaggctggaatgcggtggcacgatcacggctcactgtagcctcgacttcctggactcaagtgatcctcccatcacagcctcccaagtagctgggactacaggtgtacgccaccactcctggctaatttttactaactcactcccttttctgcctcctttcagtattcattcattcatttaacagataatgactgaacaactgttaggtattattaggtattatatattattcctgggagcagactattaagagtcactctataccatcacagtcccaagtgtcattatgaaagaggggctatacacaatacagcagtataaaactgtaaacagattttttaagcctacattttatcagtcaacatcagaaacatccaaataccgcaaactttctttttttttttttttgagacagtttcactcttgtcacccaggctggagtgcaatggcgcgatcttggctcactgcagcctccgcctcctgggttcaagtgattctcctgcctcagtctcccgagtagttgggattataggcatgcgccaccatgcctggttaatttttgtatagtagagacggggtttcactatatgttggccaggctggtcttgaactactgcccccaggtgatccacccgtcttggcctcccaaagtgctgggattacaggtgtgaaccaccacacctggccaaatgtcccaaacttaaaccaaatggactataaatcaattccctgaaaaactggtatacatgtaaaatgatgctattacattacagtaaatgatctttaaaaatttatttatagtctatatgttgactacagttttaccttttgctaaaggaaactataaagtcgcagtttcaagtatggtagggtaataaaactaagtacatcatttaaaaatacttattaaatgtttatctttgccaggcaatgttctagagaccagcagtccagcaatgaactaaacaaaatgaacaaaatacattatattttactaaaaatttgttttccatataatacaaatacacaaattaataataaagcctacgttaatgcattgtactaatctgtcttggtgagaagttcaatttatgaagatacagtagaaatgattttaatttcttttttttcccctagagaatgtgatgtttattcattaatttattcattcactcaagaaatcttcattgaatgtctactgtgtatcagttgcagtttgggattcagcagtgaacaaaacataaatgcttgcccttatggaacctacaatctgttggttttaaaaaaaatctctcctggattagtgttagatttctagaccagaagtgtcaattaagtttaacaatttcttcaggcagagaaaattgcttttgaggtatcacttaagcaaccttagtgcttctaagcaacataactgccttttgaggtatgaactgaagggaagtgaagtataagcaatagagttgggattttttttttcttgagacggagtttcattcttgtcacccaggctggagtgcaatggcaccatcttggctcactgcaacctctgcttcctgggttcaggggattctcctgcctcaccctcctgagtagctgggattacaggtgcatgccaccacagccagctaatttttgtatttttagtagagacggggtttcaccatcttggccaggctagtctcgaactcctgccctcaggttatctgcctgtgttggcctcccaaagtgctgggattacaggtgtgaaccaccacccctggctgagttgggattttttaattttccatagatttcccattaggaaggcaacctggtgtgacggtatttgagagtcaagagatctgggttctaatcctcatttcatcatgtaatactttgtgtgtttcagaattcttcacagtgatccttgcttgtcctcactcctttgtacagcttttttttttaaaggattttatccactgttgtgccagtaactatgctagtgtcactttttatgctagtaatagttctattttcaagttcattctgtttcctgagtttaaatttgtgtttcctagtgccaactgaacatttctgtcatttcatactccatttccaaaaagaaactcattattgttccgctttgaatttgcttcccttctttatgatcttttcatctcggcaaatagcattgctatgtacccagctgcctaaggaaaaaccctgagtcgtcatctttctatatttcattgaccctcattcagtcattcaaatattgcttctcttgagtatatcttgaatctatcctttatgctctaatttctgaacagaactgacactctttgtcctgcatgttctactttctttgcacagaataccttctctttagttggcaaactccaactcattggttaatgtccagctcaaatgttaactcctgtaaaaccttcaccatttcttttaggcagaattagtgattcctgagtctttgctcttagagcttgtaggtcaagtgtgtactgtatttctgttctttcacagttttttattcgattgtctgtagaatagactttattattgtcaaggggaagacagccttagcaaaattttgaaataagtcattgtgtgcctaagcataaattagtttgtgagaacatactttttaaagagaaatgtagtagggtgtatagtatgtttgagtatctttttccaataagcattgcatgtataatcattcactcattgcatgtataattattatttaaatattctttattaattaaagacctctctggtctctccagaagtcttccatggctgccctcatccccaccctccagaggaatctgaagccacatgtgctccctggcacccacagcccctgcctctcccagagcagcagtacctgagcctcagtggattccaagaatcgaaacccttggtctgctgcccctccctcccagaatgtttctgtctcattcttacctactcaaggccctttcagtagccccttggggtattctcttcctatgcaccagggcaacttctgaactcaccactttcctggaaagacccccaacatcttcggggagcggtcccctacaggcctggctgcagatgcccgtgcacacaggaacactgtttaaaaaaaaaaaaaaaggatactggtgggaggggctttgtaactgaggaggtttaggaattgaaaactcatgtgtattttctttctttcctcattgtcacttcccagctacccagagatattgaaggacagctgagctcccctacttgctgaaaatgcattgatcaaatttgtgtaggatttccacatctatttatgggctcagaacactttctagttacttgttccttcctatcagctgttttctgtttgtgttagattttttttttttctgagatgaagtctcgctcttgtcccccaggctggagtgcagtggcgcgatctcagctcactgcaacttccgcctcccggtttcaagtgattctcctgcctcagcctcccaagcagctgggattgcaggcacctgccaccacgcccggctaatttttgtatttttagtagagatggggtttcaccatgttgatcaggctggtctcgaactcctgacctcaaatccgcccacctcagcctttcaaagtgctgggattacaggcatgaacaccacacctggcctcatgcagagctttaaaaaaaaatgttaacacggctgggcgcagtggctcacgcctgtgatcccagcactttgagaggccgaggtgggtggatcacttgaggtcaggagttccagaccagcctggccaatatggtgaaaccccgtctctactaaagatacaaaaaattagccgggcgtggtggtgggcgcctgtaatcccagctactccggaggctgaagcaggagaattgcttgaacctgggaggcggaggttacagtgagccaagatggcgccattgcactctaggctgggcgacagggcaagactccgtcttaaaaaaaacaaaaataaataaataaataaaagaaagctctgcatgatacaatacagtattcatagaattttggccagtgaaaattttctgtgttagtagaataaaaatgaactcttctgatttacagatattgatagtctggtctttccatcttccatcttgactctcttttactgttaaaaaatcttagaacaaagtaaagccagttccaaatacttttcctagttctggactgtttttttttttttttttttttttaacaagacagtcatttgtccgagtacaacagaacagttcattgttaatactttggttctgtgtgctttaaatcttttttccccctctacccataatctccctttgtaaactttgaatgttttgtcagtagatcatcatcataggtaaagatactgcacatcaagactgttggtcttccccattcttcctggaatatactggaagctgtcaggactgaggttaatttctccattcctagaactatattgtaataaatattggaatactggcttttgtgtatttttttgtgttacttagtttttggagaaaggaaatatttctagactttcttgacttgtcataactttttattgttaaagaatatagtttaaaaattattaaatattagggagccttctgcttcattgggactttaaggaatgatagccataggaagggatatatcttatattatcataaagcttttaaaaaataatatatttattacaattttttaaatttattttttatttttagaaacagggtcttactctattgcccaggctggtctcaaacttctgggctcaagtgattctcctgcattgacctcccaaagtgttgggattacaggcatgagccaatgagctcagccagttgttttttttttttttttttttttttgagtgtttttttttgataacttctaattattttcttatcctatccctaatagttggcatcagttaatgaactagttttgattccccaaaagccctctcttaatctcctgtattttgctttactttctatctcttttgtttatttcttagagaaatgtcatttatgtaatcagttcttattttgtaatactgggccgtaagaaagcagggaaaagcacaaaacttggcacatagtaaattttgttgcttgaacaaattaatgaatgaatgaacttgcagcataagagtgcaccctatttagtggtactgtttaattatttcatgatccttagccaagaacgaaggcataccggaagggaacacacaacaaaaaaaggtgagggaaaagtgttaatatatcatttaagtgactgtattacaacaatattacattatgttgtcactcctgaacatgagatgtgattctcatttacatttcaagctaatttggttagctgggggaattcttaggcatctcaagagataattagatataccccttaggacctcacataattataatcactattaggggtattaaagtttttttttttttttaaacaaaaacttagggggaaaagttgtgattggtaagcgtttttttctcttagtgttctcagaatcttaggagatacgttatttgaaacagttgttcaggccgggcgcggtggctcacacctgtaatctcagcactttgggaggccaaggcgagtggatcatgaggtcaggagatcgagaccatcctgactaacacggtgaaacctcgtctctactaaaaatacaaaaaattagctgggcgcggtggcgggcgcctgtagtcccagctacacgggaagctgaggcaggagaatggtgtgaacccaggaggcggagcttgcagtgagccgagatagtgctactgcactccagcctgggtgacagagcaagactctgtctcaaaaaaaaaaaacaaaaaaaaccaaacaacacttgttcaatcaattctactttagtagaaaccagctctctatattgttctctaacattttgttatcttggcttttagatgaaatctttagcatcagttgggctcattagcacctctactaagagaagcaagcacagaatggaagggagagcaaatcataactttttaaaaagttatttctccttagttaaaagtttggaagagaagcaagttgaaactgtaggctgattatgtaaaagtttaagttatcagctctcttatatgtaatgcaggttgattataagtttctgggctgttgtgacaacaagaaaattatgtttattattaaaaacttgtttatactgcatatatgtgaagggcattatactgtgacaaagctaacaataaaacatacattttaatatttcagattccttattttatttgtttgtttagagacagggtcttaatgttgcccaggctggagtgcagtgatgcgatcatggcttactgaagcctcaaactcctgggctcaagcgatcctcctccctcactctcccaagtagctgggactacaggcatgtgacactgcgtggctgatgttttgtagaggcggcatcttattatgttgtctaggctggtctcgaactttctcccgcttcagcctcccaaagttctgggagccactgtgctgggccagattcctttttttaaattgtgggaaaatacacgtaacataaaatttactatattaaccattttaaagggtgcagttcagtggcattaggtatattcacaatgttgtacaatcatcacactacctagttccagaactactttatgatcccaatcagaaactatgcgcattatacaataactcctcattgttctcttctcccagttcctggtaagttctattacactttctgacgctatcagtttgcctgttctaagtatcatataaatggaatcatatagcatttgtctttttgtgtctggtttatttcacatagcgtacatgttatagcatatatcaaaattttattccttttggccgggttcggtggctcatccctgtaatcccagccctttaggaggccgagctgggcagatcacctgaggtcaggagtttgagaccagcctggccaacatggtgaaaccctgtctttactaaaaatacaaaaattagccgggcgtggtggctggcgtgtgcttgtaatcccagctacttgagaggctgaggcaggagaatcgcgggaacccgcggggcagaggttgcagtgagccgagatcgtgcagctgcactctagcctgcatgatagagtgagacttggtctgaaaacaaaaacaaaaacaaaaaacaccaagtttatttaaaaaaaaaaaaagcttagactttttttgagcatttttatgttatagaaaaattgagtgaaaactagagttcccatatatctacccgctgcttggtaccaccagtttcccgttactaacatcttgcattagtgtggtacatttgttacagtttatgaactgatgttaatacatttttaattaaattgcatagttttgcattagggttcactctgtgtgttgtacattctgtgggttttgacacatgtatgaaaggtatccactattacagttatcacagaatagtttcactgccctaaatactcagtgtttcatctactcatccctaaatccctgccaaccactgatctttttactgtctccttagttttgtcttttccaaaatgtcatataattggagtcatataatatgtaatcttttcagattgatttctttccattggcaatatacacttaaggttctccatggcttttcatggcttcatagctcatttctttttattgctgaatagtacactgttgtatggatgtgtaccacagtttgtttattcattgacctattaaagactatcttggttacttcccagttttggcagttatgaataaagctactataaacacttgtatgcaagtttttgtgtgcacataggtttttaactcatttgggtaaattgtatgcctttctaacttttgtatttatgcatacacttttatatagttgtaaaacttagttttatctagttgtaataattttgttttatgttttaagataacataaacaatcttcatgctgtttttgaatgctccctaatattaaaatgagaaaatgaatttattatgtactttatttttctgttactcattaattgcttccattttcccctgccttaataaatacttttgtgtgtgtagtttttcccttttgtattgctttaggataaaattcaaggagaaacagtaaagagtacaattattgggatcaaggccattttagataattcttgatatgcagtaaagtttcacaatgggaaaccttgttagatttacagtggaggtttgctgcagtttattttgtggctttctagacaagagtagcaccagttatctgtgtaattttacttagaaaaaaacaaagaacttttcactggttctttgaagtggtagtaagcttccctactgttggttttgttcaaaaatcactgtttgctatctgaatcttcttgattacttcagaactttatgtaatagtttattgaggttcttaggtacaggtagctaacacgggcttgactttatcttactatggtacaatctaaatatatatttagtgttgttaattaaggttatcattcacagatgctcccttatatataggtggagaaactgaggcttcccttgagacagcatgctcctggatataaccgtgagagcagttaaactcttgagacctgcattctttatttttcttaattatcagtacaattattggatcagagtttcttctatcttttttgtgagtcattaaattccatttttaaaaggtatcttttgaatacatacatttacaggtttggaaaatatatttctagttaaaccatgtgttcttatttatttatttattttttattctaagttttagggtacatgtgcacaacatgcaggtttgttacatatgtgtacatgtgccatgttggtgtgctgcatctattaactaatcatttaacgttaggtatatctcttaatgctatccctcccccctccctcaaccccacaacaggccctggtgtgtgatgttccccttcctgtgtccatgtgttctcattgttcaattcccacctatgagtgagaacatgtggtgtttggttttttgtccttggcgatagtttgctgagaatgatgatttccagcttcatccatgtccctacaaaggacatgaacacatccttttttatggctgcatagtattccatggtgtatatgtgccacattttcttaattcagtctgtcattgttggacatttgggttggttccaagtctttgctattatgaatagagtgccgcaataaacatacgtttgcgtgtgtctttatagcagcatgttctctaatcctttgggtatatacccagtaatgggatggctgggtcaaatggtatttctagttctagatccttgaggaatcgccacactgacttccacaatggttgaactagtttacagtccccccaacagtgtaaaagtgttcctatttctccacattctctccagtacctgttgtttcctgactttttaatgattgccattctaactggtgtgagatggtatctcattgtggttttgatttgcatttctctgatggccagtgatgatgagcattttttcatgtgtcttttggctgcataaatgtcttcttttgagaagtgtctgttcatatcctttgcccactttttgatggggtcgtttgtttttttcttgtaaatttgtttgagttcattgtagattctggatattagccctttgtcagatgagtagattgcaaaagttttctctcattctgtaggttgcctgttcactctgatggtagtttcttttgctgtgcagaagctctttagtttaattagatcccatttgtcaattttggccattgcttttggtgttttagacatgaagtccttgcccatgcctatgtcctgaatggtattacctaggttttcttctagggtttttaggtttttaggtgtaacatttaagtctttaatccatcttgaattaatttttgtataaggtgtaaggaagggatccagtttcagctttctacatatggctagccagttttcccagcaccatttattacatagggaatcctttccccatttcttgtttttgtcaggtttgtcaaagatcagatagttgtagatatgcggcactatttctgagggctctgttctggtctatatctctgttttggtaccagtaccatgctgttttggttactgtagccttgtagtacagtttgaagtcaggtagcgtgatgcctccagctttgttcttttggcttaggattgacttggcaatgtgggctcttttttgcttccatatgaactttaaagtagttttttcgaattctgtgaagaaagtcattggtagcttgatggggatggcattgaatctataaattaccttgggcagtacggccattttcacgatactgattcttcctacccatgagcatggaatgttcttccatttgtttgtgtcctcttttacttcattgagcagcggtttgtagttctccttgaagaggtccttcacgtcccttgtaagttggattcctaggtattttattctctttgaagcaattgtgaatgggagttcactcatgatttggctctctgtttgtctgttattggtttataagaatgcttgtgatttttgcacgttgattttgtatcctgagactttgctgaagttgcctatcagcttaaggagattttgggctgagacaatggggttttctagatatacaatcatgtcatctgcaaacagggacagtttgacttcttcttttcctaattgaataccctttatttccttctcctgcctaattgccctggccagaacttccaacactatgttgaataggagtggtgagagagggcatccctgtcttatgccagttttcaaaggcaatgcttccagtttttgcccattcagtatgatattggctgtgggtttgtcataaatagctcttattattttgacatacgtcccatcaatacctaatttattgagagttttagcatgaagcattgttgaattttgtcgaaggccttttctgcatctattgagataatcatatggtttttgttgttggttctgtttgtatgctggattacgtttattgatttgcgtatgttgaaccagccttgcatcccagggatgaagcccacttgatcatggtggataagctttttgatgtgctgctggattcggtttgccagtattttattgaggatctttgcatcgatgttcatcagggatattggtctaaaattctcttttttgttttgtctctgccaggctttggtatcaggatgatgctggcctcataaaatgagttagggaggattccctctttttctattgattggaatagtttcagaaggaatggtaccagctcctccttgtacctctggtagaattcggctgtgaatccatctggtcctggactttttttggttggtaagctattaattattgcctcaatttcagagcctgttattggtttattcagagattcaacttcttcctggtttagtcttgggagggtgtatgtgtcgaggaatttatccatttctcctagattttctaatttatttgcgtagaggtgtttatagtattctctgatggtactttgtatttctgtgggattggtggtatcaccacccctgtatcacaatcccctgtatcattttttattgtgtctatttgattcttctctcttttcttctttattagtcttgctagtggtctatcaattttgttgatcttttcagaaaaccagctcctggattcattaattttttgaagggtttttttgtgtctctatccccttcagttctgctctgatcttagttatttcttgttttctgctagcttttgaatgtgtttgctcttgcttctctagttcttttaattgtgatgttagggtgtcgattttagatctttcctgctttctcttgtgggcatttagtgctataaatttccctctacacactgccttgaatgtgtcccagagaatctggtatgttgtgtctttgttctcattggtttcaaaaaacatctttatttctgccttcatttcgttatgtacccagtagtcattcaggaggaggttgttcagtttccatatagtttgtttttgattcagaaaatttggtcagaattagtgtctacacaatgggcctgttttcatgtccaccaggcattatggctgagtgaaaagccagaagggcccgattttgaacttttcttccacttaccagccatgagactgagcaacctgcacaaactgtgagccttgaattcttcagttgcagaaatgaggtgttgaatccttcaggttgttatgaagaataaatgagataatgggcacctgacagcatatatttatgttaggaatatataattattgctgtttgaaattgtagcctgtatatatttgcagagtgcccaaatacttgttcctcagatacagagtttatgtacatctcaatgtaccttttatagttgaagttgttttgttttgagagaagactgaagttatttgtggggttttttgttgttgttttttttcttacagtattgtcatgtggaaaatacatgttaattatcagggtttaggttttctgctgagtgtgtttcaaattaagtcatttccctctagaattggtagtttgttgtcttcatgttatttataatcatttccaagtttaccagatagaaaaacccttaatgaaattttggaaaagaagtacaccagtgagttgtttgctaatcttcctgtagttgtcacacagactgactgtttatatattttttggagacagagtctcactctgtcacccaggctggagtgccgtggtgtgatcttggctcactgtgacctctacctcctgggttcgagcaattcttgtgccccagcctcacgcttgggtaattttttgtatttttagtagagatggggtttcaccatgttggccaggctggtcttgaactcctgacctcaagtgatctgcccatcttggcttcccaaagtgctaggattacaggcggagccactgcgcctggcccagactgaccctttacagcatcactcctgttccttgcatattcatgcttccaatttaagattttgtccattacttattacagttgccatgtatgaaggtccctttttttgataattggttcttttctcttctacatactaaggcaggggtgggaataaatgtcacagcattaccactacttgatttgattttaatgtgttactctcttgtactcctaccttattagttttagtagtttatggctgggtgtggtagctcttgcctgtagtcccagtactttgggaggctgagatggatggattgcttgagcccaggagtttgagaccggcctgggcaacatgaagaaaccctgtctctacaaaaaaaaaaaaaaaaaaaaagttaaccaggcatggtggtatgtgcctgtggtcccagctacccgggaaactgaggtgggaggatcacttgaatctgggaaattgaggctgcagtatgagctgtgattgcactactgtgcccagcttgggtgacagagtgagaccttgtctcaaaaaaaatttttagtagtttctgtgttttgtttgtaaatttaactctgtattgagccagaaaagcacaagccctgtaatttctcattttagagaaaaacattcatttttttccttccccaaggagtttctccttcttcctatttttttctttaaagagagtttggcagaattccagagcctaaaataataaaataaaggtaattaaatgaattctccagccagcagagaccatcttatcttgaattgctagtggtttatgctggtccccctttgaccttctttcactttttggaggtggatgaacttttccactcaaaactatcttgtcagcctcacacatatttattaaaggagtgagtatgcttaagagacagattttttgtgttggttgaagaaaccattttggatatttttctctgttgtgactgggactgaatttttagattgatttatatttcctttgaagaacaaaggaatatttttctcgaaaatgagaattaacaggagcattaataacaacaaaaagtctgttaaacgtatttggggagattatttgttttcaaagaataacatttcttgtgtgagtcattatattcagcaaaatttcttttgtgataattttatgctgccataaaattttttgggaaaataactttaaaaaaacttcaaaaaattttattttagaggggcagtagtacagttggaagaggaattttaagtcaaagaacagtaaaacatgaattctttcagaagacagttgaaaatttactgtgcatcaggaacattacttagctctgaacataaaaaatgatgaaggtactgctctctgcctttatgtctttgcgtgtagtctaatgggagacacaagtaaataagaaacaatgggtcaaggtcattatagtagtagttacacaggtaggtgcatatgcttctagtctagccagtgggatgctgtgtaaattgtagctatgtggagaagccttgagatttccctctctgaaagtaaaattattgccttagggcagggttagcaaactatagctagcccctgggtatctacctgtttttgtatggctcatgagcaaagattggtgtttacatttaaaaatagtttaaacaaaataatgttttgtgatagatgaatgaaaattatatgaaatggaaatctcagtgttaataaagttttattggaacacagccacattaattcatttacatattgtctgactacttttgtgctgcactagctgagttgagttttttgtaggagagaccttttgtggcccacgaaacctaaaatatttataatctggccttttattatagaaaaagtttgttgagccttctttagagtgttatgagcactgactttttatgacctcaatcacacatgcagtttccttaaattccttattatatgtttaaaagttaaattagctgggcgcagtggctcacgccagtaatcccagcactttgggaggctgaggtgggtagatcacctgtggtcaggagttcgagaccagcctggccaacatggtgaaaccccgtctctactaaaaatacaaaaaattaaccgggcgtggtggcacatgcctgtaatcctagctactcagaaggctgaggcaggagaattgcttgaacccgggaggcagaggttgcagtgaactgagattgcatcgttgtactccagcctgggcgacaagagcgaggctccatctcaaaaaaaaaaagaagttaaattttaagtggctgtataaaggattatgatgatgcctaccttgaatggtaaacttttagatgtaacttttcagttatttctgctgaaaaatgctaaagtgatgtgctattctattgaaagccaagtgtattgatttgctaggcctgccataaagtactgcagactgggtggcttaaatgacagaaatgtattttctcacagttctggaggctagagtttcaagatcaacatgtcagcaggtttggtttctcctatcacccctctccttggcttgcaattgttgccttctctctgtcctcacagggcctttcctctgggcatgtattcctccctggtgtctcttcttacaaggacatcagtcatagtggattaggcccccccaccttgtaacttcatttaaccttgattacctctctaaagcctctatttccaaatacagtctcattgtaaagtactgggggttaggacttaacatatgaattttgagaggctacaatcagtgcacaccgccagggaatttctgatagaaacttcatttgtttacacattaagcagatcttgagggtgtgtactgtgtgacagatcctgttttacgtatgtatgtgtgtgtactatgtgacagatcctgttttagatacatatggtgaacgagacaaacaagatttgacacttaagaggctggtggtttagttgagaaagacagataattaataggaaaataaatatttgagagagtgataaggactgtgaaggaaattaaactgtgtcaagtgttagaatgattagtgtgacgggtcgtcaggtaggggcttcctgaaaaggtgaccttttacctgggagcttaaatggtaagggatagctagctagttaagttcccaggcatgtttctagggcctgatgttgatcaagattggtatggaaaaggttcaaaagaatagtgagtcaattagggagctttataagttgaggttggagaggttggtggggcaaaatcattaagggccttgtgggaattatggtaaagatttggcttccttttgcacaaggaataaaatctggagggattttagcaggggagtgacatgatttttctgttcgaaagagcactttggctgctaaataaagagaggaaactgcaggtatgagagtagaggatagggaggcctgttaggggattatcaaagtattgtaggtcagagatattggtggtttagctaagatcatggcagggaagagaaaaagaaagggaaataatttgagagatattttgggagataaaatccatgtattatttactaatggattaagtatgggggcttaggaagagggaggaatatctttggcaatagtgttgtgtaatactgtccactgtagcaagtgaacctatagtgttagcaaatcagatagatgattgctgcagcagcaagttacagaatataatttggaaaggaaaaaagccttcaatcattaaataggaaaacagaaggcaggtaaacaatgctatggtttaattatgttcaacttacaggcttataactactagacaaccaatggggaattcatagtttgccatagagaatgcctttgggacacataacctgtttggaatgaaaaagccctggagagtattgtaaaataacaaatgtcaggtttgatggaatgactatgcatgcaattaagtgattttctttagtgctagagttcttttttcttaggtaaatcattttaaaaaatggaccagactttgtgaaagtagatatgttacataaaataatgttagctaaagatgaatggaataggttaagagtgtcttcttgaaaatttgaactatatctctttcaaaaagaattttattacctctaggctggacttgactgaaaagttaaaaaaaaaaaaagacaaaatcttatgattgttgaactcccaatgaggagtcagagaattgtgagagccagggtctgttttttgtcacttttctctctctctttttttaagatagcatcttgctatctgcaggctgaagtgcagtggtgcgatcacagctgcttgcagctttgaactcttaagctcaagcgatcctcccacctcagtgtcctgagtagttgggactgcaggtgcacaccaccacgcctggcttatttttgcattttttgtagagacggagtcttgccgtgttgcccaggctggtcttgaactcctgggctcaagccatctgcccacctgggcctcccaaagtgctgggattacaggcgtgagccactgtgcccagctgccacttttttctatgtttagaaatctacctctgagccttattccccatccataaggcaggtaaccactagatggctaattttgaaagagcgagagcattcagagatgcacagtaattctaagttctgagtagaatacagtcatcctttggtatccatgggggattggttccaggacctcccatggataccaaaatctgtggatgctcaagttcctgatagagaatggcatagcgtttgcatataacctaagcacatcttcctgtatattgtaaatcatgtctagattacttgtagtaccaaatacaatgtaaatgccctgtaaatagttactatactgtattgtttagggaaaaaagtctacatgttcagaatagatgtaattttaaaaatctggataatttccatctgttgttggttgaatccacagatgcagaaccccattgatatagagggctgactgaatacaaaagcaaggcaagtgtgatagcagccttcttttactttaggattcaggatttacttctttacttcaggatcaaatatttttacttttcgattttatttttttgtttgtgaagagttgggagttgctgggtttagttatgctactctctttttaagggagaatatagcagtcctagagggatttgcaaactatagctcttggggaaacattcaaaattttccttgtgtaagaaattttatgagtctcgagggccggatgtggttgctcatgcctgtagtcccagcattttgagaggccaaggctggaggatctcttgaggtcaggggtttgagactggcctggccagcatggtgaaaccccatctctaccaaaaaaaaaaaaaaaaacaaagcagtgagccaagattgtgccactgcactccagcctgggtaacagagcgagacgctgtctaaaaaaaaaaaaaaaaaaagaaattctatgagtcttaaattacatcaagtaactttggaggcaatagtttaaattctctaaaatactgggcacattagctcatgcctgtaagcactttgggaggctgagatgggaggatcacttgaggccagtagttcaaaaccagcctgggcaacagagtgagactccagtgctacaaaaaattaaaaattagctgggtgtggtgggcacacgcctgtagtctaagcttctctggatggaggctgaggcgggaggattgcttgggcacagaaattcaaggttacagtgagctgtgattgtgccactgcactccagagtgagaccctatctctaaaaatattaaattaaaaaaaacccatacaactcaaatttccaggtactgttattaatatgagttatcttgaactgttgaaaaattaatcacttattgttgatttctatctgcatattaaagatctggcttaagagtcttgcatttgatgaataggcttttctatgttatatacaagaaaaaggagataaaataatttaacttttttattacattaaaaaacaggactctgggaattgtcactttatgatcagccttatattttttccagactgtgtcattgtgtatgcctgtgccacccaacggatgagaccactgatcatgtttagctcaaatttatgttggatagaggttataggatggtccttatctgaatgcagaaaggaatgtttattttgctgttatgttttatatgggataaaatgcttgaaaagtcttttttttttctgtatttttttttttgagacggagtcttgctgtgtcgcccaggctggagtgcagtggtacgatcttagctcactgcaagctctgcctcccaggttcatgccattctcctgtcagcctcccgagtagctgggactacaggcagccgccaccacacctggctgattttttgtatttttagtagagacagggtttcaccacgttagccagaatggtcttgatctcctgacattgtgatctgcccgtctcggcctcccaaagtgctgggattgcaggtgtgagccaccacgtccagctttttttgttgttgttgttttctgagacagagtctttggctctgtcaccctggctggttagtagttggaactataggcgcgtaccgccatgaccggctaatttttgtatttttttgtagagatggggtttcaccatgttgaccaggctgatcttgaactcctgacctcaagtgatccacctgccttggcctcccaaagtgctgggattacaggtgtgagccaccacatctgacctacttgaaaattcttaaatggtgccttatactttctggtaataaaagcactattttaatgttcggaactattttcattatttgctaaccttacaattaaaaaatccttttatatttatctttggatagatatgaggggacctcaaaaagtttgtggaaaaatggaattaaaagataaaaatacaaaatataaactttattctcaatataagctctatcgagttcaagaaaattttgtaagcaataatgctagccatttagtccactcctaaagaagtcctgtaaccatatcagtatgtgcagtcttttttttgcattattaattaaagaaaaatgggtgccctttaaatttttttttaaggttcggaaacaaaaagaagtcagaaggagccaatcagcactgtttggtggatgcccagtgatttcccattgaaaccctagcaaaactgcccatttgagaggaatgagcaggaaccttgtggtggtgaaagtcaacaagattccttgagcattccaaaaagcttttgtcatgaccttcgctcttgatgggtttgcttttgctctaactggatcactttcatctcttggtacccattgctttgtttttttctttgtcttcaggatcacactggtaaagtcatgtttcatctcctgttaaaatttttcaaagaaattccacatgctcttgttgccacttgtttaaaatttgcattgaaggcactgcttttgtctgtggccgatctgagcacaatggttttagtacccatcaagcagaaagtttgttcaacttaaatttttttgttagatttgtatacgctgaatgaatttatatgtctatggtattggctgttgtttgtgctgttaatcattggatctctacatttaggttacaaacgggatgaattttttcctaaaaaatttatgttgatagtctgctactgcaggcttcatcttcaatatcatcttgtcccttcttacaatgaattatcctatttataaactgctgatttcttaggggcattgtttctataaagttttcgtaaagcaccagtgattttaccattcttccactcaaacttcaccataaatttgatgttttgttcttgcttcaattttagcagaattcatgttgctctgttagggtgcttttcaaaccaatgtcttatccttcttagtgcctcaaactacatcttgttcagatgtgttataacaagttagtaccagcttattttggtgcaaaattttattgaaattcatgcatagtgttttcataaaacatatgttacacgaactttttgaagatcccttgtacctgtagtccaaaattcagaagttactaaagaatgtaaaataaaaataagtcttcttcctatgcttgttttcacttacccagtttccttcctcatcttcctgatggattaaaaaaaaaatgacatgatctaaaaaatatgatctattttaggtctaaggacatgtataggttgaaagtgaaaggactgaaagagttatcacatgcaaatagtaactaaaagagagctgggacagtttgctaatatcagacaagatagatttttagttaaaaactcttataaacacttaagaggctgggtgtggtggctcatgcctgtaatcccagcactttgagaggctgaggtgtgtggatcacttgaggtaaaacttcatctttactaaaaatacaaagattagccgggcatggtggtgtgtgcctgtaatcccagctactcaggaagctgaggcagaagaattgcttgaaccagggagacggaggttgcagtgagccgagatcaagccattgcactccaccctgggcgacagaacaagactgcctcaaaaacaaaaaacaaaaaacaaaaaaaaccctcttataagagacaaggaagagcattaaatagaagagtcgatacaccaagaagatataacaattataaacatgtatgtactgaacatcagagctccaaataacctgaagcaaatattgacctcattgagcaacagacagatctaaaataataactggagactttaatatgccactttcaataatgggtagaacaactagacagatctagtctagttagaagtagagtggaattataggcatggtggctcacgcctataatcctaacattttacgagactgaagtgggaggattgctggagctcaggagtttgagaccagcttggataaatagtgagaccttgtctctactaaaaattaaaaaattagctgggcatgttattgcacccctgttgtcccagctacttggaaggctgaggtgggaagaggaagattgtttgaacccaggagtttgaggttgcaatgaactgtgatcatgtccttgcactctagtctgggcaacagagtaagaccttgtctcccccactaactccactgcccccccccaaaaaataaaaaaaacaaaacaaaacaaagaaataaaagactttaacaccgctgtaaaccagttgggcctaataggcatgtatactccactcagtagctatttttctcaagtgcacatagaacattcttcaggatagaccatatactatgccataaaattagtcttaataaatttaaaaagattgaaattacacaaagtgccttttctaatcacaatggaatgaaagtggaaatatgagaatgagaactggaaaattcacaaatatgtggaaattaacacacttaaccattgggtcaaaaatcacaaggtaaattagaaaatatcgagacaaaaacacagcataccaaaacttaggggttgcaatatatatatcacttcatacaattggcttttcaaaatcagttaagacaaaaggtaaacatttatataattacaggtactctttcttttttgttgggcggtggtgggtggtacgtggatttgaattgctgtttgaggtcacttgcttccaggcttttgcatttcttttaagggggatcttccaggaacaaaatctgttttattgctttttaaaatctgagaaggtctgtttcgccttcagttttgaaatgtagttttgctagatataagattggttgacaggttttttttttcttttatttgaacactttgattatgttatctcattgttttctgtctgccattgtttctgcttagaagtcagctgctaatcttactgggttccattttaaaataatgagtcgtttggttcttgctgcttttaagattttctccttgactttgactttcaacatttttactgtgaggcatctgtctggatgtccttgcctttctctgagttaaaatacattgagtttcctggatgtgtggtttattgtcttcagtacatttgtggagttttcagccattatttctttgaattttttttcccctctaccactttttctcctctccttttagagtaccaaactgtgcctattggtgtcccacatagctctaagattttccaatttactgttgagcccccccaccctttatttttaaatttccattattgtaattttcagcttcataatttttatttggttcttttttgtagtttacatctcttcattgatctctatctgaggtgatattgtcattatgctttcctttacttttattatttatttattctttttttagagacggagtctctctctgtcgcccaggctggagtgcagtggcgcgatctcggctcactgcaaactccgcctcccgggttcacgccattctcctgcctcagcctcccgagtagctggaactacaggtgcccgccaccacgcctggctaattttttatatttttattagagatggggtttcaccgtgttagccaggatggtctcaatctcctgaccttgtgatctgcccacctcggcctcccaaagtgctgggattacaggcgtgagccaccgtgcctggcctgcgttttacatttaagtctatgatccattttgagttaatttttgtatgaagtgtgacctaaatttacaggttgaagttttttttttaagttttttgtctatggatgttcaattatttcagcaccatttattgaaaaggttatccttccattattgaattgctttggatatttgtaaacgtgtttagggaaaaactctcctcaaactgtgtttttcctctactgtcataccacagcaataatcaacacagaagaagacttctgtgatcacacacatggggttgttttccccacacaccaagcagcagacagcagcctgagtgtcctctaattcagttcgacactatctccgggtcattggtgtcagatcccacaggttgagggctccgtacctaagactactcccctaacacacacaccagtcataagtcggggcctccagaacttctgaccaaccagcttcaagttggggctcccatggccccctctttgggttcattaatttgctggactggcgcacagaactcagggaaacacttatttacgcttactagtttattataaagaatattgcaaaggttacagatgaagggacgtgcagggctacgtatgggggaaggggtgtgaaactttcatgtcctccctggatgctccacccaccaggaacctctgtgtgttcagctgtcttgaagttcactgaaccctgtcctcttgggtttttatggaagcttcatgacatcaacattcctaccccaagggcatagatgggaccctacatgagagagtcttaagacccgcaatcaaaaagtcaggggaacattaagtgtggaaagcaggcaggagaaagaggcctgcctctgaggcctaacacttccaacattataatgaaagactggaataaaggctatgggagttaaggccaggaaccatggaagaagaccaatagatatcatagcaccacaataaaataacaggcatacttgtgtgggtcatttctgggttctctgttctgttctgttactctatgtgtgtattcttttgctcataccaacacagtcttgactagctatgtagttggccttcagtgaggtctttttcgattgttttagctattttggagctttgccttccatgtaagttttataatgagtttttatctacagaaatccttgctgggatcttgataggaattgtattaacctatagaacaatttggacagaattgacatctttactgagtcagtatattgctgagaacatagtataacatattgctatttgtttagttttttttttttttttttttgagaaatggggtctcatgctgttgtccaggctggagtgcagtggtgtcatcatagttcactgcagcctcaaactcctggactcaagcagttctctcaccacattctcttcagtacctagactacacaggtgcatgccactaaactcagctaacttttaatttttttttagtgacatggtcttgttgcccaggctggtctcaaactcctggtcttgaagtgatcctcttgcctcagtctcttgtatttgtttagatttctgatttatttcatcagcattttggaatttttagcatatagggcctatacgtattttgctaagcgtatacttaaagtatttaattttctatggagcagttgtaaatggtattgttaaaaatttcagtttctgcgtgtttattgttaatatatagaaaagtaaatgatggctgggcacagtggcttatacctttaatcccagcactttgggaggccaacgtgagaggatcacttgagttcaggagttcaagaccagcctgggcaacatagcgagactccatcactacaagaaattaaaaaaaaaaaaaaaaggccaggtatggtggtgtgcacctgtaatcctaggtacttggcaggtggagggcaggaggatccgttgagcccaggagttcaaggttacagtgaggtatgattgcaccattgcactccagcctgggcaacagagtgagaccctgtgtctctagaaagaaaagagaagtgattgatatttgagtgttgatcttgaattcttcctgaactcattatttctagaatttttttttgtagatcctcagtaacattttaagatttgttttgaagtgttttatcttttttctattgtgatgtattcttatttcataatgaccaaatattgtggggttttaggatgaaggctgttgaaatccatatatcaattttatgccccatcacattgatgatttttcttatttataattttttctttagttgattctcctggattttatagataaaagagtcatataatctgcaaatagttacagtttacctttttttcccccaatttttatactcatttttttccctcatttaattatctaagacttccaagatagagttaaataatgctggtaataccattttctggctttagactcttagacttgtttgctttttttttttttcttcaaggcattgcatgctagtaggagttcattttatttcagtaggtagaatgcaaatagagtaaaacatatagtaaatttcagttctctagtgtgttctagtattggggtgttctgttttgttaaatttatgggaacctgaaggttgacataaatacttttacttcaacagttctactgttgaaagtcaatattataggccttcctgctttctgtagagtggccaatatttgatttctttgtagattttgcagcactttattttcttcattcttattttatcatacttgtcaatttagaaaacagtattgataggaatgtatattgatctcaggactttactggagagttgattttcttctttttattttaaggaggatccttaaaaattattggggaactcttatttgttgtcatttgatttcttcttctgagatgaggattacaaaacaattctaactaatggagcttttcagtttttcttttttattacgagatggggtctttgctttgttactcaggctggagtgtagtaatgccatcagagctcagtgcaacctcgaactcctgggcttaagtgatcctcccacttcagcctcttgagtagctagtactacagccatgagccacgtgccaccacacccggctaattaaaaaaatttttttttttgtagaggtgggtctcactttgttgcccaagctggtttcgaactgctgggctcaagtgatcctcctgccacagctatccaaagtgctgggattacaggtgtgagccactgtgcccgcctgagcgtgcgtttatagtctcataaatgagcaacattcctccatatatagctcctgctgatgaaaagtttggtgtgctgatatttttcttttcttttctttctttcttttttttttttgagatggagttgccaggctggagtgcagtggcatgatctcggctcactgcagcctctgcctcccgggttcgagcaattctcctgcctcagcctcctaagtagctgggattacaggcatgcgccaccacacccagctaattttttgtatttttagtagagactaattttttgtatttttagtagagggtttcaccatgttggccaggatggtctagatcttctgaccttgtgatctgcccgtctcggccccccaaagtgctgggattacaggcgtgagccactgctcctggcctctttttttttttttttttttttaagataatgtctcactctgttgcccaggctggagtgcagtggcgtgatcttggctcactgcaaactccccctcccaggctcaagtaatcctctcaccttggcctccctaagtgctgggattacaggcatgagccactgtgcctagcctagtgtggtgatatttcaagaacactttacaaagctttattctacttgctacagcttcagcatcaagatcctgactctgtagttttcttgtgatataaaccaactacatttagggttcattttcttatcttttactcaccccagtcttaattttgttgcacagtggaaagcacatgaacttcatgaggttcagaaactgggtataacacagctgcttctctttgaatcctaatgaccttgaataagttatccaaactctaatcctcaaaatggaggataataatacctcagaaggctatatttaaagatgctgtgaagtaccacatgtaaattatctggtacagaatttacctttgttgattctcagtaaatgtttgctgcctctcccctgaaacaaaatgaaatggtgtgtgtgaatagctttgtgagctttaaataatataattaaatagaagccattattctgtttcctctttcagaaatgggcattaataacataatttataatctcaatagtatcatgtgcttggcccaggtacatgcccaggtttggtattctttagatttttgtttcttcttgccctacatacttcgaaagcccttggagaaatcctacagcaaattcacatgatttatgaaaccaacagttgactaattcttactctgtttctttgactaattcttagtctatgtatccacatatatgaaactaaatccacctgttaccttctttttatggtatctgggttctcctactttgccctactcctttccccactaagaaccttgaacctcatggaatttattagctagcatataccctggtctcttcctgttttggtatttctggttggattgactgtgataatccttattggatattcaactaattgtctaaaataagcttacatatcatgagttaagaggtatagtgccaaggacttgaaaagcttgaactcctaggacttacttgctgcagtccagtttagagagaaattaatgatttctgaaaatgttcttagcactcaagagcattgagatattatcaaatattcattaaacatttgtgtgcgtagcactttctgggaacccagatggatgtaaaacgtgtatgctttggtcctgagtttcttccttccagggtagtctagttagagttcagaataactcaaaaaagttgaaatgtaagacattaagccttttgaatacaaaatattatggataatatgtgcttataaatttgaagactttgtcctgtttattttttataaagtatggtgaattttgttttcctttacctgtgaactagaaataaaaataccctgtggggtcattgtgatgtttaaatgagattggatatgtaaagcccagttataatatataaacttacatatattctttctaatacatacaattagtatttagaattctaatttctaattgtatactattttttaaattaaacaagtaatatgatacatggttaaaaaaaatgcaggcagaacaaagtaagtattcttctcacttcagacccgaggttcttccatatgttcttctgtaccaagagtatggttttaaatatttttcattaattattctagaacatatgcacttacatatacttacatatttttataacatataagtgggaacatattattcatgttgtcttgtatcctgtgtttttcatttaaaaagagatttttggctatattccctgtcaacaaatatagcttttaagtacattattcttttattattgattgctgagtattctgtcgtttggatatgtgataatataactgatattttccccagcactcctttaatgcattttttcaaaactttcatcttcttaggatcaactcactatcctgaaggtccattctcccaagaagaggggacagaaagacagatctatttgtaagaaaggcttgggtatcccatgaacgagccaacagaaaaccgattggggtgcagcaggactccagagccagatataaggctcagaaaagggcaccaactggatggtacacgaagaggtgataatgacagccaccaaggagatttggagcccattttagaggcatctgttctatcttcccatcataaaaaagtaagtcaaattggaagaatgataaacaaaggaaaaaatgtggaggaagtggcagagggaggatatggggaaaaaaaagtgggggtccattgctggagggaggatggggtggtaagaaggaaacttgcacatttgtatttttttcttctggataacttgaagtctcttatctttattattattattactactactactactactactactactactactactactactactactattgagacggagtctcgctctgtcacccaggcggagtgcagtagcatgatctcggctcgctgcaacctccgcctcctgggttcaagcgattctcctgcctcgactacaggtacatgccaccatgccaggctaatttttgtagctttagcagagatgaggttttgccatgttggccaggctggtctcgaactccttacctcaggtatcctcccacctcggcctcccaaagtgctgggattacaggcatgagtcatcgtgcccagcttcttattcttaaattagcttagatattatttgattttgattttatggttaatgtctttctgctgactcttttttttttaatttcggagacggagtcttgttctgtcgccaggctggagtgcagtggtgcgatctcagctcactgcaacctctaccttccaggttcaagtgattatcctgcctcagcctcccgagtagctgggattacaggcacctgccaccacgcctggctactttttgtatttttagtagagatggagtttcaccatgttggccaggatggtctccatctcaacctccttatccacctgcctcaacctcccagagtgctgggattatagacgtgaacgactgcgcccggcctctgctgactcttaaaagcgtacttaacctgctttatcttgttgaagggagaagacgtggtctatgggtaccatataatctaatttaaccatactttagctatgacgttatctgttatttttggagaaattcgaattgcgtgttagagctacttttccagaaattgtttacctccagtgtataccttgggagtcagtagagttctgaaagctgaagcagattttattctttcatttcctagagaaaagagcaacctttaggcaactatacctatagagcagaagtatcagaaagtcattgttacagacattttgctatgtgctttggaacttccaaattatatagcatgtgtttaatgttttattactagtacagtgtttctcaaactttgggttgtacctattagtgggttttgaaattaattgtattataaagtgttaaaaattaaagatcaaaatagaataaaaacattgaagtgtgtatatagtaagtaatacacatttaaattgttttagttttatatagttatgtatataaatatgaataataatatgaaatacatttcttactacgggttgtggttaacattttgaaagcttctactgggaacagcagctgggggaagagtgtttcaaggtagaggaaacaagctagtgccaaggtccaaaggtgggaatatgcttatcttccttgccttcccctcctgcctcccaccaccacctgcgcctgcttgtatcatctttctcttctttcatcttctcctttattttattattataattatattttttttgagatagagtctcgctctgttgcccaggctggagtgcagtggcaccatctcggctcactgcaacctccgcctcccaggttcaagtgattctcctgtctcagccttccgagtagctgggactacaggcctgcgcaaccacatccggctaatttttgtatttttagtagagacggggtttcaccatgttggccaggctggtcttaaactcctgacttcaagtgatccgccaccttggccccgcaaattgctgagattacaagcgtgagccactgtgcctggccttctttttttttttttttttttaaaggaccaaaagaaggccagtgtagttagagcatgaggaactagagggaagagtgaaatgagataaggtcagtgggccagtcagagaacagattatacaggtttattcagggaatttaaattttattttaataaaattgcctggcacgtgggcagcttttggggtgtttaaacatggagacatttgtgtttttaaaagtatgactctggttgtggaatggattatttgaagacagaatggaagagggacccaggtgttagaatatagatgagagatgatgtcacttgaaacagcatggtgggagcagcagaaatggagggaaatggttggattctggatatattttgtgggtcaaatcaacaagacttcctgatggattagctgtagagtgaggaaaatagaggaatcaaggctgattctctgagctgaactgaaatggtaaagcaagagggaagagcgggtttaaggtagggaaatcaagagtggtgttttggaaatgtgagattcaagattcttgttagatatcttagaagatatattgaggagtcagtaactgtgtcacgattagatttttatttacctctgtttcttaactatttttaaaactgaactgttattaaggaatctaaggaattttcaagcttgaaagaatttcaggctagccaatttgagattcataatggcaataaaattctgtttatcatgctacattatttttaattacgtacaaagatctaacatgtcacccagggaccatttcacccactgctctgtttggtcgccagtcttttgtctttcttttcagcaatggtgaggcagatacccttttcctcagggaagagaagtctgtggtttgttgcccttgccaataacaaaaatgttggaaagttgagtggcaaagctgtttccattggcatctttcatgtgaaccacatcaaaagatccagagtgcctctctctgttggttatcacaccagttcttcccaggttagcacctacagtcaccatgcataggttaccattgtcaaacttgatgaaattggtaatcttgccaatctccaaatctgtctgaatggtatcattcaccttgatgagggaatcagggtagcggatggtgccgggcatcatgagtcaccagatgagggattccttttgtgcccccagagatttttctcactttgtatttggcctcctcaggtgtaataggatgtacagcaaagcgacccttggtgtcatagatgagacggaaattctcccccgtcttgtcaatgctgatgacatccatgaatccagcagggtaggttatatgagttcggaccttgccatcgatcttaatgaacctctgcatgtaatcttctttacttcatctcctgtcagggcttacttaagtctgttccttaggaatatgatggggagacactctctcaacttgtggggactagtggatggacgaggagcaaacacaccggtcaatttatccagcatccaatgctttggagctgctgcccacttcagatgcttcttgggaccacgcgccatggctgcgttaggcagggaaagcaaaatatatttttagagtccagaacttgcccacccaatcacttttgtgtcagatttacattaattgatttttttatgtgttgtaatactatatatttttgtttcctgtcttcactggttgaagtggtatcttctgtaaataatatttgtgttttaatttcactaataaataattaggtggatgggatttttcccatccttatgaatgaactcatttattgccctgaaagcctctctttgattttccttttatcttgagctaatgcaaaagttagcagtatgaacaaaagaaccattttttctttcatttgttttaatagatttttctatgaggagccaaggaaagaaaggatccataggttgtggaatgttagagctgaaaaggccttccaaaatcatcttgtttgccttcctcaatttaccagtgaggaaattgtgtttttattcctaatgaaaatttgcgtgtgtatgttgtttctctttggagttgaagttgtttgctgaaaaagagaatctatatgcagtactgtcccttgctgccaatttcacggttaatgtatactacacatgggagctagaaaagaagggggatagataagagttatagagaactgcagttttgagactgaaattaaaggcaaaatattgatatcttcacatgatttgaggagttccatatgctgattttttttgttgtttggagaaaggactcatttcttaaaattttcagttttgaagttgaaacatctgaactgtagactgcaaccttctggataattctagtaatatcttaattgtgtcactttcacagacattatctcatttgcttctcaaaactatactctgaaatagagcagatatgatttctttttgaaagtagacgaaaataggatttagaaaggttagtttaggaatttgcccaaggtaacatgacaagatgagagcatgtgctaagtcttctgactgctctttctgccgaacaaagctgcctttatgagaattggcaccagattgacttaatgtttctgaattccaaactcagtccctgagttgtttatattgacctagaggttagattcaacagttggtcttgttttttggcagaagttgttgcagacctcgttttctgttaggccagggtaattttgcttctcagatgtgttctaactttgtaatgggttttctgtttattttgaaagcaaatagaacgaatatatcatagtacagtcattcacatttctaacaattttgcatcattttctttgtttttttccctcttttagaagatgcagatcttaactaaattgcttaggcttcatttaaaacattatgggagcccttaggctctcttaagttgtggagattggcattcacatgccaatcatgaataaggatcaaagtaaaataggaaaagcagcaggggtgtctcgttttttattaagtggaattctgaatttataaagtaagatttaaggtaattatgtgcattacagactgtggtatctttttgtacaagtctttatttaccgccaggtttctttaaatcaataaacctattaggagtgcccacctgatcacatattgccctttatagaaaactacaaatgccaggattttattccaggcctgctgaatcagaatctttgggtcctgggtatattttttttgtttcacaagtaagatgctcactactgtttaaaaacctccttcctggccaggcgcagtggctcacacctgtaatgtcagcactttgggaggccgaggcagacagatcatgaggtcaggagtttgagaccagcgtgtccatcatgctgaaaccccgtctctacgaaaaatacaaaaattagctgggtgtggtggcaggcacctataatcccggctactttgggaggctgaggtaggagaatcgcttgaacccaggaggtggaggttgcagtgaaccgagattgcaccgctgcactccagcctgggtgacagagtaagactctgtctcggggaaaacaaaacaaacaaaacaaaacaaaaaaaacaacctccttcccgtaggagatagttaaggaaaacaaataaacaaaacccaaaccagtgcctaattttttttgatatttatttattttaaataagtattttatttaaaatagttaatgtattaaaaacttcttagcaacatgctttgcaaagaaatcttaacacattctctgaagattgttagggaaataaagttagatttatttactgtttgttaaaggaaaatatcacatttactcacagttgactcttaaaacatttttttttgagataaattttacataccataaaattaacccatttaaagtgtacaatttaatggttttattagtcacagggttgtacaaccatcacttcattctaactttaggacatttttatcatcccctaaagaaaccttaacccatttgccatcacttcccatctctacccccaaccctaggcaacctctagtctactttctgtctctatggctttgtctaatctggacttttaaataaatggaagtgtaaggcatgtggtgttttgtatctagcttcttttacttagcataatgtttttaagtttcatcaatattgtagtatgtaccagtacttcattcctctttatgactgaataatattccattgtatggatatacaggggttctttcttatttgtggtttcacattctgtggtttcagttacccatgggtcaactgagatctgaaaatattaaatggaaaattccagaaataatttctaagttttaaactttgtgctgatctgagtggtgtgatgaaatctctcaccttccttgccctgtcctgccttacccaggaaatgaatcatccctttgtccagcatatccatgctgcagatggctggctacttagttacttagtagccttctggttataaggtcacagatcacaagaagcgtgagtgcaatacactaagatattttgagtgagagagagagcatattcacataacttttttttacagtatattgttataattgttctattttattattggttgttaatctcttactgtacctaatttataaattaaactatgttataggtatgtatatataggaaaaaacacagcatatatagggtccagcagtatccatgatttcaggcatccactaggggtcttagaacatatttcctgtggatggggggaaactactgtactatagtttatccaataatgagttgatggacactggattgtttccacttttcgactgttgcaaataatgttgctattatcattcatgtagaagcttttgtatggatgtgttttcatttctgttgggtatatactcaggagggatttttctgggttatatgttaactctgtgtttaacattttgagaaactaccagactgtttttcatagcagctgcaccagtttatattcccaccaacaatgtgtgggggttccactttctccacacccacactaactattgttactatattttgaatataactatcctagtgggtgtgaaacgatatttcatcatagtcagttagctcttgagcttttatgaagagacctgcctaggttaactttagctcaggattttaagtattaacctctttgaggaatttggaaatagaagctaaattcaaacttactataaaactcaagttgtgtcaatttcacttcaaatggtctcctcatcatctgttctgccattactttaatctaagccaccaccatcttccatctggaatacacctgacaggacttctgttttgtttcttgtgctgtccaatatattcttcgcacagttcctagagagagctcttaaaaatataaagtttattatgccattcccatttaagaaattcatttgtttcccattgcacttatggtacaatcccaaattctttctataaccttcgggatcctgcgtgatctcactgctgccctatctggccaacttcatttcaggacattcttctccatgttcattgagctgcagccgcattggccttatttcagtttcttaaacaggctgggctctttccagcttcagagccttcaaacatggctctttcctctgcctggagtgttgttacacccacacctctggctaactgctgctcatccttcagtattcaggttaagttctctacttccacttcattctatttcaagtttcccattgttatctttaattaccttttattttccattacaatgcgtatcatgacatatatttgtgtgacatatatttatgtttataaacatatatataaaacatattttataaaaatttataattgtttaataattgtttttgcttgtagactcttcagttccatggtgtaagaatcttgcctttttgttttcctctgatatcctcagagtctaacctggtctttgagtggcatataacaaatagttgttgaatgaatttatcagcaacaaatataaagttctgtgtctaagtggtttcaagtagtttctttaaagagatgtacaaaaggagcacgtggggacgtatactatttagatgcttagggaagcttaactaatctacttctttaatcttagaataaatgcattcttatcaggagaataatgatagttcacttttattgatcttttacaatggatgaagtaattttttgagctcatttgaatatgttattcgttcctaaagtagccttttagaatagatattattagaatctgttttacagatgaggaaactgaggtttcatgtgaatgtatgtgtattgtacactgggactcttgtcatggggatgatatactttttctcttcaaaggggcagggttttcttttcattggtttattgtaaaaagtgcgatatggcaatttcaatggttatttgcctttaatacattttaaaatacattatttccatagatgataagaattgtggaaaaagagaccctgtgataggtgatagtgatccattgtagttgtggaattggctctcaagtaacattaaataaaatacctgtgtgtacagttagattacgtttacctgtgcataggaatcccttccttatttgaggtgttggagaggttagtctctttggttccttatgaagcaggatggatttttagaaagagtattagaaactaaatactttaaaattgaaatctctgcagtaggaatctgtatttttcacaagttcccctatgtgattgtgaggcacacttaatgatggagagccagtgatcacagttttcttttcataatgcatatacggaagttccagaccggtaggaattacatcagggtaaagctgggcctagaggaggtgggtgtttcttcttttgtaaatgcagtatgtaatagtatgcctcttgtctcacagggttgtaatgataataacttagtgcatgtaaagcagtggtaacactgcctggtacagagaaagcacttaataaatgttagatgtagcatatcattggaggtgtatatttgtgtcaaaaagaaaagacttcctctgttatatcaaaaggacaaagttaatctttctttatttctttttgtttttattagatttgtatttatcttataaaaaatagattggtggttatctgtctccactcacttgaacatgtttattctctgcatccaaaatagtccaggaatggctggacctggtggctcatgcctgtaatcctaacaactttgagaggctgaggcgggcagatcgcttgagcccgggagtttgagaccagcctggacaacatagcgaaaccttatctctgcaaagaatacaaaaattatccgggcctggtggcatgtacctgtagtcccagctacttgggaggctgagatgggagaatcacttgagcccaggaattcgaggctgcagtgagccatgattgtgccactgcactccagccttggcaacagagtaagaccctggctcaaaacaaaaaattacacatataatgtatattccaagaatattgtatttgtgcaatatttaagtaaagaaatgaatgattaaacctgagttttagtcctggctgtactgccagccagatgtgtgactttggccaattttcttgatttctttgggtttcattttccttacattaaaatggagatgtttaactgggtgataaacatctaagattttcttatcctcaaaatttctgtggtccccgacactatattaatacttttttgtaatgggataaaaaatgagtgagggtcatagtttacttttttcataaatcaatccagttaggtatctttgcttaagcagccctgtttaatgccttctttgatcacttttatcgtgcctggttttatttagtgaagccatttctgtggactagtaaataattttgaaggaaatagtttggtaaaaggaaaaaagcagggttttgtactcagagtgacctaaatttgtatccctgatctcttctgtatcattatttttcaaacttctgtgtaagaggacccttcaaagaaaaacaacaaaaaccttattaaagtattaaacacacacacacacacacacacacacacacacgtagatatattgtaagtggatagtctgataaacttttataaactagacactcctgtataaccagcactcctggaggtctctttattacctcctcatagtaactgcctattcccactcccccaaaggtaaccattaccctgacttctaatggcatggattaattttgcttgtttttatactttatctcattgtaatcattccatatgtactcttgtatctggcttcttttgctcagtatgatgtttgtgaaattcatctgtattttctgggtatagctgtagattattcatttttattggtctgtgttatttctttatattactgtaccaatttttattttatttatatgaaggcaatctgggttgtatctaacttggggctattataaatagtgttgctatgaacattccagtacttgttttttagtgaacaaatgtatgcacttccattagctacatacctgtgagtggaattactgcgttaggaagatgatcccattctccctttaaacatcaaaaatatttaacagacctcactacatgatactcacattttaaatattagtttgaaaaaaatacatgatagaaattggctatgaattcagttcatcttaagtgaatttgtattttgtgattcacgcagcacctacatgattttaaattagttacacctactttatgaatatgagacatttattcagtctacagattacatttaatgtgcatatacacttgcataatttttgaagtaattagccaagatagtggtgttaggttgggaattaaggtactacttaaagctgtgtactttctcttagcttctgtttcttcatttaccatgtttgatctagatttccacttaataaaatgggaatagaaatatactcatttcagaaaattgttatgagggtttgattaatttgtaaaaagcacttagtacaatgtctggcatgtagtgtataataaataataattgttactctgtccttcctttataatagtacttgactactccataactgtagtaagtcctgataaacacatcttgaagttatccacatatctcagccacccttgatttcaacagccttttcataaaggtgatttctaaatctatcatcttagtgcagtcttcttttttttcccccttttttcttctatagccttcaaatgagaagtgctttcttctttattggacttgtaaccatttcaaattgaactaatgttaaactaaggttaactgtccactcccccatcagcttttccttctattctaattttctaatcctaatttctcttaaatagtaccaactttctttcaggtactcagacttggttcctagactgtcaagagtggaagggatcccatcagtcatttagtttagtagacacatggatgcttttaatccttctacaaatgtttttagtttctttaccatgtgttggcactttggacttgagaagacatagtttttgcccttaagcagcatgtagtttagttggagagacaggtaaatcagtacatttgtggtgagtgctgtgtggttttatgcacagattactatggtagtaactccagttgcaaatggatttagctaagaaaggactaaaggacattttagacaaagggaacagcagatgtgaagattcagaagcagatgtaatacgatgcctgtagggaatagtaataataatagctaatgtttattgagtacacactggcacatactgatgtctgaacctgaaaaatttctagcatagctggaatgaaaggtcaaggtgtatagtggggtggaggatgtggtagtaagtaagacaagacacctggggcctgaaatctaggtctttagcctcctggtccagtattatgccatcattcaaatttctttttgcaatttatatcagtttcaaattttgtgtaatgttgtgtactggttgaaatgattagaccattgcccctaggcctctgaccctaaagcctggactcttttaactatatttgctaaagtatatagttaggaggcattctgtaagcttttggacaaaaatataagagaggcatttcaaaccaggttgtcctgatctttatttttatggctcccctctgcctccctgctccctttctgagctttcaagatattaggttgaataaagagatcagcctttatggttgtcctacttttcttctgctccagattcttctttataaattgggagataaggaacttttattcattcaaccttcatattctgttaaactggtttactttaaccaaaatcaagaatgccactatataatagtttattgtaagaaaactaacttgtaaatagactcttgtcaatcacttcattttattttcatggagaagcctgcttaagaaattcaattaagattagaggccgggtgtggtggctcacgcccgtaatcccagcacttcgggaagctgaggcgggtggatcacctgaggtcaggagttcgagaccagcctggccaacatggtgaaaccccgtctctactaaaaatataaataattagccgggcgtggtggcaggcgcctataatcccagctactcgggaggctgaggcaggagaatcgcttgaacccaggaggcagaggttgcagtgagccaagattgcgccatcacactccagcctggaggacaagagcaagacttcgtcaaaaaaaaaaaaaaaaaaaaaaaaagatagaattgctatttgagaatttttaaaaaactgagtgattttaaagatcttttccattaagctttgatggaagtcagagtatatatatttctaggcaggccaaaatgaatattcatatagactagatattatagttattcttttggagaatactgggtatacatccatcaatacttattcttcagctatcatttttttttgtagatacctttgtttccacaaacctctttgttttcatttgttagtaatatcagcttgctaatgcccttgctacacatttccaatttctaaagttaaggctggcttcaacctgtcaacttgtaaaaagaccataaaaatcacctgttgatcaaactaagccaggtttattacatactccagtaagggtgaacactgccctaaagagtcctagaagtatttcagaaagaaagtattggatatggatatttacagggttttggggtctggtttcaagtagtttaaggagattttacttggccctgttctgagtttgtgatagaattcttagaccaagtcttgataaacaaatatttgtttggtaagtgagcatgtttgcccagttgagcaatgattaaggatctaattgagcagtctgttgttcagttaaatgaatttttgagaaattcctaaagctaatgatgaagttatttactggtttgcattcttaccttcctgggcaagaatttccgagaacaaataataaagttctgttgatataggggccttagatcttagtccttataattaagctgtgtgaatgcagaaggtctcagttctcaatgacatttataaaggcagacagattttaatcaaaggtttactgtctccagccctggatattgtaccatttcttgacctttttcatctaattgtggatggtagatagtatcatgattgtgagtctttgtggcacttcttgtacttccactaacatttaaatacctcatttatgttttgctactttcctaaatgcccccgaagctctagatgaactagtctctttttctcaacatggcacttttgcttttccctcttggttattttcctttatctcatctttgcaaaatttttattcttcatgcaggctttcctgagcctcatcatgtttatctttgaactagctatacctttttttctatgaagtcatttatttggctaatttagcactttattagattgtaagtccatggaagacagagactatgttttattcatcttatttttaatcatctaataaatatttgttaaactgaattactctgttgaattgagttaggccaaaaccaataaatacccaataggtttgtgaattattttcatattagactgatcttaaagaccttttatctttatcactatggttctctgtctttccaaaggtaaagtgtactctggaaaattctcattttagattgcagtttggacagatcatcaaagtcaaatgttactatgttttgaaatacagtcagcaaagttctcacagatggactttactgaaaacagatggcgggaggggggagatatttcaaaatgtctttgtgctcatttcaaagcattgacctctacaagtttaaatatagttattcatagaaaaacctggagaattaagttttatgcctttattcttggcagctttggttttgtttaatctgtcacaatgtcatgtttcttgtcataattttgttgagggtgtggtaaggagtatgacaacagaagcagaacatagcatgtatgtgatcattgaatatagatacaatttattgttcagagaggtatctttgggtactgtggtttggtggctgttggatggaaattagaaatagtaatttcactcataataaaatagttatttggacttaatattggaagacagagactattttaaatatagcgtctagcatagtctttccaagccatcagtatgtgacatatggtataccctagggaaagtatttttttatcctgttttagccagtgtttaactctgccactgtattcctactttatcctctctctaacccagggaactgcatttaccaccacccacaccacgtggataccgcattagcaactccgttaatacttgaacacaataaatagttaattatggtctagtattttaatttatcctttgagaacatatttgtgaatattcctttatacaagggtaggaattggtcatagtacttgataaatcaacagatttattaatactttgcccttctgaaggaaatatttttgtttttgtttgtttctcttgttgcccaggctggagtgcaatggcacgatctcggctcactgcaacccccacctcctgggttcaagcgattctcctgtctcaagcctgctgagtatctgggattacaggcgctcgccacctcacctggctgattcttgtatttgtagtagagacagggtttcaccacgttggccaggctggtctcgacctcctgacctcaggtggtgcacccacgttggcctcccaaagtgctgggattacagatgtgcgacactgtgcctggccggaaataattcttttataaagattctgaatcttgctgatatattgggatggatggaaatatttatctttactaaagtaatttttttttttttttgagacagggtgtagctctgttgcccaggctggaatgcaatggcaagatcacagctcattgcaacctcgacctcctggggctcaaggagtcctccccctcagcctcttgagtagctgggactgcagacaggcacgcaacaccactcctggctaatttttatattttttgtagagatggggttttaccatgttgcccaggctggtctcgagctcctgggctcaagccatcctcctgtcttggccgctatcagtagtcttatatgtaaccagttgcacttagcctgataatcagaattactgattgtgagtaatctcactgattacttactgagtcacattgattcggagtgcagtttcatcatgtttacttaatggtagtatacattagccatattgtgttttatgtacatatctgtagttttgaaatggctgacagtaatttgtatgagaataaagactctttgtttctgtttctttggagaatcaaatacatgttgaatatgtgcaaatatctgagctattttatataacataattttatattttaaaatcggtctgctataatcatttcaagctattgcccgttttcaggttttatataattttaatggaattttgtcattttaatttcgtgtttaattaggcacagccctcattaacttattgttgaaagtctcctagagcatttattaatttagccatcatgtaagtatgttccagaagccatggggaagaagacagacacttaaaaatctagtttaggaaatgatacgctaaaaatgtaaaattctattaaacgttaaataaaacagtattagtcagtaaaagagctgtaatcagatattactgtcaaatgagaggcacagacttaagtgctaaagaagtcagaagaagtagtcagaagaaacagaagtcagaagaagcaattgctgtggattagataggttatgcagtctctaatgaaaattggttcttggtggggtaggggatgaaaaactattaaatactactatggtttggaaccccccatagggccacagtacatagatagtatatctgtgttattaaaattcatggggatgaggaaagcaatctaaaatgtcccagggccgggtgcagtggctcatacctgtaatcccaacacactggaagctgaggcaggaggattgcttgaggccaggagttcaggaccagcctgagcaacacagacctcgtctctacacacacgcatgtgcacacaacagacacagtggtgcatacctgtagtgcacgcatgcacacacacgtgcgcacagtggtgtacacctgtagccccagctattcaggaggcttaggtggaggattgcttgaggccaggaggttgaggctgcagtgagccatgatcgcaccactgtactccaggagcctgggtgacagagcaagaccctggggtctcaaagaaaaacgctgtctcaaactaaaaaaaaaaaaaggtcccggggcttgtgggtagcagtaatgaaaggttgaggaacacgagtttaggatagttaaagaagggttttaaataggaactatgaaggttagtcattgaaggatagatggtaagttttaggtaaacaaagatggagaggttattttattcaagatggttttcttggagattatgtgtttttataaaatcagactgggtgtataacatgtctcacatgccgcatcatagcctctagttagtgtcttataaactgaagaataggaaaattaaaatctcagaatttccttttaaacaagatcaaatttaaagtgttgtagtagattacccattgcctctgtgttcattcttaccaagtagttttttgattagaggtagaggatttattcatttctttgtgcagtcatgttagcagtactgtttatctaaacagactcttggttacatacggaattatggttctcttgtgctgtcttattcattgagttaaagaattatagaatttttttagagatgtagaagactgtagagattgtttaaactcctccattgatacatagggagaaaatggacctgcagtgaagatccattgatgttagtggtggagctggcctcaccactttggtctcctgacttgcagtctactgcccttcttctcacaatatcattttatctaaaccagtatgaaccttggtctggaggcagaaattttaattttagtaatttatctctttagaccataccagattgtcacttctttttccatttctctagacaaatcagtcattttctccagaatgtggaaaggcagtgggttttggagtttgacttttccacttcagctttaaccttggacaacttacttaacttctttgcctaatgctcttatatgttaaatgggaatgagaatacttacttaataaggtggttgttaggattaagttagatgacctatatataaagccttttcctctgtgttggatatatttaaaaaatttattaaatgtttttctcccctttctttctctgccactagagtacttaccacattgtgtatcacagttagttattttatgtgtcccaggtgcgagtcttggaggttttccccttagtaatttctcaactttatgtaatattattatggggtctatgacataatttttaaaaaacaaaccagtaaagccaggaatttgctactgaaacagagatgctgctatagtagacagttaattcatctgtcagagaagaattgagtactagtgttcatttttaatactaataattctataataaactgacttactatattgttgattcttgtctaaggccttgttttgaccttaagaatttcattataattccacaagggaaatgtattgattgtgtcatttggctcccagtctctgtccttgactgtttctgtgattgccttgcagagctctgaggaacatgaatacagtgatgaagctcctcaggaagatgagggctttatgggcatgtcccctctcttacaagcccatcatgctatggaaaaaatggaagaatttgtttgtaaggtaagagtgcagtttcttgtcattgtcatgctatatatttagcccttccaaaacagaaacctttaaagagagtggtagtactatagcctttggtttgctttggggaagatctgtggcaagatggggacagaagtggtcttgggactctaatcctgttgaagacagaatacatttgttggcttgtagtagccatgataaagctctgataaaactaggatatccagctgggaaacaaacatttcctaatgtaggttaacagagtaaatgtcttattggtggtttagacaaacagaatgaaacatagtcagaagtggttgacgcctggagatatgaaaatgccctccgcaggtcccttaaatctgttctgctgagtgactgcaatggcaaatgtactgttctttctagggattctgtgagtaatcaggctactcttgagagagattttccagtttccatgtacatctggtattttcagttcagctggtatacattctgtctcttaaggtttttcttccagtccattgtcttgattccctaggacctattatgggattctttttttttttttttttttctaaaatagtaattagaattgagtttgcctgcttagagaaaatagacacactggtaaacttgtaggtacttgatcaatcagatcaataagggccctggcaggtagaatctaattaatgtaagtcgatcagtctgtgtgtatacgtatgtgtatgtaaattgatttattaaacatttctggagaactctttattatttagagagaaatttttgtgacacttagcttttcatggcttttatgaaaccaaaactttttatgtacatggtcttagatccaggagactaaatataggttttcttacattcacaataatgctggaaataattgggatcagtggacttagaatgctattactttggaccagggtatgaaaagagaacattgtgatctgaacatagacatcctttccaagactaagggtccagtgggttttcagggggagtttaaacatttatgggtctgtaatagaaaattttagccagtttgaaaatgagtgaagaaacaactgaacagaacaatagattcattctagtgatcactgtctcaggagaattagcacaaagggatacaaaagattattttgtttttaaaatgcaaatttggtgtgaagaaatctctcaaagttggcaaatttagtatctgggccattaggggattttaatatttagagatcagcggggtacggtagctgacttaactgcctttttgtcctctccctttctgaacagagaacaaagaataaaacaaataaaactctaaatttaacagcaaatcagagaataaatagggcaggacatttgtttgctacaaactatgatcttctgaaggaaaaggagtgttgtaaatatcccttagcagtttgttgatgacagcagaatgaagaggcatcctgttacacagtgttgtctggtgtggctcaaccctgccatggcagtgcaactgaccacacttgaaaaccagccgtgtctagtggcacagggattctatttcttggactggaagcaagtaaacagagacagtttaatatgaaatgtgctgtggataatttagtctttagagtttccacgtttgatcttggtcactcttttttcccctagacttcagtgtttgctatttaccagcataaccaggtccatgtgggaataggatgtatgctatggggagtttatttgtttttgaaatttatttattgttgagtctcgctctgtcgctaggctggagtgcagtggcacgatcttggctcactgcaacctccaactcccgggttcaagtgattctcctgcctcagcctcccaagtagctgggattacaggcatgtgccaccatgcccggctaatttttgtatttttagtagagatggggtttcaccatgttggccaggatggtctccatctcctgacctcgtgatctgcccgcctcggcctcccatagtgctgggattacaggcatgtgccaccatgcccggctaatttttgtatttttagtagagatggggtttcaccatgttggccaggatggtctccatctcctgacctcgtgatctgcccgcctcggcctcccatagtgctgggattacaggcgtgagccactgcgtccagctgtttttttaagaaatgtttttgtgcattttatggtctaatatatccacatacccagtggattattactaagatatttgaaggtaggcagtgtctttgtcaagcttataaataaatatgtctttggctaagccgtgatcttatttttttattctagggggtaaaagaggatacctatttgtgttggaatttggctgactgtatatgactccaggttgagaatgagggtgcagtgtctgttgaatgtgtcactaattgcagtgtaattaagaaagtatagctccttgtctcagtatttagaccactagaacatttgtttagaaataccagggaaaatcactctgaaattatatgcatatataaccaataagaaactcacatattctttttgaatgtaaaaaaggaacatatgactcttttgtctgtgtggggtagcagagtagcagtttggatacagcagagctgtgtgcacattgtgaattgccttgaagatgtaagggttgatttctgtcttcctctccctctcagaaaataaaattcccccaaagtaaggtaatagcaacattctgtaaggttaatctcaacaatagtggtcttttcccaaattcaaatttgatgcaaaaaattcagtatcttggtctctctctgacactaaaagaagcagactgcagaggttgacagacttccagcagttccaacagaagggatactggatccctgatcccctttctctttctctccataaagtcctgtggaagatctcatacagctacttttgtaagtggtagtactattagcagcagatgttaaaaatatagattagtcttttggcattattttattttattgtatgttagaagcaagcatagaatggccacattagcaccagtaaccacagaccccacgttttgagttggaaagtgagggaggagaggttttgtatggctgggggcaggaagagtagggaagaggcagggggctagtttggttagagtgttaagagggacacatctgttcttaaattgttagtcctcacttcctttggatgtggagatgttacacagaggattagttcttttacaaatatctgtaaggaagtaagagagcagagaacaagaaacaggttgtggagcaaaaggaatgggcacagtagctggtgagtaagtggcagtctttccatagagtagagagagagccaagtttccatggcttacccatattgtcacaagaccctgtagtgggtagggtggaggtcattgtctgaaggtgaatcttgctgatttgcagcctggattcttgctttatagggtaatagactggaagaactgctgagcccacattagctggaagataaaccatgtgcctgccatttttccttacatctttctgtctagcagaagcaagttagtcaagattaagtgatccttgtggtgtgagctctcggggctatgagacattaaaagaagcagagtgcagaggttgacagacttcctacagaggggatatcggattcctgatcccttttcctctagaatctgatgcgggagggaccatgaggaaacgggaggaaagggctctgagggactgaaagggtggttagggctgagcttctttaccatctctctgcagcatgagaaggaagatggattagtcactaactgtctgacaggagatagcccctggggatagtcatgtactggctttgagtagatgccagggattactaactaattcatgaaatgctttgtgtcattttgtttgatcttcatgatttccctatgaagtaactgaagcgctattttacaaatgaaggagctgcaggttggaggttttttaattaattaaatctcatatagctagttaatgccaggggtccacaattttctgactcctgtgctctttcttttctgcctcatcatatttgcatagtgggaagagaagaaagagtaagatggagggagagattttagcaaaaggcacaaaagtatgattctatgtaattctgtttttaccaataggaatattaaatataaaaatgttcatattaacaccacagggatttatcagaggtaaagtctgttatataggtaggatgaaggaggtcagtatgagtgagactttggaaccttcatcccaccattagccaggttgaaacaaagtttgaaaccactcttttagggcatttcctataaatttgtttactctttccttttcctaaggcccccacttaaaagtatattttttctttctttttttttttttttttttttgaggcagagttttgctcttgttgcccaggctggagtgcaatggcgtcatctcagctcaccacaacctccgcctcccgggttcaagcaattctcctgcctcagcctcccgagtaggtgggattacaggcatgtgccaccacacccggctaattttttgtatttttagtagagatggggttcgagctcctgacctcaggtgatccacccaccttggcctcccagagtgctgggattaaagccttagccaccatgcccggcaaaaatatatttttcatcagtctttaaagaactcagaatataagacattatacattttgagatgtttgcctttttaaaaatgaattgtttttcccattataaaacagtgtgataagacacagttgttaggggtcagtgttggtaaattgacaactaaaatcacaaagatgtttgataatacctctctgccaggtatgggaaggtcggtggcgagtgatccctcatgatgtactaccagactggctcaaggataatgacttcctcttgcatggacaccggcctcctatgccttctttccgggcctgttttaagagcattttcagaatacacacagaaacaggcaacatttggacacatctcttaggtatgtaatgtcagtgatgtaatgagctggtgattcactttcttcctttttattttcatgtatttgagggtaagcacagaacttcagaaatgtatttggatttgccattttgttttctgaatttctaatgatgaattttctgactggtttactcgtagtttatcctggtttgcactatgattttgttataacttaagttatatttctttctactagattttcagatcttgtcctcaaaccaggccaagtaaaagttgctatacaaaatgtggtttcatattgtagtgaccactgagaagaaagtattatttaatagaagtaatctgatcttttaaaatgcattacttatatgcactgctaattttcagtttggcctgaagtagaatatggactgtaattttttctccataatgattgtatattaaagaaagaatactgtagaggaaagaatagtctttccaacaaatggtgctgggataactgagccatatgtaaaagaatgatcccgaactcacatatgcaaaagtgaacccaaaatggatcaaagacccaaacataagagcaaaaactataaaatccttaggagaaaacatatataaatccttgtaagtttggattaggtgactgtttctgagaattgccaccaaagaaaaaatagataaattggacttcatcaaaattaaaaacgttggtatatcaaagtgcattatcaagaaagtgaaaagacaacccatagagaagagaatatttacaaatcagctagttggtaaggatctaataaataactcctacaactcaacaataaagacaaccgattttaaaaatgggtaaaacatctgaatagatgttcttccagagaagatctaaaatggccaataagcacatgaaaagatgtttgtcttcgttaggcatttaggaaaacgcaaatcaaatgctttgagaaatgcagtgagaaatctcaacacttcacacccactaagatggctataatcaagaaaatgaaaaataacaagtcttggcaaggatgtggggaaattggaactttcatacgttgccagtaggaatgtgaaatggggcagtccccatggaaaagtctggcagttcctcaaaaagttacatagagttaccagaaatcaactcctagtatattacccaagatgactgaaaacatgttcacataaaaacatgaacacagaagtttgcagcaacattattcataatagctaaaaagtagaagcaacccagatgtctatcgactggtaatggagaaacaaaatatggtgtattcatacactggagtattatttagctgcaaaaagggatgaagcactgatacatgccacagcatgggtgagccttgaaaacatttgctaagtgaaagaagccagacacagaaggtcacatgtcgtttgattctacatgcatgaaatgtccagaacaggaaaatccataaagacagtagattagtggttgtttagggctgtaaggagtggggaatgggggatgacagcagatacagtacttctgtttggggtgatgaaaatattttggaattagttagtggtgatggttgtacaaccttgtgaatatactagtttctggccttactccatttgatttgcatttcttatcacacatttgatttccatggtgcctctaagttcctgcctgctagtatagtgagaggagagtagagggtcaaatcaagtttgttgctgacttctaattcaagggtaatttttaattaaaatgataaaacaattacttagcaaacgttgaagttgaaatttctgtgcctgccttgtgttaggtggcccctccctaagcaataggaacatggcatctttctgtgtagaacagtgtaagaattggttaggaaatggcctgagtaggcattttgtccttaactcttgaatctcctgaaccagctgatgagggccagagaaacagcatgacactccctgacagctattggtagaggaaagctgtatttcttggcagggtttaggggtattggaattcattgtaaaaacaaaagcaaaagcacaatggacctttgaaagctggactatgaggtatgtgccaaaaactgtataaatgaaactattctacatctagaggtagaagtggcttcattatatcttctaagaagtagcagcagtgtctgatttctttttagattatgcccttagataagtttttatattctgtatattcagtttatgaagctgaagtatttctgataccaacaatgttggaagtaaaccacaattttggataggcttcagttatttggaagatttttctcattgaacttttgattcagatattgttgtaatgcagaaggattgaaaacagaatcagttataatactgtctcttctaaagggtgatattttatctattttctgtgctctttttcctaggttgtgtattcttcctgtgcctggggatcttttatatgtttcgcccaaatatctcctttgtggcccctctgcaagagaaggtggtctttggattatttttcttaggagccattctctgcctttctttttcatggctcttccacacagtctactgccactcagagggggtctctcggctcttctctaagtaagtatctgtaaagtccgtattttggccaatgatttagaggtagtgcgttagggaaaaacattcagcagagttggcagaattcttaatatcatctcatgcaaacttttttttttttaaaaattaaagagcaacccagtttggctcttaggtgtcgttgatggcttatgtcatgagggattaattggaacctgtagtggccagtagtttttaaattactgcattaaatggattttttttttctgtcatagactggattactctggtattgctcttctgattatgggaagttttgttccttggctttattattctttctactgtaatccacaaccttgcttcatctacttgattgtcatctgtgtgctgggcattgcagccattatagtctcccagtgggacatgtttgccacccctcagtatcggggagtaagagcaggtaagagcacggggaggttctacattcgacattcatttattcactagttaaattcactatttattaaagttctaccatttgccaaacattatgctgtgtatttgtgatgccaagatgaagaaatcatagttcctattgttgggggtttttttatgtagtagtcgtagtagttgttgttgttagtagtgtttattaattgtcttaaagcattagggttgaggggtcatggtgagtatgaggtgatataatttgggactgttacttacttgaatttatggctaaacaccatccagaaagcggtagtctataacttttgtaaacaatgatttctcttactcattgtggagtgtactttttgtagtgattctttttttgttgtcattttctgttttgttttatatttttattttataatggataatctgcttaaggcttcacccccatgctgattatgttttgttaaagtgggcaccatatctcccttctgatgtagggataaaggggctttttatatttaggataatgcctgtttagctggagatcatagagaaagccacaagcccctcagtgccttggctgagtcttcttgtaggacatggaattagcagagatgttcttgtctcctactcagtgtcaagctcagtgcccactgagaatgaacaaggagtgtttttcctttcgtccttaaacccccaaaaccatttagtattaaaatctaaaccctggatcatccgggggtacctggaaggttagacacagaagtaacctcatgtcagtgaccactgttaaaacactctgccgtcctcctttttgtgttcatatgagcaaatgagcattggtcagggtcagctttcaacagtacttgagtagtacagtggtgttaagcctgcttgagcacatgagataagaatgtctttgctgagtacagctgagcattctgttatttacatacatcagaagaaccaaattcctgagcctttgacttagagaatgataggcagaaaccattagcagcctttgcttagcatgtagaggaaaagaaaaagtgaatagtgcaaaagtctagcaaatctttccttagaggagcagaggtaggaaatagtttttctttaggtctgcttggttcttacctgttgtgagagcagcattgccattttcctcttctcttttagatgtccattttatttgtgagtatgcatcccatagctgtgcttacatatattgctgtataccagcagtttatttggtaatgtccaaagcaagtaataggttctttatctgttgagaaagcttaccaaatgacagcttgttttgcaagggtcattgttaactctgtgtcaaaatgtaggagcagttactggcagaggcagggagctcctgtgttgctttggctgatacacgggtttcaaagtggatttagatcagtggacttcacatttttttttccatcatgacattttctattacagtcctgaacacgtgtatctatacataagttcatgtatatataaattacaaaactttaacaaaatagtatttaatcctccacgtatgttacagtctgatattttaaattttattctagttcatttaaaaaaatgctggttgagactcatttaattgatcttagaacttaggtcatatccagaagtttaaaaaaacattagatgcttctggaggcagtaaggcagtagggctgtggctgagtgatttgctagacttttgtacttgattgctatttaggggtctgggagcttatagtattctctcccatcccccacccccattaggtcatttgttaaaggcatttaacaatttttaagtgcccagttataggttatagatttagattaaattttttcttcttttttcttttcttttctttttctttctttctttcttttttttttttttttttttttttttgagatggagtcttgctttgtagcccaggctggagtgcagtggtgccatcttggctcctgacctcaagtgatctgcctgcctcggcctcccaaagtgctgggattacaggtgtgagctgagccactgcgcccagccttagattcctattaaaaataggtgatgccaggtgatgtgatcattcatcgcctctccaccccaacaacgcctgctgcttcaaagagaagatgggatacgtaaaattgcttgtattgtaacctgtgtctgtagaaggacaggctaggacagtttaaataaactcctggagctgtcctttccgaaaacatttagcaacattaattcaaaacccttgaagactgctgacttactaagctaagcctctcttagtgaggctcattgctttttttttttttttttgagacggagtttcactcctgttgcccaagctggggtgcaatggcgtgatctcggctcactgcagcctctgcccccggcttcaggtgattctcctgcctcagcctcccgagtagctgggattacaggagtgcgctaccacgcctggctactttttggatgtttagtagaaatggggtttcaccctgttagccaggctggtcccaaagtgctgggattacaggtgtgagccactgcacccggctggctccttgcatttgtaaatctttagaacatagctgtgttgtcttgtgtgacagtgtcccagagtttgaagaaccttctttttcctttaaaactggaagctaattttatatgctactttgaaatattcagtttacagagttaatggtgctgtgctgtaatactgacttctggctctttgtttctcttctgcattactttactctcttcttgtgactcctaggagtgtttttgggcctaggcctgagtggaatcattcctaccttgcactatgtcatctcggaggggttccttaaggccgccaccatagggcagataggctggttgatgctgatggccagcctctacatcacaggagctgccctgtatgctgcccggatccccgaacgctttttccctggcaaatgtgacatctgggtaagtatgtcggggtgactgagtgtgtaggtatctgctcatgagttattggcatcctgcagagtgatgcaatagaaaaagttttagacacaaagtcaggagagttgtatcctggtcctaactccctattgagctttctctggacaataagtacaataataactaacatttagtgagcacttccttggtgctcagcatggtacagagtgctttgcacggattacctcattacatacattagccctatgtggtatgataactcttgttaatgtcttcattttacaaagcaggagactaaggcttagggaggtgaggccacttgtccaagccatgtggctatttaagtggtagaacaggaatttgaattaggctagttttaatatcagaatctgtgctctttttgacaatggcaagtcattttgccattctttgtctcagtttccttatttatgaaatgaggatgatagaccagattttcaccagagccctttttaggaccagatttctatcagggtctttttttataccgaaatttagtgatttgtaatgggagactgagtgtatgtatgtctggtggaagaagagggattgtgaagaagagggaactggattgaactggattgtggtcattggtaagagaggaggaaagagaagagggaaaagaaaatataaggactgatatagatagaaaaacttataaccaaagagggcctgaaatattttcaacaagacaacattggaaatacgcttaatgtgaaatctgacctttgtaattctgttcctcagcctattgctgattattatagactaacctgatgatctcatctctttaaagtttagtttagctatctgtaaaatgaagatgatcttgaacttaagcaatctttctagcttcaaattagctaatttatctttctgattcatcatcatcattttaacattggtatggaggggtttttgccctttatcccctttctttgggggaaattaatcccctttggtttcctaggaaacatctcttgggatgtgacttgatttcttctagtttttgcttctaaaatcagtgtttttctcttgataaatactgctttgccactgtactttttcattttgagattataatgacagaatagtgtaatttttgaagtgcagcgagcaattgattacccttcttgagatgtggaatagcattcaacagaacaggtaaaatcagaaataatgtcacttgcttgtagaccagcttctgacttgagaagtaaaagggcctgtttgcttagtttttgacacattatgtagttcatgaaatttgagaatcgtagcactgaaggaaactctggggaccatatgttgtgacattccacacaccatcctggaagcctctttagtttttctaacagagagcaacatttaaatgaccatagctctagtcctcttgtccatgacaaaatgtccatcagcagtgagtaggaaaaggactgtatgtgtaatcatagcccatggctaggtgtcaagggatccttgggacctttgtggcctgttagcaagagagtctgtgtgtcatttgccactttagcaagagagtctgtgtgtcatttgccactggtttcagtgttaagtttgtagatttttatgtgtagagcttagtggcagcaccttggtttctttgggaacagctggctttcagctcagcagaatgtgttacacctaatggatgttccaggatctgtggacgccttgaatttgagctctgtcacatccattccctgccttctgctcagcataaaccaggattaaacgatcctaagaatctttagcagtcttaataatgtatgggtttctctacccccttctcttcttttttcccctccagtttcactctcatcagctgtttcatatctttgtggttgctggagcttttgttcacttccatggtgtctcaaacctccaggagtttcgtttcatgatcggcgggggctgcagtgaagaggatgcactgtgatacctaccagtctccagggactatgaccctaaaccagggcctgcggcacttgcgggcctccctgctggctactgatgccagtaccagaggagccccaaaactttgacagcctcgtgggctttgtgacggcccagtggctctgcgtggtacatgactgagaagagaaaaacaaaaataaatcatacctcaaaggatggagtgcatcaattgggagaaaaggagacatagcccaaaccctggcttattcttgggatctactgattgcgggctctgcaagacccttggcaaactggcttctgatccatatcatatttatttgtagaagatggcgaaacagtttagctggtggttctttcttctccctttctctctctctatgacaataatacaaaccaatttaagtgaacatttatatccgataaggggtgggagtgtgattttaaatgctcttttgggagaacaaagaaattaatgtaaataagatttctaactgtttaaataagactttatataaatgtttaaaacataggggtaagggagggagggagaatttttgtatagaatgaaacatgcaagtaccacacactgtttgaattttgcacaaaaagtgactgtaggatcaggtgatagccccggaatgtacagtgtcttggtgcaccaagatgccttctaaaggctgacataccttggaccctaatggggcagagagtatagccctagcccagtggtgacatgaccactccctttgggaggcctgaggtagaggggagtggtatgtgttttctcagtggaagcagcacatgagtgggtgacaggatgttagataaaggctctagttagggtgtcattgtcatttgagagactgacacactcctagcagctggtaaaggggtgctggaggccatggaggagctctagaaacattagcatgggctgatctgattacttcctggcatcccgctcacttttatgggaagtcttattagagggatgggacagttttccatatccttgctgtggagctctggaacactctctaaatttccctctattaaaaatcactgccctaactacacttcctccttgagggaatagaaatggacctttctctgacatagttcttggcatgggagccagccacaaatgagattctgacgtgtccaggtttctcctgagctcatctacatagattggtagacccttcctttggattaggaaagatgagttttacctctggtacactgtcttggtaagcctggatgtgacagacacctcggctctccttgaataagaaagccagcagaactcttaaagccagttgtagtacggagttgtcagcactcactgaacctcactttacagggataagagtggtgtggcattttaaatacaatggtatgttattgccagggagtgaggtacaagacgatggctcatgtcacaggcctacctgatacggtgtcagagaaagtggtggggaaaggatctggttcatggaattctgatcttggcccataggtgaaccaccaaaatagtgctcgagtcttaggttactgtcatcaaagacttgggatgactccattatatcctggggttgtgggtattagaactaaatatggaggtcctgagcatggggactggcgtcctcagtaggtgtttgggaatatgggaagggtctcctatttattcaatagagttttctcagttattttcctcccttgcccttgcaatctccagcaaaaggtgggatctaggaagaaagaatccagtgtagaagttgagaagaacttgaacgttttggttctggataaggtcactgtcctaggtgctaggtggaccgagcaaaagactcagtggatgaactggtgcagtgcctgacagaataaagaacagtattaatccctttgagaaagcatagtccagcaggacagtggccatttggacagaagcccacttagtttcttgggagcaacagcacgtatcagaagccagacttgctcttcggtcatgcactttgggatacagcgtataggtgcagccctgtcacaacaccaacagaagtagcagcctctgggtgcagtcacccacaccccaaagctggaaggatctggttcaacatagcacaaacccttaggaaaaatgaaattaacatcactgatgtgtaatccagtaaaatctccctttttcgggtgtgtatgtgggcatgtgcccatttctatgtgtgtgtctacgtgcagctcactaccaacagcctcatgtgcacttgacctgacagtgctcgctgagaactctcaccaggttggcgcctgaatgccttactctcagcagtcagaggcttgcttgctctgtgcagatttttaattttcttttttggccctaggctggttgggacctctacagcttcattctttcaccattaaatagtggcctttttcagtattttccctcttcccctttataaattatgctaaagccacaaagcacatttttggggatcatagaaggttggggttccagaaaggcatctgtgtgatggttccattgatgtgggatttccctacttgctgtattctcagtttctaataaaaagaaccaaatgaaatatgacctgtcgtgtgtttttttattgccgttcagcttacctccccttggcttttggcacttctctgaagctctaatttagacactgtgatttaagaaatttttggaaacttccccttcgggatgcagaaatcaaagtgcttatgcccaactcctgtttcctaactcaagctttcctcgtaagttgcaccaggtttagaatggaggggagagcagctctaaggggcagttcttatttattattgaagtttataaaaatagtctctaatgccaagggcaataggctctaccataaagcctgggtgtgtagtaggctgtaccatctaggtttgtgtaagtgcattctatgatgtttgcgtgacaacgaaatcaactaattatgaaatcacttaatgatacatttctcagaatgtatctcttttgttaagtgaaacattacaaaatttatttttttgggcacggtggctcactcctgtaatcccagcactttgggaggccaaggcgggcggatcacgaggccatctcctacaggcagtgcacattgactaagttcattccttgcttttgctgttgtttgctattataaataggattttgtcttcaattatatcttctaaaagttcttttgattggtgaaagctactgattgctgaatattggttttttaccccgaccatttgctgttttctcctcctaatcataattttttcttagttggtttccagtaataaagtcatgttgtctgagaaaaaaaaaaatagtctctaatgtaacacttgatgccaagggatgtgttagagaactgtcgatgtcatatgatcataatcacaaccctctgagccattcaggagtattttcactctcattttatagatggggaaactgaagcttaggaagtttaagaatgactctagtgccagcactgtattggagaaaagagtgaagagaaaatttaagaaaacctttctgtatttgttattctgattaaacagtgtcatatacattgacttaaaggttttggggttcattaacctagtcccttctatataagaagaggatctggttcgagttggtgtttgaaataaaaaaaaaaaaaacggaattccaacccttttgccttaggtaaatttagtttttggaagggcatttccagtatcaggataatatgttcattctgaattctcatttttaagtatagatttaactttagagtgtcccataccttacctgggcatagacattggatcattccctgcttttgacaaacagtattagctgaagcctactaataacccacttgtcttttcctcgtctcttcccaaaaggcatctgagccgggcatggtggctgacgcctgtaatgccagcactttggaaggccgaggtgggcggatcacaaggtcaggagttcgagaccaccttggccaaaatggtgaaaccccgtctctactataaatacaaaaaattagctgggtgtggtggaggcacctgtaatccagctacttgggaggctgaggcaggagactcgcttgaacctgggaggtggaggttgcagtgagctgagaccataccactgcactccagcctgggtgacagagcgagactccatctcccaaaaaaaaaaaaagtgtctgtatggttcctgatacccaggtgatgttatgtggttgtagaggaagtagataaggacaagtttggattctgactgtacagcgtgctaggctctgggtgcctagagaaccctctgggcaatgttactttaaaatcatcccagaaacaacatcttcttgaatatttggcttcaattgtagtgtttcattttgtcttcccctcagagggcatgaaccatttgctagccagcactgctccctacccaccaccaacaccctatctaatgtacaaatcttttctgccttcagctgtttgacttggtgaaacaagggcagcctgtctcactcatgtagaggctgcttatgatggcatggggcaggaaggaagcgtcacgtgcgatggcctcttgtcaccgccatcctactgtcccaccgtccctaccctgctacactacctggaaatgaaaagggtagcttcagatctcagttgtgccatttgccatgtagcatgggcaagtggccgtttgcttgtctagcctcggtgttctgttaaaatgaggccaacgatacatcctctagagcccacgctgagatgctgtggtttctatagataatttcctcaaacttcccatagtgattggctgggttttctgccaaaccctcgctccaattccgtccttttgtggacttcatttacttttctggccttatgatatgcccactttcttgcctccccgccgcccccgaaccacaacttcactgagcttaatgaagtggttgaagcattttccctgaaagctcttctattaaaatacacaacattattatgtggatttagtagttcacatatccttggtactgaggaaggaagatctgacagcatccttaagttcctggtcctccaaaggtggtgtacaacataagatttcaaaattctgcattctaatttatcaaatgacttaggttccaagttcatgcgtccgagagcaggtgatgggctttagagtgagccaggctgccacctgctgtcctttagtggttctgctaaaagtagaagaggaatcgttctgaccttcaaaggcaaagcttatgagaatattttgttctccaaggtagagaaggttagtttatcagaaatggtttttccttccaatggtcttgaaagggattcccctcggccatgcagagatatggggcccagaggaagcagacaaaaccaggccagaagctgctccagcttcctaagcttggctttttaagagttgataaatttgcctgaaagtgctctacctgtttgggtggggatgataacccttccaaaagataacactgggttatggtcctacagagactgctgtttggtgtctgtgagttggtagttggggtcaaatgcagttggaggtcagaatgtgccttaatgccaaagaatgaacggcaggtgggcggccacggccttggcaggaagtggcagggcgagagttagtgctcaccctgctgtgtgccgcacaggtggtgcctcgtgacacatggttgggacactcagtgatggcagcccaggggctagaaagggctccttgctgctttcttaaccctgctgggcctgttgaacaccttattgttcctggcatctaaattgtaatttctttgtccccacctccatgaaacaaggtaatgtaatgatcgcagttagcatgtatttatttagcgtttagtatatgccaggcagtttcaagcatgttacgtgaatacatcacattcaatcttcatacactcttccggtaatttggaggcaattttcaactctgctgcagtttctataacatgggactaatgatagtacccactttcagggggctgttgtgaggatggaatgagctgaaacatgttatttggtacatgtaaaccttagctgttctagcattattctgccacttaagctgtcttcgagctgcagtgtcttctgctgtaagggggcattaatactgccctcacagggttgttgcaagacatgacggtaagagtgacattctgtaaccgataatgtaagtgtgttttacagagttggcatttggcctactctagaggcaagttagggctatttgctggtaggaaacttgaacagctttgaacttgaactttgaagtgtggaggctgagggtgatggaggagcggggggaagcgccccaaaagccctgcatgctgtccatacccatcactggggtttgttaaatggaaagatttgaggccggcctctcccacatttctccatggctttgcccttaatgttgctctcactgtggagactgttgtctcctgggatccaggtcttggggtttggtcgggggaggaagctgcggggccttgggaccctgggggagatgtgtgtgcgctgggggtgagttcccagatagagcttccctcccagtggaccctgcttagcaagttggaatgttcccacaagttccaaccctaaggatttcactccagggacagagtcaagtagattcttttgtaaatcattggccagaaaagagcacaaccactgctctttttcactattttacagctatttttgccgtcccctgaatgtacatcaatttccatgcagcaattggatgtctcagcctattggcttttcatagatgcaaactttcctttagggctttgtaaagaagttttgggaagttcttcaatagcagcagtttggggacatcacgtgtagccctgtcgatatttcctgaatggatttcattcctttctcagtcttgaccattcccttacccaacccccaccccaacacagtcaaaaccttttcgagcagcagctaggaaggtgagtggcctcccaccatgagcctcactttggggaatctatgctttagttggggcatattcttgccttagcctcatctctgcatttggcctcattctgctgctgcttctcagtacccagaagtccttgggttctaccagagaccactccgacagaaagggtcagaaacaactgaccctttcttggctgggtctggactctggagtacaggaagaactaaattattttgtcttggtccaagctgagctcacgctggcttcccgaagaggagacaggaaggttaggtcagaagtatgctcatgttgagacctagggcagatggtaccgggcaccatgaagcatcttgaaagtggtgccaggtgattggtttcctgttccatccagcattctccggagccaggggccaccagcccaggattgagaggagcgttggcctgggggcgacccaactgcagttagctgcatcccatgtcagtgctgactttttgggatggctctggaaggatcaccttgccaaaacacaggtcaggctgggtggtgtcaccgcaggagttggggcagtagcccccaggcacacacaggcagcaggagtaggggcggcgaggctgaggtgtccgaggcgccgccgcagtcctgggcattctcctgcgaacgcagagcagaggtgacagcgcggcgctcagaggaggaccctcaaaaagggaggggcttccaccactaatttgggccaaaatcctggggaaaggggagttttggaaagccggggaagcactgctacctctcttaggcccctaccttttgggggaaaagcaaaggctggcccctcagcctgggaggggggcaccggccttctctgcttgccctcaggcttggagtggctgcgaggtcatcctctcagaatgtcctcaactctgggagcatctctgatgggggtttctggtcccagcagaccagactgtatgaagcacccggctcagggcctgacacctgggtctgggttctggatccgtaccagccactatgactattatccacgtgacggccccctcacattgctcgatcattcggaactaagtgtagcgggaatgaggacaagatgggaaaactgcattctaattcacctagggcccaagccctaggaagcgtggctcttcgttaaagcaccttgggatcctggttgacttcgtattttaatgacacaggggctctgctgtgctttcacaccagcacttcaacaaatgtgaagggtttttcccaccccactctctggacaccaactgggtgtctgtcctgaaactcaattcaagtctgacactattgagagttagcattagagctcgcaagttacagggctcggtcccacaaggtggccgtcacttcagatgccaatcgcaagccccaggtctcctgtccttctgacagactggctgtaaatctagggttcccatgagcctgtctcaggctccgtgagttgttagcgtggctcactcaactcaggaaggtgcttccctcaccatgacccgtttattataaaggacgcaggtcaggcggctgagtggaagagacacgtaggccaaggcacggggtacgcagagctcccacaccctttcaccaattcggacactctccaaacacagtggctcggggattttatggaggcctcattatgatcattgattaaatctttggccactggtgatcaactcagtctccagcccccttcctttcccagaagccaggggcttggagccaaaagtcccaaccctctaatcctaacttggtgtttcctgtggccagctctcatcctgtctagggcccccagccaccagtcatctaattagcatacaaagatacctctatcattccaagggtcttagaggcacctgtgaaggaaccagggactaagaccaaacactataacaaaagatgcacctatcaccctaatcaggaaataacaagcgttttaggagcttgccaggaatcaggacaaagactaaataaagatctgtttcttattatatcacagtgtcacagggcataaacgcgaatgtttcccccgagaagcacaggcacaggtgtgttcattggcatccctatatgctcctgtctgcaggggcacagacccaggcacagagggtttggggatgaaaatccagcccaccctcgatccgcctcaacccgcacctctggatggaaggcgtggcaggagtctggtcgccggcggagcttctgggagcgcatccggtcacatttgacagaggcattatgtgcagaagccactgttaaggtcaatatctcaggaccggagcccattctgcagactggaaaaaggtctggaagaacctgctctggtgccaccagttgccctgcaagcagctttagggtcaggaggcagcaccggggcccagggaagggttagagaacaaataacggagccctgtggaggacacgggcggtgaaggaggctggccccgctgctcaaaatctgagccctacagacacctcctacagcccccgcccccaccgcacacatccccgagcattgcaggatatatttgacttctgtcgcctcctgcagcactggtgggaagatgctgcagtcacaggtggggtctgtcaccaggaggaggaggttactgttgggaatctgctgcaccacaaataccctgcagcaaagaaagggagtcgaggatggctccgtggcgaccacgagaagggcttctagggaaggaacttctggagactttagtgtggagttagggtggcagggaagcccggaaccatgaggcagggcgggtcggggcagctaagggaacgggcctggcagatggctcagtcctactggctttgctcacaagcctttgctgctgcatttcctttcctagcagactggagacagtgaggaagtccaaactgggccgctttggagagtgcagagttgctattaatatatgcagggacgacaggcgtaaatcaagaccgtccggagcaaatgggcgggcgaggcaacacttgctctgggtgagggtggcgggggcaggcccgggcgaagcggcagcgctggagcttggccaccaggtggcgcagtggccccgcgctcggcgaggccgccctgcgaggagtggcgcggagtggggcggagcccgcccgccaggcccacggccttcccagcagtcccagcggccgcctaccgagcgcctcccctgggatgggcggtctcacacacccaacgtgcacaccacagccgtgtatactctcagccttcacggctctgtatgaggctggtgttgtgcccattttgcagatgagagaatggagactagaggaggcgaagtcacttgcccacggtcataagcgggggagcgcagagccagttttcaattcaggagtgtctactctgaagccctcgcttcactccggagcccgctggctctatcctcgcaccgagcctttgcgacaagtgcacagggaacgctaaccccagccctcaacgctgcacagaacccggggtgaaacccgcgcagcacgacgcgcagggagtgggggagaagggagccccgatgccagcgccgcaggtgccgcactgttaaatggtgtgccacgggcccgggggtctagggaccgcctggcgagcggcacgtctggctgaggcgaccctcgaggaggccgccgcattcccagcgtgggggtcctcccgggcttgggtgcggggggtcagcaggagagacccttccgcgcggcctccctgcctccccggggccagggcgggttataactagcggcttccacacagcaggaagcctgacagaggctgcccgcggggctcccagcgacccgaggacatcgcacacttccggccttctcttctcacactccctctgccttccaagttctccccacgcagctcaggcgcgggcactggcaccgccctgggcaccgcctgagcagggctgggggagacccgccgaagggcccactcgcccagagacgccgtggggtcacggctgcggcctcggcggggtgggctcgacacacagggtcaggcctcgggaccacctctccccatcccggctgtcccggtccccagccgtcctgcccgcctcagaccgtccgcctcctgcgtgtctcctccactgaggacggcgggggaagaccccgggagcgtgggctctgcacttagacgcatcccctcctcccggctgtggagccgtttcccgggggttccggggccctgcccgcacttccgccttgccgagggcgggaggagtgtggcgggacgggcggcgccgccctgcggtcttacttctggcagggcccgcactccacgatcccgttggcctcccggatggccggctggtacacgaacacggggtactccgtgtcgcagggctgcagcgggtcctgcttcttgtgtttgtgggctgcgggcggagaaaggacatcacgccaccgaaggggcctctggcctgcggtgacctcgcctcggcactcccagggccagagttcaccccagtgcatttgcagagggtccctcctcaggcagttctcaggacacgcgtgggaggagccgggcggggggtgaggggagggaagacacccggagtctctggagagggtggcttggcacggggagcgccgcaggggccctgagcctcggtggagggaaagcgcttcctctggggagcctggccctcactgagctgggggcagtgcaccatctccagggccccttccagcactcagatcccgggggagggtctgagcggggagcagaggcagtaggggcaccaaggggtggcttgggggtacttaggcccaccacccctcccttcactgtggacagacgcagtgcagttcctgggcaatcctgccctgctctcccgcacagccaggcccacactccacccattgagcctgagcgcctgcggtgggggcagccgggcaggtgggctccaggcctggggctgcggaagcccagaagccacagccacccggtgcgggactcctcgggggctgcgcgatttgccttagctccctgggacaaggacagtggactcaaccaaaggtggaggcgagtgcctcagccccgctctgcaggacccgagtccgttccctcccggagcctcctgaggtgggcacatggcagagcagggccctgccacacaggctgtctgcatggcccttctgttctcagggcctggctgtggcccccagggatagacctagaggcagggagcacaggtgtgagctaccttcccaggctcatgcagagtcctacaggagagcatgggtctccccagcttccaaatccagaagctcccagccagagctggaaacccagaggggacactagctggcgtgcagaaaactctcaccgagaggagcaagcccacagcggccagggctgtgagttttgtccccagggctgggcacagaagcctgcctgacaatggcagggttgagtggcgtccccagggtcacgcagtggaaggggctgcagcacctacgccccggcctgggactccaagtccagtgcgccttcactggggacactgacatctgtgtaggccagaccagggggaggagctccaggggacaggtaccccagagaagagaaattctgagcaggcagattgagggggatcatcttcaacttttgcaagatgcagctgctgacagagacagaggggacagtgtcctgtcactgacacctagtgactgtgaacaccccagagcagccccacccgatcgcggatgtgagtccagaaggagtctggagacacagcccgaactgccctgagcccaggggcagaggaagccccaagccaaggccagcagaagagaacgaagagcaagccagagagaggaccacagtggagatgaagagatgggataagctggggacagcccaggtggtgggaagttctggaaccccatggcaaaggttctaggaacacggaggaactgagcagagcccgctgaggcaagatggcctcctgcagtcatggagggcccaggcgcccgggcagtgagtgctttgaaaggccaggctcattgccgccctgcacgtgtggccacagccaccgggctttgtgttggggcctctcatggttgctgcctctgggggactctgtcctcagacacagggtcccgcaggggaatcatcctgagagcttcctggggcccctggaacccggagtcccgctgagggctggggtgccgctgtttgcttctccaaggggtgggctggaggttgctcactcatactggctcatggccacccggccacaccaccggcttcctcttggaatcttacgtgttctcacccaaggggaggctggagtttctcattttccctggggcctgaccgtcctcatgtgggatgagaactgggctagactccccagtcccccaacccccaggaatggtactttaaaccaggagagctcagccctgcttggggtcattcctgggacaggtcatggagggtgggttctttgtagctcctgctgcgtccccaacccaccgccagcagggatggcctcagctgggctacttacagtgatggaagacacttttggctggccggaacataagcccagcacagggtggacacggcacaggaaaacatggtggcacatgagggcaggatgtcatggggtggtgatgatggcacatggagtggcggtgtcccaagatgatgtcacacacagagccaggagtgagggatgtgatgagagaaggccacgcagggtgagatgtgaacaacgatgcttcagggtcacctatccccactgtcacccaccccacagggaatggtctcacgttagtggaccaaaatgccactgctcttcagcacatggccctgcagctccgtgcactcaccctcggcccctctgtcgtaccaggagccccagacactccactccagcaggaacctgtcagacacaggactgaatctcggagacctctgagccctcccgacttgggttcccaaggagctggagtatccctgacagcccccggcccatcccctctcctccaggacaccctcttccctgcttgagggctctggttgagcaatgaggtccctcccaggggcagcatgcagggaagaggtggcgtccatgcccagggattgtgccctccttccccggggtctggtagcaagtgggttgtcctggagaccttgcagcctcctgctcaaggacaccctcctaagccaccctcgcatgcagccctccaccagccccgtgtctacccccactcacagcaccagctcctgcagcagccacctggtcgccgtcaagaaggcagaaattggctgggaaggagagagtgcacaccgctcgcacctaggtccaagggtgagggtgccccccccccaccaccagcaccaccctcagagagaccagagagtgggctgccctgccactgggagcaggggcagcagagcacaggccagcagcccagcaccccccatccccccacctcccacctgccctgcagagctgagctagctgggaaatgcctattgcaggtcagacatctcgggtggggttggacatgcccctccaggagacgagtcaagccctcatcctggtgctgcactgctgaggatccctggtgatcaagcggcagtgtcaggggctgaggctggggcaggctgctctctctgtctccgccagaggcactgttgtgtcacaactaccagagcagtccctctgaagccccaaagccagacccagaggcctgcctgggagtgggactgagagcagtggctctgagcagaagatggagatggtcaaggtagggccctggggccagacacccaaggtgggtgagaacagggaccagtgacctcattccaggacagggcagaggactggctggctggctggcagagggaagggctgggtgacactcacgctgaccaggggctgggctgcactgtggtggtgactcgagggtttgcacatggcctgatagtcatacatagtcactctgaaaatcagaagcgggctgtgatgagtccaaagccacccccaccccctgcccctgcctcaggaggctttactagcagagcatccgtttaccgcaattcccaagggcgcctgagctaggacagcagatcagaatacagctcatgctgaaaattacatttctgcacttggaatggtttaaagacaatgaagagcaaatggcttttgccctcggagttggacggttttccctctagggtctgagctgcggagcccctcccagggatgcccacatgcccttacgccctccccacatcatgctgacgccctggctccataaaggttgcttttgcagctctttgggggcacccactgtgggttttggaggtcaggatcctgagatccgccaggaccacttagcgtcagctctcggtttgctgtgtctatttggactggggaggagtgtgccccacttactggctgaacacccccatgctgagcagctgggtcaggacagcaccatccacctcccccagaaatcttcccgtctgtgagagagggacagcagagagagagggagggagagagatggagcaggatggagccttccgagtccagcactatttattcagctcaggtcgaggcttttggtgccagttgggctttccctctgactgcctctcagcataatccagctgaacccttccacccacgcccctgtccggtcctggctccctcctcaccgtccagcctggtgcttcctcaactggaaggagtcacccgctagctgactatgagaccaacgtagaggctgcaatgtgatttttttaatggtacagaattccttggaacaattctgtgtgaatcacttgcagcaaggttaaagaatgttttatgaaactggatttgttttatatgctgtgtgtgtgtgtgtgtgtgtgtgtgtgtgtgtgtgtgtgtgtgttgggtcagatataaaacacgtttctgcaggtcaaggtcagaagcagcgcctgtctcatgcacgctggtgtcctcatcttcccagtgtgagagggctgctgtggcttttgctgtccctccatcacctcccacaatcagtgtgccctgtggatcggtcatttgtcccctccatgacaatgacctaaccggatcccttgccctgtctccattgcccacctgccctcctaactgacctctcctcttgtccctgtggcactgccagagtcatcttactcaaatgcaacatggatgggtcactcttgccttcatttccaaaaaataaaacaggaccctccctcctgcccccggctccccttttgttgtccatcagccagcactttgtggcttctgtgccctgtgccaagcagcggccccctagctggctgagaatggcctcaggccctccactctgccacctctcatggcctctgtacctgccttggcctcttgagtctgccatttcccagcatgtcagggtggcagagcttgggctttggtgttggatttcgactttaatgtaggctgtctttaaaaattggggatagcgtgtgtccggcgtgtggctcccagtaaatacttgaaaaatggcagccatcgttaggaggagaggtccggggtccggcttggctgttctccctgccgattacccttcctctgtcctcatgccaaggaacttctttcctcaccctcgctgggaagtctttctattctaactttcccagccagaacaaggccttcctctgcacaccggcagcctgtggagtcctagtctcactctgcctgataagatatttggttgaaaactgtatgagaacaggaccatgtcttactcatctctgaattccccaaagtgtcttggtacctggacatactcagtcagtgtggaatgttgatagggaagggaggtgggaaccaagctgatggccacgtcaggttcttcatggagagctttctagctgccagggcccaaaatgatgacatgtgtgataactaccaataaaaagaaatgcaaggctggtacaggagctgaggcacatgggcctggccaatctcaaggaaagccaggcattgtctgggtgcccacctggggcctgccctgggcagccaagtagactccagcaagccttctgaagggaacagagatgtattccagacaagggctaggcagaacgcacaactcctgaacacccaatgaacaaccagagaagttctctctcaaataagagatgatggtg

SEQ ID NO: 8 - DNA, 3982 bp, Homo sapiens
cgcggcggcggcagcggcggcggctacaccgggcttggccccctccctcctccgttcccccctcctcccccctcccctcagcggtggctcccaagaagtccgagacacgcggatcaactcactatcctgaaggtccattctcccaagaagaggggacagaaagacagatctatttgtaagaaaggcttgggtatcccatgaacgagccaacagaaaaccgattggggtgcagcaggactccagagccagatataaggctcagaaaagggcaccaactggatggtacacgaagaggtgataatgacagccaccaaggagatttggagcccattttagaggcatctgttctatcttcccatcataaaaaaagctctgaggaacatgaatacagtgatgaagctcctcaggaagatgagggctttatgggcatgtcccctctcttacaagcccatcatgctatggaaaaaatggaagaatttgtttgtaaggtatgggaaggtcggtggcgagtgatccctcatgatgtactaccagactggctcaaggataatgacttcctcttgcatggacaccggcctcctatgccttctttccgggcctgttttaagagcattttcagaatacacacagaaacaggcaacatttggacacatctcttaggttgtgtattcttcctgtgcctggggatcttttatatgtttcgcccaaatatctcctttgtggcccctctgcaagagaaggtggtctttggattatttttcttaggagccattctctgcctttctttttcatggctcttccacacagtctactgccactcagagggggtctctcggctcttctctaaactggattactctggtattgctcttctgattatgggaagttttgttccttggctttattattctttctactgtaatccacaaccttgcttcatctacttgattgtcatctgtgtgctgggcattgcagccattatagtctcccagtgggacatgtttgccacccctcagtatcggggagtaagagcaggagtgtttttgggcctaggcctgagtggaatcattcctaccttgcactatgtcatctcggaggggttccttaaggccgccaccatagggcagataggctggttgatgctgatggccagcctctacatcacaggagctgccctgtatgctgcccggatccccgaacgctttttccctggcaaatgtgacatctggtttcactctcatcagctgtttcatatctttgtggttgctggagcttttgttcacttccatggtgtctcaaacctccaggagtttcgtttcatgatcggcgggggctgcagtgaagaggatgcactgtgatacctaccagtctccagggactatgaccctaaaccagggcctgcggcacttgcgggcctccctgctggctactgatgccagtaccagaggagccccaaaactttgacagcctcgtgggctttgtgacggcccaggggctctgcgtggtacatgactgagaagagaaaaacaaaaataaatcatacctcaaaggatggagtgcatcaattgggagaaaaggagacatagcccaaaccctggcttattcttgggatctactgattgcgggctctgcaagacccttggcaaactggcttctgatccatatcatatttatttgtagaagatggcgaaacagtttagctggtggttctttcttctccctttctctctctctatgacaataatacaaaccaatttaagtgaacatttatatccgataaggggtgggagtgtgattttaaatgctcttttgggagaacaaagaaattaatgtaaataagatttctaactgtttaaataagactttatataaatgtttaaaacataggggtaagggagggagggagaatttttgtatagaatgaaacatgcaagtaccacacactgtttgaattttgcacaaaaagtgactgtaggatcaggtgatagccccggaatgtacagtgtcttggtgcaccaagatgccttctaaaggctgacataccttggaccctaatggggcagagagtatagccctagcccagtggtgacatgaccactccctttgggaggcctgaggtagaggggagtggtatgtgttttctcagtggaagcagcacatgagtgggtgacaggatgttagataaaggctctagttagggtgtcattgtcatttgagagactgacacactcctagcagctggtaaaggggtgctggaggccatggaggagctctagaaacattagcatgggctgatctgattacttcctggcatcccgctcacttttatgggaagtcttattagagggatgggacagttttccatatccttgctgtggagctctggaacactctctaaatttccctctattaaaaatcactgccctaactacacttcctccttgagggaatagaaatggacctttctctgacatagttcttggcatgggagccagccacaaatgagattctgacgtgtccaggtttctcctgagctcatctacatagattggtagacccttcctttggattaggaaagatgagttttacctctggtacactgtcttggtaagcctggatgtgacagacacctcggctctccttgaataagaaagccagcagaactcttaaagccagttgtagtacggagttgtcagcactcactgaacctcactttacagggataagagtggtgtggcattttaaatacaatggtatgttattgccagggagtgaggtacaagacgatggctcatgtcacaggcctacctgatacggtgtcagagaaagtggtggggaaaggatctggttcatggaattctgatcttggcccataggtgaaccaccaaaatagtgctcgagtcttaggttactgtcatcaaagacttgggatgactccattatatcctggggttgtgggtattagaactaaatatggaggtcctgagcatggggactggcgtcctcagtaggtgtttgggaatatgggaagggtctcctatttattcaatagagttttctcagttattttcctcccttgcccttgcaatctccagcaaaaggtgggatctaggaagaaagaatccagtgtagaagttgagaagaacttgaacgttttggttctggataaggtcactgtcctaggtgctaggtggaccgagcaaaagactcagtggatgaactggtgcagtgcctgacagaataaagaacagtattaatccctttgagaaagcatagtccagcaggacagtggccatttggacagaagcccacttagtttcttgggagcaacagcacgtatcagaagccagacttgctcttcggtcatgcactttgggatacagcgtataggtgcagccctgtcacaacaccaacagaagtagcagcctctgggtgcagtcacccacaccccaaagctggaaggatctggttcaacatagcacaaacccttaggaaaaatgaaattaacatcactgatgtgtaatccagtaaaatctccctttttcgggtgtgtatgtgggcatgtgcccatttctatgtgtgtgtctacgtgcagctcactaccaacagcctcatgtgcacttgacctgacagtgctcgctgagaactctcaccaggttggcgcctgaatgccttactctcagcagtcagaggcttgcttgctctgtgcagatttttaattttcttttttggccctaggctggttgggacctctacagcttcattctttcaccattaaatagtggcctttttcagtattttccctcttcccctttataaattatgctaaagccacaaagcacatttttggggatcatagaaggttggggttccagaaaggcatctgtgtgatggttccattgatgtgggatttccctacttgctgtattctcagtttctaataaaaagaaccaaatgaaatatgaaaaaaaaaaaaaaaaaa

SEQ ID NO: 9 - protein, 386 aa, Homo sapiens
MNEPTENRLGCSRTPEPDIRLRKGHQLDGTRRGDNDSHQGDLEPILEASVLSSHHKKSSEEHEYSDEAPQEDEGFMGMSPLLQAHHAMEKMEEFVCKVWEGRWRVIPHDVLPDWLKDNDFLLHGHRPPMPSFRACFKSIFRIHTETGNIWTHLLGCVFFLCLGIFYMFRPNISFVAPLQEKVVFGLFFLGAILCLSFSWLFHTVYCHSEGVSRLFSKLDYSGIALLIMGSFVPWLYYSFYCNPQPCFIYLIVICVLGIAAIIVSQWDMFATPQYRGVRAGVFLGLGLSGIIPTLHYVISEGFLKAATIGQIGWLMLMASLYITGAALYAARIPERFFPGKCDIWFHSHQLFHIFVVAGAFVHFHGVSNLQEFRFMIGGGCSEEDAL

(19) United States
(12) Patent Application Publication
(10) Pub. No.: US 2015/0301041 A1
(43) Pub. Date: Oct. 22, 2015

(54) ASSAY METHOD

(71) Applicant: THE UNIVERSITY OF BIRMINGHAM, Birmingham (GB)

(72) Inventors: George Edward Rainger, Worcestershire (GB); Parth Narendran, Birmingham (GB); Helen McGettrict, Birmingham (GB); Myriam Chimen, Birmingham (GB)

(73) Assignee: The University of Birmingham, Birmingham (GB)

(21) Appl. No.: 14/443,724

(22) PCT Filed: Nov. 21, 2013
(86) PCT No.: PCT/GB2013/053070

§ 371 Date: May 19, 2015

(30) Foreign Application Priority Data
Nov. 22, 2012 (GB) 1221011.8

(51) Int. Cl. G01N 33/564; G06F 19/00; G01N 33/569

(52) U.S. Cl. CPC G01N 33/564; G01N 33/56972; G06F 19/3431; G01N 2800/102; G01N 2333/705

(57) ABSTRACT

A method of determining the likelihood of a patient developing rheumatoid arthritis (RA) is provided, which relies on the use of AdipoR1 and/or AdipoR2 in leukocytes as prognostic markers. The method may comprise determining the expression of A dipoR1 and/or AdipoR2 in leukocytes in a sample from said patient. The method may be used for screening, within a population, one or more patients likely to develop RA.

The present invention relates to a method of determining the likelihood of a patient with joint inflammation developing rheumatoid arthritis (RA). This is useful in stratifying patients presenting with non-specific synovitis, into those patients likely to develop RA and those patients where the disease or condition is likely to resolve itself, e.g. those patients with short-term inflammation that can be successfully managed with rest and/or NSAIDs (non-steroidal anti-inflammatory drugs).

### INTRODUCTION

Rheumatoid Arthritis is a chronic inflammatory disease for which we lack a cure and good early diagnostic markers capable of distinguishing acute from persistent joint inflammation (1, 2). A window of opportunity exists during the first 3 months of RA where the disease is still evolving and is more responsive to current treatment strategies (reviewed by [3]). In light of this, there is an urgent need for a biomarker capable of stratifying patients for therapy, i.e. distinguishing those with acute resolving synovitis, from those who will develop chronic persistent joint disease (RA). Indeed, this might be considered the ‘holy grail’ of early arthritis diagnosis.

Surprisingly, we have found that changes in adiponectin receptor expression on peripheral blood leukocytes can be used as a biomarker for early diagnosis of RA, including distinguishing patients likely to develop RA from those with other conditions, for instance those with resolving disease.

In particular, patients that develop or are diagnosed with RA have a lower level of surface protein expression of adiponectin receptors-1 and/or adiponectin receptor-2 (ApidoR1 and/or ApidoR2) on certain leukocyte subpopulations compared to patients with resolving joint inflammation. Looking at gene expression, we have shown that the same patients have elevated levels of AdipoR1 mRNA and reduced AdipoR2 mRNA compared to patients with resolving inflammation.

### SUMMARY OF THE INVENTION

Thus, in a first aspect, the present invention provides the use of AdipoR1 and/or AdipoR2 in leukocytes as a prognostic marker for RA (Rheumatoid Arthritis).

In a further aspect, the present invention provides a method of determining the likelihood of a patient developing RA (Rheumatoid Arthritis), the method comprising determining the expression of AdipoR1 and/or AdipoR2 in leukocytes in a sample from said patient.

Also provided is a method for indentifying a patient likely to develop RA, comprising:
a) determining the level of the AdipoR1 and/or AdipoR2 biomarker(s) in leukocytes in a sample from the patient;
b) comparing the determined level of said biomarker(s) with a reference level; and
c) identifying the patient based on the comparison of b).

The invention also provides a screening method for identifying, within a population, one or more patients likely to develop RA, comprising:
a) determining the level of the AdipoR1 and/or AdipoR2 biomarker(s) in leukocytes in a sample from each patient;
b) comparing the determined level of said biomarker(s) with a reference level; and
c) identifying patients based on the comparison of b).

In other words, a screening method is provided for identifying, within a population, one or more patients likely to develop RA, comprising repeating the present methods to thereby identify said one or more than one patient likely to develop RA. Optionally, this may further comprise stratifying the patients according to their clinical outcome or likely clinical outcome.

The invention also extends to a prognostic or diagnostic method wherein a reduced cell surface protein expression of AdipoR1 and AdipoR2 is detected by an antibody, such as a fluorescent-labelled antibody. Accordingly, provided is a method of determining the likelihood of a patient developing RA, comprising:
a) contacting a sample with an anti-AdipoR1 antibody and/or an anti-AdipoR2 antibody;
b) comparing the binding of the antibody or antibodies to a reference level; and
c) determining that the patient is likely to develop RA based upon the reduced binding of said antibody or antibodies.

One way to potentially referencing to a standard would be to introduce an external reference measurement into the assay. This could be done for example by putting in latex beads coated with AdipoR1 and/or AdipoR2. These could then be measured using the same reagents for assessing cellular levels. It is envisaged that reference bead could be made which fell between RA and non-RA. i.e. measurements below the reference level would indicate RA, whereas reading above it would indicate no RA. Flow cytometry would be a useful tool here to distinguish such beads from blood cells based either of forward and side light scatter, or incorporation of a fluorochrome that did not interfere with measurements from labelled antibodies.

Optionally, therefore, the method may include obtaining a sample from a patient. The anti-AdipoR1 or the anti-AdipoR2 antibody are optionally those listed herein, for instance in the section herein entitled ‘Examples of Antibodies Useful in the Invention.’ The preferred conditions for these antibodies are listed therein as well. These antibodies are commercially available. The antibodies used can preferably distinguish AdipoR1 and AdipoR2 (i.e. they don't bind some sort of common epitope).

It is also optional to introduce beads for standardisation of AdipoR1 and/or adipoR2 expression. As a result, the comparing the binding of the antibody or antibodies may be to cells and to reference beads. This is optionally by flow cytometry.

Optionally, point c) above, the determining that the patient is likely to develop RA, is based upon the reduced binding of said antibody or antibodies to leukocytes compared to the reference beads. This reduced binding to the reference beads is likely due to reduced surface expression of AdipoR1 and AdipoR2 on the leukocytes of patients with RA.

It may be helpful to indicate the negative prognosis for the determining step, i.e.: determining that the patient is likely not to develop RA based upon the increased binding of said antibody or antibodies. The method, therefore, optionally includes the following step, typically at the expense of other determining steps: determining that the patient does not have RA based on binding levels equivalent to or above those seen on the standard reference bead. This is likely due to the expression AdipoR1 and AdipoR2 remaining at the levels seen in healthy donors in patients who do not have RA.

Optional reference levels can be set up using a healthy cohort of blood donors.

Optionally, reduced binding in step c) was due to a reduction in the cell-surface expression of AdipoR1 or R2 (and preferably both).

The invention also provides an in vitro diagnostic use wherein anti-AdipoR1 or anti-AdipoR2 antibodies are detected. Accordingly, provided is in vitro use of an anti-AdipoR1 antibody and/or an anti-AdipoR2 antibody for identifying a patient likely to develop RA, wherein the identified patient displays in a sample a level of AdipoR1 and/or AdipoR2 protein which is less than a reference level.

Patients not likely to develop RA have other conditions, such as resolving disease. ‘Resolving disease’ is used herein to cover a range of possible conditions, other than RA, which may be causing the pain or inflammation in the joint or joints of the patients. Principal among these conditions is inflammation that can be treated effectively with NSAIDs for instance.

The patient or patients identified may be those that are likely to develop RA or they may be those that are likely to develop resolving disease (which as mentioned above includes other, non-RA conditions causing the symptoms). The present uses and methods therefore allow patients to be stratified according to their clinical outcome or likely clinical outcome, i.e. they include, preferably as a final step, said stratification.

Where reference is made throughout this specification to a patient who is “likely to develop RA,” it will be appreciated that this includes a patient that has RA, will develop RA, or is at least likely to go on to develop RA. The same holds true, mutatis mutandis for resolving disease, so that a patient who is “likely not to develop RA” or “is likely to develop resolving disease,” “will develop resolving disease” or “has resolving disease” can be used interchangeably. Furthermore, it will also be appreciated that term “develop” may include “diagnose” so that a patient that is likely to develop RA may be one that is later diagnosed with RA according to the appropriate criteria. Here, the terms ‘RA’ and ‘early RA’ are also used interchangeably.

Increased or decreased levels of the biomarker(s) may be compared to a reference level. The reference level is an AdipoR1 reference level where AdipoR1 is measured. The reference level is an AdipoR2 reference level where AdipoR2 is measured. When both AdipoR1 and AdipoR2 are measured then each biomarker is compared to the reference level for that same biomarker. The reference levels are described further below.

The patient is preferably one that presents with joint inflammation or joint pain that results, or is likely to result, from joint inflammation.

Accordingly, AdipoR1 and AdipoR2 can be considered to be biomarkers for RA. In particular, they can be considered to be biomarkers for stratifying patients with joint inflammation into patients likely to develop RA and patients that are likely to develop resolving disease.

In particular, the sample is a body tissue or body fluid sample. As the expression of the biomarkers is in (or on) leukocytes, the sample comprises said leukocytes (white blood cells) or fragments thereof. Preferred populations of said sampled leukocyte cells are PBMCs (peripheral blood mononuclear cells), PBL (peripheral blood lymphocytes), B cells and Natural Killer cell populations, e.g. NK-T-Cells.

The level (of the, or each, biomarker) determined is expression of said biomarker. This may be the protein or polynucleotide encoding it.

As the biomarkers are both receptors, determining the level of protein expression is typically achieved by measuring the prevalence of the protein form of the biomarker on the cell surface. This may be achieved by known methods, in particular flow cytometry, but other methods such as ELISA, western blotting, or other analytical protocols which yield a quantitative measurement of protein expression are included.

The polynucleotide encoding the protein biomarkers may be DNA, but is most typically RNA, in particular messenger RNA (mRNA). This may be achieved by known methods, such as qPCR.

The levels of protein and mRNA expression can be determined independently (i.e. AdipoR1 or AdipoR2). When levels of both biomarkers are determined (i.e. AdipoR1 and AdipoR2) then, typically, either protein levels of both or RNA levels are looked at. It is considered to be advantageous to combine the determination of the levels of both biomarkers.

The following applies to any of the uses or methods provided herein. The determining of the expression of the biomarker(s) in leukocytes in a sample from said patient may optionally include identifying a number of different determinants, each of which may be used alone or in any combination thereof.

### Changes in Cell-Surface Protein Expression of the Biomarker(s)

Changes in cell-surface protein expression one or more of the biomarker(s) may be determined. Optionally, therefore, reduced cell-surface protein expression of AdipoR1 and/or AdipoR2 (being AdipoR1, AdipoR2, or AdipoR1 and AdipoR2) on said leukocytes is indicative of early RA. AdipoR1 and AdipoR2 are highly expressed in healthy patients. However, in patients with early RA, a reduction is seen.

This is, in a preferred example, indicated by a population of B cells (a sub-population of leukocytes) where only 60% of the B-cells show either marker. Thus, the reference level for AdipoR1 is that only 60% of the B-cells show the AdipoR1 marker. The reference level for AdipoR2 is that only 60% of the B-cells show the AdipoR2 marker. Above 60% B-cells AdipoR1 and/or AdipoR2 is therefore indicative of the patient being likely to not develop RA. Below 60% B-cells expressing AdipoR1 and/or AdipoR2 is therefore indicative of the patient is likely to develop RA. A range of +/−10% is envisaged as plausible, so the reference level may be set at a threshold of 50%, 55%, 60%, 65% or 70%. ‘Less than’ here includes ‘less than or equal to’.

Thus, a population of B cells where less than 50-70%, and preferably less than 60%, of the B-cells show either marker is indicative of the patient being likely to develop RA.

### Changes in Leukocyte Sub-Populations Expressing the Protein Biomarker(s)

Changes in leukocyte sub-populations expressing the protein biomarker(s) may also be determined. In another preferred example, the determination is also indicated by another sub-population leukocytes, NK-T-cells, where only 20% of the NK-T-cells show either marker. Thus, the reference level for AdipoR1 is that only 20% of the NK-T-cells show the AdipoR1 marker. The reference level for AdipoR2 is that only 20% of the NK-T-cells show the AdipoR2 marker. Above 20% NK-T-cells AdipoR1 and/or AdipoR2 is therefore indicative of the patient being likely to not develop RA. Below 20% NK-T-cells expressing AdipoR1 and/or AdipoR2 is therefore indicative of the patient is likely to develop RA. A range of +/−10% is again envisaged as plausible, so the reference level may be set at a threshold of 10%, 15%, 20%, 25% or 30%. ‘Below’ here includes ‘less than or equal to.’

These NK and B-cell determinations are illustrated, for example, in FIGS. 1-4. Identification and quantification of B cells and/or NK-T-cells may be achieved using known methods, such as FACS. Thus, determination of the levels of just AdipoR1 or just AdipoR2, or both may be made.

However, optionally, the levels of both AdipoR1 and AdipoR2 are determined (assessed). In this case, there are at least three different options, which may be used alone or in any combination thereof.

Differences in AdipoR1 and AdipoR2 mRNA Expression Levels

Optionally, one assessment is to determine the differences in AdipoR1 and AdipoR2 mRNA expression levels, i.e. by comparing said mRNA levels. Optionally, a difference between AdipoR1 and AdipoR2 mRNA expression levels is indicative of RA. This difference is preferably a relative expression (REU) above 0.14 (compared to 18S as internal standard gene) for AdipoR1 contrasted with an REU below 0.1 for AdipoR2 (compared to 18S as internal standard gene). This is illustrated, for instance, in FIG. 5. In this instance, therefore, a difference in REU (for AdipoR1 compared to AdipoR2, when 18S is used as a standard) of around 0.04 is indicative of a likelihood of RA. A range of +/−10% is again envisaged as plausible, so the difference in said REU may be set at a threshold of 0.04+/−0.004, giving preferred values of at least 0.036, 0.038, 0.040, 0.042 and 0.044. Thus, in these instances, the reference level is the REU for mRNA expression of the 18S gene. The difference between the REU this reference level and for each of AdipoR1 and AdipoR2 is determined and if that difference is at least 0.04+/−10% (or one of the values stated above), then a likelihood of RA is indicated. This can be represented by the following formula:

if X−Y≧0.036, 0.038, 0.040, 0.042 or 0.044; then RA is indicated.

where: X=REU of AdipoR1/REU of 18S

### Y=REU of AdipoR2/REU of 18S

A reference for calculating REU is Livak K J & Schmittgen T D (2001). Analysis of relative gene expression data using realtime quantitative PCR and the 2-delta Ct method. Methods 25; 402-408.

Optionally, the expression of AdipoR1 and AdipoR2 may be incorporated into other mathematical algorithms. This may optionally provide standard ranges to stratify patients. In these cases, the level of both AdipoR1 and AdipoR2 are determined and further calculations required, so a reference level in the sense described above is not required. Thus, in these cases, the ‘reference level’ is replaced and the step b) of ‘comparing the determined level of said biomarker(s) with a reference level’ is amended to ‘b) calculating one or more ratios based of expression levels of AdipoR1 and AdipoR2 to provide an output or score to compare against a threshold’.

### Other Methods

Optionally, other methods for determining whether a patient is likely to develop RA include:

(i) Ratio of AdiopR2:AdipoR1 mRNA Expression

Optionally, this comprises determining the mRNA expression ratio of AdiopR2:AdipoR1 (i.e. AdipoR2 divided by AdipoR1). This may be measured by qPCR. A score of less than or equal to 1 (100%) is indicative of early RA. This is illustrated, for example, in FIG. 6. A preferred range of +/−10% is again envisaged as plausible, so difference may be set at a threshold of 90%, 95%, 100%, 105% or 110%. In this instance, therefore, both AdipoR1 and AdipoR2 are considered and the reference level is the ratio between AdipoR2 to AdipoR1 (within the preferred ranges provided above). This may be represented by the formula:

if Z≧90%, 95%, 100%, 105% or 110%, then RA is indicated.

where Z=[(mRNA expression of AdipoR2)/(mRNA expression of AdipoR1)]×100
(ii) % Leukocytes Displaying AdiopR1 Protein×(Ratio of AdiopR2:AdipoR1 mRNA Expression)

Optionally, the method comprises determining the percentage of leukocytes displaying cell-surface protein expression of AdiopR1. This is then multiplied by the ratio of mRNA expression of AdiopR2 over AdipoR1 (see (i) above). This can be represented by the formula:

(Surface expression of AdipoR1)×ratio score of (i).

This is illustrated, for example, in FIG. 7 where measurements on B-cells are used as an exemplar.

Optionally, a score of less than a threshold of 85 is indicative of early RA, although a score of less than or equal to a threshold of 75, more preferably less than a threshold of 70, 65, 60 or 55 and a score of less than or equal to a threshold of 50 is particularly preferred.

For B-cells very early RA patients have scores of less than a threshold of 50. For PBL very early RA patients have scores of less than a threshold of 15. For NK T-cells very early RA patients have scores of less than a threshold of 10. This is shown in FIG. 8.

Thus, the percentage of leukocytes positive for surface protein expression of AdiopR1 is measured (preferably by FACs) and multiplied by the ratio from (i) above.

(iii) % Leukocytes Displaying AdiopR2 Protein×(Ratio of AdiopR2:AdipoR1 mRNA Expression)

Optionally, the method comprises determining the percentage of leukocytes displaying cell-surface protein expression of AdiopR2. This is then multiplied by the ratio of mRNA expression of AdiopR2 over AdipoR1 (see (i) above). This can be represented by the formula:

(Surface expression of AdipoR2)×ratio score of (i).

Optionally, a score of less than or equal to a threshold of 75 is indicative of early RA, more preferably less than or equal to a threshold of 70, 65, 60, 55, 50, 45, 40, 35, 30, 25, 20, 15 and score of less than or equal to a threshold of 10 is particularly preferred in some instances.

For B-cells very early RA patients have scores of less than or equal to a threshold of 30. For PBL very early RA patients have scores of less than or equal to a threshold of 15. For NK T-cells very early RA patients have scores of less than or equal to a threshold of 8. This is shown in FIG. 8.

Thus, the percentage of leukocytes positive for surface protein expression of AdiopR2 is measured (preferably by FACs) and multiplied by the ratio from (i) above.

The methods described herein may also include administration of suitable treatments to a patient. In some embodiments, therefore, they can be considered to be methods of treating said patient, whether for RA or non-RA conditions such as resolving disease, the method including the assessment of the AdipoR1 and/or AdipoR2 biomarkers described herein. Thus, in the case of patients determined to be likely to develop RA, anti-RA drugs may be prescribed and, optionally, administered. Alternatively, in the case of patients determined to be likely not to develop RA, NSAIDs may be prescribed and, optionally, administered for instance. Thus, in a still further aspect, we provide a method of treatment of RA comprising administering an anti-RA drug, for instance those known in the art, to a patient determined to be likely to develop RA by the methods described herein. Said anti-RA drug may be for the treatment and/or prophylaxis of RA. Thus, a course of treatment may be correlated to a particular genotype. Provided is, therefore, a method for the treatment or prophylaxis of RA, comprising administering a drug for said treatment or prophylaxis of RA to a patient identified by the methods described herein. Suitable drugs include Analgesics (e.g. Paracetamol, Co-codamol, Tramadol); Non-steroidal anti-inflammatory drugs (NSAIDs, e.g. Ibuprofen, Naproxen); Corticosteroids (e.g. Prednisolone, Depo-medrone); Disease modifying anti-rheumatic drugs (DMARDs, e.g. Methotrexate, Sulfasalazine, Leflunomide, Hydroxychloroquine); and Biologics (e.g. rituximab and anti TNF drugs, such as etanercept, adalimumab and infliximab). The biologics are particularly preferred, especially rituximab and adalimumab.

### BRIEF DESCRIPTION OF THE FIGURES

The invention will now be described with reference to the Figures where:

FIG. 1: The effect of treating peripheral blood lymphocytes with adiponectin on the migration of T cells across cytokine stimulated endothelial cells.

FIG. 2: The percentage of peripheral blood lymphocytes (PBL) showing surface expression of A) AdipoR1 and B) AdipoR2 as measured by flow cytometry. HC=healthy controls; Res=resolving arthritis; RA=very early unclassified rheumatoid arthritis that later fulfils criteria for RA (at 18 months).

FIG. 3: The percentage of peripheral blood B lymphocytes (B-cells) showing surface expression of A) AdipoR1 and B) AdipoR2 as measured by flow cytometry: HC=healthy controls; Res=resolving arthritis; RA=very early unclassified rheumatoid arthritis that later fulfils criteria for RA (at 18 months).

FIG. 4: The percentage of peripheral blood natural killer T-cells (NK-T-cells) showing surface expression of A) AdipoR1 and B) AdipoR2 as measured by flow cytometry. HC=healthy controls; Res=resolving arthritis; RA=very early unclassified rheumatoid arthritis that later fulfils criteria for RA (at 18 months).

FIG. 5: The mRNA expression of A) AdipoR1 and B) AdipoR2 by peripheral blood mononuclear cells as measured by qPCR. HC=healthy controls; Res=resolving arthritis; RA=very early unclassified rheumatoid arthritis that fulfils criteria for RA at 18 months.

FIG. 6: The ratio of the mRNA expression AdipoR2 over AdipoR1. HC=healthy controls; Res=resolving arthritis; RA=very early unclassified rheumatoid arthritis that fulfils criteria for RA at 18 months.

FIG. 7: The percentage of B-cells positive for surface expression of AdipoR1 multiplied by the ratio of the mRNA expression of (AdipoR2 over AdipoR1). HC=healthy controls; Res=resolving arthritis; RA=very early unclassified rheumatoid arthritis that fulfils criteria for RA at 18 months.

FIG. 8: (A-C) The percentage of leukocytes positive for expression of AdipoR1 multiplied by the ratio of the mRNA expression of (AdipoR2 over AdipoR1); D-F) The percentage of leukocytes positive for expression of AdipoR2 multiplied by the ratio of the mRNA expression of (AdipoR2 over AdipoR1) for purified populations of B-cells (A and D); PBL (B and E); NK-T-cells (C and F).

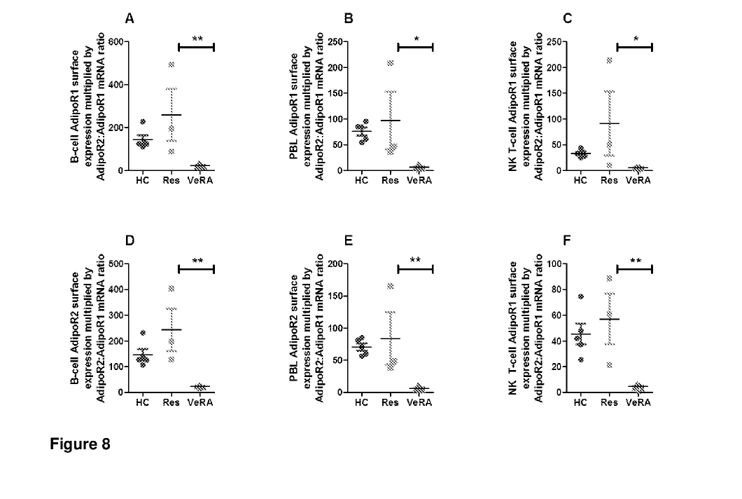

### DETAILED DESCRIPTION OF THE INVENTION

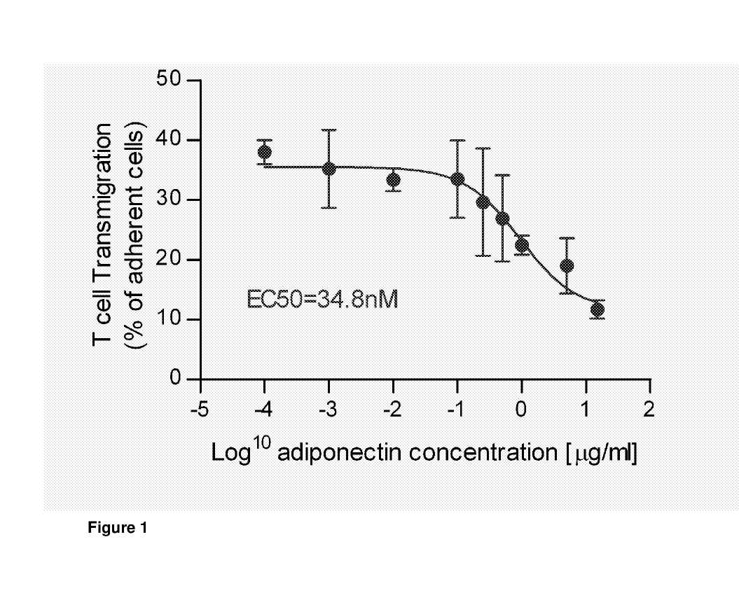

Adiponectin is an anti-inflammatory cytokine capable of regulating the recruitment of leukocytes to inflamed tissues (e.g. [4; 5]). Deficiency in adiponectin has previously been reported to augment leukocyte adhesion to TNFα-treated venules in mice, a response attributed to elevated levels of endothelial adhesion molecules (E-selectin and VCAM-1) [4]. Moreover, normal levels of recruitment were restored upon administration of exogenous adiponectin [4]. In vitro, we have shown that adiponectin inhibits human peripheral blood lymphocyte (PBL) migration across inflamed endothelium in a dose dependent manner (FIG. 1). Adiponectin has three known receptors, AdipoR1 and AdipoR2 and T-cadherin (reviewed in [6]). Here, we are interested in the first two. AdipoR1 and AdipoR2 are the products of discrete genes, although their protein sequences are ≈70% homologous. There is also significant sequence conservation in mammals with greater than 95% homology between murine and human receptors. AdipoR1 is a high affinity receptor for globular adiponectin, with low affinity for the full length molecule. AdipoR2 has intermediate affinity for both forms of adiponectin. Adiponectin receptors are expressed on many different cells and in numerous organs of the body. Importantly, in the context of the current document, these receptors are highly expressed on many subsets of circulating peripheral blood leukocytes.

For identification purposes, the sequences of AdipoR1 and AdipoR2 are provided herein as examples of AdipoR1 and AdipoR2. Two variants are listed for AdipoR1. Others may also be known or found but are only considered relevant if they code for a functional protein.

Along with mathematical algorithms which will provide ranges allowing the stratification of patients into different clinical outcome groups based on the measurements detailed. Collectively, they can be used to diagnose, i.e. provide a diagnosis for, RA in patients with very early disease where suitable biomarkers discriminating RA from other forms of joint disease are currently lacking. The invention can also be used to provide a prognosis for the likely clinical outcome for a particular patient, namely to distinguish between RA and non-RA conditions.

Where reference is made to a diagnosis or the provision of a diagnosis, it will be appreciated that this also refers to a prognosis, or the provision of a prognosis unless otherwise apparent. Accordingly, the method may comprise the diagnosis and/or the prognosis of rheumatoid arthritis. As with any method providing a diagnosis or a prognosis, the output (i.e. the diagnosis or prognosis itself) is based on an expected outcome or result. As such, an entirely definitive output cannot necessarily be provided. Instead, such methods, including the present invention, provide for a likelihood of a particular outcome. When a patient is diagnosed with RA, then particular treatments can be prescribed. Similarly, when the prognosis is that RA will develop, the patient may be treated accordingly. However, when it is established that it is unlikely that RA will develop or that the patient does not have RA, then alternative treatments can be provided. Thus, if the patient is likely to have resolving disease, then the output (i.e. the advice) may include a course of antibiotics, anti-inflammatories and/or rest. Other suitable treatments to treat joint inflammation are known, but it will be appreciated that these are not effective, especially long-term, when the underlying condition is RA. Accordingly, rapid and accurate diagnosis of RA is clinically and socially important, both from the point of view of the treating the patient and in ensuring that funds for treatment are spent in the most cost-effective way (i.e. by avoiding funding inappropriate treatments).

Low AdipoR2:AdipoR1 ratio and/or low surface expression×ratio score is indicative of an increased likelihood of development of RA. This is increased with respect to, i.e. greater than, the likelihood of the patient having resolving disease. Of course, the patient may already have RA, especially in its early forms, but has preferably not yet been diagnosed with RA. As mentioned elsewhere, to be officially diagnosed with RA, a current set of criteria must be met, even though the disease is present up until the day the decision is made. In other words, although the criteria and length of time required to meet these or receive a decision may change over time, the invention nevertheless provides a way of assessing whether a patient is in an at risk group (for RA) or not. Accordingly, the present method preferably comprises assessing whether a patient will go on to formally meet the criteria for an RA diagnosis. The method may be also be considered to be a method for (or of) assessing whether a patient is in an at risk group (for RA).

The criteria for developing RA are preferably met at the 18 month stage.

Preferably, only one of AdipoR1 or AdipoR2 is assessed at protein level. This may preferably be AdipoR2, but may equally be only AdipoR1. AdipoR2 is generally preferred as it shows less variability, but bigger diagnostic/prognostic changes. Most preferably, however, expression of both AdipoR1 and AdipoR2 is considered and the ratio of the expression levels used to define patient groups. For mRNA, both AdipoR1 and AdipoR2 will preferably be assessed in order to provide the ratio between the two which again can be used alone or with other measured parameters to provide a ‘score’ or ‘threshold’ below which patients are likely to develop RA and above which they will be diagnosed with synovitis which is not RA. Where both are considered, this is preferably at substantially the same time, as this should help to reduce errors caused by potentially changing expression patterns. A “snap-shot” is thus preferred.

The present method may also preferably include, or be used alongside, existing methods of diagnosing RA, such as but not limited to: imaging e.g. via X-rays, MRI or ultrasound; and/or blood tests e.g. test for rheumatoid factor, tests for the presence of the anti-citrullinated protein antibodies (ACPAs) or anti-CCP (cyclic citrullinated peptide) and the anti-MCV assay (antibodies against mutated citrullinated Vimentin).

RA is known to have secondary links with diffuse inflammation in the lungs, the membrane around the heart (pericardium), the membranes of the lung (pleura), and white of the eye (sclera), and also nodular lesions, most common in subcutaneous tissue. These parameters tend to occur later in pathology in some patients. Accordingly, these are secondary indicia that may be used in conjunction with, but not instead of, the main indicator: joint inflammation. The present biomarker is therefore relevant specifically to patients with very early arthritis (i.e. joint swelling and pain/inflammation of less than 3 months).

The resolving disease may be very early arthritis, but which is not rheumatoid arthritis and, therefore is likely to spontaneously resolve without treatment. However, very early rheumatoid arthritis (RA) may be considered to be a condition which ultimately fulfils the criteria for RA, for instance at an 18 month follow-up. It is preferred that those classified as likely to have or go on to develop RA satisfy the requirements of the American College for Rheumatology 2010 classification criteria.

Synovitis causes joint tenderness or pain, swelling and may go on to present hard lumps called nodules. Symptoms of synovitis can be treated with anti-inflammatory drugs, such as NSAIDs or an injection of steroids, or biological therapies such as Rituximab.

It will be appreciated that terms such as ‘determined,’ ‘measured,’ ‘detected’ or ‘assessed’ may be used interchangeably unless otherwise apparent.

The expression is optionally protein expression, optionally on the cell surface. However, expression at the transcript (RNA and most preferably mRNA) is also envisaged. Indeed, the transcript and protein expression may be measured separately. However, it is preferred that they are measured at approximately the same time. Preferably, they are measured from different aliquots of the same sample. Preferably, any measuring is made approximately contemporaneously. It will be appreciated that reference to the term ‘amount’ may also be taken to refer to the term ‘level’ of, for instance, a transcript or protein being measured.

The patient has joint inflammation, which may present as joint stiffness or pain. Most preferably, the patient has (or presents with) synovitis (although it will be appreciated that they may not have been diagnosed with this yet). The patient may present with joint inflammation or synovitis or may have been diagnosed or suspected of having said conditions by a health practitioner.

The patient is preferably a human, but it is also envisaged that non-human mammals may be used in the present method, in which case the expression of AdipoR1 and/or AdipoR2 should be determined. Indeed, it should be appreciated that reference herein to AdipoR1 and/or AdipoR2 includes non-human homologues unless otherwise apparent.

The present method preferably includes the step of stratifying the patient into one or more groups or cohorts. Preferably, these are “patients with or likely to develop early RA” and “patients with other, non-RA related, conditions.” The latter preferably includes patients with resolving disease, i.e. those patients where treatment of the underlying condition can be successfully, or largely successfully, completed, or at least the symptoms alleviated using anti-inflammatory treatments such as NSAIDs as mentioned above. However, those patients with, or likely to develop, RA, are unlikely to ultimately be successfully treated using such anti-inflammatory treatments. This is because the underlying cause of the disease is not known and is chronic in nature and requires more aggressive anti-inflammatory therapies such as the administration of, for example, methotrexate and/or anti-tumour necrosis factor therapy. Notably, such interventions still only alleviate symptoms in approximately 50% of patients with established RA, although diagnosis of patients earlier will allow more effective treatment as described above.

Indeed, the method itself may be viewed as a method of stratifying patients into (i) those having or likely to develop early RA and (ii) those unlikely to have or develop early RA (and instead having resolving disease). Such methods would include determining the expression of AdipoR1 and/or AdipoR2 in leukocytes in the sample from the patient. Of course, reduced surface expression per se, or a ratio of less than 1 of AdipoR2/AdipoR1 (AdipoR2 divided by AdipoR1), or the use of these data in other algorithms is indicative of the development of RA. In other words, the stratification of the patients may predict clinical outcome.

Patients will tend to present with joint inflammation and this will ultimately be either resolving (i.e. is cured with treatment) or persistent (i.e. where treatment is attempted to keep the symptoms in check). RA is considered to be “persistent” because we currently do not understand the cause of the disease and we do not have adequate treatments to “cure” the disease.

The expression of AdipoR1 and/or AdipoR2 may be assayed at the RNA or the protein level. Accordingly, an assay to determine the quantity or level of mRNA expression may be used, such as PCR, in particular quantitative PCR (qPCR). Other means for determining the quantity or level of mRNA are of course also envisaged. Measurements may be made in crude mixed isolates of leukocytes or on cells after fractionation into subsets.

Alternatively, the level or quantity of protein expression may be determined. This is most easily done by determining the quantities or levels of cell surface expression of the resulting AdipoR1 and/or AdipoR2 receptors, including the proportion of the cells that express the protein. This may be measured in conjunction with other surface proteins which are markers of different leukocyte subsets. Alternatively, the level of protein expressed within the cell, or in different cellular fractions can also be determined. Suitable methods include the use of flow cytometry, western blotting and/or confocal microscopy.

Accordingly, where reference is made to the expression of AdipoR1 and/or AdipoR2 in the particular cells, then it will be appreciated that this can include both detection of the polynucleotide transcript, the peptide and/or the resulting functional protein. The functional protein may be expressed on the cell surface, so in this sense, the “in” a certain cell can also include “on” said cell.

The cells in the sample are leukocytes. Preferably, these are Peripheral Blood Leukocytes. Measurements may be made of sub-populations of the leukocyte mixture, preferably peripheral blood mononuclear cells (PBMC) or peripheral blood lymphocytes (PBL) as the AdipoR1 and AdipoR2 receptors are highly expressed on many subsets of circulating PBL and PBMC. The PBL subpopulations may include T-cells, B-cells and various subpopulations of natural killer cells such as NK T-cells. Analysis may be performed on PBMC or PBL, although B cells and/or NK T-cells are in general preferred, as narrower classes affording greater accuracy.

In patients likely to develop RA or those with very early RA, the expression of AdipoR1 and/or AdipoR2 is reduced. In addition, patients likely to develop RA or those with very early RA, the mRNA expression of AdipoR1 is elevated whilst AdipoR2 mRNA expression is reduced.

It will be appreciated that these changes may be in comparison to a healthy control, preferably once suitable filters, such as age and gender, have been applied. Similarly, preferably the same changes are also seen in comparison between the RA patients and those with resolving disease. Again, it is preferred that said resolving disease patients have been filtered accordingly as mentioned above. In other words, the controls against which the change or changes are measured may be completely healthy patients, or patients with joint inflammation (resolving disease) that are ultimately cured. Mathematical algorithms based on the aforementioned changes will provide standard ranges allowing stratification of patients into different clinical outcomes. The control against which a patient is assessed may also be the same patient, sampled at a different point in time. This allows progression of the condition (or not) to be monitored in a particular individual over time. Such an individual may be one that has had a family history of RA or is otherwise identified as being in an ‘at risk group.’ Thus, the method may preferably comprise two or more assessments (determinations of AdipoR1 and/or AdipoR2 expression) over time, i.e. over pre-determined time intervals, with fluctuation preferably being identified. A reduction in AdipoR1 and/or AdipoR2 expression (or use of this information in appropriate algorithms [as above]) over time may, therefore, be indicative of an increased likelihood that the patient is beginning to develop RA, and suitable interventions may then be made or prescribed.

Unfortunately, certain therapeutic interventions such as methotrexate and/or anti-TNF therapy unbalance the Adiponectin/AdipoR system, so this latter aspect is generally not preferred, unless it is known that no therapeutic intervention has been made in the intervening period or, if it has, then that intervention is known not to unbalance the Adiponectin/AdipoR system. In view of this, internal (within the same individual) comparisons between expression of AdipoR1 and AdipoR2 are preferred, see the algorithms (i), (ii) and (iii) above.

The sample is preferably a blood sample, although other fluid samples are envisaged (for example synovial fluid is also preferred). Most preferably, the sample is peripheral blood.

Accordingly, the use and methods also preferably include any of the following steps, alone or in combination:
sampling of a body fluid, preferably blood; identification, for instance isolation, of the Leukocytes, which most preferably will be Peripheral Blood Leukocytes and most preferably the Lymphocytes discussed herein; and/or
detecting the expression of the AdipoR1 and/or AdipoR2 in or on the cells; and comparing these against control levels. A reduction in the levels of expression of AdipoR1 and/or AdipoR2 is indicative of the patient having early RA or going on to later develop RA (i.e. is prognostic of RA).

The AdipoR1 and AdipoR2 receptors are highly expressed on many subsets of circulating PBLs. By way of example only, and although it will be appreciated that a Ct value can change between qPCR runs, the following ranges should generally apply for healthy controls:
18S threshold cycle (Ct) values in the range of 16-20 are preferred;
for AdipoR1, Ct values in the range of 27-31 are preferred for healthy controls; and
for AdipoR2, Ct values in the range of 26-29 are preferred for healthy controls.

The AdipoR1 and AdipoR2 receptors are highly expressed on many subsets of circulating PBLs. By way of example only, and although it will be appreciated that a Median fluorescent intensity (MFI) value can change between flow cytometers and antibodies used, the following ranges should generally apply for healthy controls following subtraction of the isotype control antibody:
for AdipoR1, MFI values in the range of 19-45 (for PBMC), 5-9 (for PBL), 19-90 (for B-cells) and 9-43 (for NK T-cells) are preferred for healthy controls; and
for AdipoR2, MFI values in the range of 17-45 (for PBMC), 4-12 (for PBL), 20-95 (for B-cells) and 20-66 (for NK T-cells) are preferred for healthy controls.

For the expression of AdipoR1 and/or AdipoR2 on the cell surface, thresholds of 60% or less of B cells or 20% or less of NK cells are indicative of early RA (i.e. a population of B cells where only 60% of the B-cells show the marker is indicative of early RA, whilst a population of NK cells where only 20% of the NK-cells show the marker is indicative of early RA). In particular, these thresholds preferably apply to both AdipoR1 and AdipoR2, such that thresholds of 60% or less of B cells expressing both AdipoR1 and AdipoR2 is indicative of early RA, or thresholds of 20% or less of NK cells expressing both AdipoR1 and AdipoR2 is indicative of early RA. Preferably, the B-cell and NK-cell thresholds are both measured to improve accuracy. The threshold (cell %) may be assessed by Flow Cytometry, for example.

It will be appreciated that absolute flow cytometry readings, for instance, may vary depending on the reagents and machine used. However, using ratios according to algorithms set out in (ii) and (iii) will overcome this.

### Examples of Antibodies Useful in the Invention and Preferred Conditions Therefor

### AdipoR1

Link: http://www.phoenixpeptide.com/cataloq/product info.php?products id=4686

### Adipo R1 (357-375)/Adiponectin Receptor Type 1 (357-375) (Human)—Purified IgG

Antibody from Phoenix Pharmaceuticals, Inc.

### Catalog #G-001-44

Catalog #G-001-44
Standard Size 200 μg
Sequence Asn-Leu-Gln-Glu-Phe-Arg-Tyr-Gly-Leu-Glu-Gly-Gly-Cys-Thr-Asp-Asp-Thr-Leu-Leu (SEQ ID No. 1)
Species Human
Host Rabbit
Reconstitution For consistent and reproducible results, reconstitute with 110 μl of distilled water and 90 μl PBS (or 50% glycerol in PBS) for the equivalent of undiluted antibody, immediately before use.
Storage For optimal results, use the antibody as soon as possible after
Condition reconstitution. Store in lyophilized form unless needed and reconstitute immediately before use. Once reconstituted, the antibody should be stable for a few days at −4° C. For storage up to a few months, prepare small aliquots after reconstitution and freeze at −20° C. or −80° C. Repeated freeze thaw cycles should be strictly avoided.
Contents This vial contains 200 μg of rabbit IgG in 110 μl PBS (137 mM NaCl, 2.7 mM KCl, 4.3 Mm Na₂HPO₄, 1.4 mM KH₂PO₄, pH7.2) in the lyophilized form

### AdipoR2

Link: http://www.phoenixpeptide.com/cataloq/product info.php?products id=4684
Adipo R2 (374-386)/Adiponectin Receptor Type 2 (374-386) (Human)—Purified IgG Antibody from Phoenix Pharmaceuticals, Inc.

### Catalog #G-001-23

Catalog #G-001-23
Standard Size 200 μg
Sequence Phe-Met-Ile-Gly-Gly-Gly-Cys-Ser-Glu-Glu-Asp-Ala-Leu (SEQ ID No. 2)
Species Human
Host Rabbit
Reconstitution For best and reproducible results, reconstitute with 200 μl of distilled water for the equivalent of undiluted antibody, immediately before use.
Storage For optimal results, use the antibody as soon as possible after
Condition reconstitution. Store in lyophilized form unless needed and reconstitute immediately before use. Once reconstituted, the antibody should be stable for a few days at −4° C. For storage up to a few months, prepare small aliquots after reconstitution and freeze at −20° C. or −80° C. Repeated freeze thaw cycles should be strictly avoided.
Contents This vial contains 200 μg of rabbit IgG in 200 μl PBS (137 mM NaCl, 2.7 mM KCl, 4.3 mM Na₂HPO₄, and 1.4 mM KH₂PO₄, pH 7.2) in the lyophilized form.

### Example 1

### Changes in AdipoR1 and AdipoR2 Expression on Circulating Leukocyte in RA

Birmingham Early Arthritis Clinics systematically collected blood samples from patients who are categorised into the following groups based on clinical outcome and disease duration [16; 17]:
1. Resolving (Res)—very early arthritis which resolves spontaneously without treatment.
2. Very early RA (RA)—DMARD naive unclassified arthritis with ≦3 month symptom duration at time of sampling which ultimately fulfils criteria for RA by 18 months of follow-up.

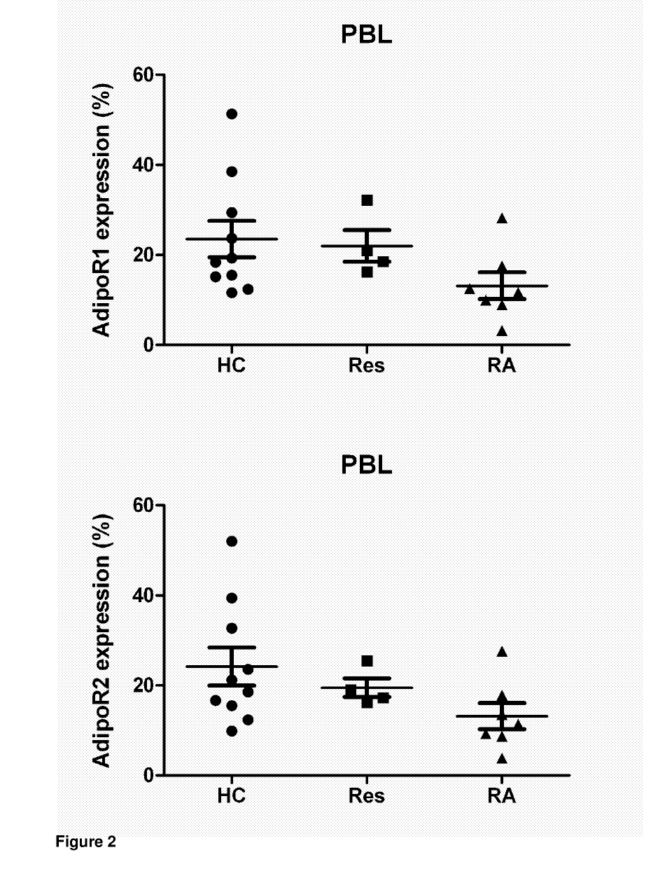

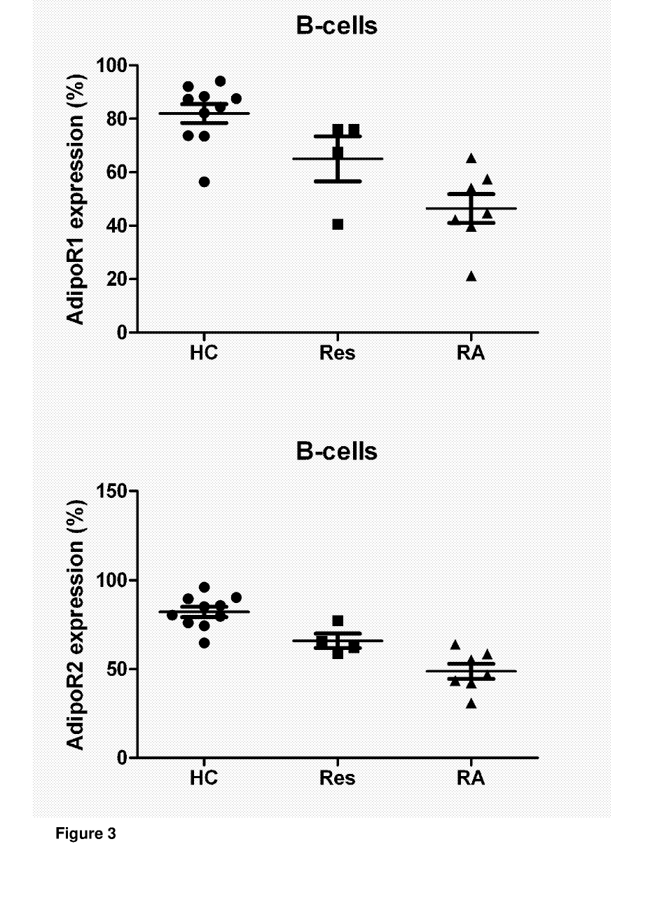

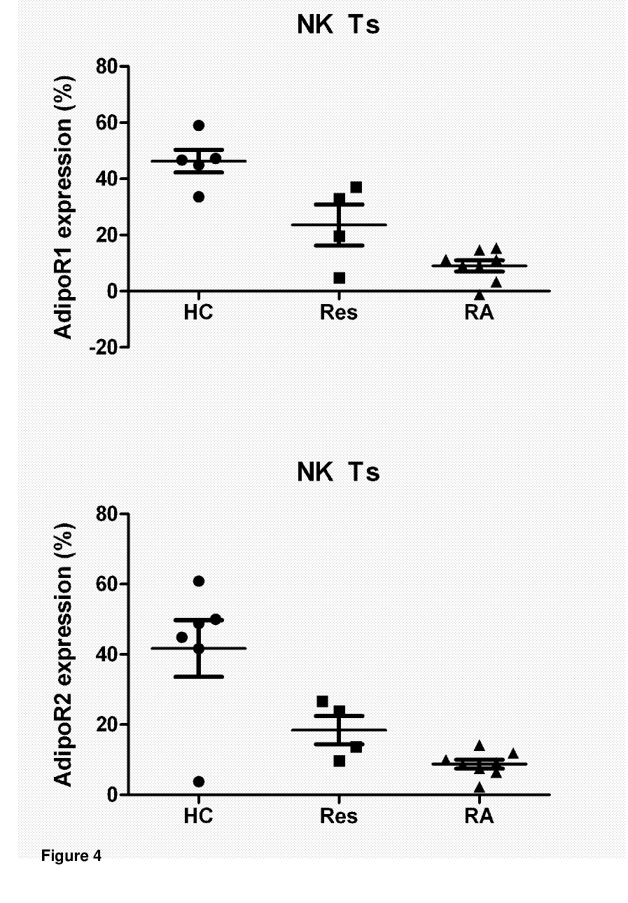

Blood samples from age and gender matched healthy controls were also obtained. Peripheral blood lymphocytes were isolated and the expression of the adiponectin receptors on individual subpopulations was assessed by flow cytometry. We observed a significant reduction in the surface expression of the adiponectin receptors on circulating leukocytes isolated from patients with early unclassified RA, compared to healthy controls and patients with resolving disease. This pattern was evident for mixed peripheral blood lymphocytes (FIG. 2). However, it is particularly marked on B lymphocytes (FIG. 3) and on natural killer T cells (FIG. 4).

It appears that, in patients with undefined synovitis (joint inflammation), a reading of less than 60% of B-cells or 20% NK-T cells positive for adiponectin receptors would be associated with increased risk of developing RA.

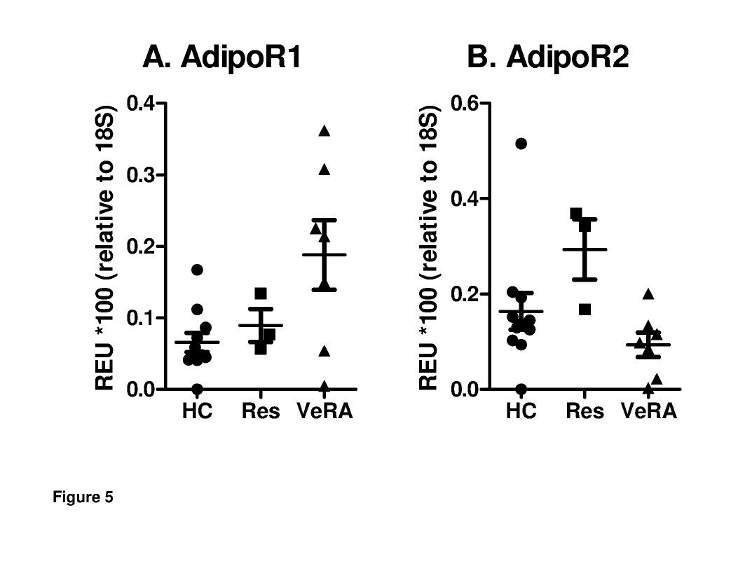

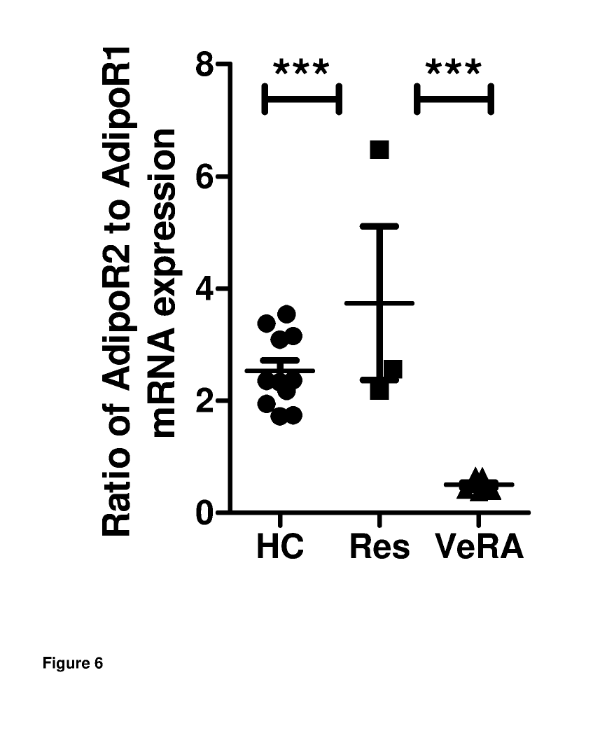

Alternatively, peripheral blood mononuclear cells were isolated and the mRNA expression of the adiponectin receptors were assessed by qPCR. We observed an elevation in AdipoR1 and a reduction in AdipoR2 mRNA expression on circulating PBMCs isolated from patients with early unclassified RA, compared to patients with resolving disease (FIG. 5). When we analysed the ratio of AdipoR2 to AdipoR1, we observed a significant reduction in the receptor ratio of patients with early unclassified RA, compared to patients with resolving disease (FIG. 6). In addition, those patients with resolving disease exhibited a significantly higher ratio compared to healthy controls.

It appears that in patients with undefined synovitis, a AdipoR2:AdipoR1 ratio of less than 1 would indicate/be associated with increased risk of developing RA.

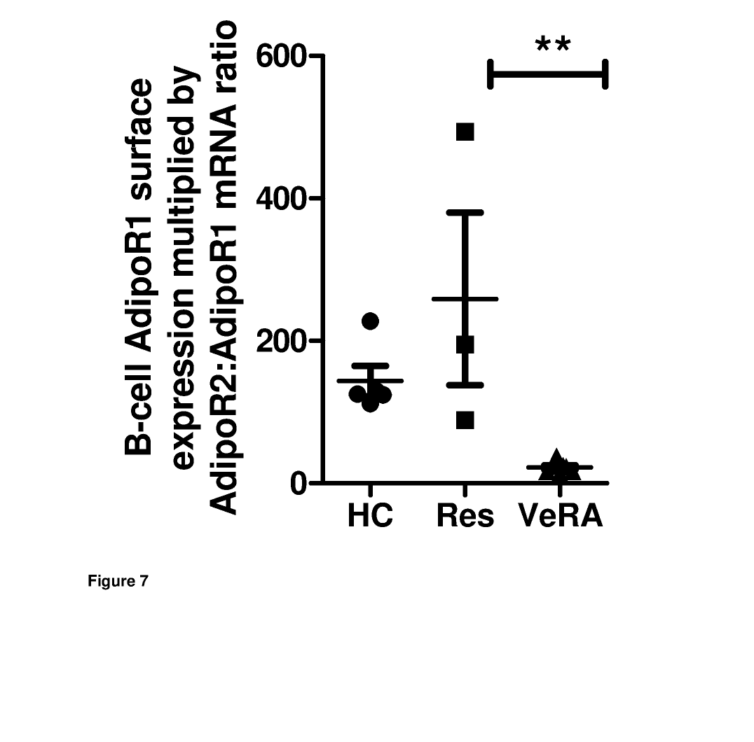

The surface and gene expression of adiponectin receptors can also be evaluated together to provide additional power in the data. Here the surface expression of adiponectin receptor 1 and/or 2 on individual lymphocyte subsets is multiplied by the AdipoR2:AdipoR1 mRNA ratio. Using the surface expression of AdipoR1 on B-cells for example, we observed a significant reduction in the score of patients with early unclassified RA, compared to patients with resolving disease (FIG. 7).

It appears that in patients with undefined synovitis, a surface expression*ratio score of less than 50 would indicate/be associated with increased risk of developing RA.

Thus, expression of adiponectin receptors on lymphocytes has great potential utility as a biomarker in early arthritis clinics to diagnose those patients with RA earlier than is currently possible.

This has the added advantage of distinguishing them from a wider cohort of patients with uncharacterised resolving disease. Current clinical practice requires patients to fulfil the American College of Rheumatology 2010 classification criteria to be diagnosed with RA. As such, patients can often wait several months before receiving a confirmed diagnosis, by which point it is too late, as the crucial window of opportunity for an early intervention has closed.

Accordingly, early detection and diagnosis of RA by this invention will allow earlier clinical intervention that will improve the long-term outcome for patients. Our data indicates that adiponectin receptors on lymphocytes are the first biomarker capable of stratifying patients within days of them presenting to clinic, allowing improved patient-based therapeutic intervention and disease control.

### Example 2

### Diagnosing RA at an Early Arthritis Clinic

1. Patient presents to health care worker with joint inflammation;
2. Peripheral blood sample is taken;
3. Peripheral blood mononuclear cells and/or peripheral blood lymphocytes are isolated from sample, preferably immediately;
4. mRNA levels are determined for adiopR1 and adipoR2 from peripheral blood mononuclear cells sample;
5. Protein expression (e.g. cell-surface) for AdipoR1 and/or AdipoR2 is detected on individual lymphocyte subpopulations;
6. Data is incorporated into a mathematical algorithm and a score is outputted. For example:
   a. mRNA ratio of adipoR2 to adipoR1 (adipoR2/adipoR1) on B lymphocytes of less than 1=patient is likely to develop RA. Ratio of AdipoR2 to AdipoR1 on B cells of greater than 1=patient is likely to resolve.
   b. The surface expression of AdopR1 protein multiplied by the ration of the mRNA expression of adipoR2/adipoR1, where scores of less than 50=patient is likely to develop RA.
   c. Other examples of a suitable calculation are provided herein.
7. Determination is made based on the output compared to a reference level, or on the score or ratio outputted and compare to a threshold, and the patient is stratified into likely or not likely to develop RA

### REFERENCES

1. Firestein, G. S. and Zvaifler, N. J., Immunopathogenesis of rheumatoid arthritis. Immunology Series. 54:141-61, 1991.
2. VanderBorght, A., Geusens, P., Raus, J., and Stinissen, P., The autoimmune pathogenesis of rheumatoid arthritis: Role of autoreactive T cells and new immunotherapies. Seminars in Arthritis and Rheumatism 2001. 31: 160-175.
3. Raza, K., The Michael Mason prize: early rheumatoid arthritis—the window narrows. Rheumatology. 49(3):406-10, 2010.
4. Ouedraogo, R., Gong, Y., Berzins, B., Wu, X., Mahadev, K., Hough, K., Chan, L., Goldstein, B. J., and Scalia, R., Adiponectin deficiency increases leukocyte-endothelium interactions via upregulation of endothelial cell adhesion molecules in vivo. J Clin. Invest. 2007. 117: 1718-1726.
5. Ouchi, N., Kihara, S., Arita, Y., Maeda, K., Kuriyama, H., Okamoto, Y., Hotta, K., Nishida, M., Takahashi, M., Nakamura, T., Yamashita, S., Funahashi, T., and Matsuzawa, Y., Novel Modulator for Endothelial Adhesion Molecules: Adipocyte-Derived Plasma Protein Adiponectin. Circulation 1999. 100: 2473-2476.
6. Brochu-Gaudreau, K, Rehfeldt, C, Blouin, R, Bordignon, V, Murphy B. D and Palin M F. Adiponectin action form head to toe. Endocrinolgy 2010. 37:11-32

Sequences for Adiponectin receptors gene, transcript and protein sequence

Adiponectin receptor 1 (AdipoR1/AR1) (SEQ ID No. 3):
Gene sequence:
>gi|224589800: c202927700-202909960 Homo sapiens chromosome 1, GRCh37.p9
Primary Assembly
ATCACGCACTTCAGGCTGCGATAACGAGGTGGAGTGTAGAATCCGACGAACGTCCTCTACCCCGGGGCGG
GTGGAGGTTACCTGGCTGCGGCGGGGAGGTGGGCAGGCGGCAGGGAAGGCGGGGCAGCAGGGCGGGGCCC
GGGGCGGGAGGTGTGGCCGGCGGGCGCGGGCCTGTGATTGGGACTCCGCGAGAGGGCCTAGGATTGGAGG
GCCCAGGCAGAGCCCGCGAGGAGGTGACGCGGCTGCGGAGGTGACGCGGGAGGTCGCGCGCCCCTTCCGG
CGCGGGGAGGGCGCTGAAGATCGGGGCCGCTCGGCCGCAGGCCGCCTCCAGCGCCGCGGGATGTAGCGCG
GGGGACCGCGGCCCCCAGCAGAGCCCGCCTGCCCGGCTGTAAGTCTCCCAGGGGCTGCGCGGTGGGCAGG
GACTCGGCGGGAGCGCGGGGACCCCGGGGGCCCCGACCCAGGCCTGCGGAGGAGACAGCTGTTGGGGGAA
CCCGCTCCGACTTGCCAGGGGGCTCGGGCCCCAGCCAAGGAGCCGCCTGGCCTCCAGGCATCCAGCCGGT
CAGGTTGTAAACATTTCCGGCCCCTCCCACTGCGCGCCGCCTCCGAGGCGGCCTAGGTGCGGTGGGCGCT
CGTCCCCGGCTGTCCCCCGACTCCCGGCAGTCCTGTGAGTGCTGCGCCTTTAGCCCCACGGGGTGGAGAT
GCATGGGTTCCCCAGATCGGGACCAGAGAGCCAGAGTTAAGCCTGGCCCCGAGGCCGCCGCGCGTGGGGA
GGGAGGGCTGCGGGCCTAGGTCGCGAACCGCCTCTCAGCGGGGAACAGCCGCATTGGGTTGCCAGGAAGC
CCTAAGGTTCCTCCACCAACAATCTTTGGCAGGTCAAGTCCTGACAGTGCCCTCACTTTGATGGGGTGGG
AAGGGAGGTTGGTGATCGCTCTGCAGGGTTAAGGTAATCCTGAAGGCTGAAATGCGGGGCAGAATCGAAG
GTACTTTGGGGGAAAATGAAATTTCATTACCTTAGGTATCTAGGATTGATGTATTCTATCTTCTGTTTGA
GAATTTTGGTCTTCAAGCCTTCGATTTTTAAGAAAATAGATAAGTGTTTTTTTTCCCTCAGTTTCTGGTG
CTCTTTGGGAGAATGGGAAACTGACAAGGCCAATTTCAGTTTACACCGTTTCAGATGTCAAAGGAGCGCT
AGGAGGCCATGGTACTTTACTAGATTAACCATAGTACTTCCCTCAAGTGATTCAGAAGACGGGCTAGAAA
ATACAGCGGAGAATACATAGGCTGCCATCTACAAAACAAATGATTGTAATGGCTGGTTCTTTTCTGAAAT
GAAGTAATAGTGGGGTCGGTTCACTGCTTTTCTCTTTTCCTAGTAGTTGGGTAGGCTACGATGGTGCAAC
TTCGGTTCCTGAGGTTGTATACTATCTGTATACTGCCAGGAATTTAGGGTGGTGCTAAACAGATTGCAAT
GTCAGGGTACCGGCTTGCAGCTACTAAATGTATTTACTCCAGGAATAACTTTTATGTACCCATATTGTGA
TTTGCACTTGGGAAATCAGTCATAAAAACTGATGTTACCTGTGAAGGCCCATGTTATGGTAGACCTCCGC
AAAACTATACCTAATTGTAGAGTAGAAACTGAAAGCTTTCTATTCTCAATGGTCAAGACTTTGCCTGCAG
GTAGGAGAGAATCTCAGAAAAATAACAGGGAGCAACATTAATCCACTGATCCCCATTGCTGATTTATAAA
GTACACCACCTTAAGTAGGCTTTTAGGCTATTTTCAGTTTTTTAAGTCTGGCCTCATTTTACCTTTCCAG
GACTCTATGGAAAAATGAATTGTTTACTGCCCTGGGGACTGTGTTCTTTCATGGCATGTTCACACCATAT
ACCCTCCTGGAATGCCCTGTTTCCATCTCTATCTATCAATCTCCTGTCATAAAATCTTCCACAAAACTTT
TTCTGATCACTTCAACTAAAGTTGCACTTTTCTTTTGAATTCCTCCTGCACATAATGGCACTAAGCCTTG
TAGTGATATTAGGAAAGTTATTTCCCCCATTTAGAGGCCAACAACCATTTCAACCCTCTGACTCCTCTTT
GCCTTGTCTCTGTGGCCACAACACCATGCGTATTGTAAGTAATCAATCGGTATTCTCAGATTAATGAAGG
TTAGAAATTGCCCTTCTTTCTAGGCCAGTGTATTTGTTCTCTATTCCCATAGGAAATTTCCTTTGACTTC
TAATACAAAGCCAAACTGAAACCCTTTAACACTCCTGCTACATTATCCTGTCCCATCTACTTATTATCAC
ACAGGTAAGATAAGCAGTCGTCTTTATATCTAGACGCTTTGTGAATTATGGTGAGTTTTTTTTCTTGGAC
CAGTACTCATCAACGTTGTTATGTCCATTTGTCCCTAATGTCACCTCCTGGATTTTGTTCCTCTCACCAT
GCTAAAAGTTGATTCCCATTTTCCTGCTCAGAAATGTTGGGGGCATAGGCCGGGCACAGTGGCTCATGCC
TCTAATCCCAACACTTTGGGAGGCTGAGGCGAGTGGATCACCTGAGGTCAGGAGTTCGAGACCAACCTGG
TCAACATGGCAAAACCCTGTCTCTACTAAAAATACAAAAATTATCCGGATGTGGTGGCAGGTGCCTGTAA
TCCCAGCTACTTGGGAGGCTGAGGCAAGAGAATTGCTTGAACCTGGAAGGCGGAGGTTGCAGTGAGCAGA
TCATGCCACTGCACTCCACCCTGGGGGATAGAGCAAGGCCCTTGTCTCCAAAAATAAAAGTGGGGGCTGG
CATTGGCTGGGCACGGTGGCTCACACCTGTAATCCCAGCACTTTGGGAGGCTGATGCAGGAGAATTGCTT
GAGTCCAGGAGTATAGGCTGCAGTAAGCTATGATCGTGCCACACACTCTAGCCTGGGCCTGGAACTGGCT
GAACTTGGGAGCAAGACCCTGACTCCAAAAAGAAAAAATGGGCTGGCATTAAAATCCTTCATATGCCTGT
ATACTGTTCACTTTTTTTTTTGTCATGACATTAGCAAAGTTTGCCTGTCTGTCAGGGCTCAAAAACACCT
CTTCAGCCAGGCACAGTGGCTCATACCTGTAATCCCAGCACTTTGGGATGCCAAGGCTGGGCAACATAGT
GAGACCTTGTCTCTACAAAAAAATAAAATTAGCTGGGCATGGTGGCACATGCCTGTAGTCCCGGTTACTA
AGACTTGGGAAGCTAAGGTGGGAGGATCGCTTGAGTCTGGCACATTGAGGCTACAGTGAGCTGAGATCAC
ACCACTGCACTCCAGCCTGAATGACAGAGCAAGACCCTTTTAAAAAAAAAAAAAGACTTCTGTTGGGGAA
GAGCTACAAAGGAAGTCAGACTGTTTTTCCACTTCAGAGTCTGGGACGCTTGGCATCGAGTAGGAGAGCT
GAGGCCCTATATACTGTAGTTCGGTGATTTCAAACATGTGCCTTTGCTTTGATTCATATTTCTCCAACAA
AGCTGTGAAATTATCCTGCCTAAGAGACAGTAGATAGGGGAAAAATGGCTGTATTCTTTATCTATACTGC
TAGACTGGTCTTGCTTTAGGACAAGAATCAAGCTAGGTATGGATAGAGGCTACCCATAGACTCTCTAAAG
GCCCAGGAACTAAACTCTTAGAAACTTTGTGTCTTTGCCTCCAGGAAAGGTGTGCCGTAGCCTGGAAAAC
TTGGGAATTGGTTCACAATATAAATGTCTCCAAAAACTATAAGTGAAATAACTCTCCAGCTAACTGGCTG
GTGATTAAAAATCAATCTTTTCCCCATGTTTGGCACAGGATTATCTTATTTATAGGAGTACAAACAACCT
CCCTGCTCCACAGCTTTACTCTCTAATATCCTAGGTTTACCCAGTTGGAAAGCTTAGTGGTAAAGGTTTA
TTAGCTTGAGTTGAAGAAATCTCATCAAAAATGCACTCAGTCGAGTTTGGCTCAAATATAATTTGCAGTA
TTGGGTGTGGCAAGCTTGAGGGGAAAGGCATTTCCTCTAAAATACGTAAGGACATAGAAAGCTGGCAGAA
GGTTGGCTCACCTAGGATCGTTCTAACACCTTAGGGGTTTGTTCTGATTTTACACCATAGACTAAAACAC
ACTTGTAATTCTCCATTGTTTGTGAGTGATCCACCCCATAGAAGTGGTATTTTCAGATCCTAGTATTATT
TGCTCTTCTTTCTTGCCAGACGTCCGATCCCTATCTCTCCCTTTCCTGTCTCTGTGTTACTACAGTTTAG
AAGACCAAACTGGACAATGGACTTTGCTCACTATGATGACATGATCTCCATTGACCTCCATTGTATGTAC
TTTAATGCATGATGATATATTTTGTATGATGAGTTGTAGCTGATGGCTGGTTTGTGAGTTAACTGGGGTC
TCAGGTTTTGGTGACTCTGGGTCTAGAAGATGTAGAGAGGTGGTATAGGAGCTAGTGATAGAGGAGCTAG
AGGGAATCGGAAGAACTAATAGTAAATGATTTGGACTGAAAAAGGAAAAGACTTTGATGATAGCCAGAAG
ACTTGAGGGCATAAAAACTTTTCAGATCAAATGATGAAATTAGTAAGAATGCCAATCTGGATTCTGCACA
TGATCTTTGGACCCTAATTGCCCAACTTAAATTTTCTCTATATTATTCTCATTGCCTCTAGTAAAATAGT
TACATAGCACTCAAAGTTACTAAATTTAGTGTGTGACCATTCTAGTTGAGGGGTTCTCTCCCTCCACTGC
CCTCCTGGAGCAATCAAATTGTATCACTTATTTTTGTGCATATCACAAACTACCTATGTTATCTGGCTCT
TTGAAAACAATACTATAACTTGTTCAACTTGTATTTTGCACAGTGTCTTGTATTTTGCACAGTGTCTTAG
AATGCCTTAGGGTGCTAGTCCTAAATACACTTGCTAAATATTCAATGAATGTGTTGACTTACTCTGGGGA
AAGGGATAAGAGAAGCCACCACAAGCACAAGAAAATGCACATAAGCCCTTATCTAAGTGGCTAGAAGCTA
AGTCATATGCTGGCAAGCTGGAATCTGCCCCATTCATTCATTCATTTTGAAATGGGTTCTAACTAAAATA
AAGTATATTTATTATTTTATGATCCATGTATTGCATCTCAGAACCAGAATTTTGTTTTGTTTTGTTTTGT
TTCTGCCCTGGTAGCCTGATCTAAGTTACTTCCAACAGTAACTTAGTAAGTAACAAGCTAAGTGTGCCCC
TCTTTTCAGATTTATCTAAGATGCCCAAAAATATTTATGTTTTTTGCTTGTGATATAAAGTTGTTTTTTG
GTTTTTTTTTTTTTGCTTGCGATATAAAGTATTTTTTAGTAAAGCTGCTGATTCAGTAGTACTTCACTTT
GAGGAAATATGTTTTGAGGTCAGCCTATTGAGAAAGATTTCCTTGTGTCCCTCTCACACGGGTTGCCCAT
CCTCGAGGTTTTCATAGGCTTCCAGCTTCCTGTTCTCCTTAGCTCAATTGTTCACTATGACTGTGTGCTG
GCATCTCGTCAGAGTCTTGCACATTTAAAGGAAGAGGAACTGTCTATGTGTTGACACCATATAAAGGTCT
GCTGTATAGCAAGTTATTGTTTTTGTCTCTCTTGGACTAGATTGCCGGTTGCTTTCAACTGTGCTTGTGG
GTTGAGCATAGCATGACTCATGACTGTTTAGAAGCTTGAGGATTGCCCTTACCTGATGTGTTAGTAAGTG
TTCTCCAGAAAGCACCAATAGGATACATACATATGGATAGAGATGGGATTTATTCGGGGAATTGGCTCAT
GAGATTATGGAGGCTAAGTCCCACACTATGCCATCTCAAGCTAGACAATGAGGAAAGTCCATAACTGGTT
CAGTTTAAGGCTGAAGGCCTGAGAACCCAAGGGGCTATTGGTGCACGTCCCGGAATCCCAAGGCAGGAGA
ACCTGGAGTTTCTGATGTCCAAGGGCAGGAAAAGAAGGGTGTCCCAGCTCCAGGAGGGAAGGAGGGGGAG
AGACAGAGAGAGTTAGGGAGTCAGAGAAAGAGATTTATAATCCCTGAGAAGGAATTGTGATTACTCTTAG
ATAAAAAAGAACTGACACAAGATATCAAAGATATTTGTATATAGTGAATAGCCATGGATGATATAGTGTC
TCCCTCAGGAGCAAAGTTATGTTTACTGCCCAATATAAAAGATTGGGGCTCCCTAAGCTTAAGTTTCCTC
TCCCGGAATGCAACCCACTCCATGTGCAGTTGTTACCTGGCCCTCCCTGCATTGCAATGTGGTTATTTGG
GTTTGGGTGGGGCTTGGGTAACCAATGCAAGAAAATGCTAATATTCTGGCTACTGCTATTGCTATAACTA
ATAAAGTCCTTTGTATGGGGGTAGAGGTGGGGAGAGAACTAATAATTTACAGCATAGTGGAAATCTTTGG
AAACTTTCTCCCTCTTTCCCTCTTTTTTTATTTGCATTTCCCTGAGGCCTGTTTAAAGACTACTGTGAAA
ATCCAACCCAGGAAAACCTAAAGTGTCTGAGATTTTTCCTAGAAAATGGAATTCTGGGTTTTTCTGATTT
TGTGTGTGCCTGTCTCTAGTTTCTGAAATACTCATTTGAGTAGGGCTTAGATTATGGGTTGTGGTGGTTG
AAACAAGTTATTGTGGAGCTGGTTATTAGGTATGTGTGTGGCAAAGGGTAAATTGGGGCTGCCTCCTTGT
CTTCACCTTCCTGCTTCCAGGTAAGTTCTTGCTTGCTTGCAGGACAGATTGTGATGAAAGCTTGTTATCC
CAGGGTTGAACTCAGGCTATAATCCTGTAATAGAATTAGTAGACTTCGCAGAGAAATTAACCACTAGGCA
TCTGTCCCACAACCAAGTTCCTTTTGTATGTATGACACATTTATCAGAGCATTTGTAGATAAGGTCTAAC
TGAGTTCAGTTGGCATTTAATTCATTTCTAAGCCTGGATGATCTTGTAGTAATTGCAGCCAGATGTTGGC
TTTATATTTTTATTCCTCGGTCTCAGAGCTAGGCCTTTCTTGGGCAGAGTGAGCTTTGGGAGTGATAGAT
GAGATTCTTCTCACTAAATGGTTCCATACCTGTGGGAGGGAGTTGAGGGTGGGTAGCTGTTTTTCTATGG
TGACAGTTTGGTGCAGTAACCATAGATCATATGAAAGCAAGCAACTAGGTGTTACATTTTCAGCCTTACC
TATAAGATTAGTGAGGTTCTGGGTAAAGGTTGACATTAAAGAGGGAGGACAGGAATAGATTTCATTTAAG
CAATTAACTGGCCTTTAGAATTGGGGACAGGATATGAGTTGATTAGCTTCTTATTCAGTATTAGAGTATA
GAAATGAAACTGTAAGAGGAATAAATTGTCAATCCTTCATGTTTGTATTTTCTTTTAGTGTCTACCATCA
GAGGGAGATCTCTGCCCCCTGGGGCTGAGAGACCCCAACCTTTCCCCAAGCTGAAGCTGCAGGGTATTGA
GGTACCAGCCAGATGTCTTCCCACAAAGGATCTGTGGTGGCACAGGGGAATGGGGCTCCTGCCAGTAACA
GGGAAGCTGACACGGTGGAACTGGCTGAACTGGGACCCCTGCTAGAAGAGAAGGGCAAACGGGTAATCGC
CAACCCACCCAAAGTAAGTCATCTTCCCAGGAGTCATCCCCTTGTTCAGTTTCTCTCCTTAGAACTCTGT
CATTTTCTCTTTTTTAAGAGCTCACCGTCCAAAAGGAGGAACATTGCATCCTGGAAATGTCTCTATCCAA
GTCCCTATGATACTGTTATAAACATCTTGTTGTCTGCTTTCCCCATTGTAAGCTTTTGGGAATGCACTCA
TCTTTGTAACACCAGTGCTTAGCACAAGGCCTGCAGTCCAATAAATAATCAGTAAATATCTGAATGAATG
AAGGAAGCAGTCAATTAGGATTAGGTTTGACTTCATATAACACACAAAAATCCTTACAGTTTTGACAGGA
CAGAAGTCTCTCAAGCAAAAGAATTTCAGAAGTAACCAATCCAGGGCTGATATGGAGGATCCACCACTGT
CAGGCACCTGTGTTGCTCTCCCATCACTCTTAATATGTGCCCATCATACCCACATTCTACCCCCCATGAA
AGGGAAACGAGAAGAAAGGCCCTCTCTTTCCCTTTTATTTTTATTGAGATAAGATCTCGCCATGTTGCCT
AGGCTGATCTTAAACTCCTAGGCTCAAGTGATCCTCCTGCCTCAGCCTCCCAAAGTGTTGGAATTACAGG
TATGAGCCACTGTGCCCGGCCAACTCTTTGCCTTTTAAAGGGACTATCAGGGCCGGGCACGGTGGCTCAC
ACCTGTAATCCCAACACTTTGAGAGGCCGAGGCAGGTGGATCACCTGAGATCAGGAGTTCGAGACCAGCC
TGGCCAACATAGTGAAGCCCCCTCTCTACTAAAAACACAAAAATTAGCTGGGTGTGGTGGCAGTCGCCTG
TAATCCCAGCTACTCGGGAGGCTGAGGCAGGAGAATCGCTTGAATCTGGGAGGCAGAGGTTGCAGTGAGC
CGAGATCGTGCCATTGCACTCCAGCCTGGGCAACGAGTGAAACCCCCTCTCAGAAAAAACAAAAATAAAT
AAAGGGACTATCAGGAAGTACCTCATAAGACTTCGGCCAGAACTTACTTACATAACTATATTCAGCTGCA
GTGGAGGTTAGAAAGTGCAGCCTGAGTAAAATTGCAGTAACATTGCCACCCTGAACAAAACCGTGGGCTT
TGTTACTTAGGAAGAGAGAGAATGGATGTTAGGGTAGACTGGCTGGAAGCTGTAGCACAAAATCTAAGAT
GGACCATGTGTTTACTAGTGACCATCAGCAACAGGAGAGAAATAGCCCAGAAAAGGAGTTGAACTGCCAG
GCTTTCTATAGCCATGATTCGGAGCCTTGGGATAGAGAGAGAGAAAGTTCTCTCCCTTTCACAGGCAAGT
GCTCTCCTGGGTGTGTTTTTTTTTTTTTTTTTTGGAGACAGAGTCTCGCTGTCGCCCTGGCTGGAGTGCA
GTGGCACGTTCTCGGCTCACTGCAGGCTCCGCCCTCCAGGGTTCATGCCATTCTCCTGCCTCAGCCTCCC
GAGTAGCTGGGACTACAGGTGCCCGCCACCTCGCCCGGCTAATTTTTTGTATTTTTAGTAGAGACAGGGT
TTCACCGTGTTAGCCAGGATGGTCTCGATCTCCTGACCTCGTGATCCGCCCGCCTCGGCCTCCCAAAGTG
CTGGGATTACAGGCGTGAGCCACCGCGCCCGGCCCTCTGGGTGCGCTTTTTTAGGCCCTGGAGTCAGTAC
CGCTGTTTTCCATGGAACCAGTTCTGAGGCCCTGGTACTCCTGGGAGCTGCTGTAGATACAAAGTTTCAA
TCATGACAGCATCCCTAGAGTCAGTGAACAGGGAACTCTGAACAGAATTATTGTTATGGAACTCTCTGAG
ACTCTGTGTCTTTTTCGTTGTTTTGAAAACGTAGGCTGGGCATGGTGGCTCAGCCTGTAATCCCAGCACT
TTGGGAGGCCGAGGCAGGAGGATCACGTGAGGTCAGGAGATTGAGAACAGCCTGGCCAACATGGTGAAAC
CCCATCTCTACTAAAAATACAAAAATTAGTCAGGCATGGTGGTGGGCGCCTGTAATCCCAGCTACTTGGA
GGCCGAGGCATGAGAATTGCTTGAACCCGGGAGGCGGAGGTTGCAGTGAACAGAGATCGCACCACTGTAC
TCCAGCCTGGGTGACAGAGGGATATTCCATTTCAAAAAAAAAAAAAGAAAGAAAGAAAATGTAATGGCTT
AGAAAAGAGAACTCTTTGGGAAACTGAGGAAAGTTAAGAACCACTATCCTAAAGAAAAGGATGTCATTCA
GTACCTAGAGCTGGTGTATTCCCCCAATGCTAGTCTCTGTGACAGTCCTTCTTTTACGCACTGCATAGAA
TGTTCACTCCAGCATTTACGAAGTATTAATAAATTTCTAACTAGTGTTGTAAACTCAGTGTGTATGCTTT
TGGAGAAGAGACCACAAGACCCTCTGTAGGAAAGAGGCTGGGCAGAAGGCATTAGGTTTGGGTTTTTCTG
GGTCTCTCTGCTCTGGAATAGTACAATTTATGAGATGACTCCCTGGATTGTTGTGGGTTTTTTCCCCCAT
AGGCTGAAGAAGAGCAAACATGCCCAGTGCCCCAGGAAGAAGAGGAGGAGGTGCGGGTACTGACACTTCC
CCTGCAAGCCCACCACGCCATGGAGAAGATGGAAGAGTTTGTGTACAAGGTATGGCCTATGAGCTTCTGT
CCTGGAAGGGGAAGGGCAGCAGGCCTGTCTGGGATGTCCCCAGCCAGCAAGCTTGCACTGGGTCGAAGAG
CTTTTATTCCTGGGAAGGAGTGGATTTGTTTTTTGGTTAAGATGAGGTAAATCAATGTGAAGACTGGCTA
AATTTTTACTGCCTACTCTGTGAGGCATTGTTCAAAATATATTCTTAAAACGGATTCTTTTCTATGCAAG
GGGCGCCTTTGGAAGTCATCAAAATATTTCAATATGACAGTAATATGAATGCCTTTGTTAACACATTAAA
TATACATGAATGATGAGCACTAATGTATTTTATTAAAATTTGTAATTTCTAAGTAATGATGAATAGAAAC
AGTATTTTGAGATACCTGCAGAACTAATGTGTGAGAGTATCTGATTTCTATTGGTGACAAAGCCACAAGT
TCTACTAATACTGTGGTTTGTTGCTTATGCTTATAATTGAGGAAAATGCTCAATTTACTTAGAGGTAAAT
GGAAATACAATTTTTTCCATCTATTCCCTGGATCCTGTCCATAGTGCATGGATCCTAGATTAAGAACCCC
TACTCTAAAAAGATGTGGTAAGAACTTAGAATCTAATTGGGGGAGGGATGAGACATGAGATAGAATCTCC
TGGAGAGGGGCAAAGGAGGGATCTGTACGTTTTTTAAAACAACGTTTATAATTTTTTTGTTTTTATTCAG
TATATCAATTTTGAAATTTCAAGTAGCCTAAAGGAGGTTGTACCATATATATATATATATTTTTTTGAGA
CAGAGTTTTGCTCTTGTCTCCCAGCTGCAGTGGCACGATCTCAGCTCACTGCAACCTCTGCCTGCCAGGT
TCAAGCACTTCTCCTGCCTCAGCCTCCCGAGTAGCTGGGATTACAGGCATGTGCCACCACGCCTGGCTTA
TTTTTGTATTTTTAGTAGAGACGGGGTTTCTCCATGTTGGTCAGGCTAGTCTCGAACTCCCAACCTCAGG
TGATCCGCCCGCCTCAGCCTCCCAAAGTGCTGGGATTACAGGCGTGAACCACTGCGCCCAGCCTGGTTGG
ACCATATTTTCTTAAAAGGTTGAGAAACACTTACCTAAAACTTCTCCAGATAAACTAACATGAAACAAAG
ACAAAGAGATTTTTTGTTTGTTTGTTTTTGTTTTTTTGTTTTTTTTGAATACAGAGTCTTGCTCGGTCGC
CCAGGCTGGAGTGCAGTGGTGCAATCTCGGCTCACTGCAACCCCCGCCCGCCAGGTTCAAACCATTCTCC
TGCCTCAGCCTCCTGAGTAGCTGGGATTACAGGTGCCCGCCACCATGCCCGGCCAATTTTTTGTGTTTTT
AGTAGAGACAGGGTTTCACCATGTTAGTCAGGCAGGTCTTGAACTCCTGACCTCAGGTGATCCACCTGCC
TCAGCCTCCCAAAGTGCTAAGATTACAGGCATGAGCCGCTACACTCAGCCAGGATTTTTTTTTTTTTTTT
AATGGTCAAAATATGAGTCAGGACTCTGCTAGCCCCATTCATGCTATGTCTTGGATATCCCCCAGTCCAG
GCTGGTTCTCCACTATTACCTAGGACCATTCAGAGCCTGACTGACATCTTCTTAGACTAGTTCTCCATCA
CTGCTTGTCCTTGGGGAAGGTACTATCTAGTGCCCTACCCAGAGAAAGTGTATATGTTCTAGGTCTGGGA
GGGACGTTGGAGGGTCATCCCATATGATGTGCTCCCTGACTGGCTAAAGGACAACGACTATCTGCTACAT
GGTCATAGACCTCCCATGCCCTCCTTTCGGGCTTGCTTCAAGAGCATCTTCCGCATTCATACAGAAACTG
GCAACATCTGGACCCATCTGCTTGGTGAGTGATTTGGATGGGGAAAGGTGGAATTTGTTGTCTCCCTAAC
TGCGAAGGGATTATAGAGCCCAACAAAGTTGTTCAGATCGATTGGCAGCTGGGATTCTACCTTTAAACTA
CTAATGACTTTAAGCTAATAACCTTGGTTGACCCTTGATCACTGATCTTTAAGACTGAGAGTAAAAGGCC
TAGAAACCAAAGAAATTGGGACTAGATATTTTTTTGTGATCCCTGGCAGAACTGTTCTAGGTGGAGACAA
TTCCTTGTACTTCGTAATTTCCAACTGTAAAAGTAAAATGCCAACATTTCAAAAAATGGTCTTTTTTGTC
TGGGTTTCTGTGACCAGTGCCACTTTCTTTGAAACATTGGACTGCAATTACACAGCTAAACCTCATAGCT
TTCTGATTTTTTTTTTATTTCAATAGCTCTTGGGTTACAAAGGGGTGGGGGGGGGTTTGGTTACATGGAT
GAATTATATACTGGTGAATTCTGAGATTTTAATGCACCCATCACCTTAGTAGTGTACATTGTACTCAATA
TGTAGTTTTTCTGTCTCACACCCCTATCTCCCCCTCCCGCTTCTAAGTCTCCATAGTCCATTATATCATT
CTATATGCCTTTGCGTACTCATAGCTTAGCTCCCACTTATAAGTGAGAACATATACAGTATTTGATTTTT
CATTCCTAAGTTACTTCACTTAGAATAGTGGCCTTCAGCTTCATCCAGGTTGCTGCAAAAGACATTATTG
CATTCCTTTTTATGGCTGAGTAGTATTCCATGGTGTACATACACCACATTTCCTTTATTCACTCATTGGT
CAATGGACACTTAGGTTGGTTCCATATCTTTAGAGTTGTGAATTGTGCTACAATAAACATACATGTGCAT
GTGTCTTTTTCATATGACTGCTTTTCCTTTGGGTAGACACCTGGTAGTGGGATTGGTGGATCAAATAGTA
GCTCTACTTTTAGTTCTTTAAGGAATCTCTATACCCTTTTCCATAGAGGTTATACTAGTTTATAATTTAC
ATTCCCACAAGGAGTGTATAAGCATTCCCCTTTTACCATATCCATGCCAACATCTGTTGTTTTTTGACTT
TTTAATAATGGCCTTTCTTGCAGAAGTAAGGTGGTATTTCATTGTGCTTTTAATTTGTATTTCCCTGATG
ATTAGTGATGTTGCCTGTAGCTCTCTGATTCAAAAACATCAACCATCAAAGTGTACACATTCCCTTTTTT
CTTACAGGTTTTTTTTATTTTACCCTCCTGCAGGTTTCGTGCTGTTTCTCTTTTTGGGAATCTTGACCAT
GCTCAGACCAAATATGTACTTCATGGCCCCTCTACAGGAGAAGGTGGTTTTTGGGATGTTCTTTTTGGGT
GCAGTGCTCTGCCTCAGCTTCTCCTGGCTCTTTCACACCGTCTATTGTCATTCAGAGAAAGTCTCTCGGA
CTTTTTCCAAGTGAGTTAAAAGAACACCATAGGAAAATACTGTGCACTGTTGTCACATCTTGAGATCCCA
GTGCTTACTGGGTGATAGGAGCTCATATCACTCCTAAAGGTTTCTTGATATGCCAGAGGAAAGGCTCTCT
GTTCTGTGGCGATAGTGGGGTGATGAAGCTTTTCTCTTAAACACTTCATGAACAGACTTACTGCATCCTC
TTCAACAGATGGAGAGACCAATTGTGGAATATATTTTCTACCTGAAAAGCACTATCTTTTGTGAGGCTTA
GAGTGCCAGTGCACTGGCTGCTTTCCAAGACCTTGTACAAGCGTAACTTACTATTAGCCCAAGCCAAAGT
AGGGTGTGGTAAATAGTCTATGCTTCATTCTGCTGGTGCTCCTACCCAGCAGTCAAGTAAAGCTTATCTT
TGCTGCCCATGCATTGCTGGAAGCTCCAGATGTCCATCATCGTGTGCTTTCTCTCTTGCCCAGCCGTGAG
CCTGAGGTTCTATTTGTTTGCTTGTTTTTGTTGTTATTGTTGGTTTTGGTGGCGACAGTGGTGTATTATT
TTGTTTTTTCTAACAACACCAGTAAGAAGGTACATTTTCCTCATTTAAAAAGTCAACTAAGATAGAAAAA
GCTAGAGTATCCCTTAATACTACTCCCAATTTAAGAATTTTGTTTTTAGAACTCATTCTATCAGAATAGG
AGAATTTTCAAAGCCTTTCCTGAGTGTGATGCTGGGAACCAGCTGGATTGTGATGGAATTTCTAAATTCA
AGTGTCTACTATGGGAGATACGGCTGTTCACTAGTAAGGGAAAGGGATAGAGTTCTTTGAATGGGGATTC
TTAGAGATTTTTCCATCGTAAAATAGAAGTGACCTTGTGGAATTTTAATCCAGAATACAGGATAGATTGT
ATGGGAGTCTACAATACAGGATAGATTGAGGGAATGGTTAAATCTGGGAGCAGGTGGAGAACAGAAAGAG
CTAGTTAAATGAGCCATACAAATTCATTTCCCCCACTGATTTGTTTCATTTTTGTGGTTCTTTTTAGACT
GGACTATTCAGGGATTGCTCTTCTAATTATGGGGAGCTTTGTCCCCTGGCTCTATTATTCCTTCTACTGC
TCCCCACAGCCACGGCTCATCTACCTCTCCATCGTCTGTGTCCTGGGCATTTCTGCCATCATTGTGGCGC
AGTGGGACCGGTTTGCCACTCCTAAGCACCGGCAGACAAGAGCAGGTAGGTCGGAAGATGTACGTACACC
TCAGAGGTATTTAGACCTTAGGCCTGATAGGCTCAAACCTATTTGAGCCTTAAGGTTTGCAGCTTGAGAA
CCCCACCCAAACCCTGATTTGTGTTTCAGAACTTTCAAAACAGCATCAATTTCAAAGCTCAGTTCTCAAC
CCCCAGTGTCATACTATTTCAGTAGAATGCCAATAGGACAGATCATTACCCAGCTGGTCAATTAACTGCA
AGCACAGATGTAGCTACTCTTATACTTGGTTTGTGACAGTTTAAAAATGTAGCAAGCACAGTAATTTTAG
TGACCCGAGTCCCAAGCTCAGACCAAGGGATTAAAATTAAAACACTCAGAGGCATTTTACTAAGGGAATG
GAATACCTGGTTTGGTGAATGAAAATACCTTGAGCATAAACAGGTAACAAAGTAATAGCTACAAGCATAA
ACATCTTTGTGTTGTCTCATCTCATCTGGACTCTTATTTTAAGCTTTTATTACTTGCAGCAACACCACCA
ATTACCACTAGTCTTTAGTCCCTTTACAATTAATATCTGCTATGTTTTAACCTTCAGTTAAAATAATTAA
ATATTGTCCGGGAGCAGTGGCTCACGCCTGTAATCCCAGCACTTTGGGGGGCCGAGGCAGGCGGATCACT
TGAAGTCAGGAGTTTGAGACCAGCCTGGCCGACATGGTAAAACCCTGTTTTTATTTAAAATATAAAAATT
AGTCGGCGTTGTGGCAGGTGCCTGTAATTCCAGCTACTTGAGAGGCTGAGGCGGGAGAATCACTTGAACC
TGGTCGGGGGCAGAGGTTGTAGTGAGCCAAGATCACGCCACTGCACTCCAGCCTGGGCAACAAAGCAAGA
CTCTATCTCAAAAAAAAAAAAGAAAGAAAGAAAGAAAAGAAAAAAATAATAATAATTAAATATTTATCAA
GCACCAGCCACGTGCCATGCACTGTGCTTAAACAAAGGTATTTACAAAGATGGAGAGGTGGTCCCTGCCC
TTCAGTTATGTGTAGTGTAATGATGAGACACATACCCATTCTTCCCTTGCTCTTGCCTTACTTGTGTGTC
CCAGCAGCTACAGGATAACTGCTAATCTGGGTATATCTGTCTACTCAGGAGGTCAGAGTTCTGGTTATGT
AAGACTTTTATGATCTTAACAGGAAGCCTATCCTATGCTTCTCTGACTTGGGAGGCAACTGAAGCCTTGA
CATCTAAGTTCTGTACCCAAGTTAGCCATCAGAGAGTCTTGACATACCATTTTTACCTAAATTCCTTAAC
AGATAATGAAGAATATGAGATCTTTGTTCTTGGTGCTTAAAAGGTAGTAAGCACTTACACAGCTTCTTCC
CATTCAGGACATAGAAGAAATATCTGTTGAGTGTTATCCATCTTGTAGCTAAGTTTCCTAAACATGTACT
ATTCATCCAAAAATAGCTAATTATCATAAACATTGAAATTCCAGAGTAAGACAGCAGTGCCTTCCATGCG
GTGGCTGTTTCCTTGTTTGACAGTCTTATGTCTGTTCTGTTCAGGCGTGTTCCTGGGACTTGGCTTGAGT
GGCGTCGTGCCCACCATGCACTTTACTATCGCTGAGGGCTTTGTCAAGGCCACCACAGTGGGCCAGATGG
GCTGGTTCTTCCTCATGGCTGTGATGTACATCACTGGAGCTGGCCTTTATGCTGCTCGAATTCCTGAGCG
CTTCTTTCCTGGAAAATTTGACATATGGGTAAGAAATGAATCTTCCAGCAGGTCCTGTGATGGTGAGTGA
ACAGCCCAAAGTTAAAGGGCTGAGGAAAAGGTGGTATGAGACTGGGATCATATAGTGTTTGGAGGTACAT
ATATGTGCATTGAAGGTAAATCATCAATATTAACATTTTGGTGTATCTCTGACAAGGACTGTGTTTTTAA
AACTTTCTAAATATACCACTTTAATGTTATTAACAAATTTGTTTAATTAAGGATAAACTAGAAGAAGCAA
ATGCTTTCTCCTACTTGTTTCTTATTCCTCCTTGCATTATCCTAAAGGCTCTTTTCTTTTTATTCCTACA
GTTCCAGTCTCATCAGATTTTCCATGTCCTGGTGGTGGCAGCAGCCTTTGTCCACTTCTATGGAGTCTCC
AACCTTCAGGAATTCCGTTACGGCCTAGAAGGCGGCTGTACTGATGACACCCTTCTCTGAGCCTTCCCAC
CTGCGGGGTGGAGGAGGAACTTCCCAAGTGCTTTTAAAAATAACTTCTTTGCTGAAGTGAGAGGAAGAGT
CTGAGTTGTCTGTTTCTAGAAGAAACCTCTTAGAGAATTCAGTACCAACCAAGCTTCAGCCCACTTTCAC
ACCCACTGGGCAATAAACTTTCCATTTCCATTCTCCTAGCTGGGGATGGGGCATGGTCAAACTTAGCCAT
CCCCTCCTCAGCAAGGCATCTACCGGCCCCTCACAGAGACAGTACTTTGAAACTCATGTTGAGATTTTAC
CCTCTCCTCCAACCATTTTGGGAAAATTATGGACTGGGACTCTTCAGAAATTCTGTCTTTTCTTCTGGAA
GAAAATGTCCCTCCCTTACCCCCATCCTTAACTTTGTATCCTGGCTTATAACAGGCCATCCATTTTTGTA
GCACACTTTTCAAAAACAATTATATACCCTGGTCCCATCTTTCTAGGGCCTGGATCTGCTTATAGAGCAG
GAAGAATAAAGCCACCAACTTTTACCTAGCCCGGCTAATCATGGAAGTGTGTCCAGGCTTCAAGTAACTT
GAGTTTTAATTTTTTTTTTTTCTTGGCAGAGTAATGTAAAATTTAAATGGGGAAAGATATTTAATATTTA
ATACTAAGCTTTAAAAAGAAACCTGCTATCATTGCTATGTATCTTGATGCAAAGACTATGATGTTAATAA
AAGAAAGTACAGAAGACACTTGGCATTCAAA
AdipoRl Transcript sequence (SEQ ID No. 4):
Transcript variant 1:
>gi|373251186|ref|NM_015999.4| Homo sapiens adiponectin receptor 1
(ADIPOR1), transcript variant 1, mRNA
ATTGGGACTCCGCGAGAGGGCCTAGGATTGGAGGGCCCAGGCAGAGCCCGCGAGGAGGTGACGCGGCTGC
GGAGGTGACGCGGGAGGTCGCGCGCCCCTTCCGGCGCGGGGAGGGCGCTGAAGATCGGGGCCGCTCGGCC
GCAGGCCGCCTCCAGCGCCGCGGGATGTAGCGCGGGGGACCGCGGCCCCCAGCAGAGCCCGCCTGCCCGG
CTTGTCTACCATCAGAGGGAGATCTCTGCCCCCTGGGGCTGAGAGACCCCAACCTTTCCCCAAGCTGAAG
CTGCAGGGTATTGAGGTACCAGCCAGATGTCTTCCCACAAAGGATCTGTGGTGGCACAGGGGAATGGGGC
TCCTGCCAGTAACAGGGAAGCTGACACGGTGGAACTGGCTGAACTGGGACCCCTGCTAGAAGAGAAGGGC
AAACGGGTAATCGCCAACCCACCCAAAGCTGAAGAAGAGCAAACATGCCCAGTGCCCCAGGAAGAAGAGG
AGGAGGTGCGGGTACTGACACTTCCCCTGCAAGCCCACCACGCCATGGAGAAGATGGAAGAGTTTGTGTA
CAAGGTCTGGGAGGGACGTTGGAGGGTCATCCCATATGATGTGCTCCCTGACTGGCTAAAGGACAACGAC
TATCTGCTACATGGTCATAGACCTCCCATGCCCTCCTTTCGGGCTTGCTTCAAGAGCATCTTCCGCATTC
ATACAGAAACTGGCAACATCTGGACCCATCTGCTTGGTTTCGTGCTGTTTCTCTTTTTGGGAATCTTGAC
CATGCTCAGACCAAATATGTACTTCATGGCCCCTCTACAGGAGAAGGTGGTTTTTGGGATGTTCTTTTTG
GGTGCAGTGCTCTGCCTCAGCTTCTCCTGGCTCTTTCACACCGTCTATTGTCATTCAGAGAAAGTCTCTC
GGACTTTTTCCAAACTGGACTATTCAGGGATTGCTCTTCTAATTATGGGGAGCTTTGTCCCCTGGCTCTA
TTATTCCTTCTACTGCTCCCCACAGCCACGGCTCATCTACCTCTCCATCGTCTGTGTCCTGGGCATTTCT
GCCATCATTGTGGCGCAGTGGGACCGGTTTGCCACTCCTAAGCACCGGCAGACAAGAGCAGGCGTGTTCC
TGGGACTTGGCTTGAGTGGCGTCGTGCCCACCATGCACTTTACTATCGCTGAGGGCTTTGTCAAGGCCAC
CACAGTGGGCCAGATGGGCTGGTTCTTCCTCATGGCTGTGATGTACATCACTGGAGCTGGCCTTTATGCT
GCTCGAATTCCTGAGCGCTTCTTTCCTGGAAAATTTGACATATGGTTCCAGTCTCATCAGATTTTCCATG
TCCTGGTGGTGGCAGCAGCCTTTGTCCACTTCTATGGAGTCTCCAACCTTCAGGAATTCCGTTACGGCCT
AGAAGGCGGCTGTACTGATGACACCCTTCTCTGAGCCTTCCCACCTGCGGGGTGGAGGAGGAACTTCCCA
AGTGCTTTTAAAAATAACTTCTTTGCTGAAGTGAGAGGAAGAGTCTGAGTTGTCTGTTTCTAGAAGAAAC
CTCTTAGAGAATTCAGTACCAACCAAGCTTCAGCCCACTTTCACACCCACTGGGCAATAAACTTTCCATT
TCCATTCTCCTAGCTGGGGATGGGGCATGGTCAAACTTAGCCATCCCCTCCTCAGCAAGGCATCTACCGG
CCCCTCACAGAGACAGTACTTTGAAACTCATGTTGAGATTTTACCCTCTCCTCCAACCATTTTGGGAAAA
TTATGGACTGGGACTCTTCAGAAATTCTGTCTTTTCTTCTGGAAGAAAATGTCCCTCCCTTACCCCCATC
CTTAACTTTGTATCCTGGCTTATAACAGGCCATCCATTTTTGTAGCACACTTTTCAAAAACAATTATATA
CCCTGGTCCCATCTTTCTAGGGCCTGGATCTGCTTATAGAGCAGGAAGAATAAAGCCACCAACTTTTACC
TAGCCCGGCTAATCATGGAAGTGTGTCCAGGCTTCAAGTAACTTGAGTTTTAATTTTTTTTTTTTCTTGG
CAGAGTAATGTAAAATTTAAATGGGGAAAGATATTTAATATTTAATACTAAGCTTTAAAAAGAAACCTGC
TATCATTGCTATGTATCTTGATGCAAAGACTATGATGTTAATAAAAGAAAGTACAGAAGACACTTGGCAT
TC
AdipoR1 Transcript variant 2 (SEQ ID No. 5):
>gi|374858065|ref|NR_046083.1| Homo sapiens adiponectin receptor 1
(ADIPOR1), transcript variant 2, non-coding RNA
ATCACGCACTTCAGGCTGCGATAACGAGGTGGAGTGTAGAATCCGACGAACGTCCTCTACCCCGGGGCGG
GTGGAGGTTACCTGGCTGCGGCGGGGAGTTTAGAAGACCAAACTGGACAATGGACTTTGCTCACTATGAT
GACATGATCTCCATTGACCTCCATTTGTCTACCATCAGAGGGAGATCTCTGCCCCCTGGGGCTGAGAGAC
CCCAACCTTTCCCCAAGCTGAAGCTGCAGGGTATTGAGGTACCAGCCAGATGTCTTCCCACAAAGGATCT
GTGGTGGCACAGGGGAATGGGGCTCCTGCCAGTAACAGGGAAGCTGACACGGTGGAACTGGCTGAACTGG
GACCCCTGCTAGAAGAGAAGGGCAAACGGGTAATCGCCAACCCACCCAAAGCTGAAGAAGAGCAAACATG
CCCAGTGCCCCAGGAAGAAGAGGAGGAGGTGCGGGTACTGACACTTCCCCTGCAAGCCCACCACGCCATG
GAGAAGATGGAAGAGTTTGTGTACAAGGTCTGGGAGGGACGTTGGAGGGTCATCCCATATGATGTGCTCC
CTGACTGGCTAAAGGACAACGACTATCTGCTACATGGTCATAGACCTCCCATGCCCTCCTTTCGGGCTTG
CTTCAAGAGCATCTTCCGCATTCATACAGAAACTGGCAACATCTGGACCCATCTGCTTGGTTTCGTGCTG
TTTCTCTTTTTGGGAATCTTGACCATGCTCAGACCAAATATGTACTTCATGGCCCCTCTACAGGAGAAGG
TGGTTTTTGGGATGTTCTTTTTGGGTGCAGTGCTCTGCCTCAGCTTCTCCTGGCTCTTTCACACCGTCTA
TTGTCATTCAGAGAAAGTCTCTCGGACTTTTTCCAAACTGGACTATTCAGGGATTGCTCTTCTAATTATG
GGGAGCTTTGTCCCCTGGCTCTATTATTCCTTCTACTGCTCCCCACAGCCACGGCTCATCTACCTCTCCA
TCGTCTGTGTCCTGGGCATTTCTGCCATCATTGTGGCGCAGTGGGACCGGTTTGCCACTCCTAAGCACCG
GCAGACAAGAGCAGGCGTGTTCCTGGGACTTGGCTTGAGTGGCGTCGTGCCCACCATGCACTTTACTATC
GCTGAGGGCTTTGTCAAGGCCACCACAGTGGGCCAGATGGGCTGGTTCTTCCTCATGGCTGTGATGTACA
TCACTGGAGCTGGCCTTTATGCTGCTCGAATTCCTGAGCGCTTCTTTCCTGGAAAATTTGACATATGGTT
CCAGTCTCATCAGATTTTCCATGTCCTGGTGGTGGCAGCAGCCTTTGTCCACTTCTATGGAGTCTCCAAC
CTTCAGGAATTCCGTTACGGCCTAGAAGGCGGCTGTACTGATGACACCCTTCTCTGAGCCTTCCCACCTG
CGGGGTGGAGGAGGAACTTCCCAAGTGCTTTTAAAAATAACTTCTTTGCTGAAGTGAGAGGAAGAGTCTG
AGTTGTCTGTTTCTAGAAGAAACCTCTTAGAGAATTCAGTACCAACCAAGCTTCAGCCCACTTTCACACC
CACTGGGCAATAAACTTTCCATTTCCATTCTCCTAGCTGGGGATGGGGCATGGTCAAACTTAGCCATCCC
CTCCTCAGCAAGGCATCTACCGGCCCCTCACAGAGACAGTACTTTGAAACTCATGTTGAGATTTTACCCT
CTCCTCCAACCATTTTGGGAAAATTATGGACTGGGACTCTTCAGAAATTCTGTCTTTTCTTCTGGAAGAA
AATGTCCCTCCCTTACCCCCATCCTTAACTTTGTATCCTGGCTTATAACAGGCCATCCATTTTTGTAGCA
CACTTTTCAAAAACAATTATATACCCTGGTCCCATCTTTCTAGGGCCTGGATCTGCTTATAGAGCAGGAA
GAATAAAGCCACCAACTTTTACCTAGCCCGGCTAATCATGGAAGTGTGTCCAGGCTTCAAGTAACTTGAG
TTTTAATTTTTTTTTTTTCTTGGCAGAGTAATGTAAAATTTAAATGGGGAAAGATATTTAATATTTAATA
CTAAGCTTTAAAAAGAAACCTGCTATCATTGCTATGTATCTTGATGCAAAGACTATGATGTTAATAAAAG
AAAGTACAGAAGACACTTGGCATTC
AdipoR1 protein sequence (SEQ ID No. 6):
Variant 1 only, transcript variant 2 is non-coding.
>gi|21361519|ref|NP_057083.2| adiponectin receptor protein 1 sapiens+
MSSHKGSVVAQGNGAPASNREADTVELAELGPLLEEKGKRVIANPPKAEEEQTCPVPQEEEEEVRVLTLP
LQAHHAMEKMEEFVYKVWEGRWRVIPYDVLPDWLKDNDYLLHGHRPPMPSFRACFKSIFRIHTETGNIWT
HLLGFVLFLFLGILTMLRPNMYFMAPLQEKVVFGMFFLGAVLCLSFSWLFHTVYCHSEKVSRTFSKLDYS
GIALLIMGSFVPWLYYSFYCSPQPRLIYLSIVCVLGISAIIVAQWDRFATPKHRQTRAGVFLGLGLSGVV
PTMHFTIAEGFVKATTVGQMGWFFLMAVMYITGAGLYAARIPERFFPGKFDIWFQSHQIFHVLVVAAAFV
HFYGVSNLQEFRYGLEGGCTDDTLL
Adiponectin receptor 2 (AdipoR2/AR2) (SEQ ID No. 7):
Gene sequence:
>gi|224589803: 1785590-1912499 Homo sapiens chromosome 12, GRCh37.p9 Primary
Assembly
TTTTTTTTCCCTAGACAGAGTCTTGCTCTGTCACCCAGGCTGGAGTGCAGTGGCGTGATCTTGGCTCACT
GCAACCTCTGCCTCCCAGGTTCAAGCAATTCTTCTGCCTCAGCCTCCCGAGTAGCTGGGACTACAGGTGT
GTACCACCACGCCTGGCTGATTTTTGTATTTTTAGTAGAGATGGGGTTTCACCATATTGGCCAGGCTGGT
CTCAAACTCCTGACCTAAAATATCCACCCGGCTCGGCCTCCCAAAGTGCTGGGATTACAGGCGTGAGCCA
CGGCACCCGGCCGCCAAATCTCTATTTTCTGTTTCTCATTTTATGCTGTCTTCTGGGTAAATTCAACCAG
CTGATTTGGCTGTGTCGAATCTGCCGTTTAACTTATTCACTAAGTTTTATTTATAATTTCTGGAAGTTTG
ATTGTTTTGCATTTCTACCTGTTCTTATTTCATTCACTCTTGTTTATTCTTGCATTTCTTCTTTTACACA
TTTTAAATGTGTAAAAATACACATACACATTAAAAATACTAACTTTATCGTATATCCTCTATCATATTGT
TCTAAAAGTAAACCTTAAGCTCACATAAGGCACAGGCTCATGGTTATTAATTCCTCAGGGAAGGTTTTTT
TTTTTCCCCTTGTCCCCTACCTTCTAGAGCAAATGAAGCTTGAATTCACAGGTACTGTAGTGGTCTTGGT
TCCCTTTGTGTTTCAGGCACCTAGTTATTTAACTTTCTTTCTTGAGTGTTCAGCTTTGTATTTATTTTAG
TTTTTGGAGACAGAATCTCATTCTGTCACCCAGGCTGGAGTGTAGTGGCATATCCATAGCTCACTGCAAC
CTCAAGCTACTGGGTTCAAGAGATCCTCCCACCTAAGTCTCCCAAGGAGCTAGGCCTACGGGTACATGCC
ACCATGCTTGTTTTGTAGAGATGGGGGTCTTGCTATGTTGTCTAGGTTGGTCTCGAACTCCTGGCTTCAA
GTGATCCTCCTGCCTTGGGCTCCCAAAGTGTGCTGGGATTACAGGCAGGAGCTACTGCACCCAACCTGGC
TTTTTTTTCCCCCTGAGATGGAGTCTCACTCTGTCGCCCAGGCTGGAGTGCAGTGGTGCGATCTCGGCTC
ACTGCAACCCCCCGCTCCCGGGTTCAAGCGTTTCTCTTGCCTCAGCCTCCCAAGTAGCTGGGATTACAGG
TGCCCACCACCACACCTGGCTAATTTTTGTATTTTTAGTAGAGATGGGGTTTCACCATGTTGGCCAGGCT
AGTCTTAAACTCCTGACCTCGTGATCTGCCCGCCTTGGCCTCCCAAAGTGCTGGGATTACAGGTGTGCAC
CACCCCGCCCGGCCCTGGCTTTGTATTTAAAAGAAGCCTTATTGAGTTTACTCAGCAGTTGTAGGTGTTT
GTAGCAGGATAATTTTCAGATTATCTGGTCTGCCCATTTGTTAAACTTGAGATCTATTAATTAACTCTAT
GTTTTCATTAAGCCATTTTTTATTATGGTAAAATACACATACCATAAAAATTATTAATTAACATTTAATA
GGATGAGTAAAATTGCAGACAGAGAGCCCATTTCAGTCGTGCCACTGCACTCCAGCATGGGCAACAGAGC
GAGACTCTCTCTCAAAAAAAAAAAAGAGCCCATTTTAGTTGCTGTGTGAGGAAGGACTTCGTAATAGGCT
AGTAGGAAGCAAAGGATTTCCAAGGAAGGAGAGAGAGGCAGAAAGTGAAGAATATATTTTGGGAATGGTA
AATAGTTGGTTTGGCAGGAGCTTGGTACGTGGTACATGTAATAATAATGTGAGTGAATTTCTGAAATATT
AAGTTAGGGCCAGATTGTGGAGAGCCTTGGATTTCAGCTTAAAGGAATTCGGACTTTGAGAGCTTTTGAA
AGTTTTTGAACTTAACTCATTTTGAAAGTGGGTCTTGGCAGCAGTAGATGGAGTGGATTGGAGGATGTTG
AGATTGACGACAGGGAGACCAATTAGGAACATATTGGAGTAGTCCAGGGAGGCATGAAGGCACAGAATAG
GGATAAGGTCTGACAAAGGCTCAGAGCTGTTGAAATGACCAAAGCATAATTGTAGTTATTAACCAAATTG
GGGACAAGAGGAGATGCCAATACAGACATATGACAGCATTCTCTAGAGTAAGTCACTTCCCAGCGGAATA
GCATTATGAACTCTTAGAGACTTGCTGGTGTCTTCTAATGAAGTCACATGCTGTTTATAAAATGTCGGCT
AATTGGCCTATGGAGCCATCCAGAGCCCTCCTTTGATCTGAACCCAGACTTGAAAAAGCATGCGTCAGTG
CAATTTGAAAAACAGTATTAAAATAGAGAAAGTAGAGTAAAACAGATAGATCTGTAGCTCTACAGAGTAT
AAACAACCGTTTTTAGCATCAGCTCAAAGAGTTTTAAGTGGTCTCCCGTTATTTGCTTCTACCCTTAATA
AGATCTTCTAAAATCATTGCTCTGGACTTTCAATCTTCAAATTTCTTAGCAGGACAATTTAATCATCACC
TATTTCTATTGGGTTATATATCTTTTTTTTTTTTTTTTTTTTTTTGAGACAGAGTCTTGGTCTGTTGCCC
AGGCTGGAGTGCAGTGGCGCCATCTCGGCTCACCGCAACCTCCGCCTCCCGGGTTGAAACAATTCTCCTG
CCTCAGCCTCCTGAGTAACTGGGATTACAGGCATGCACCACCTCGCCCAGCTACTTTTTGTATTTTTAGT
AGAGACCGGGTTTCACCATGGTGGTCAGGCTGGTCTCAAACTCCTTACCTTGTGATCTGCCTGCCTTGGC
CTCCCAAAGTGCTGGGATTATAGGCATGAGCCACCGCACCCAGCCTGGGTTTTATATCTAGTTAGCATGG
TGACAACCCAGATAACTTGGTTTGCATTTGTTTTCCCATTGCTCTAGTATGTTCCAAGGTCGTTTTCATA
TATATCCTGTATGTGAATTCCCCTTTCCCAAATTGTTGTGGATTGTATCATAACTTGTCAATTAGCTAAA
TGTAAATGAGCATCAGCTTTCTAGAGATTTAATTTTTTTTTAGCGTTTAATTGCTGAATGGAAAGCAATA
GCACTATGAAGCCAAGAAGTTATTTGGAGAAGAACTTGAATTGCTTGATTCTGAGGATGGGTAATCAAAA
TGTGATTGTGAAGAAATGGAGTTATTGTATATGTTATATATAATTTATTATATATGTTATATATAATTTA
TTACATATATATTTAAAACTTGAATGATATATTGAAATTACATTGAGCGTACTAATAAAAAGCATTCAGA
TACACTTGGCTCCCTTTTTCCCCTTTCCTTCCTTCTATTAACAATTATTGAGCACTCAGTGTGGGCCAGG
CACTGTGTTATATGCTGGAAGTTATAGTGGTTTATTCTTCAATTTTGAAATAATTTGGCAGTTGATAAAA
ATCTTTAAAGTTTGTTTTTTTTAAAATAGTCAGTTCTGGCCGGGCGCGGTGACTCACTCCTGTAATCCCA
GCACTTTGGGAGGCCAAGGTGGGTGGATCACCTGAGGTCGAGAGTTCGAGATTAGCCTGGCTAACATGGT
GAAACCCTGTCTCTACTAAAAATGCAAAATTAGCTGGGCATGGTGGTGCACGCCTGTAATCCCAGCTACT
CGGGAGGCTGAGGCAGGAGAATTGCTTGAACGGGGGAGGCGGAGGTTGCAGTGAGCTGGAATTGTGCCAC
TGCACTCTAGCCTGGGCGACAGAGTAAGACTCCGTCTCAAAAAAAGTCAGTTCTTGGATTTTGATGACAT
GGTACTATGTAGGGCTTACAGGGTTATTAGTGCCCTGAGTCCACACTTCACAGAGTTATTAGATTTAAAT
TTTCAAATTTTATTTTAGAGACAGGGTCTTTTTCTGTTGGCCAGACTGGAGTGCAGTGGTGCAATCATAG
CTGACTGAAGCCTGGAACTCCTGGGCTCAAGCAATGCTCCCACTTCAGCCTCCTGAGTAGGTCGGACTAC
AGGTATGCACCACTACACCTGGCTAGTTAAAAAAAATTTTTTTTTGTAGAGACAAGGGCCTTACCATTTG
CCCAGGCTGGTACTGAACTCCTAGGTTCAGGCAATCTTCCCACCTCGTCTTCCCAAAGTGTTGGGATTAC
AGGCATGAGCCACCTTGCCCGGCCTCATCATTAGACTTAAAGATCTTTTTGTCTTTGGTAGTTGTTTTAC
AATTTTAAACAACTAGTGGCTGTAGTAGGCATTCTTAACCAGGAATAAAGTCATAAATGTAAATGTATGG
TCTCGGAGGAGCAGGGGCAGTCCTCTGTGAATCTGTAAATATTTGGTGTATATAGTTATTTTTCTGAGGG
TATAGATGCTCAAAGGAGCCTATAACCCAAAAAAGGTTCAGAGCAACTGTTTGCCTTGCTTCATTCAGCT
TCCAGAATAGTGGCCTTTGTTACGTAGACTAGTGACTATGTGCTTGGGCATTACAGTCACACTCCCTGTA
TTTGAATCCTGGCTCTGCTATGTAATAGCTGTGTGACCTTAAACAAGTCATTTAACCCCCTGTGCCTCAG
TTTCCTCATCTGTAAAATGGCGATAACAATAGTATTTATGTCATGGCGTTGTGGTGAGGATTACATGAGT
TAACGTATATAAATTACTCAAAAAATGCTCAGCACATAGTAAATATTCTATACATATATCAGCTATTGTT
ATTATAATAATTACATGGGTATGGCATTGATGGTAATGATGGTGAAAACCATGATGACTTCTGCCTTCCT
GAAGCCACTTAAGAGTGAGTAACAACAGCCAAAGTTACTTAGGAATAAAAGATTAAAAAAAAAAAAAGGA
GCATGACAATGGACGGCCACAATAAATGTTACCCAGCTGGTCATTCTAGGAAAAGCTGATACCGCCCTTC
CCTTCCTGTTCTAGTTTATGGCCAGCCAGGCTCTGAAGGTTCGACAGTCTGACTCAACCACTGAGAGGAA
TATATTCCAGTTGAAGCACAGTGGCAAGAGCAATTTGTTTTTTAGATTTAGCTGAAATATATTCTGATTC
TATTTTTTAGCCTTGGATGGAACTGGCTGTTTGCCACGGTTTGTTTTTTCCCCATGGAATGTGTGTTGTT
GATGCTCTACCATGATAGACAGCAAGTGGGGAAATGGGAACTTTTATTAGCTGACCTAAAGCTCGTCTCT
CCTAGTTCTGCCACAAAACACTCTGGCAGTCTTAAGAGACATGTCCTGTATCTCCATCTCCACCTACTGA
CCTCCCCAGCTGAAAGGGTTAGGCAAAGTAAATCAGCTTAGAATTTATTCCAAAGGTCTTTGACTTTTTC
TAGCTTTTAATGACCAAAGTTTTCAGATAATTGAGTTTCAGGGCTATCTGATCACCTTTGTTGGGTCAAT
TTTGCCTCTCCTCAAATGCCTGAGAATGATGTTTTTTTCATGTATTATTTTTGGGGTCTCTACTTTTTTT
CTTTATCCCTTCCTTGCACAAATAACTGCCAGTTCCTGAAGCCACTGCCCCCAGGAATTTGCTTCCTGTA
ACAACAGGTACTTCTCTGTCTGGGCTCTCCCTCTGGAAAAAGCTCATTCAAGGATCCCCTGATTCCAAAG
TGCCAAGTAGAAATTGTATAAATTGAATTAATTCAGATGCTGTGTATCAGGCTTAAAATCCAATAGATAT
GTAGGTAATTGTCTGAATATTTTCCGCAGTAAAAGACTGAAGTCTAAGGTCTAACTCCCATTGCAGTACA
TAAATTGCAGTGCCCAGTGCAAAGGGAAAATGAAGGGTCTCTTATTCAAAAGTTATTAAAAATTTCAAGA
CAGTAACAACAGAGCATCAAACCAAGAATAGGGGCCAGGCGCGGTGGCTCATGCCTGTAATCCCAGCACT
TTGGGAGGCCGAGGTGGGCAGATCACCTGAGGTCAGGAGTTCAAGACCAGCCTGGCCAACATGGTGAAAC
CCCATCTCTACTAAGAATACAAAAAATTAGCTGGGTGTGGTGGTGCATACTTGTAATACCAGCTACTTGG
GAGGCTGAGGCAGGAGAATCGCTTGAACCTCAGAGGCAGAGGTTGCAGTGAGGTGAGATCGCACCATTGC
ATGACAGCCTGGGCAAGAAGAGCGAAATGTGGTCTCAAAAAAAAAACACAAAAAACAAAACCCAAGCATA
GGGCACTTTCGAGTACAGGGCCCCGAGCAACTAACTGCATAGGTCATCCACCTAGGAAGCCAGGCCTGCA
TGGAGCCCACGAATCAGAATCTTTGGAATCTGACTGGAAAAAAAAAATCTGACTTTTGGGGCCTACCAGA
ATCTCTGGGATAATATCTGGGAATTGCCTTTTTTTTTTTTTTTTTTTTTTTTTTAAATACAGGATCTTGC
TCTGTTGCTCAGGCTGGAGTGCAGTGGCTTGATCATGGCTCACTGCAGCCTCAATCTCCTGGGCTCAAGC
AAACCTCCAACCTCAGCCTTCCAAGTAGCTGGGACTACAGGTGTGCTCACACTGGTTATTTTTAAAAGGG
TCTCACTATGTTGCTCAGGCTGGTCTCGAACTCCTGGACTCAAGTGATCCTCCTGCCTCAGCCTCCCAAA
GTGCTGGGATTACAGGTGTGAGCCACCGTGCCCAGCTGGAATTAACATTCTTAATAAGCTCTCTAGATGT
TGACTTTTGAGAATCTCACACCTATTTTCTTCATTTGCTTGGGAAGAAGGAAGAGCAATGTTGGGTTTTT
GTTTGTTTGTTGTTTGTTTGTTTTTGAGATGGAGTCTCGCTCTGTTGCCCAGGCTAGAGAGCAATGGCGC
AATCTCGGCTGACTGCAACTTCTGCCTCCCAGGTTCAAGCAATTCCCCTGCCTCAGCCTCCGGAGTAGCT
GGGACTACAGGTGCACCACCATGCCTGGCTAATTTTTTGTATTGTTTAGTAGAGACAAGGTTTCACCATG
TTGGCCAGGATGGTCTTCATCTCCTGATCTTGTGATCTGTCCACCTTGGCCTCCCAAAGTGCTGGGATTA
CATGCATGAGCCACTGCGCCTGGCTCATTTTTTTTTTTAAGAGACAGAGTCACCCTCTGTCACCCAGGCT
GGAGTGCAGTGGCATGATCATAGCTCAATGTAGTATCGATCTTCTGGGCTCAAGTGATCCTCCCACCTCA
GCCTCCTGAGTAGCTGGGACTACAGGTGTGTGCCAGCCACCATGCCTGGCTTTTTATTTTAATTTCTGTA
GAGATGGAGTCTCACTATGCTGCCCAGGCTGGTCTTGAACTCCTGGGCTCAAGTGATCCTCCTGCCTTGG
CCTTCCAAAGTGCTGGAATGACAGGTGTGAGCCACCATGCCTGGCCAATTATCTTTTCTTTTCTTTTCTT
TTTTCTGAGACAGAGTCTGGTTCTGTTGCCCAGGCTGGAGTACAGTGGTGGGAACTCAACTCACTGCAGC
CTTAGCCTCCTGGTTTCAAGCAATTCTTGTGCCTCAGCCACGGAGTAGCTGGGATTATAGGTGTGTGCCA
CCATGCTTGGCTAATTTTTGTATTTTTATTAGAGACAGGGTTTCGACATGTTGGCCAGGCTGGTCTCAAA
CTCCTGACCTTAAGTGATCCGCCCGCCTCTGCCTCTCAAAGTGAGCCACTGTGCCTGGCCCCAATTCTCT
TTTAAAAGTGTTCGGCTTGCTTTCCTAGATGGGCAACTGACAGCACTTATGTTGCATCTCTAGCAGCATT
TCTTTGAGCTCTTTTACTCTTTTTCTGTTTTCCTCTTTCTCCCCTATCCTCTTTTGTTTTACGCTTGTCT
TGGGCCTGCCCGCAATACCTCAGGTGTGGCTAGACTTGGCTGACGACATCCAAGGCATATGAGATTATTA
GAGAAAGTTTGAGGGGAAGGAGTAAGACTTTTGATAGTCTGGATTTTTCATTAAGAATTTTATTTTTGCT
ATAGCCTGCTTTTCCTACCCAGTTTTGCTTTTGCTTCTAATCTAAACCAAAAGAAAAGGAAAAAAAAATT
TTGAGAAACAAAGTTATGAGAACAATCTCAACCCCTACCTCAGTGATTATTTAGTCACAATCATTGGAAT
TCAATTCCAGATCAGTTCTTCTTCCTCCACCGTAAATGCCTGCCAGTCCTTGCAACTTATTTCTGCAAAT
TGTCACTTCACCAGATTGTGACCTCAAGGGCTCAGGGATTCATCCTGTATAACTTGATGAAAGTATTTAT
ATCAAACTATCCTGACTAAACCTGGTCCTGAGTTCTGAGTTTAGCTATACATCATTGAGATATTGCAAGG
TTAATAAACCTCTTTAGATTATGGATTTAAAATTTTAGCACAACAAATAAAATGCTCTTTTTTTGGCTTA
CAGTTCTGTGTGTGTGGGTGAGCGCATAAGCAGTCACTGAGTAGAACAGATGCTGTCTTGAATTTTTTCT
TAATAAAAAGATGACAGATTTTTAATTACTGGGTGCTTCTAAATTACTTAAGAAATGACATTTTTCTTTA
TTGTTTTCACCTGTATTTCCTGTTGTTCCGTAGTTGTAGCTTCTAATACGCATCTTTATTACTCTCTATT
TTTAAGGTAATGAAAACTTTGTGTTTCTATTTTCATTCTCACAGCAAAGGAGAGATTAAATGGAAACAAC
CCTTTATAACTTTGAAATATTATGTAATCCTGGCATTCTTAAAAGAATTTTTTGCTTGCCTTTTACTTTG
GCAAAATACTTGTATCTAAAGATATATAAAGAACACTGATGGCCAGGCATGGTGACTCACGTCTGTAATC
CAAGCACTTTGGGAGGCCGAGGCAGGCAGATCGCTTGAGGTCAGAAGTTCGAGACCAGCATGGTCAACAT
GGTGAAACCCTGTCTCTACTAAAAATACAAAAATTAGCCTGGTGTGGTGGCACACACCTGCGATTCCAGC
TATTTGGGAGGCTGAGGTATAAGAATTGCTTGAACCCAGGAAGCGGAGGTTGCGGTGAGGAGAGATTGCA
CCACTGCACTCCAGCTTGGGTGAAAAAGTGAGACTCTGTCTCAAAAAAAAAAAAAAAAAAAAGAACACTT
ATGGCTCAATAATAAGAGGGAAAACAGCCCATTTTAAAAAATAGGTAAAAAGTTTGAACAGACACATCAC
AAAAGAAAATATAAGAAAGGCCAATAAACACATGAAAAGTCTCAACATCATTAACAATCAGAAAAAGGCA
AATTAAAACCACAATGAGATAACACTGACATACCCACTAGAATAGCTAAAATAAAAAATTTGACAATACT
AAGGGTTAGCAAGGATGTGGAACAACTGGAAATCTCATATATTGCTGGTGAGGGTGTAAAATGGGGAAAC
AATTTGGCAGTTTCTCATAAAGTTAAACATAAATTTATATGATTTTGCTAATTCACCCCCTTATCAAAAG
TAAATGAAAACACATGTCCTCTAAAGATTTGTACATAAATGTTCACAATACCTTTATTTATAACAATCAG
GGACTAGACGTTGGGCACAGTGGCTCACATTTGTAATCCCAGCACTTTCGGAGGCCAACATGGGAGGATC
GCTTGAGGCCAAGAGTTTGAGGACAGCCTGGGCAATTTAGCAAGACCCTGTCTTTACAAAAAATAATAAA
ATTAGCGAGGCATGGTGGCACATGCCTGTAGTCCCTGCTACTTGGGAGGCTGATATAGGAGGATTGTTTG
TGCTCAGGAGATCAAGGCTGCAGTGAGGTACGACTGCATCACTGCGCTCCATCCTGGGTGACAGAGTGAG
ACCCTGTCTCACAAAACAACAACAACAAAAACAAGAAATTACCCAATCATTCATCAATAGGTGAATGGAT
AAACAAATGGAATACTACCTAGCAACGGAAAGTGACGAACTATGATGAATGCAATAGCATGAATGAAACT
CAAAAACCCCATGCTGAGAGAAAGTCGACAGAGCAAAAGAGTACATATCGCACGATTCCATTAATGGGAA
ATTCTAGAAGAGGCAAAAGCATTCTATAGTGGCAGAAAGAATATCAGTGGTTGCTTGGAGCCAGGCTTGG
GAGAGGAAATACTGACTCCAAATAGGCACAGGGGGATTGCGGGGGGTGTTGGAGATGTTGTATATTTTGA
CTGAGGTTATGCTTCCACGGGAGTATACATTTTTCAAACCTCATTTGAACTGTACACTTAAAAAGAGGGT
GCATTTTATATAAAATATACTTCAATAAAGTTGTTTTTCTCAACTACTAAAAAAGTTCAGAGAAGCCTCT
AATATATGTCCATGTACATACCACCTAGGATTCATAAATGTTAAATGTTTTGTTACTTGCTTCATCTTTT
TCATATAGAAGAAATGAAATATTACAGATAAAGATGATCTCTCCATAGCTCCCAGGCTTATTCTTCACCC
CTTCCCACCGGCAAGTACATTCCTAAAGTTGTATTTATCCTTTCTGTCCATCTAAAACATGTATTCACCG
ACATCTTCATTTTATTGATGAAGGTATCAAAGCTTAACAAGAGCTAATGCATAAGCCATAGATAAACAAT
GCCTTTGTCAGCAGCACAGCCAGTAATGATGTTCTATCTTAAGTTCAATCTGCAGGACTATGTTATCTAA
TCTGCGTTTGAGGAGCTGCGAATGTAATTTATGTAATTATTGGAGTGTTTATACTGGTCTTTCTCTTCAG
TTTTTTTTTTTGTTTTGTTTTGTTTTTTTTTTTTTTTTTTGGAGACGGAGTCTCACTCTGTCGCCCAGGC
TGGAGTGCAGTGGCGCCATCTCTGCTCACTGCAAGCTCCGCCTCCCGGGTTCACACCATTCTCCTGCCTC
AGCCTCCCAAGTAGCTGGGACTACAGGCGCCTGCCACCACGCCTGGCTAATTTTTTTGTATTTTTAGTAG
AGATGGGGTTTCACTGTGTTAGCCAGGATGGTCTCGATCTCCTGACCTCATGATCCGCCCGCCTCGGCCT
CCCAAAGTGCTGGGATTACAGGTGTGAGCCATGGCGCCCGGCCCTGATTAATGTAGTTTTAAACAACATA
ACAATTTGATATCAGTGATTCGTTTAATTGCAATTAAGATGGAATACAGATATTTATGTATTTCCCTCTA
ATTTTCTCAAGTAGATTACTAGACTATTTGCAAAGCATCTAACACCACTGAATCCTAAATAGCTGGCTGG
ATTTCACTGTTCTATGGACACTATATGAGACAACCAGAGGGACTGATGTGACGACATTCCTTGCCCTTTC
TTTTGATTTATCTTTTTTATGGTGTATCTTCTATGTTGTCCAGATTTCCCCCTCAGTTCTAATGTTTATA
TTTTGAAAGCTGATAAATATTGGAGCTGAAAAGAAGAGTTTAGGCTAGAATTAAGTATTTTGTAATAACG
AAGCTGTTAATTTCCCTTTGTAATTTTTTTTTTTTAAACTCACAGACACAGGTCTCTGAAAGAACACTCT
AATTCTGGTTTTCACTGGCGAAGTAATTTGTCTTTTTTCCTTCTTAAAAATAGATACGATTCTGATAAGA
AAAATGATAGCTTGTCTTTTCTCTATAAGAGAAGCAATATAAACCAGTTTCAAACTCAAGTTTAAATTTG
CACTGGGATCTAGGGTATTTTTTCTTTCAGAATTTGAAAGTTGGCTCGGTACAATGGCTCACACCTATAA
TCCCAGCACTTTGGGAGGTAGGAGGATTGCTTGAGCCCAGGAGTTTGAGACCAGCCTGGGCAACATAGTG
AGACTCTCTTTTTATGAAAAAAAAAAAAAATTTAAAGCTGAACGTTCATTTGCTTTTAGCTTTAGATACA
TATTCTTCTTCTTCTTTTTTTTTTTTTTTTTAGAGATGGGGTCTCACTCTGTTGCCCAGGCTGGAGTGCA
GTGGTGCCATCATAGCTCACTGCAGCCTGGAACTCCTGAGCCCAGTGATCCTCCAGCCTTGGCCTCTGGA
GTAGCTGGGACTACAGGTGCGCACTACCATGCCTTAGATACATATGCGAATCTCATTGGCTATTGCCTTG
GATATCACAATGGAGTTTAACTCTGTCCATCATGTCAGCAATATACTATAGTGTCACTGGAGCCACAACT
GGATTATGGATGTGACTCACCTAGTGATGCGCCATCCTTTTCAATAACATCTGATAATTGCAGAGATGAC
TACATCAGACTAGTTGAGCCACAGGGTACTGTTGGGTCACTGGTGACTGGCATGTGGGATAGGGGTGGAA
GGGAATCTTTTTTTGGTGCTGCAGAAGATAAAGGGCTCTACCCTCCTGTGGAGTAGTGTGATACCCCAAG
AATGGAGAGAACAGAAATTCTAAGATACGCAGCTGATGCTGGAGGTTAGAGAATTTTACTGAGTTTTTCT
TGTTTTTTGGACAGCTAATCAGAAGACAATATATAAAGTTTAGTAACTGAAGTATGGTCAGGGAGAGGGC
CTGAAAATTCTGCTTTGGAATATTTAATGGTGACATTTAGATAAGGGTTAAGGGTGAAGCCAGCCTTATG
GATGTGGGGATCAGGGGAATGTTCCTCAGGTGCTCAGGTCAGGGACAGTGGCAACATCCTCAATAGTTAA
GGAGGTGTGGGTATTTTGAGGAAATGATGTGGTTTGTTTCATCACTATATAAGCATCAGGAACTGGCAGT
AGTGATATGTGAATGGGATCAAGGTAACCGAGAACAAGATGTGAAACTTTAGGGAAATGGTAAGTCTGTC
TTTCATCTGGTTCAGCACACGGTGAACTGTTCCAGAGGTAGCTTCTTAAAGCCCTTCGTTTTGTCAAACC
AGAATCTTTTTTTTTTTTTTTTTTGAGACAGAGTCTTGCTCTGTCACACAGGCTGGAGTGCAGTGGCACG
GTCTCAGCTCACTGCAACCTCTGCCTCTCAGGTTCAAGTGATTCTCCTGCCTCAGCCTCCAGAGTACCTG
GGATTACAGGCACGCGCGACCATGCTTGGCTAATTTTTTGTATTTTTGTAGAGACGGGGTTTCGCCATGT
TGGCCAGGCTGGTTTTGAACTCCTGACCTCAAGTGATCTGCCCATCTCGGCCTCCCAAAGTGCTAGGATT
ACAGGTGTGAGCCACCACGCTTGGCCTGGAAACCAGTCTTTAAGAGAAAGGGATGCAGGAAAGATTGGAG
ATCTTTGAAGAATCTCTTTTTAAGAAAATTATCTTTTTCGCTCTAGTTCTGTCACCTTTAGTAGACTCTT
TATTAAGCCTAAGAATCTTATACTTCTTATCTCTGTTCTATGTGAGTTGGTATAACGGGTCAAATTCACA
ATCCAGAAATGCCTGTATTGTTTCTGATGTGCTGAGGAGGTGTACAATTGTCTTCCTTGATTTCAACCTC
AAAAGCTTTACATCTTTACCTGGGAACATAGAAGTCATTATTATTATGTCTTTCACAAATTATGTAAACT
TAAAAAAATTATATTTTCAATGTAAACCACTCCTTGGAGGAAATTAGTTCCTTTAACATTTATTTCCTAA
TTTTAATTTCTACTAGTTTTATCTCTTTCAAGCTTAAATGAGTGGGCCTTAGTTCTGTATTATTGCCATC
TTAATCATGTTCTTTGTGATTTTAGTAGCTTTACTTTTTAAAACTTACATTTCCATAATGAAGACTTCTG
TATTTTTGTTTTTTTCGTCATATATCCGACTCCAGCTCCCTCAAGACCTGTCAGCTTTTCCCTGCACCCT
TTGTTATCTCTTGCTGTGGTCCAAAGGTAAGGACAGTAAGCCGTGGGCACAGACTTTCCCTAAATCGTAT
GGATTTCCTTTAGTCAATAACAAGAATTTTGATAGTTAAGGCATTTACGCCTTAAGAATGTGATAAATTC
CCCCTCCTACTGAATGTAAAGAACTATTTTTCTACGCAGACTAACTCTTATATTCACTTTTCTTATAGAA
TAAGTACGTAACCAAATCTGTTCTTGGCTTCTAAGAAGTTAGAACTCAACGTAAAGATGACTTGTGGAAA
ATTACTTACTCATTCTGGGTAAATGGACGCAGGTATTTAGTGCTTAAGTAGGTTCTGATATACCATTACA
TTAACATCATTTTAAAAATCGCTCTGAATTATATGTATTTTAAGAGTGACTGGGTTTTAAAAAAAATAGA
AGTGGTAGAGCATAAATCATAATCTTTAATAAGGTAATAAAAGGCAAACACTATTGCTCTGGGCAAAACT
GGGCTAATGCTTTTGGATTTCTTTCCGCAGTCTTCACAGACGACGCTCAGCATCCTATCAGCCCTTGGCA
GAGACACACGATCCAGCCTAAATCCTCAAATTCTGGCTCTTGCCCAAAGTGTGTAGCCAGTATCCATAGA
GACTGCGAAAAGCATCCGCGAGCCGGGGCGGCCCCCTCCTCCTCAGCTCCAAATTGCTTCCTTTTTCGGT
GGGAGAAAAAACTCCGAGGAGTTGTGTGAGCGCCTAAGGAGCGCACGCTCCTCCGGCCCTCAACACCCAG
TATAGTTTTCGCTAAGTTCCTGGCCGAACCACAGCTCCCACGATGCACCCAGGTTTTAGAGCGGTCAGAG
GGGCGGCTTCCGGTTTAGGACAGCGCGGCTGCGCACGGCGTGTGGTCTTATTGGCTCGAACCGCCTCGTG
CGATGCGCGTCACGGCGACGTGCGGTCGTGGGGGCGGGGAGACGCGGCGGCGGCGGTCGCTGTCGCGGCG
GCGAGAGCGCGATTCCAGAAGCGGCATCGCGGCGGCGGCAGCGGCGGCGGCTACACCGGGCTTGGCCCCC
TCCCTCCTCCGTTCCCCCCTCCTCCCCCCTCCCCTCAGCGGTGGCTCCCAAGAAGTCCGAGACACGCGGT
GAGGCGGCGACGGGCTCCGGGTGAGGGTCTCTGGAGTGTGGGCGCCGTTTCCTGGGCGCAGAGGGAGAGC
TTGGGGTCGGGGGGAGGTGAGAGCTGAGGGGCGAATGTCCCTGGCGCGGGCGGGAGAGCGCGCTCTCGGC
CCGGCCCGCAACTTGATTCCTAACTCTGACCTGCCGCCCGGCGCGCCTTGGCCTGAGGGTCGGCCGTGTG
GGGACCTGGAGCCGCCCTCCTTGCGACCGCTGTCGGAGGAGTCCGGCGCCCGCTGTTCCCCTCGCCTCGC
CTTTCGCGGCGGAGGGTCGGAGCCTGCTGCCCTCTGCTCACCTTCGGGTGCAGTTGCTCCAGCTCCCTGC
CCGCGGAGAGGATCTGCCCGCCCCTCTTCGTCTCTTGTCTGTTTTTATCACTTCTAGGAATTTGTTTGGT
GGAGGCTTGGGATGGGCTCTCTCAGCGCTACTTTCACAGTGACTGGGGTGGGGAAGAGTTCCCTCACCTT
TTTCAGGTGGCGCGTCAGGCAGCCTGCTTGGTACCTGGAGTCCTGTTTTCCCAAGCCCCCGTCCGTCAGT
GTTCGCTTCTTCTAAAATTACCGATGACAGGGTTATAAATCAGTTTTTAGGGGGCTCGGAGAGAGTTTGG
GTTGGAAACTGTTAGCTCTTTTTTCCTCCTGGCTTTTGATCTTGATTTAAGCGTTTGCCTCTGAAATTTT
GACAACTTCATAAGATGGTCCAAAAGTGAATTTTGGTAGAAAGATTTAAATGCACAGTTGGGATTTTATG
GGGGTACGGTGACTTCATTGCAGGTTGTTGACTTTTTTTTTAAAATAAGATGTTCTTTTAATTTTTTGAC
TCTGGGTTTCTGAGCCTTGGTTATTGTCCCCCATTTCTGTTCCAAAAGCAGACTTTTTTTTTTTTTATCA
CCCCTTCCCCTCACCCAGAGAATCCCTTTAAACGCCAAGAAAGCTGTCATAACATTTAGCATTATTTGAT
CACAGAACTTTTCCAAATGAAATGCTAAAAATTCCTTCAGAAAAAAGCATCCATCTACCCATAGCATTAT
TTTTTGGGGGGAGGCTATACGTCTTGTCAGTTCTGAAAGTTTGAAGTGACCTCTTATCAAAGGCCACCCT
TATGTCTCCTTAAACGACTCTTAGGTAATTCCTAGATGTTTTCATCCGTTCTGATAAATTTGTTGTCAGA
GTGTTGATTGTGATGATCTGCAACTAAAATTATTTCATCAGTAAAGATTTCCACACATGTGGCAGCTGCT
TGTTGGTGAATTATGAAATTTGAAAAAATTGCAAATTAAATGTGAAAGGCATATTTCTTTTAACTTCTGA
TTGTAACTGTCTGTCCTCCAGTATACGTTCCTATCTAGACTTTGGTCTGCTTAGCAGATTTCCTCTTTTC
ACTTGACACTAGGAATGGGAGTAGGATTTCCAGAGCCTAAAAGAGAGTTTGTTTTTTTTTTCTATCCCTA
GGCTCCTTGTATGAATTTATAAATAAATGGTTTTTTCTATCAATAGTATCAATTTAGCTGGTGCACTTTT
TTTTTTTAGTATAAATCACATTTTGTTTCCTAAAATGAAAATAATTTTGTTTTATTCTTTATTTCAGGGA
TTGACAGATTTTAAAGCCTGCAGGCCCAGTCCTGCCAGTTGCCTCTTTTTGTAAATAAAGATTTGATTAG
GCCTGGTGCGATCGCTCACGCTGGTAATCCCAGCACTTTGGGAGGTCGAGGCGGGCAGATAACCTGAGAT
CAGGAGTTTGAGACCAGCTTGGCCAACATGGTGAAACCCCGTCCCTATTAAAACTACAAAAATTAGCCGG
GTGTGGTGGCGCACTCTTGTTATCCCAGCTGCCCGGGAGGCTGAGGCAGGAGAATCGTTTAAACCCGGGA
GGTAGAGGTTGCAGTGAGCCAAGATTGCACCATTGCACTCCAGTCTGGACAACAAGAGCGAAACTCCGTC
TTAAAAAAAAGAGGAAGAAAAAAGTTTTTATTAGAACATAGCCATATGCAGTCGTTTAGGTATTGTCTGT
AGTTTCTTTTGGGGTACAGCAGCAGAGTTGAGTAGTTGTGCCAGAGAGCCATGTGGCCTGTAACACCTTA
TATTTATTTACTATGTGGACCTTAATATAAAAAGTTTGCCGAGCTCCATTAAAAATGATACATAGTCAAT
GTTGAAAATTTAAATGAGTCAGAAAATTGTAAAGGAGAAAGTAAAAGTTATTTACAAGTACACCACTTGG
GGCTTTTTTTCTGTGATTATAGAATTTGTTGGTGATTATTTTTTATATAAATGAACTCAAATTGATAAAT
GTTGGTTTGTAATCTGCTTTTTTTTTTTTTTGTTCATTTTGTTTTTTGAGACGGAGTTTCGTTCTTGTTG
CCCAGGCTGGAGTGCAATGGCACGATCTTGGCTCACTGCAACCTCTGCCTCCTGGGTTCAAGCAATTCTC
CTGCCTCAGCCTCCCTAGTAGCTGGGATTACAGGTGTGCGCCACCAAGCCCGGCCAATTTTTTTTTTGTA
TTTTTAGTAGAGACGGGGTTTCACCATGTTGGCCAGGCTGGTCTCGAACTCCTCATGTGATCCACCTGCC
TCACCCCTCAAAGTGCTGGGATTACAGGTGTGAGCAACCGTGCCCAGCCCTGTAATCTGCTTTTTTAAGA
AGTATTTCATTATTTGTATTTGGATTTTGATTGCTTTTGTATTTTCTGCATTGGAGGTCTTTCTAGAAAG
GGGCTTATGAACCGGGTTTTCATGATTAGTCTTACCAGTCAAGATTTAAAAAGAAAATCCAGGAATAATA
AATGCTTGGTAATGAAGATGCTGGTTAACATTTATTGAGCACTTACTATGTACTAGACAATTTCCACTAC
TTTACGTGTATGAAATTATTTAATTGCTGTGGCAGCTCTGTGAGTGTATACTATGATTTCTGTTTTACAG
GTGAGGAAACTAAGGCAAGGAGAAAAATTAAGTAACTGTTGGAAGTTTATCAGCTAGCAAATGGTGGAGC
TAGGATTTGATTTCATGCAGTCTGATTTGAGAAACCCAAATTCTTAACAATATGCTACAGAGCCTCTACT
TACATTATTGCTCAGTGTAGAAAGTTTAGACAGTCCAGAGATATACTGTCATCCCTTGGTGTCTGTAGGG
ACTGCTTCTAGGACCTTTGGTGAATATCAAGATGCACAGATGCTCAAATCTCTGATATAAATGGCATAGT
ATTTGCATATAACCTGTGCATATCCTCCCAAATACTTTATATCATTTCTAGATTACTTATAATACCTAAT
AGAATATAAATGCTATGTAAATAGTTGTTATACTGTATTGTTTGGGGAATAAAGACAAGAAAAAAAAGTC
TGCATGTTTCAAATACAGATGCTATATTAAACTTTTTGGATCCAAGCTTGGTTGAATCCACAGACATAAG
AGCCCATGGATATGGAGGGCCAACTGTATAATGTAAAAAAATTCCTTCTCATGCGCTAGTTTCCAGTTTT
CATCCTTTGTAGATAACCAGGGGCAACAGTTTGATATAGGTCTTTTCACCTCTCTTTGTGTGCATATACA
AGTAATTATTTGCAGATTTGTCTGCATATTCAGAATTTAAAAATTACACTTTTTTTTAGCAGCTTTAGAT
GTACAGAAATTTGAGCCGATAGCACAGAAAGTTCCCATATTCCCTTCCCTTCCCTCGTTTCTCCTATTGC
AAACATCTTGCATTAATTTGGCACATTTATTGCATTTGATGAGCCCATATCAATATGTTGTTATTTATTG
GTCTATAGTTTACATAGGGCTTATTCTTTGTGTTGCAGTTTTTTTTTTTTTTTTGTTTTGTTTTTTTAAG
ATCTCACTCTGTATCTCAGGCTGGAGTGCACTGGTGCCGTCATGGCTCTGCAGCCTCGACCTCCCGGGCT
CAAGCGATCCTCCCACCTTAGCCTCCCAAAGTGTTAGGGTTACAGACATGAGCCACTGCACTTGGCCGAG
AGTTATTTTTTACAAAAATGGGATCAAGCCCAGTGGTGGCTTGCGCCTGTAGTTCCAGCTACTTGGGAGG
TTGAGGTGGGAGGATTGCTTGAGGCTAGGAGTTTGAGACCAGTCTGGGCAACACAGCATGACTCTGACCA
GTCTGGGCAACACAGCATGACTCTGTCTCTTAAAAAAAAAAAAAAGTGGGCTGAGGGTGGTGGCTAACGT
CGGTAATCCTTGCACTTTGGGAGGCCAAGGCAGGTGGATCACCTGAGGTCAGGAGTTCGGGACCAGCCTG
GCCAACATGAATGAAACCCCCTCTCTACCAAAAATATATTAAAAAAAAAATTATCTGGGTATGGTGGCGC
GCGCCTGTAATCCCAACTACTCAGGAGGCTGAGGCAGGAGAATCGCTTGAAACTGGGAGGTGGAGGTTGC
AGTGAACCGAGATTGCACCACTGCGCTCTAGCCTGGGCAACAGGAGCAAAACTCCATCTCAAAAAAAAAA
AAAAAAAATGACGAAACCCGTCATTCAGCAGTCATTCACCTTTTTTGGTGGTTTTCAGGATATAGTGCTT
TTGGACTTTCTAATGTAAACATTCTTGATTTACTAATTTTCAGATTAATGACAATTTATTTTGGGAATGG
AAGGCTGGCTTGGATGTTACTTTTTCTCTCTTTTTATTTTTCTATTTTTTTTTTTAAGTGTATCTCATAG
TGGCAGCAGCTTTTCCCCAAAGGAATGCTTATTTTTTTTGACGGGACATTCTGGCTATACCTAATCATCA
AAGTTCTTCTTTTTTCTCTTTATCTCTGGATTTGGAAAATGATTCATTTTGGTTTTCTTCCTCATAAATT
CTTTTTGGTCTGTCCAGCCATTGCAGTTGCTAGTCATGTCTCCTTGTCAGACTTTGGCTTTTTATGACTG
TCATCAATGTTAGAAAAAATTACTTGCTCCTTATCACTATTTCAATACATTTCCTCTTGGGCTTTCCCTG
GGGGCAGCACTGAATTTTTTCCTTAGTTGAGCCATGCAGATTTTCTAGAAGTCATCGTTTAGTTTGGCTT
TTCACTCCTCCATGGGTGCTGTTCAGCTTCTAGGAGATTTCTTGCAGTTTCTACTTGTCAACAGATTCAC
CAGCAGCACCCTCCTCCTCACTGAGACTGGTGCTTCCCCACCCATCTTTATCATTTTCAGCCTTCTCTTT
TTCTTTCTCTTTTCTTTTTTCCTTCTTTTTTTTTTTTTTGGTTACAGCGTCTTACTCTGTTGCCCAGACT
GGAGTGCAGTGGTGCAATCGTAGCTTACTGCAACCTCCGCTTCATGGGCTCAAGTGATCCTCCCACTTCA
GCTTCCCAAGTAGCCGGGATTACAGGCATGCACCACCATGCCTGGCTAATGTTTGTATTTTTTGTAGAAA
CAGGGTTTTGCCATGTTGTCCAGGCTGGTCTTGAACTCCTGGCCTCGAGCGATCCTCCTCCCTCAGCCTC
TTGAATAGCTGGATTTAACAGGCAGGAGCCATTGTCCCTGGCCATATTCTTGCACTGTTGATTTTACTGG
GGCCTCTGTCAGCTTACCCTGGAATTCTGCATCATCTGAGGATTTGGAAGAACTGAATCACAGTTATAGT
AGACAGCATCATATTCTTATCCTTGTGTGTTTTATACTGGGACAGGTCTTGAGGAAGTTCTTCTGTCATG
GCTAGGGGACATTGAGCTATTACCTCTTATAGCACTGATTTGGACTGTCATGTCTTCCCTAAAGTTCTTG
TCCATAACATAATGGATGGTCACAGTCATGAGCAATAACTGAGTGATCTCTGGGACTAACAGGTGATGAA
ATGCATGTTTAGCAAACAGTGATTTCTGTTACTTCTCTGTGGTGCAGCAGCAGAAACTTTTGCCCAAAGG
CATAGAAGTTAGAGGGCTTATGAATTTCTGCCAATCTGGAGATAAGATTTATGAGCATCATGTTAACTCA
AACCTCTGCTTAGAGATTTCTAGCTGTGTGCTTCAGGAGTGTTTTGGTAAAACTTTGAGGATCATGAATA
AAGCTAATAGCTTAAAAGTTAAACACTGTGAGTTTTTTCTTCTTTTGTTTCCTGGGCACTTTTCATAACC
TTTTCAGTGTTTTGTGTTTGGAGCCACTCTTCCCAGTGACATACTGCACTTGTAGGTCTCTTGTCACTTT
ATCCTCAGATTCTGAGTCTGTCCTATTTGTTTCTCTTCTTCATCTTATCCTAGGAAGAATGTCAAGGTGG
CAACCAATATCTCAAAACATGCAGTTGTGATGACATTGACAGTTTTGGCATCAGTCCGGATGTTTCTTGT
AGAGTTCAGTCATCACATCCAAAGATATCTTGGCTGCAGTTACATTGCTGTCTCTTAACACGATGTATAT
GAAATTCTGCAGTACTATATCCACTTTATTCTTGTGTTTTACAGTTATATTCTTGCTATCAGTCACAACA
TGAGTATGTAAAGTCTCAAAAGCTTATTATGGCAACTTGGAAGTTCAAAAAAGAGCTCTAGCAAACTTGA
TGGATGGATATGATACTTCTCAGCAAGATCAAAGCTTTGTAAAATGTTATTAGATGATTTTGATTCAAGA
CAGTACAGTATAATTGTAGGAAAGTAGATCTTTTAGCTCTTGAGGAAAGTTACTTAGATGTTCTGGGTAA
CAGTGACCAATCTGTGCCATAAATATCACCAGCTCTGTCAATTCTTTGCCAGGTTTATTGGTTTTAATTT
GAGAATCTCCATATTGGATTTGTAGTGTATATAGTACTGCTGTAGTAACCCTTTAATGTCAGCCAGTGGC
TCCACTTTATTAGATTCTATAACTGCTGCTTGTTACTGGGCAGCTAGTTTCTGCAGGACATGTTGGCTGT
AGACTGGATCCACAGTGAGTAGTTCTCAGAGAACCCGTACTGCTGACATTTGCAAACCCATTTCAGCTGT
GGTGTGGAGCTGTGGCGTTGTACTTCTGGATGTGACCAGAAACAGTGTTTGGGAGCTCAGCAGGTGGTGC
TGTCTTGGCTGATTGTGTGTGTGTGTGTGTGTGTGTGTGTGTGTGTATTTTATTTAATTTCTGAGACAGG
GTCTTGCTCTGTCACCCAGGCTGGAGTGCCATGGCACAATCACGGCTCACTGCCCTCTCGAACTCAAACA
GTCCTCCCAACTGAGACTCCCACGTAGCTGGGACTATAGATGCACGCCAGGCTAATTTTTGCATTTTTAA
TGGAGACGAGGTTTTGCCATGTTGCCCAGGCCGGCCTTGAACTCCTGGACTCAAGTGATCCTCTTTGCCT
TAGCTTCTCAAAGTGCTGGGATTATAGGCTTGAGCCACCATGACAGGCCCTGTGTGTGTGTTTTTAAAAT
TAATATTTGTGGCTTACTTTACATCTGCATCTACTCTCTTCTTTTTAGTAGTTCTCTAGTGTAGTATTAT
TAAATGGATATGCTATAACTTAATACTGCTCTTACTGATGGATATTTAGATTATATAAATGGATTGTAAA
TTGCCCATACATTTTATGTATATTTATTGCAAGCTAATTCCCCTTTAGTAATTTTGTAATAGTTGAAGAA
AACACATTTTTCATTTTGATGTGTGTTATCACATTGCCACCCAGAAAGGTCACAGCAATGTAAACTCTCA
CCAGAAATGTGGGTGGAGTCTAGCTTCTCTGTGTCATTACAAATTAAAAAAAAAAATCTTTGTTTATCTG
ATAGGAGGTTAAGCATTTGCAGTATATTCTATCTAACTGGGAGATATGCTTTGTTTCAATAGCATTCTTT
TAAAGTTGATTTACGATTACTTCAGGTGGAGCATGCATTCTGTTCCCGGGCCTCCCTTAGGTGGAGCTGC
TTCACCTATTTACATTCTCTGCTTGGCTCTTGAAAGCATATGGTTTTGTTATCACTGATCAAAAAGAATC
TGGGTGCCTGCCTAATAGACTATATATTTCTTGAGGGTAAGGAACGTGTAACTTTCTTGATAATCTACTG
GCAGTTCCGGTATATAGCAGGTGGTAAGTAAATGGTCTTTTAAAAATCTAAATTGGTTCATGAACCAGTA
ACTGTTGGGGTTACTGCTGAGTGAAGTGCTTTAATTTTGTTAAAAATAATATTTTCAAATTGTGAAATAC
AAACTTGAAAAACTGCAGTGCATGCTGGGTGCGGTCGCTCAGGCCTGTAATCCCAGCACTTTGGGACGCC
AAGGTGGGCGGATCACCTGAGGTTGGGAGTTTGAGACCAGCCTGGCCAACATGGCGAAACCCCCGTCTCT
ACTGAAAATACTAAAACAAAACAAAACAAAAAAAACCAAAAACAAAAATTAGCCAGGCATCATGGCCACT
GCCTGTAATCCCTGCTACTTGGGAGACCGAGGCAGGATAATCACTTGAACCCGGGAGGCAGAGGTTGCGG
TGAGCCGAGATAGCACCATTGCACTCCAGCCTGGGCAACAAGAGCGAAACTCCGTCTTAAAAAGAAAAGA
AAAACTTCATTATAAAATTGTCACTTTGGCATATGTTGTCTCTCATCTTTCAACTCTAAGATAAGTTAAA
TAGGACGAACTGATATCTTAGGACCTGTTAGGAAATAATTAGCCTATTTTATAAGTACATTTGGTATTTA
GAAATTGGAAGTTGAGGAATTTATTAAAAAGGGGATTTGTTTAACATTACTTATGGTTTTGAGTTTTCTG
GATTGGAGTATACCATGTGTTAGAAGGGGATATATGTTGGCCAATGATATTATAGATTGGCTTAATTAGA
CAAGTGTCCAGACAGAAGGAAAAGGGAAGCTAAATTCTGAGAATTGCCAGATTATCTTCAGGCATGGGAA
AAATAACTGGAAAAGGACACTTAAGTTTTTTAATGGAAGACTACAACAAAATGATATTTTCTTTATAACT
GATCATATTACACATTCTTACTGGATTTATTGAATATAAGAATATTCTTATTTCCACAATGTCTGTGTAC
TAGAGAACAAAACACAAAGCATATGGTCTTAAAAGGGGCAAAATTAGTTAGCTTATTACACTTTGGCCAT
TTAGGCCTTTCTTTTGTATGTTTGTTGTTAGTATATCAGCTGATTATTTTTCATTGACAGCATATTCACT
TTAATCTTGTATGTTTTGAAATTAAGTTTAGTGAACATGTATGCCTAGTTGTATCCATGTATCCATTCCT
TTGGTTAAGGAGGATGTCTTTGTGGTAGGGGTTACATTATGAAAACTTCAGCAGAATGGCACAGAAGATT
GACTTTATTTTCTTGAAATTTATTTTATCCTATTTCTTCCAGAAAGGATATCTGGTAACTTAAAATAAAA
GATCTGGAAGTGTAATAAAATATGTGTAAAAAGAAGAAATCAGGGAAAGGACAAAGCATATATTCTTTTC
CCAAGTGACAGCATAATTCAATGACTAAGAATAAAAATTGGGTTTGAGTTTGCTTGGCAGCGAGGGTAAA
AATGGAAGTATGGTAAAATACATAGCACTAAACTAAAAGGAGGAACAAGACCAATTTTTCAGGGGTGAAA
TCCTCTCTTCACAGATTTCTAAGAAACTATTTAAACTGGACATTATATAAAAGCTGTTTTGTTGTTGTTA
GTGGCCCTAGATTTAAAAAAAAAAAAAAGGTGAGGGCTAAGCATTAAGATTTCAAGCCAATGAAATACAT
TTTAGGGGTTGATTTGAATTTAGGCTGCAGATGTATGTTTTATTTGGCCAATATATGTTTTTTTTCCTTA
AAATATTTCAAACATACATGAGACTACAGAGACCAATATAATTAACATTCATATATTAACCATACAGCTG
TAGCATATCTTAGCATGTTCCCATATTTCTCATTTTTTTGAGGAAAAAAAATCATGTACAGTTGAAGCAA
CTTCTTGGATTACAGTTTACTTTCTTCCCAGAAGTTAACTATTATTCTGAGTATGGTGTTGATCTTTTTT
TTTTTTTTTTTTTGGAAACAGGGTCTCACTCTGTCACCCAGGCTAGAGTACAGGGGTATGATCATGGCTC
ACTGCAGCCCATCTCCTTTGCTTGAGCAGTCCTCCCACTTCAGCCTTCCAAGTAGCTGGGAACTCAGGTT
TACACCACTGTGCCCAGCTAATATTTGTATATTTTGTAGAGATTGGGTTTCGCCTGTTGCCCAGGCTGGT
CTCCTGTGCTCAAGCAGTCCATCCACCTCGGCCTCCCAAAAGTGCTGGGATTACAGGTGTGAGCCACCAC
TCCTGGCCTGATCATTTTTAATGCATGTTTTTATGTAATTACTACATGGGTATATATTCACAAATCATAG
TATTGCTTTGCATATTTTCAGATTTGATGTAAATGATATATTCTGCTTATTCTATAATTTGTCTTTCATG
AAAATTTTTTTGATTTATATGAAACTGCTGATACGTTACCATTTTGACCTACAAAAATGACAGTTTCATA
TGGCCCACCCTAATAGTCATACTTTTATCTGCTGTAAAATTATTTTCTATTATATGAATATAGCCAGATA
AAATTTATTTATTCTCATGTTGATAGGCATTTAGATTGTTTCCAGGTTTTCATGTATTAAAAATAATGTT
ACATTGAACATTCCTTATTAGGAATCATTGTGCTTACATTTAAGATTTTTGGCAAGTTTTAAAAACTGCA
TGTTGTGGCTGGGCACTGTGGCTTGAGCCTGTAATATCAGCACTTTGGGAGGCTGAGGTGGGCGGATCAC
CTGAGGTCAGGAGTTTGAGACCAGCCTGACCAACATGGTGAAACTCCATCTCTCCTAAAAATACAAACAT
TAGCTGGGCGGGGTGGCGCGTGCCTGTAATCCCAGCTACTCAGGAGGCTGAGGCAGGAGAATCGCTTGAA
CCTGGGAGGCAGAGGTTTCAGTGAGCCGAGATCACGCCATTGCACTCCAGCTTGGGTGATGAGCGAAACT
CCATCTTAAAAAACAAATGAACAAACAGAAAAAACCCCACATGTCGCGACTCCCTAATGGATTGTGAAAT
CAATTCAGGAATTAGAAAAATATCAGGTTGTATTGCAAACAATAAGGATAAAATGTTTTGTGAAATTTTT
ATTTCTGTGTGTATGTATGTGGTTATTAAGTTTGAAATGAAGCCCTCTATGGTGAATTACCCAGAAGTGC
AATTATTGGGCTGAAGATTTTGGAGGTTATGGGAATTTTTTTAGCTGTATTGGATATTGTCAAATTTGCT
GTCACTTTGCATATTTTAGCACTAGAGTATGAGAATTTGCCATCTTCCGATTGTTATGATTAATGGCATT
GTCAGATTTTTTACATGTTTACAAATCAGATGAGTGTGAAATGTTACCACATTGTCTTAATTTTTATTTT
TGATTATGGGGTTGAGTATGTTTTCAAATGTTAGATGACTATTCAAGTGTTTGTTTCTTGAATTGGCTGT
TCTTTTCCTTCCACAGCCATTTGGGTGGTCTTTCTAAATTGATTTTAGAAAGTTTTAAGTTCATTACATA
CTTCGATTATGTTTGTTAAGTGAATTGCAACTGTCTTTTCTCAGTCTGTGTGGCTTATCTCTTAACTTTA
TATGGTTTGTTTTGTTAAATAGAAATGATTATTATAAAATATATCACTATTTTCTTGTTTGTGTTTTATT
TAAGAAACCTGGGGTGATCACTCTGTGGTTCTCCTCATGCACATTAACAAGTTTGTTACTTCATTTCTTC
AGTTGATCTCCCTTTTGTGAGGTGGTTTTTAAGTGAATCATTAAAGAGCAAAGGAGAAGTTTTCCCGTGG
CTCCCACGCTGCTATTCAAAACTTCCTGGGTTAGTAAGCCTTCTTGTGAATTGTCTTTAAAATGTTTATT
AAAAACTCTTAGGAACTCTCATATAAAGTCTTAAATTTGTGTACAAAGATGCTCTCTGACATTGCTTGAC
TAGTGAAGAAAGTTAAAACAATCTAAATGTTAAAAATATATTATATGTATCCAGACTTCAGAATTTTTTT
GTAGACATTAAGAGAATAAGCTAGAGCTATACGTTGTGACATGAAAATGCCTACTATATACTAGTTAAGT
TTTTTAAAAAGTATTTTGCAGAGAAAATATGCACAGTATAATCTTACAGTGCAAAATTTTTGCAGGCACA
TATGTATATGATATATGCAATCTATATATGCATATAGAAGGGTCTGAAAATATATAATAGTTGCCACTGG
AGTGGTATGTGTGTTTGTGTTGCTTTAATTTTTTTCAGTACACATTTAAACAGAAGTAAAAAACAATTTA
AAAGAAATTTGGCTGGGTGTGGTGGCTCATGGCTGTAATCTCAGTACTTTGGGAAGCTGAGATAGGAAGT
TTGCTTGAGCCCAAGGGTTCGAGACCAACCTGGGCAACAGAGTGAGACCTGTCTCTACAAAAAACTTAAA
AAATTAGCCAAGTGTGGTAGTGTGTGCCGGGACTGAGGTGGGAGGATAGCTCGAACCTGGGAGGTCAAGG
CTGCAGTGAGCTGTGATTGAGCCACTGCACTCCAGTCTGGGTGACAGAGTGAAACTCTGTCTCACGAATA
AATAAATAAATAAATAAATAAATGGGAGAATCTTAATAACCTTGGAGTATGGAATAGATTTGTTTGTTTT
TTTGTTTGCGACAGGGCTCAAGTAATCCCACCACCCCAGCCTCTGTAGTAGCTAAGACTAGAGGCATGTG
CCACCACACATGATTAATTTAATTTAGTTTTGTGAGGACAGGATCTCACTATGTTGCCCAGGCTGGTCTC
AAACTCCTGGCCTGACTGATCCTCCTGCCTCAGCCTCCCAAAGTGCTGGGATTACAGATGTGAACCGCTA
TGCCCCCACCAGTTTTTTTTTTTTTTTTTTTGGGTCTTGGTTCTACCTGGAATTTTTTTTTTTTTTTTTT
TTTTGGTGTGTGATGTGAGGCAGGGATCAAATTTAATTTTATTATATGTATACCCAATAACCATATTACT
GTTAACAGAGTGTTTTGAATTTTTGAGTTAATAATTTTTAAAAGCAGCTCATGGTTTCTTGTTAAGCACA
ATTGCAGTCTTTTTGTCCTTCACCAGGCTGCTTCATATATTCAAGTTTGTGATCTCTGTGACCAGAGTGA
TATGATTGGTGGATGACTTGATCCAGAAATAGAACCTAGAAGAGTAGATACAGAGTATATTTTGTAGGTC
TCACTCACATGTCATTTCTCTTACTATTTAAATGGAAATTAGATTGCACTTTCTTTCTTCTCTAATAAAC
TTGCTTTCACTTAAAAAAGAAATTGGATTGCAGACAATTTGAGGATATACATTAATGAGGAAACAGATTG
CTGGTTTTTTGCATTGCTGGCTACAGACCTTGGAAACTAGAGAAGCACATGGTTGATTGATTGATATCTT
GTTCTAAAACTTGAGCATACTTAAATGTATTTGGTCATCCAGCTATTATCACATATACTAACGTTTGAGA
CTTTTAATTTTGACTTTAGGGTACATGTATCAATACAGTTGTCTTAACTTTCTAAGATGTTTGATATAAA
ACACTGTAAATCACTTAGTTTAAGTATGAAGACCATTTTTGTTTCCTCTTAAGAGGGTTTTTATTACACT
TTTAGTCTAAGAAGTGTTGCTGCTGAGGCCTTCAGATGTATATTCTAAATTTTTCTTTTTTCTTTTTATT
CCTTTTCTTTCTCATACTGGTGGTCAGAAGAAGGCCTTATGATTTAGTAGAATTGAGCAATATTGATAAA
GATTGTAGATACAAGTTTATTTTTAGATTTCTTAGACTTGTAAAATTGAGAAAGATAATTAACCACTTCC
TTGAATTTTCCATTTGCAGCATTATAAAGCAAAACAAACAAACAGACAAAACAGACTTGTGCTCTGGAGG
GTAATATGTGAGATGAGATAGAGCCCCGAATGATTGTTATTTAATAATGGGAGAAAGGTGCTTTATCACT
TCAAAGTAGGGTTTTGATTTTGCCTGAAAGGTTAACTGTACAGGCCACATGTTTTATCTGAACAGATTTT
ATTTTTGAAGTAGTTTAAACAGTGGGCTATGTGGTATTATAGTACAGATGATTTATGGAAGGAAGTCTTT
ACATATTTGATATATTTTGGCACAAAATATATAATCCTAGATTTTTCAGGATTTCCCTGACTTTAATATT
TAGAACTGTTGTTCCTTGTATTAGCATTTTTATGTACAGATTGAGCATCTCTAATCCAAAAATCTGAAAT
CTAAAATGCTCCCAAACCTGAAACTATTCGACGCCACAAGTGGAAAATTCCATACCTGACTTCCAGTGAC
AAGTTGCAGTCAGAACTTTGTTTCATGCACACAATTACAAAACATTGTATAAAATTACCTTCAGGCTATG
AAACAAATATATTTTGTGTTTAGACTTGGGTTATATCCCCAAGATATTTTATTATGTATATACAAATATT
CCAGAATCTGAAAAAATTCCAAATCCAAAACACTTCTGGGTACTAATCTTTGTTTCAAAAACAATAGTCA
TTATTATAGATCTTGGGAAATGAGGATAACCGTTGTAATATGGCTGTAAAAGAGTTGTAGAAAGACTTTT
CTCCTTTAAAGACATTGTTTTTGTAGCTTTGACCACTACCAGTGGCTATAATGCATTGTTTGTGGCTTTA
TTGAAGTCCTATAAGATGGTGATAAAAGCATGGACTTTGAAGTCAGATAGATAAAGATTTGAATTCAGGG
TCTTCCACTTAAGTTTGTGTTTTGATGAATTGCTTGATCTCCGTGAACCTCAGTTTCTTCATCTATAAAA
TGGGACCCCACTTCATAAGGATTGTTGGAGATGATAGGTGAAGGGTCAACTACTGGGCCTTGTTCAAAGT
TGTCTTCAGTAGTAAAGTAACAATTCACCTTCTCCTACAGAGAGATAACATCATGATGTGGGAAGAAGGG
AACTACTTACTTTTGTGGGAGGCAGTGTTGTATAATAGGCCATTTGTTGCTTCTCTGTTTTGTACGTTGT
GTAACCTTAGGGAGATTATCCAGAAAGTTTACCCTCAGTCTTCTCATTGGTAAGTAGAGATAATGTTACC
TATGTAACAGCAGAGTTTTAAAGCTAAATGAGAATATTTATAAAAGCTCACTTTATCTGGCACATGGTAA
CATGGTAACTTCTATTAATAAAATTTTATGGTAATAAGCAATATTATGCTTCTGAAGTAGGTCACAAGAA
GGGCTTTTGGTTGGAGAAACCAGAATTGTTATGAATTAGTCCGTGTTTACTTTGAGGGAGTAAAGACTAG
GGAGTATTCATTGAAGGAAGACTGTTTAGGAGGCTACTATTACTTGGCTAAAATACAAATAATGTGACTC
AATAGTTTCTCAAAATTTTGTGTAATCTAGGCTTATGTAAATAAACTTTACTGTTTTATTAGGATTTAAA
ATCAACTTTATTGAAATATAATTTAAACAATGAAATATACTCATTTTAGGGGTGCAGTTAGATGACTTTT
GATAATGTACATACCCACCCAGATAACCATCACCCTAATCAAGGTAATGGAATTTCTGTTATCTCCAAAA
CCTTCCTCGTGTCCCTCACAGTCAGTGCACCACACCCCTCCCCTCCTCTGGTTCAAGGAAACCACTGATT
TGCATACATTCATTTTGTCTCTTAGAATTTCATGTAAGTGGGCTGGGCAAGGTGGCTCATGCCCATATTT
CCATCACTTTGAGAGGCTGAGGTGGGAGGATCTCTTGAGCCAGGAGTTCAAGGCTGCAGTGGGCCAGGAT
CACACCACTGCAGTCCAGCTTAGTGACAGTGTGAGACCCGGTCTCAAGAAAAAAACTTTGTGTGTGTAAA
TGGAATCATACTGTACATATTCTTTAATGCCTGGCTGCTTTCACTCCGCATGTTTTTGAGAGTCCTCCAT
GTTGCTTTGTGTTCCAGTAGTTTGTTGCCTTTTATTGCTGAGTAGTATTCACTGTGGTATGGCTATACCA
CAATTTGTTTCTTCATTCACCTGATATTTGGGTTTCCAGTTTTTGGCTATTATGATTAAAGTGGCAATGA
ACATTGATGTACAAGTCTTTTTGTGGACTTAAGTTTTTATTTCTCTTGGGCAAATAGGAGTAGAATAGCT
GAGTCATATGGTGTATATTTTGCAGTTTCTTAATAAGTTAAACAGTTTTTCAAACTGATTGTGCCATTTT
ATCTTCCTGTAATCTCATTTACATTATATGCTGTGTTTTTAATTTTAACTTTTTTTTTTTTAGTGTGAAT
TGATATCTTATTGTGGTTTTTATTTACATTTTTCTTTTTAAAAGTTTTGTTTTTTGTTGTGAGACAGGGT
CTTGTCTGTTGTCCAGGTTGATGTGATCCTGGCTCACTGCAGTCTTGACTTCCCAGGCTCAAGGAATCTT
TCCACCCCAGCCTTCCAAGTAGCTGGGACTACAGGTGCATGCCACTACATCCAGCTAATTTTATTTTGTA
AAAATTTTTATGTAGAGATGAGATCTTGCTGTGTGCCCAGGCTGGTCTTTAAATCTGGGGCTCAAGTGAT
CTTCCCGCCTCAGTCTCCCAAAGTGCTGGGATTATAGGTGTCAGCCACCATGCCTTGCCTTATTTGCATT
TTTCTGATAGCAAATGATGTTGAACACTTCTTCATGTGTTCATTGGCCATTTGTATATCCTTTTATTAGA
TGAGGTGTCTAATTCTTAATTCATTTTTAAAAAAGCTTTTAAAAATTACTGAGTTACAAGTTCTTTATAT
GTTGTGGATACAAGTACTTTGTCAGCTGTGTGAATTTTGAATATACAGTCAGCGCTCCTTGTCTGAGGGT
TCCACATCCACAGATTTAACGAATCTAAGATGGAAAATGTTTGGTAAAAAAATAACAATATGCGAATAAA
AAATAATACAAATAAAAAATACAGTATAACAGCTATTTATATAGCATTTAATCATATTAGGTATTATAAA
TAATCTAGAGATTGATTTAAAGTATAAGGAAGATGTGCATATGCAAATATGGGTTTTTTTTTTTTTTAAA
TAAGGGACTTGAGCATCCTCGAATTTTGGTGTTCAGTTTTGGGTGGATTAGGAGGGTAGAGAGGGGTTCT
TGAAACCAGTCCACCAGGGATACCGAGAGATGACTGTATTCTTTTGTTCTGTGGCTTGCCTTTCATTTTC
TTAACAGTCTTTGGAAGAGCAGATGTTTCTTAATTTTGTTGAGTCCAGTTTATCACTTTTTACTTATGGT
TAGTGCTTTTTGTGTCTTAAGAAGTATTTAGCTACACCAAGGTTGTAAAGATTTTCACCTGTGTTTATTT
TGGAAGCCTTACAGTTTTAGACATTACATTTAAGCCTATGATCTATTTCAAGTTAATTTTTGTGTAGGAT
GTGAGGTAAAAGTTCAGGTTCTCCTCTCCCATATGAATATCCAGTTGTTCAAGCACCATTTGGTAAAAAG
ACTATCCTTTCCTCATTTAATTACTTTGACCTTTGTTGAAAATCAGTTGTCCATAAATGAGTGGGTCTTT
TTATTATTGCATTGATTTGTATACCTATACTTTTTTTTTTTTTTGAGACAGAGTCTTGTTCTGTCATCCA
GGCTGGAGTGCAGTGGCACAGTCTTGGGTCACTGCAACATCCGCCTCCCAGGTTCAAGTGATTCTCCTGC
CTCAGCCTCCCGAGTAGCTGGGATTACAGGTGTGCACCACCACGCCCGGCTAATTTTTGTATTTTTAGTA
GAGATGGGGTTTCACCATGTTGGCCAGGCTGGTCTCGAACTCTTGACTTCAGGTGATCCACCTGCCTATA
CCTCCCAAAGTGCTGGATTACATGTGTGAGCCACTGTGCCTGGCCCTGCATATCTATACTTATGCCAGTA
CTACACTTGATTACTGTAGCTTTATGGTAAATTTTGGTTACTCTAGATTCTTTGCATTTCCATAAATTTT
GGTATTAATAGTGTCAGTTTCTATCAAAAAGCCTGCTGGAATTTCAGTTTGTGTTGTATTAAATATACAG
ATCATTAGGGAGAGAATTGTCGTTTAAAAAATATTGAGTCTTCTGGTCCTTGAAAATGGTGTATCTTATT
ATTTTTTAAGGTTCTTTAGTTTCTCTCAGCAGTGTTTTGTAGTTTTCAGTGTAGGAAGTGTTGGACATCT
TTTATTGGATTTATTCCTAATTATTTTAAGTGCTTACATGGGAATTTTTTTAAAGTTTCATTTTTTCCAA
TTTGGTGCTAGTATGTAGATTACAATTGATTGGTGGTCTGTCTAGCATGTAGAAATTCCTTAATAAGTAG
TCAGATGGAATTTAATTTGTTCTCATCTAACTTTCTGGAGAAAATAATCATCTAAAAGAAAAAAGATTAT
CTCTGAATTTGAATGGGTTTTAAATTTGTATGACTGTGGCTTATTTTAATTTCTCTGAATTTGTAACGTA
GAAGTACATTTGGATTTTATGGTAAAATATACGATATTGGATATAAGTGGAGACATGTAGAGATCATTCA
ACAGATGAGAGGATATAGCTATAGAAAAGCAGAGTTGAAAATTTTTAGATGGTAAAATATTCAATTTTAT
ATTAAATATAATATTTTAAAGTAGAAAGTGCTTGAGTAGTATGATTAAAAAGAGGAAACTTGTTTTCATT
TGAATACATTTTGGCCTACAAGAATGTCTGTTACTGGCTTAAGTATTTTTGTTTGTGTATACCGTCAGAT
GATTTACCATTTCCTAGCCAAACCTGATTTGTTGGCCCAGTACAACTCTCTTCTCTCTTTTAAGCTGTGT
CTTTGATAGTATTTCTGTTCATCCTGCCTAGAAGATTGTGTTCTCACACAGTTTTCTGTTGTTGGTTCTA
AAACATGGTGTAAATGAGAGGATTGTAAAATGCGCCAATCAGCACTCTGTAAAAATGCACCAGTCAGTGC
TCTGTGGCTAGCTAGAGGTTTGTAAAATGGACCCCTCAGCACACTGTAAAATGGACCAATCAGTGCTTTG
TAAAATGGACCAATCAGCAGGATATGGGCGGGGATAAATAAGGGAATAAAAGCTGGGCACCCCAGCCATC
AGCTGCAACCCAGTCAGGTCCCCTTCCATGCTGTGGAAGCTTTTTTCTTTCGCTCTTCACAATAAATCTT
GCTGCTGCTCACTCTTTGGGTCCGTGCCACCTTTAAGAGCTGTAACACTCACCGTGAAGGTCTGCGGCTT
CATTCTTGAAGTTAGCGAGACCACGAGCCCACTGGAAGGAACCAACTCTGGACACATCTTGGTGACCCAG
ACGGGACCATTGGACCCCTTTTGCTTGCTATTCTGTCCTATTTTTCCTTACAATTCAGGGGCTAAACACC
AGGGCACCTGTCGGCCAGTTAAAAGCAACTAGCGCGGCCACCGGACTAAAGACACGGGTGTTAGGCTTTC
TGGGAAAGGGCTCTCTAACAACCCCCGACTCTTCAGAGTTGGGAGCGTTGGTTTGCCTGGAACCAGCTTC
CTCTTTGCCTGTACTTCTGGGCTGAGCCAAGGGTTGACAAGAGGGGAAAGCCATTCAGCTGCGGGGTCCC
AACAAAAAGTTGGTTGACCCTGTAGCCATGAGGGGAACTCTGTAGGTCAGCCAAAGGTTCAGTGGGTCGG
TCCAGGGGTCCTTGGTAGAAGTTGTTAGTTGAACTCATTTGGGGTTCCATTTGTAAGACCATCTATAGCT
TGATGGCATCGATCCTGGAGGAAACAAATTTGACAAGGAGTTTAAAAATACGGGCCTGAAGGCGAGTAAT
AGCAAGATGGCTGTTACGGGACCTAGAAAGGGGAGAAGCCATGTTGCCCAGCTCCAGAGGTTGGTATAAG
AGTTTGAAAGGCGTTGTCTGATTTCAGAAGCCTTTTCCTGTAAACGCCAGGTGGCGTCTCATACTATCCC
TCACTGGTTAGTGTAAAAGCAACATTCTTCCCCTAAGAAGGTGCATAGTCCTCCTTTCTCAGCAGTGAGG
AAGTCTAGGCCTTGGCGGTTTTGGAGAGTCACTGCTGCCAAAGAGTCTATCTGGGATTGTAGAGGAAGGA
TAGATTTTGTTATTTCTTGCAAGCTGTCTGAGAAATCCTTTGAGAGTGTGTGGTAGTAGGATAATGAAGT
AGATAAATCTGCTATTCCAATTCCTGTAGCAGTGGCCATTCCTAACCCTATAAGTAGGGGTATTAGTTGT
ATGGCCCTGTGCTGATGGACTTGAGCTTTGAAGGGGCACTGATAGAGTCTGATTTCCTGGGGCAATGTTA
ATGTTGGGACTTAGGAAGACTAAGGTGTAGGTGCCTGTCCAGTTGGTGGGGAGGCAGATACAGGTTGAAG
TTCCACTTAAGAAGAATATGCCTTGGCTGGGTAGACAGAAATTTACCCTGGCTTTTAAAGGAATAGGGTA
CACTGTTTTTTCTTTACTACTTCTATCTCTGTGTCTCTCTTTGTCTCTTCCTCTCTCTCTCTCTCTCTCT
CTCTCTCTTTCTCTCTTTCTGACTTCTTTTCTGTCTCTTCCTCTCTCTGTTTCTCTCTTTCTGACTCCCT
CTTTGTCTGTCTCTTCCTCTCTCTCTCTTTCCTCTCTGCTGGTCTTTCCCTGCCTCTGCCAGCTGCTTAT
GCTGCTGTTCTCCCCTCTCCTTTCAGCAGTGTAAGACTGCCACCTCCTTGGGTTTTTGCACTGTGTGCAA
TAACTCCGTGATTTCCTTATGGTATTTAATGGGGTTCACCCAGAGGTTAGGAACTCCCTTTCTTTCCATA
TTGCATCATGGGCACGAAGGATTAGATAAGCATACTTGTTATCTATACACATTTATTCTTTTTCCCTTTC
CCAGTTCTAAGGCTTGGGTAAGTGCCACTAGTTCTGCTATCTGGCCGCTGGTCCCTGGGGGAGGAGGCTT
ACTTTCAAGTACTGTTACATCACTAACTATGGCATAACCTGCCCTTTGTATCCCATTCTTCACAGATGAA
CTGCCATCAGTGTATAGGTTAAGGTCAGGATTAGCTAAGGGGACTTCTAAGAGATCCTCTTGGGTGGCAT
AAGTCTGGACTATAATTTGTTGGCAGTCATGCTCGATTGGTTCCCCATCCTCTGGGAGAAAAGTGGCAGG
GTTGAGAGCCGCACAGATGCATATTTGAAGCATCGGTCCCTCAAGGAGTAGCACCTGGTATCCAAGCAGG
CGGTTGTCTGATAGCCATAAACTTCCTTTGGCACCTAGTGTGCCATTTACATCATGAGTAGTCCAGACAG
TGAGATCCTTTCCTAGTATTATTTTGATAGCCTCTGATATAAGACGGCCACTGCTGCAACTACACGTAAA
CAGTGAGGCTAGCCTTTTGCCACTGGTTGTAGGGTTGTCCCGTGAGTCTGAGTAAGGACTTCAAGAGCTA
TTCCTGCTCTCTCTCTGACGTATAAAGAGAAGTTTTGTCTTGTGGGAAGGCTTAAGGCTGGAGCTTGTAC
TAGGGCCTGCTTTAAGGATTTGAAGGCTGTTTCTGCCTCTGGTTCCCATTCTACTAGATGAGTATTTGCC
CTCTGGGTCTCCTTTATTAGAGTATAGAGTGGCCTGGCCATCTTGCTGTATCCAGGGATCTGTAGTCGGC
AAAAGCTGGTGATTCCAAGGAACCCCCGCAACTGTTTTAATGTCTTAGGGCGAGGATAAGCCAGTATAGG
CTGTATTCGTTTCTTGCTGAGGGCCCTGGTTCCTCTGGCTAAGATTAGGCCTAGATATTTGACTTGTTGT
AGGCAGAGCTGGGCCTTTGATTTAGATGCCTTGTACCCTTGATTAGCTAGAAAGTTCAAGAGATCTCGAG
TAGCTTGCTGGCATGAGGCTTCCGAACTGGCAGCCAAAAGTAAATCATCCACAAACTGAAGGACCAGAGT
GCCTGGACTTGAGAAGTGGCCTAGATCTTGGACCAGTGCCTGACCAAACAGGTGAGGGCTATCCCTGAAC
CTTTGGGGCAAGACTGTCCACGTAAGTTGGGACGTGTGATCTGTAGGATCCTCAAAGGCAAAGAGAAACT
GGGAGTCAGAGTGCAGGGGAATACAGAAGAAGGCATCCTTGAGGTCCAGAACTGTGAACGATTCTGCTTC
CTTTGGTATTTGAGAGAACAGGGTATAGGGCTTGGGTACAACTGGATATAGAGGAATTACTGCTTCATTG
ATGAGTCTAAGATCTTGCACTAGTCTCCACTGACTGGTTTTTGTACTCCTAGAATTGGGGTGTTGCAGGG
ACTGCTGCATTTTCTTACTAGGCCTTGAGCTTTTAAATGTCTAACAATATCCTGTAATCCTTTATGAGCT
TCAGGCCTTAAGGGATATTGCCTTTGATAAGAAAAAGTGGTGGGATCTTTTAGCCTTATTTGGAGTGGGT
GGGCATATTTTGCCCTTCTGAATTGTCCTTCCAATGCCCAGGCTTCAGGGTTGATTCCCTCCTCAAGCAG
GGGACAACAAATTGGTAACTTGTTCCCCATATTCATGTAGATAATAGCTCCAGCTTTGGCTAATATGTTC
CTCCCTAATAAGGGTATGGGACTTTCAGGCATAACAAGAAAGGCATGTGAAAAGAGCAAAGTCTCCCAAT
TACCACTGAGAAGGTGGGAGAAATACCTGGTTACAGACTGTCCCAGGATTCCTCGGATGGTAATGGACCT
TGAGGACAGCTGTCCGGGACAGGAGATTAACACTGAGCCTCGGATGGTAACGGACCTTGAGGACAGCCGT
CTGGGACAGGAGATTAACACTGATAAAGCTGTACCGGTGTCCAGGAGGAAGTCAATTTCCTGGCCCTCAA
TGGTTAAACATACCCGGGGTTCAGTGAGGGTGATGACATGAGCTGGCGCTTGCCCCGGGCACCCTCAGTC
CTGTTGGATCATCTGGTTGGGGGCTTCTGGCCCAGAGAACCTTTGTCCTTTGGGGCAGTGCACCTTCCAG
TGATTGCCTTGGCATAGTGGACATGGGCGAGGGGGCAGCTTGTTTCTCGTTGGACAATCTTTTTTAAAGT
GTCCTTGCAAACCAAATTGATAACAAGCCTAACCGGGTGATTGGCCTGCTCCATTTTCTGTCCTCTCTGA
ACCACCAAGGTCTGTTTGTCTGAGGGCCATGACTAAGGCTGTAGCCTTTCTCTTATCTCACTTTTCCTTT
TCAGCCTGTTCTTCTCGATCCCTACTGTAGAACACGGAGGTTGCCAGGTTTAATAATGCGTCCAAATTTT
GTTCAGGGGCCAGGGCTAGCTTTTGGAGCTTTATCCTGATATCTGCAGCTGACTGGGTAATAAACTTATC
TTTTAGGATCAATTGACCCTCGAGTGAGTCGGGTAACAGGGAAGTATATTTTCTTAAGGCCTCCTGTAGC
CGCTCGAGGAAGGCGGTAGGATTTTCTTCCTTTCCCTGAGTTATGGTGGATGTCATTGAATAATTCATGG
GCTTTTTCCTAATTCTGCTTAGTCCTTCTAGGACACAGGTTAACAGATGTTCACGACTCCAGTCCCCATG
ATCTGAGTCGAGGACCCGGTGGGGATCCACACTGAGAATGGCTTGCTGACCAGTAGGGAATTTGTCCCTT
TCTTCGACTGTCATTCTGTCATTTACGTGACTGAGATGCCAGGTATCTCCAAACTCTTGGGCTGCAACTA
AAGCTGCATTCTTTTCATTAAAGGCCAGGGTTTGATCTAACAATAGCATGACATCTCTCCAAGTGAGGTT
GAAGGTTTGCCCTAGACCCTATAGGACATCTATATACCTATCAGGATCATCTGAAAACTTCCCCAGGTCT
ACTGTCATCTGCTTTAAATCAGAGAGAGAGAGAAGGGGACATGTACCTGGGTTGGGCCAAATTCCCCTCC
CCCTACAGCTTGAAGGGGACATAACCGATAGCCTGGGAGTTTTTGTGGTCCTTTGGAGATTTCTTTGCTT
GTTTCCTTCTGGGTGGTGGAGATTAGAGGAGGCTTATCATTAATAGGAAGGGGAGCTGTAGGAAGGCTAG
GATATGGGGGTAAGCTGAGAGGTCCTCCTCCCCAATATTGCAAGCTTTGCATAGTTGTGGATTATCCTTC
AGTGAAAAGAAAGCTTGGACATAAGGTATTTCACTCCGTTTGTCTTCCCTCTTACAGAAAAGGTCAAGCT
GCAGGATAGTATTGTAATTTATATTTCCCTCAGGTGGCCATTTTTGACCATCAGAGAGAGAATATTGGGG
CCAGGCCATAGTGCAGAAAAAAATGAGCCGCCTCTTTTTCAGGGTTTGCAGGTCAAATTGGTCCCAGTGG
CTTAGGATGCATTTCAAGGGTGAGCCTGTTGATGCCTGTGTGTTCCCCATCTGAAAGAAAAAACCTCCTG
TGGTTTTGGTTTGTTCCCCGCTACCCTCTCAACCCCGAGTTATCGTGGCTTTAAAAACCGGACCATTGTC
GCTTTGGCTGGCCTATAGAATTCCCCTCTGGAGGACACTACCACTGCAGGGCCCCTTCTTTACCCCTATC
CAGCAGGAAGTAGCTAGAGTGGGCATCGCCCAATTCCTAGCAGCAGTTGGGGTGTCCTGTTTAGAGGGGG
GATTGAGAGGTGAAGCCAGCTGGACTTCTGGGTTGGGTGGGGACTTGGAGAACTTTGATATCTTACAAGA
GGATTGTAAAATGCGCCAGTCAGCGCTCTGTAAAAATGCACCAGTCAGTGCTCTGTGGCTAGCTAGAGGT
TTGTAAAATGGACCAGTCAGCACCCTGTAAAATGGACCAATCAGTGCCCTGTAAAATGGACCAATCAGCA
GGACATGGAGGGGGACAAATAAGGGAATAAAAGCTGGCCACCCCAGCCAGCAGCGGCAACCTGGTCGGGT
CTCCTTCCACACCGTAGAAGCTTTGTTCTTTCGCTCTTCACAGTAAATCTTGCTGCTGCTCACTCTTTGG
GTCCGTGCCACCTTTAAGAGCTGTAACACTCACCACGAAGTTCTGCGGCTTCATTCTTGAAGTCAGCGAG
TCCATGAACCCACTGGAAGGAACCAACTCCGCACACATAAAGATGAGGAAAATTGTTCTTTTGGTTTTGA
CGACTTTACTATGTGCAGTTAGCATTGCTGAATGTGGTGGTCTCTGAGAGCAGTGTAATTCTGATGATAC
ATACAGGCATTTGTCTATTAAAAATGGGATGAAATTGAAGATGGTTTATTGCCTTTTATATCATCTTTTG
GATTTGTGATAAAGACAAACCTTTGAAAATGTTTGTAGTTATTAATATGACCAGAAACTGAAACTGTTTC
AGTTTGGCACAGGAGAGATTAGTAGTAGTATTGCTGTTAGTCATTATGTAAGAGATGATAATGTTTGGAT
TTAACCTTCGATTGTTTGTAATATGATATTCCTAACATTCCTGACTAAAAGGCTAGTGAATAAAAAATAA
TGCATTTACAACTATATTAAAACTGCATTGCAGAAAGCATATGAAATTTCAAGTTTAGGAAATTTCCTGC
TTTGAAACTTTTTTCTTCACACTTCACGCTTTCAGCACTTATGTGTGATGCATCTAGAAGATGCATCTGG
TTTTGCCAAATACAGTACTGCACCTGAATTACATTTTCGGTATTTATTCAGTATTTATTTTCAGTCTTTA
TTTCCTAAAGGTTAATTTTCTTTAAAACCTTAGCAATTTTTCATCCAGCCCTTTTCATGTTATAATCATT
TGCTTGTGTGATTTTACCACACACTGTATCCAACTAGGTAGCATTACTGGAAAACTTATTTGATTTTTTG
CCAAATGGGAGATGGCTGGAATATTTTTTGTAGTAGAGAATATGTGGAGAAAATGCTTTAATTGATCCCT
GACCAAATACAGCAAGAAAAACTTAAGTAAACCTTAATAAGCAGGCAATAGAAGTTAAGGTGATAATATT
CTGACTTTGGATATAAACACTCAAACATGAGCTCTGCTACTGGAAAGTAAAAAATAAAATTAGTGTTAAG
TTGAGATGAGTCAACTTGTTGATCATCTGCCTAGCCTACTGTGATAGGAATGGTTGTAAGACTTGGAAGT
TTTTCCTTCCTAAGTTGATAAGCATTTGAGTGGAAAGAAAGTCACTTTTCTCTTGATGTTTTTTCACTTT
GGAATTTTCAGACTGTTTAAAGTCTTTTAAAATTTTCTGAGTCACTTCTTTATTTTCTAGGCTGCTCATA
TTAAGAGAAGTATTGCCTTTTTATCCTTTAAAATGTAATACCCTCATCTTTAGTGGTTGTCCCTGCTTAG
GTTATATTGCTTAGAACATAACGTTTATGTTTGGTTAAAATTTCACTGAGCCACCGGGCACGGAGGCTCA
CACCTGTAATCCCAGCACTTTGGGAGGCAAGGCAGGTGGATCACCTGAGGTCGGGAGTTCGAGACCAGCC
TGATGAACATGGAGAAACCCTGTCTCTGCTAAAAATACAAAATTAGCCAGGCGTGGTGGCACATGCCTGT
AATCCCAGCTACTTGGGAGGCTGAGGCAGGAGAATTGCTTGAACCTGGGAGGCGGAGGTTGTGGTGAGCT
GAGATCGTGCCATTGTACTCCAACCTGGGCAATGAGAATGAAATTCCATCTTAAGGAAAAAAAAAAAATT
CAGCTTCTGAAAAGCACTAGGGACTAAGCAAGTTGACTATGTAGGCTTTATTATTAGATTTGTGTGTTTG
TATTATAAGGAAAACAAATTTTTAGTAAAATATAATGTATTTTGTTCATTGTTTAGTTCATTGCTGGATT
GCTGGTCTCTAGAACTTTTCCTGGTACAGAATAAACATATAATAAGTATTTTTTAAATGATGTACTTAAT
TTTATTACCCTACCATGCTTGAAACTGTGACTTTATATTATAGTTTCCTTTAGCAAAAGGTGAAACTATA
GTCAATATATAGATTATAAGTAAATTTTTAAAAATCTTCATTTACCGTAATAGCATCATTTTACATGTAT
ACCTGCTTTTCAGATTTGTAACTCATTTGGTTTAAGTTTGGGGCATTTGGATGTTTCTGATGTTGAATGA
TAAAATGTAGGCTTAAAAAAATTATCCTTCGAGTTTTATGCTGCTGTATTGTGTATAGCCTCTCTTTCAT
AATGATACTTGGACTGTGATGGTATAAAGTGACTCTTATTTTAATCTGAATGCATTCGAGATATTTCAAG
TTCTTGAAAGACTGTTACACTTCTTGGGATGGTCATCATCATATTTTTGAAGCAGAGATCCTAAAACTTA
AAAACCTCAGAGAAAGTCCAGCATCTATTTTCAACTCCTTGTTTTTTAAAGCTTTAGAACTTGACTTTGT
ACTCTTTGGTGAGGTGACAAAACAAATTCAGATTTTTTTTTTTAACTGAGGACTTCAAAAAATTTTTGTG
ATGTTTATAGTATTTTGTCTCTTTTTAGAAAGTCAAATTTGTTGAGGTATAGTTTACACTCAGTGAAATT
CACCCATTTTAGATGTATGGTTCTGTGAATTTTGACAAACATTACTGAAATGGCTAAAGTCGACTGCTAT
CTAGCAGACAGTGCTAAAATAGCAACTTAGCAGTTAAAATGTCATCTTTTTATCACTTTGGGAAGATTCT
CATTGGTATCTTTCCATGGGTACAATAAGAGACCAGCAGTGTGGCAGGCTTTCACTGTCTTTTTCCCTCA
ACTCTACCTCACATAAAGGTGAGATTTTCCTATTTTCTTAGGAAAGTCAGCTAGTGGGGTAGGGTGATAA
GCTAGGACGGGTTCCTTGTGATTTTATTTAAAGGTTGGTCTACTTAATGCTGGGGGAAAAAAGAAGTGTA
GTGATACAACATGTTGCCTACCTTTACATTTTCAAAATAATTTTAAGGAAATAAACTGCTGTTTGATTCG
AAATGATTCTTTGTGTTATGGCAGTTAAGATTTTATTAACACCAGGGCTCGAGAATGCCTGTTAAAAATC
TAGGTGTTGCTCCTTCTCAGCTACCAACTATATCTTTTTTTAAGGGCCATATTAGTAACATATGTTGGGA
ACTTTTGCCATGTTACCAATTATGCTTTTTCAGTTTTTTTTTTGAGACTTTTCTACTCAAAAAGGTTCTG
AGTATTTAGAAAGTACTCCAATTGTAATCATTCTTTTTATTGCTTCTGGAATGTAGCAGGGCTCCATGTT
CTTCCCCATTTCTTGACAGTTTCTTAATATCTTGGAACATTGTTGTAGAGTATAATGTCCTATCTGTGTT
GAAATACTTGGCTTTGGCATTTAAGTGCTATCATGCATTTATTTGAGAACAGTTTATGGCTCATTCAGAC
ATTGTGGATAAGAGCATTGACTTAGGTGTCAGGCAGAATAAATTTAATCTGACTCTGCCATTTAGTAACT
ATATGAACATGGATAAGTTAATTATAACTGCTCTAGCTGTTTTGTCATCTGCCAAATGGGGAAAATAATC
ATATGTATCTTCCAGGATTGTTGTAAGGATTAAAGAGATAATCCTTGGGAAATATTTTATTGCATGTAGT
AAACACTCAGAGCATGTTAGCTGCTCCTGTCATCCTCACTCAGCAGAAGTTTTTTTTTGTTTTCATCTTT
TTTTTTTTTTTGGAGATAGTCTTGCTCTGTCCCCAGGCTGGAGTGCAGTGGTGCAGTCTCAGCTCACTGC
AACCTCCGCCTCCTGGGTTCAAGCAGTTCTCCTGCGACAGCCTGTGGAGTAGCTGGGATTACAGGTATGT
ACCACCATGCCCAGCTAATTGTTATAGTTTTTTAGCAGAGATGGGGTTTTACCATGTTGGCCAGGCTGGT
CTCGACCTCCTGACCTCAAGTGATCTGTCTGCCCCGGCCTCCCAAAGTGTTGGAATTACAGGCTTGAGCC
ACCGTGTCTGTCCTCTTTTCATCCCAGACCATGCTTGGTGTTGTGGATACAAAGTGAATTCTAAAGAAGG
CCTCCCCATCTTAAGAAGTTTCATAGTTCAAGAAGGGAAGACAGGCATGTAAACAGATAAATGCAATAAG
TTGTGACATATGCTATATCAGAAACAAATGAAAAGAATAAAGGAGTTAGTATCATAAGCCTGGGGAGGTC
AGGGAAAGACATTTCTATCCCTGACTTATTTTTGATGTGTGAATAGTAGTTTCCTGTTGACTTATTTGAC
AGATATTTACTGAATACACTTTACTTTTGCCAGGCACTATTTTAGGTGCTGGGAATACCACTGGTTTCAT
GGAAGACAATTTTTCCATGGACTGGTGGGCAGGGGGTGGAGGGGATGGTTTTGGGATGATCCAAGCACAT
TACATTTATTGTGCACTTTATTTCTATTATTACTACATTGTAATATATAATGAAATAATTATATAACTCA
CCATAATGTGGAATCAGTGGGAGCCCTGAGCTTGTTCTTTCACAGCTAGACTGTCCCATATGGGAGTGAT
GGGAGACAGTGGCAGATCATCAGGCATTAGATTCTCATAAGGAGCGTGCAACCTAGATCACAATAGGGTT
CAGGCTTCTATGAGAATCTAATGCCACCGCTGATTTTACAGGAGGCGGAGCTCAGGTGGTAATGCAAGCA
GTGGGGAGCAGCTGTAAACACAGATGAAGCTTTTGAGCCCCTCCTGCTGTGAGGCCAGGTTCCTAACAGG
CTATGGAATTGGCTTGTAGCCTAGGGGTTGGGGACCCCTGCTGTATAGGAGTTGTGAAAGGAAATTAAAT
CTTGGGGCCCTCAATCCCTAAACTAAAGGAAAAAGTCAAGTGGGGAACTGCTTAGGGCTAACCTGCCTTC
CATTCTATTCAAAATCATCCCTCTGCTCACTGAGATAGATACATATCTGATTGCCTCCCTTGGAAAGGCT
AATCAGAAATGCAGCCATTGGTCTCTCACCTAACTTTAACCTGGAAGCTCCCTCCCTGATTTGAGTCTTC
TTGCCTTTGCTTTAAGTTGTCCTGCCTTTCCAGACCAAACCAATGTACTTCTTACATATATTGATCGGTG
TCTCAAGTCTCCCCGAAATGTATAAAACTAAACTTTGCCCCGGCCACCTTGAGCACATGTTGTCAGGACT
TCCTGAGGCTGTGTCATGGGAGCGTCCTCAACCTAGGCAAAATAAAATAAACTTTTTTTTTTTTTTTTTT
TTTTTTTTTTTTGAGGTGGAGTCTCACTGTCATCCAGGCTGGAGTGCAGTGGCACGATCTCGGCTCACTG
CAACCTCCACCTCCCGGGTTCAAGTGATTCTCTTCCCTCAGCCTCCTGAGTAGCTGGGATTACAGGCACA
TGCCACCACACCTGGCTAATTCTTGTATTTTTAGTACAGATGGGGTTTCACCATGTTGGCCAGGCTGGTC
TTGAATTCCTGACCTCAACTGATCCGCTTGCCTTGGCCTGCCACAGTGCTGGGATTACAGGCGTGAGCCA
CTGTGCCCAGCCTGCAAAATAAACTTTCTAAATTAACTGAGACCTGTCTCAAATTTTCAGGGTTCACAGA
GTAAAGAAGACAGATAATGTCTCTACTCTCAGGAGCTTTCAGTCTAGTGGGAAAAGATCAGTAGTAAACA
AAAATATCAGAGACAAGTGCTATGAAAGAGACTAAAAGGTGATGTAATAAGGAGAGACTGAGGGGTTGCT
TTACATTGCATTGTCAGGGAAGGATCCTTTGAGTAGGTAACCTTTGAGATCTTCCTACAAAAGAGTCAGC
CATGGCAAAGATTAGAGGGATGAGCATTCTACCATAGGGAACCATGTTACACAAAGCCCCTTGAGCTCTT
GGAGTATTTAAGGAGCAGACAGGTCAGTGTGGCTGGAATGTAATGGGCAAGGGGTAGAGTGGTGCAAAAT
GAGATCAGAGATAGTCAGGGTCTAAATCATTTAAGGCTTTGTAAGCCAGAGTAAGGAGTTTCTAGGTGTA
AACGAAAGCCAGTGGAGGGCTGTAGCAGGGGGAACGCCTTTTGAAAAAGATTACTGTCGCTGCCGTGTGA
AGACTGGATTCTGAGGATAAGGGTAACAAGGGTGGAATTTAGGAGACCTCTGGAGGCTGCTGTATTAGTA
TAGGTGAGAGGTGGTGGTGGCTTGGGCCAGAGAGTAAATTAGTGGAGAAAAATAGGTTGATTTGGGCTGT
GTTTTGAAGATTGAATCAACAGGACTTGCTGACTGCATGTGATCAAGGCAAATCCTAGGACTTGAGCACT
TAGAAGATTGGTGGTGCAGTTTACTGAAATGGATAAGACTGGAGGGGAGAGCAAGTTTGGTGGGACAATC
ATGAGTTCTGTTTTGTTAAGTTTGAGGTGCCAATTAGACATCTATGTAGAGATTTCATACTTGGTGGTAG
GTAATAGTAGTACTGTGGAATTCAGTGAGAGTTTAGGGCCAGGGATAAATATTTAGGAGTAACTGATGTA
TGTCTGCTAGATTTGGTAAGTGACCTTGACAAGTGTGGTCTTAGTGGGAATGGAAACTAGATTGTAGAGA
AGTATCAGGTAAAGGATGGAATGAGAATATGAAGTTGGGGAATGGAGACAGTGACAATAGACTCCTAAGA
AATTTTCCTGTGAAAAGGGAAGCAGAAAAATGGTTGTGACCACAAGTAAGGCCCAGATTCGAATCTTACT
TTTATACTTGCCTGGATACAGTTATTATTTGAGTGCATTCTAAAGTTTAAGATATTTTATGTACCCTACG
CAGTCCTCTCTACAAATATTGAGGTAGATGATACTTTTCCTTCTGTGACATGAGAGTCCTTAGTTTAGGG
AAGTTAAGTGACAGTCCAGGTAGAACCTGTATTTGGACTCACTCACTTTGACTGTAGATGCAGAGTATTT
TTCATTATACCAAAATGCTCCTTAAACTTTTATTATCTTGGTCAAATCCAATCTACCTTAGAGTTTTTGG
AAGGATTACAAGATGAGAGATGTGAAAATTCTTTGCAAGCTCTAAATTGTTACACAAATATAGAAATTCC
TTTCTTTAAAATGCCCCATTATGTCTTCTTTGTGCATGACTTTTTAGGATATAGTAGGTAGTGGCCTTGA
AGTTCATTTTGGATCATAGCCTATCTCATTAGAGCCCAGAGAATGTAAGCAGATTGTTAAAGACATAGAA
CCTGTCAAAGCTATCTATGGCATATGAATCGAACATGGATCATGTTTAATTGAACATTTTTACCCTGGTT
TAATGATCTTGTCTCCTTCGCATGTTCCATGAGGTCTCATAATCCTCCCCAAAGGAGCTCTTCCCAAAAA
TAATTCCTTCTAAAAATGTGGAAGGGCTGGGCACAGTGGCTCATGCCTGTAATCCCAGCACTTTGGGATG
CTGAGGCAGGTGGATCACCTGAGGTCAGGAATTGGAGCCTGGCCAACATAGTGAAACCCCGTCTCTACTG
AAAAAAAAAAAAAAAAAAAAAAAATTAGCCAGGCGTGGTGGTGGGTGCCTGTGCCTGTACTCCCAGTTAC
TCAGGAGACTGTCGGAAGAATTGCTTGAAGCGAGAGGTGGAGGTTGCAGTGAGCCAAGATCGTGTCACTG
CATTCCAGTCTGGGTGACAGAGCAAGACTCCATCTCAAAAACAAAACAAAACAAAACAAAACAACAACAA
CAAAAGTGGAAGTAATTTTAAAAAAACTGCAAAAAAGCCAATCCTGAAAGGTTACAAACTATATCATTCC
AAAAGCCGTGGAAAACAGTAACGTGAGTCCTCAAAGAATTAAAAATAGAATTACCCTGTGATCCAGCAAT
TCTACTTCTGGGTATATACTCAAAAGAACTGAAAGCAGCAGGGTCTTGAAGGGGTATTTCTACACCCATG
CTCATAGCAGCATTATTGACAAATGCTGGAAGTAGTCCAGCTGTCCCCAAATGGAGGAACAGATAAGCAA
AATGTTACTGGTATACATACAAAATGGAATCTTTTTTTTTTTTTGGGAGACAGAGTCTTGCACTGTTGCC
CGGGCTGAAGTGCAATGGTGTGATCTTGGCTCACCGCAACCTCTGCCTCCCGGGTTCAAGCGATTCTCCT
GCCTCAGCCTCCCTAGTGGCTGGGATTACAGGTGCCCGCCACGACGCCCAGCTAATTTTTTGTATTTTTA
GTAGAGACGGGGTTTCACTGTGTTGGCCAGGCTGGTCTTGAACTCCTGACCTCGTGATCCACCTGCCTTG
GCCTCCCAAAGTGCTGGGATTACAGATGTGAGCCACCGCACCCGGCCACAAAGTGGAATCTTATTTCACC
TTTCAAAGGAGAGAAATTCTGACATGCTACAACCTGGATGAATCTTGAGAACAATACACTAAGTGAAACT
GGCCAGTGAAACCAGTCACAAAAAGACAAATACTGTATGATTCCACTTTAATGAGGTACTTAGAGTAGTA
CAAATCACAGAGACAGAAAGAATGGTAATTGCCAGGGGTTGAGGAGAAGAGGGAATGGAGAGTTACTGTT
TAACGGGTATAGTTTCAGTTTTGCAAGTTGTAGAGTTCTAGAGCTAGATGGTTGTAAGGGTCACACAACA
ATATAAATGTACTCAGTAACATTAACTGTGCACTTAAAGATAATTTAGATGGTAAATTTTATGTTATGTG
TATTTTACTATGCCATCATGCCTGGCTAATTTTTTTGTTTTGTTTTGATTTTCTGGTGACAGGGTCTCAC
TCTGTCGCCCAGGCTGGAATGCGGTGGCACGATCACGGCTCACTGTAGCCTCGACTTCCTGGACTCAAGT
GATCCTCCCATCACAGCCTCCCAAGTAGCTGGGACTACAGGTGTACGCCACCACTCCTGGCTAATTTTTA
CTAACTCACTCCCTTTTCTGCCTCCTTTCAGTATTCATTCATTCATTTAACAGATAATGACTGAACAACT
GTTAGGTATTATTAGGTATTATATATTATTCCTGGGAGCAGACTATTAAGAGTCACTCTATACCATCACA
GTCCCAAGTGTCATTATGAAAGAGGGGCTATACACAATACAGCAGTATAAAACTGTAAACAGATTTTTTA
AGCCTACATTTTATCAGTCAACATCAGAAACATCCAAATACCGCAAACTTTCTTTTTTTTTTTTTTTGAG
ACAGTTTCACTCTTGTCACCCAGGCTGGAGTGCAATGGCGCGATCTTGGCTCACTGCAGCCTCCGCCTCC
TGGGTTCAAGTGATTCTCCTGCCTCAGTCTCCCGAGTAGTTGGGATTATAGGCATGCGCCACCATGCCTG
GTTAATTTTTGTATAGTAGAGACGGGGTTTCACTATATGTTGGCCAGGCTGGTCTTGAACTACTGCCCCC
AGGTGATCCACCCGTCTTGGCCTCCCAAAGTGCTGGGATTACAGGTGTGAACCACCACACCTGGCCAAAT
GTCCCAAACTTAAACCAAATGGACTATAAATCAATTCCCTGAAAAACTGGTATACATGTAAAATGATGCT
ATTACATTACAGTAAATGATCTTTAAAAATTTATTTATAGTCTATATGTTGACTACAGTTTTACCTTTTG
CTAAAGGAAACTATAAAGTCGCAGTTTCAAGTATGGTAGGGTAATAAAACTAAGTACATCATTTAAAAAT
ACTTATTAAATGTTTATCTTTGCCAGGCAATGTTCTAGAGACCAGCAGTCCAGCAATGAACTAAACAAAA
TGAACAAAATACATTATATTTTACTAAAAATTTGTTTTCCATATAATACAAATACACAAATTAATAATAA
AGCCTACGTTAATGCATTGTACTAATCTGTCTTGGTGAGAAGTTCAATTTATGAAGATACAGTAGAAATG
ATTTTAATTTCTTTTTTTTCCCCTAGAGAATGTGATGTTTATTCATTAATTTATTCATTCACTCAAGAAA
TCTTCATTGAATGTCTACTGTGTATCAGTTGCAGTTTGGGATTCAGCAGTGAACAAAACATAAATGCTTG
CCCTTATGGAACCTACAATCTGTTGGTTTTAAAAAAAATCTCTCCTGGATTAGTGTTAGATTTCTAGACC
AGAAGTGTCAATTAAGTTTAACAATTTCTTCAGGCAGAGAAAATTGCTTTTGAGGTATCACTTAAGCAAC
CTTAGTGCTTCTAAGCAACATAACTGCCTTTTGAGGTATGAACTGAAGGGAAGTGAAGTATAAGCAATAG
AGTTGGGATTTTTTTTTTCTTGAGACGGAGTTTCATTCTTGTCACCCAGGCTGGAGTGCAATGGCACCAT
CTTGGCTCACTGCAACCTCTGCTTCCTGGGTTCAGGGGATTCTCCTGCCTCACCCTCCTGAGTAGCTGGG
ATTACAGGTGCATGCCACCACAGCCAGCTAATTTTTGTATTTTTAGTAGAGACGGGGTTTCACCATCTTG
GCCAGGCTAGTCTCGAACTCCTGCCCTCAGGTTATCTGCCTGTGTTGGCCTCCCAAAGTGCTGGGATTAC
AGGTGTGAACCACCACCCCTGGCTGAGTTGGGATTTTTTAATTTTCCATAGATTTCCCATTAGGAAGGCA
ACCTGGTGTGACGGTATTTGAGAGTCAAGAGATCTGGGTTCTAATCCTCATTTCATCATGTAATACTTTG
TGTGTTTCAGAATTCTTCACAGTGATCCTTGCTTGTCCTCACTCCTTTGTACAGCTTTTTTTTTTAAAGG
ATTTTATCCACTGTTGTGCCAGTAACTATGCTAGTGTCACTTTTTATGCTAGTAATAGTTCTATTTTCAA
GTTCATTCTGTTTCCTGAGTTTAAATTTGTGTTTCCTAGTGCCAACTGAACATTTCTGTCATTTCATACT
CCATTTCCAAAAAGAAACTCATTATTGTTCCGCTTTGAATTTGCTTCCCTTCTTTATGATCTTTTCATCT
CGGCAAATAGCATTGCTATGTACCCAGCTGCCTAAGGAAAAACCCTGAGTCGTCATCTTTCTATATTTCA
TTGACCCTCATTCAGTCATTCAAATATTGCTTCTCTTGAGTATATCTTGAATCTATCCTTTATGCTCTAA
TTTCTGAACAGAACTGACACTCTTTGTCCTGCATGTTCTACTTTCTTTGCACAGAATACCTTCTCTTTAG
TTGGCAAACTCCAACTCATTGGTTAATGTCCAGCTCAAATGTTAACTCCTGTAAAACCTTCACCATTTCT
TTTAGGCAGAATTAGTGATTCCTGAGTCTTTGCTCTTAGAGCTTGTAGGTCAAGTGTGTACTGTATTTCT
GTTCTTTCACAGTTTTTTATTCGATTGTCTGTAGAATAGACTTTATTATTGTCAAGGGGAAGACAGCCTT
AGCAAAATTTTGAAATAAGTCATTGTGTGCCTAAGCATAAATTAGTTTGTGAGAACATACTTTTTAAAGA
GAAATGTAGTAGGGTGTATAGTATGTTTGAGTATCTTTTTCCAATAAGCATTGCATGTATAATCATTCAC
TCATTGCATGTATAATTATTATTTAAATATTCTTTATTAATTAAAGACCTCTCTGGTCTCTCCAGAAGTC
TTCCATGGCTGCCCTCATCCCCACCCTCCAGAGGAATCTGAAGCCACATGTGCTCCCTGGCACCCACAGC
CCCTGCCTCTCCCAGAGCAGCAGTACCTGAGCCTCAGTGGATTCCAAGAATCGAAACCCTTGGTCTGCTG
CCCCTCCCTCCCAGAATGTTTCTGTCTCATTCTTACCTACTCAAGGCCCTTTCAGTAGCCCCTTGGGGTA
TTCTCTTCCTATGCACCAGGGCAACTTCTGAACTCACCACTTTCCTGGAAAGACCCCCAACATCTTCGGG
GAGCGGTCCCCTACAGGCCTGGCTGCAGATGCCCGTGCACACAGGAACACTGTTTAAAAAAAAAAAAAAA
GGATACTGGTGGGAGGGGCTTTGTAACTGAGGAGGTTTAGGAATTGAAAACTCATGTGTATTTTCTTTCT
TTCCTCATTGTCACTTCCCAGCTACCCAGAGATATTGAAGGACAGCTGAGCTCCCCTACTTGCTGAAAAT
GCATTGATCAAATTTGTGTAGGATTTCCACATCTATTTATGGGCTCAGAACACTTTCTAGTTACTTGTTC
CTTCCTATCAGCTGTTTTCTGTTTGTGTTAGATTTTTTTTTTTTCTGAGATGAAGTCTCGCTCTTGTCCC
CCAGGCTGGAGTGCAGTGGCGCGATCTCAGCTCACTGCAACTTCCGCCTCCCGGTTTCAAGTGATTCTCC
TGCCTCAGCCTCCCAAGCAGCTGGGATTGCAGGCACCTGCCACCACGCCCGGCTAATTTTTGTATTTTTA
GTAGAGATGGGGTTTCACCATGTTGATCAGGCTGGTCTCGAACTCCTGACCTCAAATCCGCCCACCTCAG
CCTTTCAAAGTGCTGGGATTACAGGCATGAACACCACACCTGGCCTCATGCAGAGCTTTAAAAAAAAATG
TTAACACGGCTGGGCGCAGTGGCTCACGCCTGTGATCCCAGCACTTTGAGAGGCCGAGGTGGGTGGATCA
CTTGAGGTCAGGAGTTCCAGACCAGCCTGGCCAATATGGTGAAACCCCGTCTCTACTAAAGATACAAAAA
ATTAGCCGGGCGTGGTGGTGGGCGCCTGTAATCCCAGCTACTCCGGAGGCTGAAGCAGGAGAATTGCTTG
AACCTGGGAGGCGGAGGTTACAGTGAGCCAAGATGGCGCCATTGCACTCTAGGCTGGGCGACAGGGCAAG
ACTCCGTCTTAAAAAAAACAAAAATAAATAAATAAATAAAAGAAAGCTCTGCATGATACAATACAGTATT
CATAGAATTTTGGCCAGTGAAAATTTTCTGTGTTAGTAGAATAAAAATGAACTCTTCTGATTTACAGATA
TTGATAGTCTGGTCTTTCCATCTTCCATCTTGACTCTCTTTTACTGTTAAAAAATCTTAGAACAAAGTAA
AGCCAGTTCCAAATACTTTTCCTAGTTCTGGACTGTTTTTTTTTTTTTTTTTTTTTTTAACAAGACAGTC
ATTTGTCCGAGTACAACAGAACAGTTCATTGTTAATACTTTGGTTCTGTGTGCTTTAAATCTTTTTTCCC
CCTCTACCCATAATCTCCCTTTGTAAACTTTGAATGTTTTGTCAGTAGATCATCATCATAGGTAAAGATA
CTGCACATCAAGACTGTTGGTCTTCCCCATTCTTCCTGGAATATACTGGAAGCTGTCAGGACTGAGGTTA
ATTTCTCCATTCCTAGAACTATATTGTAATAAATATTGGAATACTGGCTTTTGTGTATTTTTTTGTGTTA
CTTAGTTTTTGGAGAAAGGAAATATTTCTAGACTTTCTTGACTTGTCATAACTTTTTATTGTTAAAGAAT
ATAGTTTAAAAATTATTAAATATTAGGGAGCCTTCTGCTTCATTGGGACTTTAAGGAATGATAGCCATAG
GAAGGGATATATCTTATATTATCATAAAGCTTTTAAAAAATAATATATTTATTACAATTTTTTAAATTTA
TTTTTTATTTTTAGAAACAGGGTCTTACTCTATTGCCCAGGCTGGTCTCAAACTTCTGGGCTCAAGTGAT
TCTCCTGCATTGACCTCCCAAAGTGTTGGGATTACAGGCATGAGCCAATGAGCTCAGCCAGTTGTTTTTT
TTTTTTTTTTTTTTTTTTGAGTGTTTTTTTTTGATAACTTCTAATTATTTTCTTATCCTATCCCTAATAG
TTGGCATCAGTTAATGAACTAGTTTTGATTCCCCAAAAGCCCTCTCTTAATCTCCTGTATTTTGCTTTAC
TTTCTATCTCTTTTGTTTATTTCTTAGAGAAATGTCATTTATGTAATCAGTTCTTATTTTGTAATACTGG
GCCGTAAGAAAGCAGGGAAAAGCACAAAACTTGGCACATAGTAAATTTTGTTGCTTGAACAAATTAATGA
ATGAATGAACTTGCAGCATAAGAGTGCACCCTATTTAGTGGTACTGTTTAATTATTTCATGATCCTTAGC
CAAGAACGAAGGCATACCGGAAGGGAACACACAACAAAAAAAGGTGAGGGAAAAGTGTTAATATATCATT
TAAGTGACTGTATTACAACAATATTACATTATGTTGTCACTCCTGAACATGAGATGTGATTCTCATTTAC
ATTTCAAGCTAATTTGGTTAGCTGGGGGAATTCTTAGGCATCTCAAGAGATAATTAGATATACCCCTTAG
GACCTCACATAATTATAATCACTATTAGGGGTATTAAAGTTTTTTTTTTTTTTTAAACAAAAACTTAGGG
GGAAAAGTTGTGATTGGTAAGCGTTTTTTTCTCTTAGTGTTCTCAGAATCTTAGGAGATACGTTATTTGA
AACAGTTGTTCAGGCCGGGCGCGGTGGCTCACACCTGTAATCTCAGCACTTTGGGAGGCCAAGGCGAGTG
GATCATGAGGTCAGGAGATCGAGACCATCCTGACTAACACGGTGAAACCTCGTCTCTACTAAAAATACAA
AAAATTAGCTGGGCGCGGTGGCGGGCGCCTGTAGTCCCAGCTACACGGGAAGCTGAGGCAGGAGAATGGT
GTGAACCCAGGAGGCGGAGCTTGCAGTGAGCCGAGATAGTGCTACTGCACTCCAGCCTGGGTGACAGAGC
AAGACTCTGTCTCAAAAAAAAAAAACAAAAAAAACCAAACAACACTTGTTCAATCAATTCTACTTTAGTA
GAAACCAGCTCTCTATATTGTTCTCTAACATTTTGTTATCTTGGCTTTTAGATGAAATCTTTAGCATCAG
TTGGGCTCATTAGCACCTCTACTAAGAGAAGCAAGCACAGAATGGAAGGGAGAGCAAATCATAACTTTTT
AAAAAGTTATTTCTCCTTAGTTAAAAGTTTGGAAGAGAAGCAAGTTGAAACTGTAGGCTGATTATGTAAA
AGTTTAAGTTATCAGCTCTCTTATATGTAATGCAGGTTGATTATAAGTTTCTGGGCTGTTGTGACAACAA
GAAAATTATGTTTATTATTAAAAACTTGTTTATACTGCATATATGTGAAGGGCATTATACTGTGACAAAG
CTAACAATAAAACATACATTTTAATATTTCAGATTCCTTATTTTATTTGTTTGTTTAGAGACAGGGTCTT
AATGTTGCCCAGGCTGGAGTGCAGTGATGCGATCATGGCTTACTGAAGCCTCAAACTCCTGGGCTCAAGC
GATCCTCCTCCCTCACTCTCCCAAGTAGCTGGGACTACAGGCATGTGACACTGCGTGGCTGATGTTTTGT
AGAGGCGGCATCTTATTATGTTGTCTAGGCTGGTCTCGAACTTTCTCCCGCTTCAGCCTCCCAAAGTTCT
GGGAGCCACTGTGCTGGGCCAGATTCCTTTTTTTAAATTGTGGGAAAATACACGTAACATAAAATTTACT
ATATTAACCATTTTAAAGGGTGCAGTTCAGTGGCATTAGGTATATTCACAATGTTGTACAATCATCACAC
TACCTAGTTCCAGAACTACTTTATGATCCCAATCAGAAACTATGCGCATTATACAATAACTCCTCATTGT
TCTCTTCTCCCAGTTCCTGGTAAGTTCTATTACACTTTCTGACGCTATCAGTTTGCCTGTTCTAAGTATC
ATATAAATGGAATCATATAGCATTTGTCTTTTTGTGTCTGGTTTATTTCACATAGCGTACATGTTATAGC
ATATATCAAAATTTTATTCCTTTTGGCCGGGTTCGGTGGCTCATCCCTGTAATCCCAGCCCTTTAGGAGG
CCGAGCTGGGCAGATCACCTGAGGTCAGGAGTTTGAGACCAGCCTGGCCAACATGGTGAAACCCTGTCTT
TACTAAAAATACAAAAATTAGCCGGGCGTGGTGGCTGGCGTGTGCTTGTAATCCCAGCTACTTGAGAGGC
TGAGGCAGGAGAATCGCGGGAACCCGCGGGGCAGAGGTTGCAGTGAGCCGAGATCGTGCAGCTGCACTCT
AGCCTGCATGATAGAGTGAGACTTGGTCTGAAAACAAAAACAAAAACAAAAAACACCAAGTTTATTTAAA
AAAAAAAAAAGCTTAGACTTTTTTTGAGCATTTTTATGTTATAGAAAAATTGAGTGAAAACTAGAGTTCC
CATATATCTACCCGCTGCTTGGTACCACCAGTTTCCCGTTACTAACATCTTGCATTAGTGTGGTACATTT
GTTACAGTTTATGAACTGATGTTAATACATTTTTAATTAAATTGCATAGTTTTGCATTAGGGTTCACTCT
GTGTGTTGTACATTCTGTGGGTTTTGACACATGTATGAAAGGTATCCACTATTACAGTTATCACAGAATA
GTTTCACTGCCCTAAATACTCAGTGTTTCATCTACTCATCCCTAAATCCCTGCCAACCACTGATCTTTTT
ACTGTCTCCTTAGTTTTGTCTTTTCCAAAATGTCATATAATTGGAGTCATATAATATGTAATCTTTTCAG
ATTGATTTCTTTCCATTGGCAATATACACTTAAGGTTCTCCATGGCTTTTCATGGCTTCATAGCTCATTT
CTTTTTATTGCTGAATAGTACACTGTTGTATGGATGTGTACCACAGTTTGTTTATTCATTGACCTATTAA
AGACTATCTTGGTTACTTCCCAGTTTTGGCAGTTATGAATAAAGCTACTATAAACACTTGTATGCAAGTT
TTTGTGTGCACATAGGTTTTTAACTCATTTGGGTAAATTGTATGCCTTTCTAACTTTTGTATTTATGCAT
ACACTTTTATATAGTTGTAAAACTTAGTTTTATCTAGTTGTAATAATTTTGTTTTATGTTTTAAGATAAC
ATAAACAATCTTCATGCTGTTTTTGAATGCTCCCTAATATTAAAATGAGAAAATGAATTTATTATGTACT
TTATTTTTCTGTTACTCATTAATTGCTTCCATTTTCCCCTGCCTTAATAAATACTTTTGTGTGTGTAGTT
TTTCCCTTTTGTATTGCTTTAGGATAAAATTCAAGGAGAAACAGTAAAGAGTACAATTATTGGGATCAAG
GCCATTTTAGATAATTCTTGATATGCAGTAAAGTTTCACAATGGGAAACCTTGTTAGATTTACAGTGGAG
GTTTGCTGCAGTTTATTTTGTGGCTTTCTAGACAAGAGTAGCACCAGTTATCTGTGTAATTTTACTTAGA
AAAAAACAAAGAACTTTTCACTGGTTCTTTGAAGTGGTAGTAAGCTTCCCTACTGTTGGTTTTGTTCAAA
AATCACTGTTTGCTATCTGAATCTTCTTGATTACTTCAGAACTTTATGTAATAGTTTATTGAGGTTCTTA
GGTACAGGTAGCTAACACGGGCTTGACTTTATCTTACTATGGTACAATCTAAATATATATTTAGTGTTGT
TAATTAAGGTTATCATTCACAGATGCTCCCTTATATATAGGTGGAGAAACTGAGGCTTCCCTTGAGACAG
CATGCTCCTGGATATAACCGTGAGAGCAGTTAAACTCTTGAGACCTGCATTCTTTATTTTTCTTAATTAT
CAGTACAATTATTGGATCAGAGTTTCTTCTATCTTTTTTGTGAGTCATTAAATTCCATTTTTAAAAGGTA
TCTTTTGAATACATACATTTACAGGTTTGGAAAATATATTTCTAGTTAAACCATGTGTTCTTATTTATTT
ATTTATTTTTTATTCTAAGTTTTAGGGTACATGTGCACAACATGCAGGTTTGTTACATATGTGTACATGT
GCCATGTTGGTGTGCTGCATCTATTAACTAATCATTTAACGTTAGGTATATCTCTTAATGCTATCCCTCC
CCCCTCCCTCAACCCCACAACAGGCCCTGGTGTGTGATGTTCCCCTTCCTGTGTCCATGTGTTCTCATTG
TTCAATTCCCACCTATGAGTGAGAACATGTGGTGTTTGGTTTTTTGTCCTTGGCGATAGTTTGCTGAGAA
TGATGATTTCCAGCTTCATCCATGTCCCTACAAAGGACATGAACACATCCTTTTTTATGGCTGCATAGTA
TTCCATGGTGTATATGTGCCACATTTTCTTAATTCAGTCTGTCATTGTTGGACATTTGGGTTGGTTCCAA
GTCTTTGCTATTATGAATAGAGTGCCGCAATAAACATACGTTTGCGTGTGTCTTTATAGCAGCATGTTCT
CTAATCCTTTGGGTATATACCCAGTAATGGGATGGCTGGGTCAAATGGTATTTCTAGTTCTAGATCCTTG
AGGAATCGCCACACTGACTTCCACAATGGTTGAACTAGTTTACAGTCCCCCCAACAGTGTAAAAGTGTTC
CTATTTCTCCACATTCTCTCCAGTACCTGTTGTTTCCTGACTTTTTAATGATTGCCATTCTAACTGGTGT
GAGATGGTATCTCATTGTGGTTTTGATTTGCATTTCTCTGATGGCCAGTGATGATGAGCATTTTTTCATG
TGTCTTTTGGCTGCATAAATGTCTTCTTTTGAGAAGTGTCTGTTCATATCCTTTGCCCACTTTTTGATGG
GGTCGTTTGTTTTTTTCTTGTAAATTTGTTTGAGTTCATTGTAGATTCTGGATATTAGCCCTTTGTCAGA
TGAGTAGATTGCAAAAGTTTTCTCTCATTCTGTAGGTTGCCTGTTCACTCTGATGGTAGTTTCTTTTGCT
GTGCAGAAGCTCTTTAGTTTAATTAGATCCCATTTGTCAATTTTGGCCATTGCTTTTGGTGTTTTAGACA
TGAAGTCCTTGCCCATGCCTATGTCCTGAATGGTATTACCTAGGTTTTCTTCTAGGGTTTTTAGGTTTTT
AGGTGTAACATTTAAGTCTTTAATCCATCTTGAATTAATTTTTGTATAAGGTGTAAGGAAGGGATCCAGT
TTCAGCTTTCTACATATGGCTAGCCAGTTTTCCCAGCACCATTTATTACATAGGGAATCCTTTCCCCATT
TCTTGTTTTTGTCAGGTTTGTCAAAGATCAGATAGTTGTAGATATGCGGCACTATTTCTGAGGGCTCTGT
TCTGGTCTATATCTCTGTTTTGGTACCAGTACCATGCTGTTTTGGTTACTGTAGCCTTGTAGTACAGTTT
GAAGTCAGGTAGCGTGATGCCTCCAGCTTTGTTCTTTTGGCTTAGGATTGACTTGGCAATGTGGGCTCTT
TTTTGCTTCCATATGAACTTTAAAGTAGTTTTTTCGAATTCTGTGAAGAAAGTCATTGGTAGCTTGATGG
GGATGGCATTGAATCTATAAATTACCTTGGGCAGTACGGCCATTTTCACGATACTGATTCTTCCTACCCA
TGAGCATGGAATGTTCTTCCATTTGTTTGTGTCCTCTTTTACTTCATTGAGCAGCGGTTTGTAGTTCTCC
TTGAAGAGGTCCTTCACGTCCCTTGTAAGTTGGATTCCTAGGTATTTTATTCTCTTTGAAGCAATTGTGA
ATGGGAGTTCACTCATGATTTGGCTCTCTGTTTGTCTGTTATTGGTTTATAAGAATGCTTGTGATTTTTG
CACGTTGATTTTGTATCCTGAGACTTTGCTGAAGTTGCCTATCAGCTTAAGGAGATTTTGGGCTGAGACA
ATGGGGTTTTCTAGATATACAATCATGTCATCTGCAAACAGGGACAGTTTGACTTCTTCTTTTCCTAATT
GAATACCCTTTATTTCCTTCTCCTGCCTAATTGCCCTGGCCAGAACTTCCAACACTATGTTGAATAGGAG
TGGTGAGAGAGGGCATCCCTGTCTTATGCCAGTTTTCAAAGGCAATGCTTCCAGTTTTTGCCCATTCAGT
ATGATATTGGCTGTGGGTTTGTCATAAATAGCTCTTATTATTTTGACATACGTCCCATCAATACCTAATT
TATTGAGAGTTTTAGCATGAAGCATTGTTGAATTTTGTCGAAGGCCTTTTCTGCATCTATTGAGATAATC
ATATGGTTTTTGTTGTTGGTTCTGTTTGTATGCTGGATTACGTTTATTGATTTGCGTATGTTGAACCAGC
CTTGCATCCCAGGGATGAAGCCCACTTGATCATGGTGGATAAGCTTTTTGATGTGCTGCTGGATTCGGTT
TGCCAGTATTTTATTGAGGATCTTTGCATCGATGTTCATCAGGGATATTGGTCTAAAATTCTCTTTTTTG
TTTTGTCTCTGCCAGGCTTTGGTATCAGGATGATGCTGGCCTCATAAAATGAGTTAGGGAGGATTCCCTC
TTTTTCTATTGATTGGAATAGTTTCAGAAGGAATGGTACCAGCTCCTCCTTGTACCTCTGGTAGAATTCG
GCTGTGAATCCATCTGGTCCTGGACTTTTTTTGGTTGGTAAGCTATTAATTATTGCCTCAATTTCAGAGC
CTGTTATTGGTTTATTCAGAGATTCAACTTCTTCCTGGTTTAGTCTTGGGAGGGTGTATGTGTCGAGGAA
TTTATCCATTTCTCCTAGATTTTCTAATTTATTTGCGTAGAGGTGTTTATAGTATTCTCTGATGGTACTT
TGTATTTCTGTGGGATTGGTGGTATCACCACCCCTGTATCACAATCCCCTGTATCATTTTTTATTGTGTC
TATTTGATTCTTCTCTCTTTTCTTCTTTATTAGTCTTGCTAGTGGTCTATCAATTTTGTTGATCTTTTCA
GAAAACCAGCTCCTGGATTCATTAATTTTTTGAAGGGTTTTTTTGTGTCTCTATCCCCTTCAGTTCTGCT
CTGATCTTAGTTATTTCTTGTTTTCTGCTAGCTTTTGAATGTGTTTGCTCTTGCTTCTCTAGTTCTTTTA
ATTGTGATGTTAGGGTGTCGATTTTAGATCTTTCCTGCTTTCTCTTGTGGGCATTTAGTGCTATAAATTT
CCCTCTACACACTGCCTTGAATGTGTCCCAGAGAATCTGGTATGTTGTGTCTTTGTTCTCATTGGTTTCA
AAAAACATCTTTATTTCTGCCTTCATTTCGTTATGTACCCAGTAGTCATTCAGGAGGAGGTTGTTCAGTT
TCCATATAGTTTGTTTTTGATTCAGAAAATTTGGTCAGAATTAGTGTCTACACAATGGGCCTGTTTTCAT
GTCCACCAGGCATTATGGCTGAGTGAAAAGCCAGAAGGGCCCGATTTTGAACTTTTCTTCCACTTACCAG
CCATGAGACTGAGCAACCTGCACAAACTGTGAGCCTTGAATTCTTCAGTTGCAGAAATGAGGTGTTGAAT
CCTTCAGGTTGTTATGAAGAATAAATGAGATAATGGGCACCTGACAGCATATATTTATGTTAGGAATATA
TAATTATTGCTGTTTGAAATTGTAGCCTGTATATATTTGCAGAGTGCCCAAATACTTGTTCCTCAGATAC
AGAGTTTATGTACATCTCAATGTACCTTTTATAGTTGAAGTTGTTTTGTTTTGAGAGAAGACTGAAGTTA
TTTGTGGGGTTTTTTGTTGTTGTTTTTTTTCTTACAGTATTGTCATGTGGAAAATACATGTTAATTATCA
GGGTTTAGGTTTTCTGCTGAGTGTGTTTCAAATTAAGTCATTTCCCTCTAGAATTGGTAGTTTGTTGTCT
TCATGTTATTTATAATCATTTCCAAGTTTACCAGATAGAAAAACCCTTAATGAAATTTTGGAAAAGAAGT
ACACCAGTGAGTTGTTTGCTAATCTTCCTGTAGTTGTCACACAGACTGACTGTTTATATATTTTTTGGAG
ACAGAGTCTCACTCTGTCACCCAGGCTGGAGTGCCGTGGTGTGATCTTGGCTCACTGTGACCTCTACCTC
CTGGGTTCGAGCAATTCTTGTGCCCCAGCCTCACGCTTGGGTAATTTTTTGTATTTTTAGTAGAGATGGG
GTTTCACCATGTTGGCCAGGCTGGTCTTGAACTCCTGACCTCAAGTGATCTGCCCATCTTGGCTTCCCAA
AGTGCTAGGATTACAGGCGGAGCCACTGCGCCTGGCCCAGACTGACCCTTTACAGCATCACTCCTGTTCC
TTGCATATTCATGCTTCCAATTTAAGATTTTGTCCATTACTTATTACAGTTGCCATGTATGAAGGTCCCT
TTTTTTGATAATTGGTTCTTTTCTCTTCTACATACTAAGGCAGGGGTGGGAATAAATGTCACAGCATTAC
CACTACTTGATTTGATTTTAATGTGTTACTCTCTTGTACTCCTACCTTATTAGTTTTAGTAGTTTATGGC
TGGGTGTGGTAGCTCTTGCCTGTAGTCCCAGTACTTTGGGAGGCTGAGATGGATGGATTGCTTGAGCCCA
GGAGTTTGAGACCGGCCTGGGCAACATGAAGAAACCCTGTCTCTACAAAAAAAAAAAAAAAAAAAAAGTT
AACCAGGCATGGTGGTATGTGCCTGTGGTCCCAGCTACCCGGGAAACTGAGGTGGGAGGATCACTTGAAT
CTGGGAAATTGAGGCTGCAGTATGAGCTGTGATTGCACTACTGTGCCCAGCTTGGGTGACAGAGTGAGAC
CTTGTCTCAAAAAAAATTTTTAGTAGTTTCTGTGTTTTGTTTGTAAATTTAACTCTGTATTGAGCCAGAA
AAGCACAAGCCCTGTAATTTCTCATTTTAGAGAAAAACATTCATTTTTTTCCTTCCCCAAGGAGTTTCTC
CTTCTTCCTATTTTTTTCTTTAAAGAGAGTTTGGCAGAATTCCAGAGCCTAAAATAATAAAATAAAGGTA
ATTAAATGAATTCTCCAGCCAGCAGAGACCATCTTATCTTGAATTGCTAGTGGTTTATGCTGGTCCCCCT
TTGACCTTCTTTCACTTTTTGGAGGTGGATGAACTTTTCCACTCAAAACTATCTTGTCAGCCTCACACAT
ATTTATTAAAGGAGTGAGTATGCTTAAGAGACAGATTTTTTGTGTTGGTTGAAGAAACCATTTTGGATAT
TTTTCTCTGTTGTGACTGGGACTGAATTTTTAGATTGATTTATATTTCCTTTGAAGAACAAAGGAATATT
TTTCTCGAAAATGAGAATTAACAGGAGCATTAATAACAACAAAAAGTCTGTTAAACGTATTTGGGGAGAT
TATTTGTTTTCAAAGAATAACATTTCTTGTGTGAGTCATTATATTCAGCAAAATTTCTTTTGTGATAATT
TTATGCTGCCATAAAATTTTTTGGGAAAATAACTTTAAAAAAACTTCAAAAAATTTTATTTTAGAGGGGC
AGTAGTACAGTTGGAAGAGGAATTTTAAGTCAAAGAACAGTAAAACATGAATTCTTTCAGAAGACAGTTG
AAAATTTACTGTGCATCAGGAACATTACTTAGCTCTGAACATAAAAAATGATGAAGGTACTGCTCTCTGC
CTTTATGTCTTTGCGTGTAGTCTAATGGGAGACACAAGTAAATAAGAAACAATGGGTCAAGGTCATTATA
GTAGTAGTTACACAGGTAGGTGCATATGCTTCTAGTCTAGCCAGTGGGATGCTGTGTAAATTGTAGCTAT
GTGGAGAAGCCTTGAGATTTCCCTCTCTGAAAGTAAAATTATTGCCTTAGGGCAGGGTTAGCAAACTATA
GCTAGCCCCTGGGTATCTACCTGTTTTTGTATGGCTCATGAGCAAAGATTGGTGTTTACATTTAAAAATA
GTTTAAACAAAATAATGTTTTGTGATAGATGAATGAAAATTATATGAAATGGAAATCTCAGTGTTAATAA
AGTTTTATTGGAACACAGCCACATTAATTCATTTACATATTGTCTGACTACTTTTGTGCTGCACTAGCTG
AGTTGAGTTTTTTGTAGGAGAGACCTTTTGTGGCCCACGAAACCTAAAATATTTATAATCTGGCCTTTTA
TTATAGAAAAAGTTTGTTGAGCCTTCTTTAGAGTGTTATGAGCACTGACTTTTTATGACCTCAATCACAC
ATGCAGTTTCCTTAAATTCCTTATTATATGTTTAAAAGTTAAATTAGCTGGGCGCAGTGGCTCACGCCAG
TAATCCCAGCACTTTGGGAGGCTGAGGTGGGTAGATCACCTGTGGTCAGGAGTTCGAGACCAGCCTGGCC
AACATGGTGAAACCCCGTCTCTACTAAAAATACAAAAAATTAACCGGGCGTGGTGGCACATGCCTGTAAT
CCTAGCTACTCAGAAGGCTGAGGCAGGAGAATTGCTTGAACCCGGGAGGCAGAGGTTGCAGTGAACTGAG
ATTGCATCGTTGTACTCCAGCCTGGGCGACAAGAGCGAGGCTCCATCTCAAAAAAAAAAAGAAGTTAAAT
TTTAAGTGGCTGTATAAAGGATTATGATGATGCCTACCTTGAATGGTAAACTTTTAGATGTAACTTTTCA
GTTATTTCTGCTGAAAAATGCTAAAGTGATGTGCTATTCTATTGAAAGCCAAGTGTATTGATTTGCTAGG
CCTGCCATAAAGTACTGCAGACTGGGTGGCTTAAATGACAGAAATGTATTTTCTCACAGTTCTGGAGGCT
AGAGTTTCAAGATCAACATGTCAGCAGGTTTGGTTTCTCCTATCACCCCTCTCCTTGGCTTGCAATTGTT
GCCTTCTCTCTGTCCTCACAGGGCCTTTCCTCTGGGCATGTATTCCTCCCTGGTGTCTCTTCTTACAAGG
ACATCAGTCATAGTGGATTAGGCCCCCCCACCTTGTAACTTCATTTAACCTTGATTACCTCTCTAAAGCC
TCTATTTCCAAATACAGTCTCATTGTAAAGTACTGGGGGTTAGGACTTAACATATGAATTTTGAGAGGCT
ACAATCAGTGCACACCGCCAGGGAATTTCTGATAGAAACTTCATTTGTTTACACATTAAGCAGATCTTGA
GGGTGTGTACTGTGTGACAGATCCTGTTTTACGTATGTATGTGTGTGTACTATGTGACAGATCCTGTTTT
AGATACATATGGTGAACGAGACAAACAAGATTTGACACTTAAGAGGCTGGTGGTTTAGTTGAGAAAGACA
GATAATTAATAGGAAAATAAATATTTGAGAGAGTGATAAGGACTGTGAAGGAAATTAAACTGTGTCAAGT
GTTAGAATGATTAGTGTGACGGGTCGTCAGGTAGGGGCTTCCTGAAAAGGTGACCTTTTACCTGGGAGCT
TAAATGGTAAGGGATAGCTAGCTAGTTAAGTTCCCAGGCATGTTTCTAGGGCCTGATGTTGATCAAGATT
GGTATGGAAAAGGTTCAAAAGAATAGTGAGTCAATTAGGGAGCTTTATAAGTTGAGGTTGGAGAGGTTGG
TGGGGCAAAATCATTAAGGGCCTTGTGGGAATTATGGTAAAGATTTGGCTTCCTTTTGCACAAGGAATAA
AATCTGGAGGGATTTTAGCAGGGGAGTGACATGATTTTTCTGTTCGAAAGAGCACTTTGGCTGCTAAATA
AAGAGAGGAAACTGCAGGTATGAGAGTAGAGGATAGGGAGGCCTGTTAGGGGATTATCAAAGTATTGTAG
GTCAGAGATATTGGTGGTTTAGCTAAGATCATGGCAGGGAAGAGAAAAAGAAAGGGAAATAATTTGAGAG
ATATTTTGGGAGATAAAATCCATGTATTATTTACTAATGGATTAAGTATGGGGGCTTAGGAAGAGGGAGG
AATATCTTTGGCAATAGTGTTGTGTAATACTGTCCACTGTAGCAAGTGAACCTATAGTGTTAGCAAATCA
GATAGATGATTGCTGCAGCAGCAAGTTACAGAATATAATTTGGAAAGGAAAAAAGCCTTCAATCATTAAA
TAGGAAAACAGAAGGCAGGTAAACAATGCTATGGTTTAATTATGTTCAACTTACAGGCTTATAACTACTA
GACAACCAATGGGGAATTCATAGTTTGCCATAGAGAATGCCTTTGGGACACATAACCTGTTTGGAATGAA
AAAGCCCTGGAGAGTATTGTAAAATAACAAATGTCAGGTTTGATGGAATGACTATGCATGCAATTAAGTG
ATTTTCTTTAGTGCTAGAGTTCTTTTTTCTTAGGTAAATCATTTTAAAAAATGGACCAGACTTTGTGAAA
GTAGATATGTTACATAAAATAATGTTAGCTAAAGATGAATGGAATAGGTTAAGAGTGTCTTCTTGAAAAT
TTGAACTATATCTCTTTCAAAAAGAATTTTATTACCTCTAGGCTGGACTTGACTGAAAAGTTAAAAAAAA
AAAAAGACAAAATCTTATGATTGTTGAACTCCCAATGAGGAGTCAGAGAATTGTGAGAGCCAGGGTCTGT
TTTTTGTCACTTTTCTCTCTCTCTTTTTTTAAGATAGCATCTTGCTATCTGCAGGCTGAAGTGCAGTGGT
GCGATCACAGCTGCTTGCAGCTTTGAACTCTTAAGCTCAAGCGATCCTCCCACCTCAGTGTCCTGAGTAG
TTGGGACTGCAGGTGCACACCACCACGCCTGGCTTATTTTTGCATTTTTTGTAGAGACGGAGTCTTGCCG
TGTTGCCCAGGCTGGTCTTGAACTCCTGGGCTCAAGCCATCTGCCCACCTGGGCCTCCCAAAGTGCTGGG
ATTACAGGCGTGAGCCACTGTGCCCAGCTGCCACTTTTTTCTATGTTTAGAAATCTACCTCTGAGCCTTA
TTCCCCATCCATAAGGCAGGTAACCACTAGATGGCTAATTTTGAAAGAGCGAGAGCATTCAGAGATGCAC
AGTAATTCTAAGTTCTGAGTAGAATACAGTCATCCTTTGGTATCCATGGGGGATTGGTTCCAGGACCTCC
CATGGATACCAAAATCTGTGGATGCTCAAGTTCCTGATAGAGAATGGCATAGCGTTTGCATATAACCTAA
GCACATCTTCCTGTATATTGTAAATCATGTCTAGATTACTTGTAGTACCAAATACAATGTAAATGCCCTG
TAAATAGTTACTATACTGTATTGTTTAGGGAAAAAAGTCTACATGTTCAGAATAGATGTAATTTTAAAAA
TCTGGATAATTTCCATCTGTTGTTGGTTGAATCCACAGATGCAGAACCCCATTGATATAGAGGGCTGACT
GAATACAAAAGCAAGGCAAGTGTGATAGCAGCCTTCTTTTACTTTAGGATTCAGGATTTACTTCTTTACT
TCAGGATCAAATATTTTTACTTTTCGATTTTATTTTTTTGTTTGTGAAGAGTTGGGAGTTGCTGGGTTTA
GTTATGCTACTCTCTTTTTAAGGGAGAATATAGCAGTCCTAGAGGGATTTGCAAACTATAGCTCTTGGGG
AAACATTCAAAATTTTCCTTGTGTAAGAAATTTTATGAGTCTCGAGGGCCGGATGTGGTTGCTCATGCCT
GTAGTCCCAGCATTTTGAGAGGCCAAGGCTGGAGGATCTCTTGAGGTCAGGGGTTTGAGACTGGCCTGGC
CAGCATGGTGAAACCCCATCTCTACCAAAAAAAAAAAAAAAAACAAAGCAGTGAGCCAAGATTGTGCCAC
TGCACTCCAGCCTGGGTAACAGAGCGAGACGCTGTCTAAAAAAAAAAAAAAAAAAAGAAATTCTATGAGT
CTTAAATTACATCAAGTAACTTTGGAGGCAATAGTTTAAATTCTCTAAAATACTGGGCACATTAGCTCAT
GCCTGTAAGCACTTTGGGAGGCTGAGATGGGAGGATCACTTGAGGCCAGTAGTTCAAAACCAGCCTGGGC
AACAGAGTGAGACTCCAGTGCTACAAAAAATTAAAAATTAGCTGGGTGTGGTGGGCACACGCCTGTAGTC
TAAGCTTCTCTGGATGGAGGCTGAGGCGGGAGGATTGCTTGGGCACAGAAATTCAAGGTTACAGTGAGCT
GTGATTGTGCCACTGCACTCCAGAGTGAGACCCTATCTCTAAAAATATTAAATTAAAAAAAACCCATACA
ACTCAAATTTCCAGGTACTGTTATTAATATGAGTTATCTTGAACTGTTGAAAAATTAATCACTTATTGTT
GATTTCTATCTGCATATTAAAGATCTGGCTTAAGAGTCTTGCATTTGATGAATAGGCTTTTCTATGTTAT
ATACAAGAAAAAGGAGATAAAATAATTTAACTTTTTTATTACATTAAAAAACAGGACTCTGGGAATTGTC
ACTTTATGATCAGCCTTATATTTTTTCCAGACTGTGTCATTGTGTATGCCTGTGCCACCCAACGGATGAG
ACCACTGATCATGTTTAGCTCAAATTTATGTTGGATAGAGGTTATAGGATGGTCCTTATCTGAATGCAGA
AAGGAATGTTTATTTTGCTGTTATGTTTTATATGGGATAAAATGCTTGAAAAGTCTTTTTTTTTTCTGTA
TTTTTTTTTTTGAGACGGAGTCTTGCTGTGTCGCCCAGGCTGGAGTGCAGTGGTACGATCTTAGCTCACT
GCAAGCTCTGCCTCCCAGGTTCATGCCATTCTCCTGTCAGCCTCCCGAGTAGCTGGGACTACAGGCAGCC
GCCACCACACCTGGCTGATTTTTTGTATTTTTAGTAGAGACAGGGTTTCACCACGTTAGCCAGAATGGTC
TTGATCTCCTGACATTGTGATCTGCCCGTCTCGGCCTCCCAAAGTGCTGGGATTGCAGGTGTGAGCCACC
ACGTCCAGCTTTTTTTGTTGTTGTTGTTTTCTGAGACAGAGTCTTTGGCTCTGTCACCCTGGCTGGTTAG
TAGTTGGAACTATAGGCGCGTACCGCCATGACCGGCTAATTTTTGTATTTTTTTGTAGAGATGGGGTTTC
ACCATGTTGACCAGGCTGATCTTGAACTCCTGACCTCAAGTGATCCACCTGCCTTGGCCTCCCAAAGTGC
TGGGATTACAGGTGTGAGCCACCACATCTGACCTACTTGAAAATTCTTAAATGGTGCCTTATACTTTCTG
GTAATAAAAGCACTATTTTAATGTTCGGAACTATTTTCATTATTTGCTAACCTTACAATTAAAAAATCCT
TTTATATTTATCTTTGGATAGATATGAGGGGACCTCAAAAAGTTTGTGGAAAAATGGAATTAAAAGATAA
AAATACAAAATATAAACTTTATTCTCAATATAAGCTCTATCGAGTTCAAGAAAATTTTGTAAGCAATAAT
GCTAGCCATTTAGTCCACTCCTAAAGAAGTCCTGTAACCATATCAGTATGTGCAGTCTTTTTTTTGCATT
ATTAATTAAAGAAAAATGGGTGCCCTTTAAATTTTTTTTTAAGGTTCGGAAACAAAAAGAAGTCAGAAGG
AGCCAATCAGCACTGTTTGGTGGATGCCCAGTGATTTCCCATTGAAACCCTAGCAAAACTGCCCATTTGA
GAGGAATGAGCAGGAACCTTGTGGTGGTGAAAGTCAACAAGATTCCTTGAGCATTCCAAAAAGCTTTTGT
CATGACCTTCGCTCTTGATGGGTTTGCTTTTGCTCTAACTGGATCACTTTCATCTCTTGGTACCCATTGC
TTTGTTTTTTTCTTTGTCTTCAGGATCACACTGGTAAAGTCATGTTTCATCTCCTGTTAAAATTTTTCAA
AGAAATTCCACATGCTCTTGTTGCCACTTGTTTAAAATTTGCATTGAAGGCACTGCTTTTGTCTGTGGCC
GATCTGAGCACAATGGTTTTAGTACCCATCAAGCAGAAAGTTTGTTCAACTTAAATTTTTTTGTTAGATT
TGTATACGCTGAATGAATTTATATGTCTATGGTATTGGCTGTTGTTTGTGCTGTTAATCATTGGATCTCT
ACATTTAGGTTACAAACGGGATGAATTTTTTCCTAAAAAATTTATGTTGATAGTCTGCTACTGCAGGCTT
CATCTTCAATATCATCTTGTCCCTTCTTACAATGAATTATCCTATTTATAAACTGCTGATTTCTTAGGGG
CATTGTTTCTATAAAGTTTTCGTAAAGCACCAGTGATTTTACCATTCTTCCACTCAAACTTCACCATAAA
TTTGATGTTTTGTTCTTGCTTCAATTTTAGCAGAATTCATGTTGCTCTGTTAGGGTGCTTTTCAAACCAA
TGTCTTATCCTTCTTAGTGCCTCAAACTACATCTTGTTCAGATGTGTTATAACAAGTTAGTACCAGCTTA
TTTTGGTGCAAAATTTTATTGAAATTCATGCATAGTGTTTTCATAAAACATATGTTACACGAACTTTTTG
AAGATCCCTTGTACCTGTAGTCCAAAATTCAGAAGTTACTAAAGAATGTAAAATAAAAATAAGTCTTCTT
CCTATGCTTGTTTTCACTTACCCAGTTTCCTTCCTCATCTTCCTGATGGATTAAAAAAAAAATGACATGA
TCTAAAAAATATGATCTATTTTAGGTCTAAGGACATGTATAGGTTGAAAGTGAAAGGACTGAAAGAGTTA
TCACATGCAAATAGTAACTAAAAGAGAGCTGGGACAGTTTGCTAATATCAGACAAGATAGATTTTTAGTT
AAAAACTCTTATAAACACTTAAGAGGCTGGGTGTGGTGGCTCATGCCTGTAATCCCAGCACTTTGAGAGG
CTGAGGTGTGTGGATCACTTGAGGTAAAACTTCATCTTTACTAAAAATACAAAGATTAGCCGGGCATGGT
GGTGTGTGCCTGTAATCCCAGCTACTCAGGAAGCTGAGGCAGAAGAATTGCTTGAACCAGGGAGACGGAG
GTTGCAGTGAGCCGAGATCAAGCCATTGCACTCCACCCTGGGCGACAGAACAAGACTGCCTCAAAAACAA
AAAACAAAAAACAAAAAAAACCCTCTTATAAGAGACAAGGAAGAGCATTAAATAGAAGAGTCGATACACC
AAGAAGATATAACAATTATAAACATGTATGTACTGAACATCAGAGCTCCAAATAACCTGAAGCAAATATT
GACCTCATTGAGCAACAGACAGATCTAAAATAATAACTGGAGACTTTAATATGCCACTTTCAATAATGGG
TAGAACAACTAGACAGATCTAGTCTAGTTAGAAGTAGAGTGGAATTATAGGCATGGTGGCTCACGCCTAT
AATCCTAACATTTTACGAGACTGAAGTGGGAGGATTGCTGGAGCTCAGGAGTTTGAGACCAGCTTGGATA
AATAGTGAGACCTTGTCTCTACTAAAAATTAAAAAATTAGCTGGGCATGTTATTGCACCCCTGTTGTCCC
AGCTACTTGGAAGGCTGAGGTGGGAAGAGGAAGATTGTTTGAACCCAGGAGTTTGAGGTTGCAATGAACT
GTGATCATGTCCTTGCACTCTAGTCTGGGCAACAGAGTAAGACCTTGTCTCCCCCACTAACTCCACTGCC
CCCCCCCAAAAAATAAAAAAAACAAAACAAAACAAAGAAATAAAAGACTTTAACACCGCTGTAAACCAGT
TGGGCCTAATAGGCATGTATACTCCACTCAGTAGCTATTTTTCTCAAGTGCACATAGAACATTCTTCAGG
ATAGACCATATACTATGCCATAAAATTAGTCTTAATAAATTTAAAAAGATTGAAATTACACAAAGTGCCT
TTTCTAATCACAATGGAATGAAAGTGGAAATATGAGAATGAGAACTGGAAAATTCACAAATATGTGGAAA
TTAACACACTTAACCATTGGGTCAAAAATCACAAGGTAAATTAGAAAATATCGAGACAAAAACACAGCAT
ACCAAAACTTAGGGGTTGCAATATATATATCACTTCATACAATTGGCTTTTCAAAATCAGTTAAGACAAA
AGGTAAACATTTATATAATTACAGGTACTCTTTCTTTTTTGTTGGGCGGTGGTGGGTGGTACGTGGATTT
GAATTGCTGTTTGAGGTCACTTGCTTCCAGGCTTTTGCATTTCTTTTAAGGGGGATCTTCCAGGAACAAA
ATCTGTTTTATTGCTTTTTAAAATCTGAGAAGGTCTGTTTCGCCTTCAGTTTTGAAATGTAGTTTTGCTA
GATATAAGATTGGTTGACAGGTTTTTTTTTTCTTTTATTTGAACACTTTGATTATGTTATCTCATTGTTT
TCTGTCTGCCATTGTTTCTGCTTAGAAGTCAGCTGCTAATCTTACTGGGTTCCATTTTAAAATAATGAGT
CGTTTGGTTCTTGCTGCTTTTAAGATTTTCTCCTTGACTTTGACTTTCAACATTTTTACTGTGAGGCATC
TGTCTGGATGTCCTTGCCTTTCTCTGAGTTAAAATACATTGAGTTTCCTGGATGTGTGGTTTATTGTCTT
CAGTACATTTGTGGAGTTTTCAGCCATTATTTCTTTGAATTTTTTTTCCCCTCTACCACTTTTTCTCCTC
TCCTTTTAGAGTACCAAACTGTGCCTATTGGTGTCCCACATAGCTCTAAGATTTTCCAATTTACTGTTGA
GCCCCCCCACCCTTTATTTTTAAATTTCCATTATTGTAATTTTCAGCTTCATAATTTTTATTTGGTTCTT
TTTTGTAGTTTACATCTCTTCATTGATCTCTATCTGAGGTGATATTGTCATTATGCTTTCCTTTACTTTT
ATTATTTATTTATTCTTTTTTTAGAGACGGAGTCTCTCTCTGTCGCCCAGGCTGGAGTGCAGTGGCGCGA
TCTCGGCTCACTGCAAACTCCGCCTCCCGGGTTCACGCCATTCTCCTGCCTCAGCCTCCCGAGTAGCTGG
AACTACAGGTGCCCGCCACCACGCCTGGCTAATTTTTTATATTTTTATTAGAGATGGGGTTTCACCGTGT
TAGCCAGGATGGTCTCAATCTCCTGACCTTGTGATCTGCCCACCTCGGCCTCCCAAAGTGCTGGGATTAC
AGGCGTGAGCCACCGTGCCTGGCCTGCGTTTTACATTTAAGTCTATGATCCATTTTGAGTTAATTTTTGT
ATGAAGTGTGACCTAAATTTACAGGTTGAAGTTTTTTTTTTAAGTTTTTTGTCTATGGATGTTCAATTAT
TTCAGCACCATTTATTGAAAAGGTTATCCTTCCATTATTGAATTGCTTTGGATATTTGTAAACGTGTTTA
GGGAAAAACTCTCCTCAAACTGTGTTTTTCCTCTACTGTCATACCACAGCAATAATCAACACAGAAGAAG
ACTTCTGTGATCACACACATGGGGTTGTTTTCCCCACACACCAAGCAGCAGACAGCAGCCTGAGTGTCCT
CTAATTCAGTTCGACACTATCTCCGGGTCATTGGTGTCAGATCCCACAGGTTGAGGGCTCCGTACCTAAG
ACTACTCCCCTAACACACACACCAGTCATAAGTCGGGGCCTCCAGAACTTCTGACCAACCAGCTTCAAGT
TGGGGCTCCCATGGCCCCCTCTTTGGGTTCATTAATTTGCTGGACTGGCGCACAGAACTCAGGGAAACAC
TTATTTACGCTTACTAGTTTATTATAAAGAATATTGCAAAGGTTACAGATGAAGGGACGTGCAGGGCTAC
GTATGGGGGAAGGGGTGTGAAACTTTCATGTCCTCCCTGGATGCTCCACCCACCAGGAACCTCTGTGTGT
TCAGCTGTCTTGAAGTTCACTGAACCCTGTCCTCTTGGGTTTTTATGGAAGCTTCATGACATCAACATTC
CTACCCCAAGGGCATAGATGGGACCCTACATGAGAGAGTCTTAAGACCCGCAATCAAAAAGTCAGGGGAA
CATTAAGTGTGGAAAGCAGGCAGGAGAAAGAGGCCTGCCTCTGAGGCCTAACACTTCCAACATTATAATG
AAAGACTGGAATAAAGGCTATGGGAGTTAAGGCCAGGAACCATGGAAGAAGACCAATAGATATCATAGCA
CCACAATAAAATAACAGGCATACTTGTGTGGGTCATTTCTGGGTTCTCTGTTCTGTTCTGTTACTCTATG
TGTGTATTCTTTTGCTCATACCAACACAGTCTTGACTAGCTATGTAGTTGGCCTTCAGTGAGGTCTTTTT
CGATTGTTTTAGCTATTTTGGAGCTTTGCCTTCCATGTAAGTTTTATAATGAGTTTTTATCTACAGAAAT
CCTTGCTGGGATCTTGATAGGAATTGTATTAACCTATAGAACAATTTGGACAGAATTGACATCTTTACTG
AGTCAGTATATTGCTGAGAACATAGTATAACATATTGCTATTTGTTTAGTTTTTTTTTTTTTTTTTTTTG
AGAAATGGGGTCTCATGCTGTTGTCCAGGCTGGAGTGCAGTGGTGTCATCATAGTTCACTGCAGCCTCAA
ACTCCTGGACTCAAGCAGTTCTCTCACCACATTCTCTTCAGTACCTAGACTACACAGGTGCATGCCACTA
AACTCAGCTAACTTTTAATTTTTTTTTAGTGACATGGTCTTGTTGCCCAGGCTGGTCTCAAACTCCTGGT
CTTGAAGTGATCCTCTTGCCTCAGTCTCTTGTATTTGTTTAGATTTCTGATTTATTTCATCAGCATTTTG
GAATTTTTAGCATATAGGGCCTATACGTATTTTGCTAAGCGTATACTTAAAGTATTTAATTTTCTATGGA
GCAGTTGTAAATGGTATTGTTAAAAATTTCAGTTTCTGCGTGTTTATTGTTAATATATAGAAAAGTAAAT
GATGGCTGGGCACAGTGGCTTATACCTTTAATCCCAGCACTTTGGGAGGCCAACGTGAGAGGATCACTTG
AGTTCAGGAGTTCAAGACCAGCCTGGGCAACATAGCGAGACTCCATCACTACAAGAAATTAAAAAAAAAA
AAAAAAGGCCAGGTATGGTGGTGTGCACCTGTAATCCTAGGTACTTGGCAGGTGGAGGGCAGGAGGATCC
GTTGAGCCCAGGAGTTCAAGGTTACAGTGAGGTATGATTGCACCATTGCACTCCAGCCTGGGCAACAGAG
TGAGACCCTGTGTCTCTAGAAAGAAAAGAGAAGTGATTGATATTTGAGTGTTGATCTTGAATTCTTCCTG
AACTCATTATTTCTAGAATTTTTTTTTGTAGATCCTCAGTAACATTTTAAGATTTGTTTTGAAGTGTTTT
ATCTTTTTTCTATTGTGATGTATTCTTATTTCATAATGACCAAATATTGTGGGGTTTTAGGATGAAGGCT
GTTGAAATCCATATATCAATTTTATGCCCCATCACATTGATGATTTTTCTTATTTATAATTTTTTCTTTA
GTTGATTCTCCTGGATTTTATAGATAAAAGAGTCATATAATCTGCAAATAGTTACAGTTTACCTTTTTTT
CCCCCAATTTTTATACTCATTTTTTTCCCTCATTTAATTATCTAAGACTTCCAAGATAGAGTTAAATAAT
GCTGGTAATACCATTTTCTGGCTTTAGACTCTTAGACTTGTTTGCTTTTTTTTTTTTTCTTCAAGGCATT
GCATGCTAGTAGGAGTTCATTTTATTTCAGTAGGTAGAATGCAAATAGAGTAAAACATATAGTAAATTTC
AGTTCTCTAGTGTGTTCTAGTATTGGGGTGTTCTGTTTTGTTAAATTTATGGGAACCTGAAGGTTGACAT
AAATACTTTTACTTCAACAGTTCTACTGTTGAAAGTCAATATTATAGGCCTTCCTGCTTTCTGTAGAGTG
GCCAATATTTGATTTCTTTGTAGATTTTGCAGCACTTTATTTTCTTCATTCTTATTTTATCATACTTGTC
AATTTAGAAAACAGTATTGATAGGAATGTATATTGATCTCAGGACTTTACTGGAGAGTTGATTTTCTTCT
TTTTATTTTAAGGAGGATCCTTAAAAATTATTGGGGAACTCTTATTTGTTGTCATTTGATTTCTTCTTCT
GAGATGAGGATTACAAAACAATTCTAACTAATGGAGCTTTTCAGTTTTTCTTTTTTATTACGAGATGGGG
TCTTTGCTTTGTTACTCAGGCTGGAGTGTAGTAATGCCATCAGAGCTCAGTGCAACCTCGAACTCCTGGG
CTTAAGTGATCCTCCCACTTCAGCCTCTTGAGTAGCTAGTACTACAGCCATGAGCCACGTGCCACCACAC
CCGGCTAATTAAAAAAATTTTTTTTTTTGTAGAGGTGGGTCTCACTTTGTTGCCCAAGCTGGTTTCGAAC
TGCTGGGCTCAAGTGATCCTCCTGCCACAGCTATCCAAAGTGCTGGGATTACAGGTGTGAGCCACTGTGC
CCGCCTGAGCGTGCGTTTATAGTCTCATAAATGAGCAACATTCCTCCATATATAGCTCCTGCTGATGAAA
AGTTTGGTGTGCTGATATTTTTCTTTTCTTTTCTTTCTTTCTTTTTTTTTTTTGAGATGGAGTTGCCAGG
CTGGAGTGCAGTGGCATGATCTCGGCTCACTGCAGCCTCTGCCTCCCGGGTTCGAGCAATTCTCCTGCCT
CAGCCTCCTAAGTAGCTGGGATTACAGGCATGCGCCACCACACCCAGCTAATTTTTTGTATTTTTAGTAG
AGACTAATTTTTTGTATTTTTAGTAGAGGGTTTCACCATGTTGGCCAGGATGGTCTAGATCTTCTGACCT
TGTGATCTGCCCGTCTCGGCCCCCCAAAGTGCTGGGATTACAGGCGTGAGCCACTGCTCCTGGCCTCTTT
TTTTTTTTTTTTTTTTTTAAGATAATGTCTCACTCTGTTGCCCAGGCTGGAGTGCAGTGGCGTGATCTTG
GCTCACTGCAAACTCCCCCTCCCAGGCTCAAGTAATCCTCTCACCTTGGCCTCCCTAAGTGCTGGGATTA
CAGGCATGAGCCACTGTGCCTAGCCTAGTGTGGTGATATTTCAAGAACACTTTACAAAGCTTTATTCTAC
TTGCTACAGCTTCAGCATCAAGATCCTGACTCTGTAGTTTTCTTGTGATATAAACCAACTACATTTAGGG
TTCATTTTCTTATCTTTTACTCACCCCAGTCTTAATTTTGTTGCACAGTGGAAAGCACATGAACTTCATG
AGGTTCAGAAACTGGGTATAACACAGCTGCTTCTCTTTGAATCCTAATGACCTTGAATAAGTTATCCAAA
CTCTAATCCTCAAAATGGAGGATAATAATACCTCAGAAGGCTATATTTAAAGATGCTGTGAAGTACCACA
TGTAAATTATCTGGTACAGAATTTACCTTTGTTGATTCTCAGTAAATGTTTGCTGCCTCTCCCCTGAAAC
AAAATGAAATGGTGTGTGTGAATAGCTTTGTGAGCTTTAAATAATATAATTAAATAGAAGCCATTATTCT
GTTTCCTCTTTCAGAAATGGGCATTAATAACATAATTTATAATCTCAATAGTATCATGTGCTTGGCCCAG
GTACATGCCCAGGTTTGGTATTCTTTAGATTTTTGTTTCTTCTTGCCCTACATACTTCGAAAGCCCTTGG
AGAAATCCTACAGCAAATTCACATGATTTATGAAACCAACAGTTGACTAATTCTTACTCTGTTTCTTTGA
CTAATTCTTAGTCTATGTATCCACATATATGAAACTAAATCCACCTGTTACCTTCTTTTTATGGTATCTG
GGTTCTCCTACTTTGCCCTACTCCTTTCCCCACTAAGAACCTTGAACCTCATGGAATTTATTAGCTAGCA
TATACCCTGGTCTCTTCCTGTTTTGGTATTTCTGGTTGGATTGACTGTGATAATCCTTATTGGATATTCA
ACTAATTGTCTAAAATAAGCTTACATATCATGAGTTAAGAGGTATAGTGCCAAGGACTTGAAAAGCTTGA
ACTCCTAGGACTTACTTGCTGCAGTCCAGTTTAGAGAGAAATTAATGATTTCTGAAAATGTTCTTAGCAC
TCAAGAGCATTGAGATATTATCAAATATTCATTAAACATTTGTGTGCGTAGCACTTTCTGGGAACCCAGA
TGGATGTAAAACGTGTATGCTTTGGTCCTGAGTTTCTTCCTTCCAGGGTAGTCTAGTTAGAGTTCAGAAT
AACTCAAAAAAGTTGAAATGTAAGACATTAAGCCTTTTGAATACAAAATATTATGGATAATATGTGCTTA
TAAATTTGAAGACTTTGTCCTGTTTATTTTTTATAAAGTATGGTGAATTTTGTTTTCCTTTACCTGTGAA
CTAGAAATAAAAATACCCTGTGGGGTCATTGTGATGTTTAAATGAGATTGGATATGTAAAGCCCAGTTAT
AATATATAAACTTACATATATTCTTTCTAATACATACAATTAGTATTTAGAATTCTAATTTCTAATTGTA
TACTATTTTTTAAATTAAACAAGTAATATGATACATGGTTAAAAAAAATGCAGGCAGAACAAAGTAAGTA
TTCTTCTCACTTCAGACCCGAGGTTCTTCCATATGTTCTTCTGTACCAAGAGTATGGTTTTAAATATTTT
TCATTAATTATTCTAGAACATATGCACTTACATATACTTACATATTTTTATAACATATAAGTGGGAACAT
ATTATTCATGTTGTCTTGTATCCTGTGTTTTTCATTTAAAAAGAGATTTTTGGCTATATTCCCTGTCAAC
AAATATAGCTTTTAAGTACATTATTCTTTTATTATTGATTGCTGAGTATTCTGTCGTTTGGATATGTGAT
AATATAACTGATATTTTCCCCAGCACTCCTTTAATGCATTTTTTCAAAACTTTCATCTTCTTAGGATCAA
CTCACTATCCTGAAGGTCCATTCTCCCAAGAAGAGGGGACAGAAAGACAGATCTATTTGTAAGAAAGGCT
TGGGTATCCCATGAACGAGCCAACAGAAAACCGATTGGGGTGCAGCAGGACTCCAGAGCCAGATATAAGG
CTCAGAAAAGGGCACCAACTGGATGGTACACGAAGAGGTGATAATGACAGCCACCAAGGAGATTTGGAGC
CCATTTTAGAGGCATCTGTTCTATCTTCCCATCATAAAAAAGTAAGTCAAATTGGAAGAATGATAAACAA
AGGAAAAAATGTGGAGGAAGTGGCAGAGGGAGGATATGGGGAAAAAAAAGTGGGGGTCCATTGCTGGAGG
GAGGATGGGGTGGTAAGAAGGAAACTTGCACATTTGTATTTTTTTCTTCTGGATAACTTGAAGTCTCTTA
TCTTTATTATTATTATTACTACTACTACTACTACTACTACTACTACTACTACTACTACTACTACTATTGA
GACGGAGTCTCGCTCTGTCACCCAGGCGGAGTGCAGTAGCATGATCTCGGCTCGCTGCAACCTCCGCCTC
CTGGGTTCAAGCGATTCTCCTGCCTCGACTACAGGTACATGCCACCATGCCAGGCTAATTTTTGTAGCTT
TAGCAGAGATGAGGTTTTGCCATGTTGGCCAGGCTGGTCTCGAACTCCTTACCTCAGGTATCCTCCCACC
TCGGCCTCCCAAAGTGCTGGGATTACAGGCATGAGTCATCGTGCCCAGCTTCTTATTCTTAAATTAGCTT
AGATATTATTTGATTTTGATTTTATGGTTAATGTCTTTCTGCTGACTCTTTTTTTTTTAATTTCGGAGAC
GGAGTCTTGTTCTGTCGCCAGGCTGGAGTGCAGTGGTGCGATCTCAGCTCACTGCAACCTCTACCTTCCA
GGTTCAAGTGATTATCCTGCCTCAGCCTCCCGAGTAGCTGGGATTACAGGCACCTGCCACCACGCCTGGC
TACTTTTTGTATTTTTAGTAGAGATGGAGTTTCACCATGTTGGCCAGGATGGTCTCCATCTCAACCTCCT
TATCCACCTGCCTCAACCTCCCAGAGTGCTGGGATTATAGACGTGAACGACTGCGCCCGGCCTCTGCTGA
CTCTTAAAAGCGTACTTAACCTGCTTTATCTTGTTGAAGGGAGAAGACGTGGTCTATGGGTACCATATAA
TCTAATTTAACCATACTTTAGCTATGACGTTATCTGTTATTTTTGGAGAAATTCGAATTGCGTGTTAGAG
CTACTTTTCCAGAAATTGTTTACCTCCAGTGTATACCTTGGGAGTCAGTAGAGTTCTGAAAGCTGAAGCA
GATTTTATTCTTTCATTTCCTAGAGAAAAGAGCAACCTTTAGGCAACTATACCTATAGAGCAGAAGTATC
AGAAAGTCATTGTTACAGACATTTTGCTATGTGCTTTGGAACTTCCAAATTATATAGCATGTGTTTAATG
TTTTATTACTAGTACAGTGTTTCTCAAACTTTGGGTTGTACCTATTAGTGGGTTTTGAAATTAATTGTAT
TATAAAGTGTTAAAAATTAAAGATCAAAATAGAATAAAAACATTGAAGTGTGTATATAGTAAGTAATACA
CATTTAAATTGTTTTAGTTTTATATAGTTATGTATATAAATATGAATAATAATATGAAATACATTTCTTA
CTACGGGTTGTGGTTAACATTTTGAAAGCTTCTACTGGGAACAGCAGCTGGGGGAAGAGTGTTTCAAGGT
AGAGGAAACAAGCTAGTGCCAAGGTCCAAAGGTGGGAATATGCTTATCTTCCTTGCCTTCCCCTCCTGCC
TCCCACCACCACCTGCGCCTGCTTGTATCATCTTTCTCTTCTTTCATCTTCTCCTTTATTTTATTATTAT
AATTATATTTTTTTTGAGATAGAGTCTCGCTCTGTTGCCCAGGCTGGAGTGCAGTGGCACCATCTCGGCT
CACTGCAACCTCCGCCTCCCAGGTTCAAGTGATTCTCCTGTCTCAGCCTTCCGAGTAGCTGGGACTACAG
GCCTGCGCAACCACATCCGGCTAATTTTTGTATTTTTAGTAGAGACGGGGTTTCACCATGTTGGCCAGGC
TGGTCTTAAACTCCTGACTTCAAGTGATCCGCCACCTTGGCCCCGCAAATTGCTGAGATTACAAGCGTGA
GCCACTGTGCCTGGCCTTCTTTTTTTTTTTTTTTTTTTAAAGGACCAAAAGAAGGCCAGTGTAGTTAGAG
CATGAGGAACTAGAGGGAAGAGTGAAATGAGATAAGGTCAGTGGGCCAGTCAGAGAACAGATTATACAGG
TTTATTCAGGGAATTTAAATTTTATTTTAATAAAATTGCCTGGCACGTGGGCAGCTTTTGGGGTGTTTAA
ACATGGAGACATTTGTGTTTTTAAAAGTATGACTCTGGTTGTGGAATGGATTATTTGAAGACAGAATGGA
AGAGGGACCCAGGTGTTAGAATATAGATGAGAGATGATGTCACTTGAAACAGCATGGTGGGAGCAGCAGA
AATGGAGGGAAATGGTTGGATTCTGGATATATTTTGTGGGTCAAATCAACAAGACTTCCTGATGGATTAG
CTGTAGAGTGAGGAAAATAGAGGAATCAAGGCTGATTCTCTGAGCTGAACTGAAATGGTAAAGCAAGAGG
GAAGAGCGGGTTTAAGGTAGGGAAATCAAGAGTGGTGTTTTGGAAATGTGAGATTCAAGATTCTTGTTAG
ATATCTTAGAAGATATATTGAGGAGTCAGTAACTGTGTCACGATTAGATTTTTATTTACCTCTGTTTCTT
AACTATTTTTAAAACTGAACTGTTATTAAGGAATCTAAGGAATTTTCAAGCTTGAAAGAATTTCAGGCTA
GCCAATTTGAGATTCATAATGGCAATAAAATTCTGTTTATCATGCTACATTATTTTTAATTACGTACAAA
GATCTAACATGTCACCCAGGGACCATTTCACCCACTGCTCTGTTTGGTCGCCAGTCTTTTGTCTTTCTTT
TCAGCAATGGTGAGGCAGATACCCTTTTCCTCAGGGAAGAGAAGTCTGTGGTTTGTTGCCCTTGCCAATA
ACAAAAATGTTGGAAAGTTGAGTGGCAAAGCTGTTTCCATTGGCATCTTTCATGTGAACCACATCAAAAG
ATCCAGAGTGCCTCTCTCTGTTGGTTATCACACCAGTTCTTCCCAGGTTAGCACCTACAGTCACCATGCA
TAGGTTACCATTGTCAAACTTGATGAAATTGGTAATCTTGCCAATCTCCAAATCTGTCTGAATGGTATCA
TTCACCTTGATGAGGGAATCAGGGTAGCGGATGGTGCCGGGCATCATGAGTCACCAGATGAGGGATTCCT
TTTGTGCCCCCAGAGATTTTTCTCACTTTGTATTTGGCCTCCTCAGGTGTAATAGGATGTACAGCAAAGC
GACCCTTGGTGTCATAGATGAGACGGAAATTCTCCCCCGTCTTGTCAATGCTGATGACATCCATGAATCC
AGCAGGGTAGGTTATATGAGTTCGGACCTTGCCATCGATCTTAATGAACCTCTGCATGTAATCTTCTTTA
CTTCATCTCCTGTCAGGGCTTACTTAAGTCTGTTCCTTAGGAATATGATGGGGAGACACTCTCTCAACTT
GTGGGGACTAGTGGATGGACGAGGAGCAAACACACCGGTCAATTTATCCAGCATCCAATGCTTTGGAGCT
GCTGCCCACTTCAGATGCTTCTTGGGACCACGCGCCATGGCTGCGTTAGGCAGGGAAAGCAAAATATATT
TTTAGAGTCCAGAACTTGCCCACCCAATCACTTTTGTGTCAGATTTACATTAATTGATTTTTTTATGTGT
TGTAATACTATATATTTTTGTTTCCTGTCTTCACTGGTTGAAGTGGTATCTTCTGTAAATAATATTTGTG
TTTTAATTTCACTAATAAATAATTAGGTGGATGGGATTTTTCCCATCCTTATGAATGAACTCATTTATTG
CCCTGAAAGCCTCTCTTTGATTTTCCTTTTATCTTGAGCTAATGCAAAAGTTAGCAGTATGAACAAAAGA
ACCATTTTTTCTTTCATTTGTTTTAATAGATTTTTCTATGAGGAGCCAAGGAAAGAAAGGATCCATAGGT
TGTGGAATGTTAGAGCTGAAAAGGCCTTCCAAAATCATCTTGTTTGCCTTCCTCAATTTACCAGTGAGGA
AATTGTGTTTTTATTCCTAATGAAAATTTGCGTGTGTATGTTGTTTCTCTTTGGAGTTGAAGTTGTTTGC
TGAAAAAGAGAATCTATATGCAGTACTGTCCCTTGCTGCCAATTTCACGGTTAATGTATACTACACATGG
GAGCTAGAAAAGAAGGGGGATAGATAAGAGTTATAGAGAACTGCAGTTTTGAGACTGAAATTAAAGGCAA
AATATTGATATCTTCACATGATTTGAGGAGTTCCATATGCTGATTTTTTTTGTTGTTTGGAGAAAGGACT
CATTTCTTAAAATTTTCAGTTTTGAAGTTGAAACATCTGAACTGTAGACTGCAACCTTCTGGATAATTCT
AGTAATATCTTAATTGTGTCACTTTCACAGACATTATCTCATTTGCTTCTCAAAACTATACTCTGAAATA
GAGCAGATATGATTTCTTTTTGAAAGTAGACGAAAATAGGATTTAGAAAGGTTAGTTTAGGAATTTGCCC
AAGGTAACATGACAAGATGAGAGCATGTGCTAAGTCTTCTGACTGCTCTTTCTGCCGAACAAAGCTGCCT
TTATGAGAATTGGCACCAGATTGACTTAATGTTTCTGAATTCCAAACTCAGTCCCTGAGTTGTTTATATT
GACCTAGAGGTTAGATTCAACAGTTGGTCTTGTTTTTTGGCAGAAGTTGTTGCAGACCTCGTTTTCTGTT
AGGCCAGGGTAATTTTGCTTCTCAGATGTGTTCTAACTTTGTAATGGGTTTTCTGTTTATTTTGAAAGCA
AATAGAACGAATATATCATAGTACAGTCATTCACATTTCTAACAATTTTGCATCATTTTCTTTGTTTTTT
TCCCTCTTTTAGAAGATGCAGATCTTAACTAAATTGCTTAGGCTTCATTTAAAACATTATGGGAGCCCTT
AGGCTCTCTTAAGTTGTGGAGATTGGCATTCACATGCCAATCATGAATAAGGATCAAAGTAAAATAGGAA
AAGCAGCAGGGGTGTCTCGTTTTTTATTAAGTGGAATTCTGAATTTATAAAGTAAGATTTAAGGTAATTA
TGTGCATTACAGACTGTGGTATCTTTTTGTACAAGTCTTTATTTACCGCCAGGTTTCTTTAAATCAATAA
ACCTATTAGGAGTGCCCACCTGATCACATATTGCCCTTTATAGAAAACTACAAATGCCAGGATTTTATTC
CAGGCCTGCTGAATCAGAATCTTTGGGTCCTGGGTATATTTTTTTTGTTTCACAAGTAAGATGCTCACTA
CTGTTTAAAAACCTCCTTCCTGGCCAGGCGCAGTGGCTCACACCTGTAATGTCAGCACTTTGGGAGGCCG
AGGCAGACAGATCATGAGGTCAGGAGTTTGAGACCAGCGTGTCCATCATGCTGAAACCCCGTCTCTACGA
AAAATACAAAAATTAGCTGGGTGTGGTGGCAGGCACCTATAATCCCGGCTACTTTGGGAGGCTGAGGTAG
GAGAATCGCTTGAACCCAGGAGGTGGAGGTTGCAGTGAACCGAGATTGCACCGCTGCACTCCAGCCTGGG
TGACAGAGTAAGACTCTGTCTCGGGGAAAACAAAACAAACAAAACAAAACAAAAAAAACAACCTCCTTCC
CGTAGGAGATAGTTAAGGAAAACAAATAAACAAAACCCAAACCAGTGCCTAATTTTTTTTGATATTTATT
TATTTTAAATAAGTATTTTATTTAAAATAGTTAATGTATTAAAAACTTCTTAGCAACATGCTTTGCAAAG
AAATCTTAACACATTCTCTGAAGATTGTTAGGGAAATAAAGTTAGATTTATTTACTGTTTGTTAAAGGAA
AATATCACATTTACTCACAGTTGACTCTTAAAACATTTTTTTTTGAGATAAATTTTACATACCATAAAAT
TAACCCATTTAAAGTGTACAATTTAATGGTTTTATTAGTCACAGGGTTGTACAACCATCACTTCATTCTA
ACTTTAGGACATTTTTATCATCCCCTAAAGAAACCTTAACCCATTTGCCATCACTTCCCATCTCTACCCC
CAACCCTAGGCAACCTCTAGTCTACTTTCTGTCTCTATGGCTTTGTCTAATCTGGACTTTTAAATAAATG
GAAGTGTAAGGCATGTGGTGTTTTGTATCTAGCTTCTTTTACTTAGCATAATGTTTTTAAGTTTCATCAA
TATTGTAGTATGTACCAGTACTTCATTCCTCTTTATGACTGAATAATATTCCATTGTATGGATATACAGG
GGTTCTTTCTTATTTGTGGTTTCACATTCTGTGGTTTCAGTTACCCATGGGTCAACTGAGATCTGAAAAT
ATTAAATGGAAAATTCCAGAAATAATTTCTAAGTTTTAAACTTTGTGCTGATCTGAGTGGTGTGATGAAA
TCTCTCACCTTCCTTGCCCTGTCCTGCCTTACCCAGGAAATGAATCATCCCTTTGTCCAGCATATCCATG
CTGCAGATGGCTGGCTACTTAGTTACTTAGTAGCCTTCTGGTTATAAGGTCACAGATCACAAGAAGCGTG
AGTGCAATACACTAAGATATTTTGAGTGAGAGAGAGAGCATATTCACATAACTTTTTTTTACAGTATATT
GTTATAATTGTTCTATTTTATTATTGGTTGTTAATCTCTTACTGTACCTAATTTATAAATTAAACTATGT
TATAGGTATGTATATATAGGAAAAAACACAGCATATATAGGGTCCAGCAGTATCCATGATTTCAGGCATC
CACTAGGGGTCTTAGAACATATTTCCTGTGGATGGGGGGAAACTACTGTACTATAGTTTATCCAATAATG
AGTTGATGGACACTGGATTGTTTCCACTTTTCGACTGTTGCAAATAATGTTGCTATTATCATTCATGTAG
AAGCTTTTGTATGGATGTGTTTTCATTTCTGTTGGGTATATACTCAGGAGGGATTTTTCTGGGTTATATG
TTAACTCTGTGTTTAACATTTTGAGAAACTACCAGACTGTTTTTCATAGCAGCTGCACCAGTTTATATTC
CCACCAACAATGTGTGGGGGTTCCACTTTCTCCACACCCACACTAACTATTGTTACTATATTTTGAATAT
AACTATCCTAGTGGGTGTGAAACGATATTTCATCATAGTCAGTTAGCTCTTGAGCTTTTATGAAGAGACC
TGCCTAGGTTAACTTTAGCTCAGGATTTTAAGTATTAACCTCTTTGAGGAATTTGGAAATAGAAGCTAAA
TTCAAACTTACTATAAAACTCAAGTTGTGTCAATTTCACTTCAAATGGTCTCCTCATCATCTGTTCTGCC
ATTACTTTAATCTAAGCCACCACCATCTTCCATCTGGAATACACCTGACAGGACTTCTGTTTTGTTTCTT
GTGCTGTCCAATATATTCTTCGCACAGTTCCTAGAGAGAGCTCTTAAAAATATAAAGTTTATTATGCCAT
TCCCATTTAAGAAATTCATTTGTTTCCCATTGCACTTATGGTACAATCCCAAATTCTTTCTATAACCTTC
GGGATCCTGCGTGATCTCACTGCTGCCCTATCTGGCCAACTTCATTTCAGGACATTCTTCTCCATGTTCA
TTGAGCTGCAGCCGCATTGGCCTTATTTCAGTTTCTTAAACAGGCTGGGCTCTTTCCAGCTTCAGAGCCT
TCAAACATGGCTCTTTCCTCTGCCTGGAGTGTTGTTACACCCACACCTCTGGCTAACTGCTGCTCATCCT
TCAGTATTCAGGTTAAGTTCTCTACTTCCACTTCATTCTATTTCAAGTTTCCCATTGTTATCTTTAATTA
CCTTTTATTTTCCATTACAATGCGTATCATGACATATATTTGTGTGACATATATTTATGTTTATAAACAT
ATATATAAAACATATTTTATAAAAATTTATAATTGTTTAATAATTGTTTTTGCTTGTAGACTCTTCAGTT
CCATGGTGTAAGAATCTTGCCTTTTTGTTTTCCTCTGATATCCTCAGAGTCTAACCTGGTCTTTGAGTGG
CATATAACAAATAGTTGTTGAATGAATTTATCAGCAACAAATATAAAGTTCTGTGTCTAAGTGGTTTCAA
GTAGTTTCTTTAAAGAGATGTACAAAAGGAGCACGTGGGGACGTATACTATTTAGATGCTTAGGGAAGCT
TAACTAATCTACTTCTTTAATCTTAGAATAAATGCATTCTTATCAGGAGAATAATGATAGTTCACTTTTA
TTGATCTTTTACAATGGATGAAGTAATTTTTTGAGCTCATTTGAATATGTTATTCGTTCCTAAAGTAGCC
TTTTAGAATAGATATTATTAGAATCTGTTTTACAGATGAGGAAACTGAGGTTTCATGTGAATGTATGTGT
ATTGTACACTGGGACTCTTGTCATGGGGATGATATACTTTTTCTCTTCAAAGGGGCAGGGTTTTCTTTTC
ATTGGTTTATTGTAAAAAGTGCGATATGGCAATTTCAATGGTTATTTGCCTTTAATACATTTTAAAATAC
ATTATTTCCATAGATGATAAGAATTGTGGAAAAAGAGACCCTGTGATAGGTGATAGTGATCCATTGTAGT
TGTGGAATTGGCTCTCAAGTAACATTAAATAAAATACCTGTGTGTACAGTTAGATTACGTTTACCTGTGC
ATAGGAATCCCTTCCTTATTTGAGGTGTTGGAGAGGTTAGTCTCTTTGGTTCCTTATGAAGCAGGATGGA
TTTTTAGAAAGAGTATTAGAAACTAAATACTTTAAAATTGAAATCTCTGCAGTAGGAATCTGTATTTTTC
ACAAGTTCCCCTATGTGATTGTGAGGCACACTTAATGATGGAGAGCCAGTGATCACAGTTTTCTTTTCAT
AATGCATATACGGAAGTTCCAGACCGGTAGGAATTACATCAGGGTAAAGCTGGGCCTAGAGGAGGTGGGT
GTTTCTTCTTTTGTAAATGCAGTATGTAATAGTATGCCTCTTGTCTCACAGGGTTGTAATGATAATAACT
TAGTGCATGTAAAGCAGTGGTAACACTGCCTGGTACAGAGAAAGCACTTAATAAATGTTAGATGTAGCAT
ATCATTGGAGGTGTATATTTGTGTCAAAAAGAAAAGACTTCCTCTGTTATATCAAAAGGACAAAGTTAAT
CTTTCTTTATTTCTTTTTGTTTTTATTAGATTTGTATTTATCTTATAAAAAATAGATTGGTGGTTATCTG
TCTCCACTCACTTGAACATGTTTATTCTCTGCATCCAAAATAGTCCAGGAATGGCTGGACCTGGTGGCTC
ATGCCTGTAATCCTAACAACTTTGAGAGGCTGAGGCGGGCAGATCGCTTGAGCCCGGGAGTTTGAGACCA
GCCTGGACAACATAGCGAAACCTTATCTCTGCAAAGAATACAAAAATTATCCGGGCCTGGTGGCATGTAC
CTGTAGTCCCAGCTACTTGGGAGGCTGAGATGGGAGAATCACTTGAGCCCAGGAATTCGAGGCTGCAGTG
AGCCATGATTGTGCCACTGCACTCCAGCCTTGGCAACAGAGTAAGACCCTGGCTCAAAACAAAAAATTAC
ACATATAATGTATATTCCAAGAATATTGTATTTGTGCAATATTTAAGTAAAGAAATGAATGATTAAACCT
GAGTTTTAGTCCTGGCTGTACTGCCAGCCAGATGTGTGACTTTGGCCAATTTTCTTGATTTCTTTGGGTT
TCATTTTCCTTACATTAAAATGGAGATGTTTAACTGGGTGATAAACATCTAAGATTTTCTTATCCTCAAA
ATTTCTGTGGTCCCCGACACTATATTAATACTTTTTTGTAATGGGATAAAAAATGAGTGAGGGTCATAGT
TTACTTTTTTCATAAATCAATCCAGTTAGGTATCTTTGCTTAAGCAGCCCTGTTTAATGCCTTCTTTGAT
CACTTTTATCGTGCCTGGTTTTATTTAGTGAAGCCATTTCTGTGGACTAGTAAATAATTTTGAAGGAAAT
AGTTTGGTAAAAGGAAAAAAGCAGGGTTTTGTACTCAGAGTGACCTAAATTTGTATCCCTGATCTCTTCT
GTATCATTATTTTTCAAACTTCTGTGTAAGAGGACCCTTCAAAGAAAAACAACAAAAACCTTATTAAAGT
ATTAAACACACACACACACACACACACACACACACACGTAGATATATTGTAAGTGGATAGTCTGATAAAC
TTTTATAAACTAGACACTCCTGTATAACCAGCACTCCTGGAGGTCTCTTTATTACCTCCTCATAGTAACT
GCCTATTCCCACTCCCCCAAAGGTAACCATTACCCTGACTTCTAATGGCATGGATTAATTTTGCTTGTTT
TTATACTTTATCTCATTGTAATCATTCCATATGTACTCTTGTATCTGGCTTCTTTTGCTCAGTATGATGT
TTGTGAAATTCATCTGTATTTTCTGGGTATAGCTGTAGATTATTCATTTTTATTGGTCTGTGTTATTTCT
TTATATTACTGTACCAATTTTTATTTTATTTATATGAAGGCAATCTGGGTTGTATCTAACTTGGGGCTAT
TATAAATAGTGTTGCTATGAACATTCCAGTACTTGTTTTTTAGTGAACAAATGTATGCACTTCCATTAGC
TACATACCTGTGAGTGGAATTACTGCGTTAGGAAGATGATCCCATTCTCCCTTTAAACATCAAAAATATT
TAACAGACCTCACTACATGATACTCACATTTTAAATATTAGTTTGAAAAAAATACATGATAGAAATTGGC
TATGAATTCAGTTCATCTTAAGTGAATTTGTATTTTGTGATTCACGCAGCACCTACATGATTTTAAATTA
GTTACACCTACTTTATGAATATGAGACATTTATTCAGTCTACAGATTACATTTAATGTGCATATACACTT
GCATAATTTTTGAAGTAATTAGCCAAGATAGTGGTGTTAGGTTGGGAATTAAGGTACTACTTAAAGCTGT
GTACTTTCTCTTAGCTTCTGTTTCTTCATTTACCATGTTTGATCTAGATTTCCACTTAATAAAATGGGAA
TAGAAATATACTCATTTCAGAAAATTGTTATGAGGGTTTGATTAATTTGTAAAAAGCACTTAGTACAATG
TCTGGCATGTAGTGTATAATAAATAATAATTGTTACTCTGTCCTTCCTTTATAATAGTACTTGACTACTC
CATAACTGTAGTAAGTCCTGATAAACACATCTTGAAGTTATCCACATATCTCAGCCACCCTTGATTTCAA
CAGCCTTTTCATAAAGGTGATTTCTAAATCTATCATCTTAGTGCAGTCTTCTTTTTTTTCCCCCTTTTTT
CTTCTATAGCCTTCAAATGAGAAGTGCTTTCTTCTTTATTGGACTTGTAACCATTTCAAATTGAACTAAT
GTTAAACTAAGGTTAACTGTCCACTCCCCCATCAGCTTTTCCTTCTATTCTAATTTTCTAATCCTAATTT
CTCTTAAATAGTACCAACTTTCTTTCAGGTACTCAGACTTGGTTCCTAGACTGTCAAGAGTGGAAGGGAT
CCCATCAGTCATTTAGTTTAGTAGACACATGGATGCTTTTAATCCTTCTACAAATGTTTTTAGTTTCTTT
ACCATGTGTTGGCACTTTGGACTTGAGAAGACATAGTTTTTGCCCTTAAGCAGCATGTAGTTTAGTTGGA
GAGACAGGTAAATCAGTACATTTGTGGTGAGTGCTGTGTGGTTTTATGCACAGATTACTATGGTAGTAAC
TCCAGTTGCAAATGGATTTAGCTAAGAAAGGACTAAAGGACATTTTAGACAAAGGGAACAGCAGATGTGA
AGATTCAGAAGCAGATGTAATACGATGCCTGTAGGGAATAGTAATAATAATAGCTAATGTTTATTGAGTA
CACACTGGCACATACTGATGTCTGAACCTGAAAAATTTCTAGCATAGCTGGAATGAAAGGTCAAGGTGTA
TAGTGGGGTGGAGGATGTGGTAGTAAGTAAGACAAGACACCTGGGGCCTGAAATCTAGGTCTTTAGCCTC
CTGGTCCAGTATTATGCCATCATTCAAATTTCTTTTTGCAATTTATATCAGTTTCAAATTTTGTGTAATG
TTGTGTACTGGTTGAAATGATTAGACCATTGCCCCTAGGCCTCTGACCCTAAAGCCTGGACTCTTTTAAC
TATATTTGCTAAAGTATATAGTTAGGAGGCATTCTGTAAGCTTTTGGACAAAAATATAAGAGAGGCATTT
CAAACCAGGTTGTCCTGATCTTTATTTTTATGGCTCCCCTCTGCCTCCCTGCTCCCTTTCTGAGCTTTCA
AGATATTAGGTTGAATAAAGAGATCAGCCTTTATGGTTGTCCTACTTTTCTTCTGCTCCAGATTCTTCTT
TATAAATTGGGAGATAAGGAACTTTTATTCATTCAACCTTCATATTCTGTTAAACTGGTTTACTTTAACC
AAAATCAAGAATGCCACTATATAATAGTTTATTGTAAGAAAACTAACTTGTAAATAGACTCTTGTCAATC
ACTTCATTTTATTTTCATGGAGAAGCCTGCTTAAGAAATTCAATTAAGATTAGAGGCCGGGTGTGGTGGC
TCACGCCCGTAATCCCAGCACTTCGGGAAGCTGAGGCGGGTGGATCACCTGAGGTCAGGAGTTCGAGACC
AGCCTGGCCAACATGGTGAAACCCCGTCTCTACTAAAAATATAAATAATTAGCCGGGCGTGGTGGCAGGC
GCCTATAATCCCAGCTACTCGGGAGGCTGAGGCAGGAGAATCGCTTGAACCCAGGAGGCAGAGGTTGCAG
TGAGCCAAGATTGCGCCATCACACTCCAGCCTGGAGGACAAGAGCAAGACTTCGTCAAAAAAAAAAAAAA
AAAAAAAAAAAGATAGAATTGCTATTTGAGAATTTTTAAAAAACTGAGTGATTTTAAAGATCTTTTCCAT
TAAGCTTTGATGGAAGTCAGAGTATATATATTTCTAGGCAGGCCAAAATGAATATTCATATAGACTAGAT
ATTATAGTTATTCTTTTGGAGAATACTGGGTATACATCCATCAATACTTATTCTTCAGCTATCATTTTTT
TTTGTAGATACCTTTGTTTCCACAAACCTCTTTGTTTTCATTTGTTAGTAATATCAGCTTGCTAATGCCC
TTGCTACACATTTCCAATTTCTAAAGTTAAGGCTGGCTTCAACCTGTCAACTTGTAAAAAGACCATAAAA
ATCACCTGTTGATCAAACTAAGCCAGGTTTATTACATACTCCAGTAAGGGTGAACACTGCCCTAAAGAGT
CCTAGAAGTATTTCAGAAAGAAAGTATTGGATATGGATATTTACAGGGTTTTGGGGTCTGGTTTCAAGTA
GTTTAAGGAGATTTTACTTGGCCCTGTTCTGAGTTTGTGATAGAATTCTTAGACCAAGTCTTGATAAACA
AATATTTGTTTGGTAAGTGAGCATGTTTGCCCAGTTGAGCAATGATTAAGGATCTAATTGAGCAGTCTGT
TGTTCAGTTAAATGAATTTTTGAGAAATTCCTAAAGCTAATGATGAAGTTATTTACTGGTTTGCATTCTT
ACCTTCCTGGGCAAGAATTTCCGAGAACAAATAATAAAGTTCTGTTGATATAGGGGCCTTAGATCTTAGT
CCTTATAATTAAGCTGTGTGAATGCAGAAGGTCTCAGTTCTCAATGACATTTATAAAGGCAGACAGATTT
TAATCAAAGGTTTACTGTCTCCAGCCCTGGATATTGTACCATTTCTTGACCTTTTTCATCTAATTGTGGA
TGGTAGATAGTATCATGATTGTGAGTCTTTGTGGCACTTCTTGTACTTCCACTAACATTTAAATACCTCA
TTTATGTTTTGCTACTTTCCTAAATGCCCCCGAAGCTCTAGATGAACTAGTCTCTTTTTCTCAACATGGC
ACTTTTGCTTTTCCCTCTTGGTTATTTTCCTTTATCTCATCTTTGCAAAATTTTTATTCTTCATGCAGGC
TTTCCTGAGCCTCATCATGTTTATCTTTGAACTAGCTATACCTTTTTTTCTATGAAGTCATTTATTTGGC
TAATTTAGCACTTTATTAGATTGTAAGTCCATGGAAGACAGAGACTATGTTTTATTCATCTTATTTTTAA
TCATCTAATAAATATTTGTTAAACTGAATTACTCTGTTGAATTGAGTTAGGCCAAAACCAATAAATACCC
AATAGGTTTGTGAATTATTTTCATATTAGACTGATCTTAAAGACCTTTTATCTTTATCACTATGGTTCTC
TGTCTTTCCAAAGGTAAAGTGTACTCTGGAAAATTCTCATTTTAGATTGCAGTTTGGACAGATCATCAAA
GTCAAATGTTACTATGTTTTGAAATACAGTCAGCAAAGTTCTCACAGATGGACTTTACTGAAAACAGATG
GCGGGAGGGGGGAGATATTTCAAAATGTCTTTGTGCTCATTTCAAAGCATTGACCTCTACAAGTTTAAAT
ATAGTTATTCATAGAAAAACCTGGAGAATTAAGTTTTATGCCTTTATTCTTGGCAGCTTTGGTTTTGTTT
AATCTGTCACAATGTCATGTTTCTTGTCATAATTTTGTTGAGGGTGTGGTAAGGAGTATGACAACAGAAG
CAGAACATAGCATGTATGTGATCATTGAATATAGATACAATTTATTGTTCAGAGAGGTATCTTTGGGTAC
TGTGGTTTGGTGGCTGTTGGATGGAAATTAGAAATAGTAATTTCACTCATAATAAAATAGTTATTTGGAC
TTAATATTGGAAGACAGAGACTATTTTAAATATAGCGTCTAGCATAGTCTTTCCAAGCCATCAGTATGTG
ACATATGGTATACCCTAGGGAAAGTATTTTTTTATCCTGTTTTAGCCAGTGTTTAACTCTGCCACTGTAT
TCCTACTTTATCCTCTCTCTAACCCAGGGAACTGCATTTACCACCACCCACACCACGTGGATACCGCATT
AGCAACTCCGTTAATACTTGAACACAATAAATAGTTAATTATGGTCTAGTATTTTAATTTATCCTTTGAG
AACATATTTGTGAATATTCCTTTATACAAGGGTAGGAATTGGTCATAGTACTTGATAAATCAACAGATTT
ATTAATACTTTGCCCTTCTGAAGGAAATATTTTTGTTTTTGTTTGTTTCTCTTGTTGCCCAGGCTGGAGT
GCAATGGCACGATCTCGGCTCACTGCAACCCCCACCTCCTGGGTTCAAGCGATTCTCCTGTCTCAAGCCT
GCTGAGTATCTGGGATTACAGGCGCTCGCCACCTCACCTGGCTGATTCTTGTATTTGTAGTAGAGACAGG
GTTTCACCACGTTGGCCAGGCTGGTCTCGACCTCCTGACCTCAGGTGGTGCACCCACGTTGGCCTCCCAA
AGTGCTGGGATTACAGATGTGCGACACTGTGCCTGGCCGGAAATAATTCTTTTATAAAGATTCTGAATCT
TGCTGATATATTGGGATGGATGGAAATATTTATCTTTACTAAAGTAATTTTTTTTTTTTTTTGAGACAGG
GTGTAGCTCTGTTGCCCAGGCTGGAATGCAATGGCAAGATCACAGCTCATTGCAACCTCGACCTCCTGGG
GCTCAAGGAGTCCTCCCCCTCAGCCTCTTGAGTAGCTGGGACTGCAGACAGGCACGCAACACCACTCCTG
GCTAATTTTTATATTTTTTGTAGAGATGGGGTTTTACCATGTTGCCCAGGCTGGTCTCGAGCTCCTGGGC
TCAAGCCATCCTCCTGTCTTGGCCGCTATCAGTAGTCTTATATGTAACCAGTTGCACTTAGCCTGATAAT
CAGAATTACTGATTGTGAGTAATCTCACTGATTACTTACTGAGTCACATTGATTCGGAGTGCAGTTTCAT
CATGTTTACTTAATGGTAGTATACATTAGCCATATTGTGTTTTATGTACATATCTGTAGTTTTGAAATGG
CTGACAGTAATTTGTATGAGAATAAAGACTCTTTGTTTCTGTTTCTTTGGAGAATCAAATACATGTTGAA
TATGTGCAAATATCTGAGCTATTTTATATAACATAATTTTATATTTTAAAATCGGTCTGCTATAATCATT
TCAAGCTATTGCCCGTTTTCAGGTTTTATATAATTTTAATGGAATTTTGTCATTTTAATTTCGTGTTTAA
TTAGGCACAGCCCTCATTAACTTATTGTTGAAAGTCTCCTAGAGCATTTATTAATTTAGCCATCATGTAA
GTATGTTCCAGAAGCCATGGGGAAGAAGACAGACACTTAAAAATCTAGTTTAGGAAATGATACGCTAAAA
ATGTAAAATTCTATTAAACGTTAAATAAAACAGTATTAGTCAGTAAAAGAGCTGTAATCAGATATTACTG
TCAAATGAGAGGCACAGACTTAAGTGCTAAAGAAGTCAGAAGAAGTAGTCAGAAGAAACAGAAGTCAGAA
GAAGCAATTGCTGTGGATTAGATAGGTTATGCAGTCTCTAATGAAAATTGGTTCTTGGTGGGGTAGGGGA
TGAAAAACTATTAAATACTACTATGGTTTGGAACCCCCCATAGGGCCACAGTACATAGATAGTATATCTG
TGTTATTAAAATTCATGGGGATGAGGAAAGCAATCTAAAATGTCCCAGGGCCGGGTGCAGTGGCTCATAC
CTGTAATCCCAACACACTGGAAGCTGAGGCAGGAGGATTGCTTGAGGCCAGGAGTTCAGGACCAGCCTGA
GCAACACAGACCTCGTCTCTACACACACGCATGTGCACACAACAGACACAGTGGTGCATACCTGTAGTGC
ACGCATGCACACACACGTGCGCACAGTGGTGTACACCTGTAGCCCCAGCTATTCAGGAGGCTTAGGTGGA
GGATTGCTTGAGGCCAGGAGGTTGAGGCTGCAGTGAGCCATGATCGCACCACTGTACTCCAGGAGCCTGG
GTGACAGAGCAAGACCCTGGGGTCTCAAAGAAAAACGCTGTCTCAAACTAAAAAAAAAAAAAGGTCCCGG
GGCTTGTGGGTAGCAGTAATGAAAGGTTGAGGAACACGAGTTTAGGATAGTTAAAGAAGGGTTTTAAATA
GGAACTATGAAGGTTAGTCATTGAAGGATAGATGGTAAGTTTTAGGTAAACAAAGATGGAGAGGTTATTT
TATTCAAGATGGTTTTCTTGGAGATTATGTGTTTTTATAAAATCAGACTGGGTGTATAACATGTCTCACA
TGCCGCATCATAGCCTCTAGTTAGTGTCTTATAAACTGAAGAATAGGAAAATTAAAATCTCAGAATTTCC
TTTTAAACAAGATCAAATTTAAAGTGTTGTAGTAGATTACCCATTGCCTCTGTGTTCATTCTTACCAAGT
AGTTTTTTGATTAGAGGTAGAGGATTTATTCATTTCTTTGTGCAGTCATGTTAGCAGTACTGTTTATCTA
AACAGACTCTTGGTTACATACGGAATTATGGTTCTCTTGTGCTGTCTTATTCATTGAGTTAAAGAATTAT
AGAATTTTTTTAGAGATGTAGAAGACTGTAGAGATTGTTTAAACTCCTCCATTGATACATAGGGAGAAAA
TGGACCTGCAGTGAAGATCCATTGATGTTAGTGGTGGAGCTGGCCTCACCACTTTGGTCTCCTGACTTGC
AGTCTACTGCCCTTCTTCTCACAATATCATTTTATCTAAACCAGTATGAACCTTGGTCTGGAGGCAGAAA
TTTTAATTTTAGTAATTTATCTCTTTAGACCATACCAGATTGTCACTTCTTTTTCCATTTCTCTAGACAA
ATCAGTCATTTTCTCCAGAATGTGGAAAGGCAGTGGGTTTTGGAGTTTGACTTTTCCACTTCAGCTTTAA
CCTTGGACAACTTACTTAACTTCTTTGCCTAATGCTCTTATATGTTAAATGGGAATGAGAATACTTACTT
AATAAGGTGGTTGTTAGGATTAAGTTAGATGACCTATATATAAAGCCTTTTCCTCTGTGTTGGATATATT
TAAAAAATTTATTAAATGTTTTTCTCCCCTTTCTTTCTCTGCCACTAGAGTACTTACCACATTGTGTATC
ACAGTTAGTTATTTTATGTGTCCCAGGTGCGAGTCTTGGAGGTTTTCCCCTTAGTAATTTCTCAACTTTA
TGTAATATTATTATGGGGTCTATGACATAATTTTTAAAAAACAAACCAGTAAAGCCAGGAATTTGCTACT
GAAACAGAGATGCTGCTATAGTAGACAGTTAATTCATCTGTCAGAGAAGAATTGAGTACTAGTGTTCATT
TTTAATACTAATAATTCTATAATAAACTGACTTACTATATTGTTGATTCTTGTCTAAGGCCTTGTTTTGA
CCTTAAGAATTTCATTATAATTCCACAAGGGAAATGTATTGATTGTGTCATTTGGCTCCCAGTCTCTGTC
CTTGACTGTTTCTGTGATTGCCTTGCAGAGCTCTGAGGAACATGAATACAGTGATGAAGCTCCTCAGGAA
GATGAGGGCTTTATGGGCATGTCCCCTCTCTTACAAGCCCATCATGCTATGGAAAAAATGGAAGAATTTG
TTTGTAAGGTAAGAGTGCAGTTTCTTGTCATTGTCATGCTATATATTTAGCCCTTCCAAAACAGAAACCT
TTAAAGAGAGTGGTAGTACTATAGCCTTTGGTTTGCTTTGGGGAAGATCTGTGGCAAGATGGGGACAGAA
GTGGTCTTGGGACTCTAATCCTGTTGAAGACAGAATACATTTGTTGGCTTGTAGTAGCCATGATAAAGCT
CTGATAAAACTAGGATATCCAGCTGGGAAACAAACATTTCCTAATGTAGGTTAACAGAGTAAATGTCTTA
TTGGTGGTTTAGACAAACAGAATGAAACATAGTCAGAAGTGGTTGACGCCTGGAGATATGAAAATGCCCT
CCGCAGGTCCCTTAAATCTGTTCTGCTGAGTGACTGCAATGGCAAATGTACTGTTCTTTCTAGGGATTCT
GTGAGTAATCAGGCTACTCTTGAGAGAGATTTTCCAGTTTCCATGTACATCTGGTATTTTCAGTTCAGCT
GGTATACATTCTGTCTCTTAAGGTTTTTCTTCCAGTCCATTGTCTTGATTCCCTAGGACCTATTATGGGA
TTCTTTTTTTTTTTTTTTTTTTCTAAAATAGTAATTAGAATTGAGTTTGCCTGCTTAGAGAAAATAGACA
CACTGGTAAACTTGTAGGTACTTGATCAATCAGATCAATAAGGGCCCTGGCAGGTAGAATCTAATTAATG
TAAGTCGATCAGTCTGTGTGTATACGTATGTGTATGTAAATTGATTTATTAAACATTTCTGGAGAACTCT
TTATTATTTAGAGAGAAATTTTTGTGACACTTAGCTTTTCATGGCTTTTATGAAACCAAAACTTTTTATG
TACATGGTCTTAGATCCAGGAGACTAAATATAGGTTTTCTTACATTCACAATAATGCTGGAAATAATTGG
GATCAGTGGACTTAGAATGCTATTACTTTGGACCAGGGTATGAAAAGAGAACATTGTGATCTGAACATAG
ACATCCTTTCCAAGACTAAGGGTCCAGTGGGTTTTCAGGGGGAGTTTAAACATTTATGGGTCTGTAATAG
AAAATTTTAGCCAGTTTGAAAATGAGTGAAGAAACAACTGAACAGAACAATAGATTCATTCTAGTGATCA
CTGTCTCAGGAGAATTAGCACAAAGGGATACAAAAGATTATTTTGTTTTTAAAATGCAAATTTGGTGTGA
AGAAATCTCTCAAAGTTGGCAAATTTAGTATCTGGGCCATTAGGGGATTTTAATATTTAGAGATCAGCGG
GGTACGGTAGCTGACTTAACTGCCTTTTTGTCCTCTCCCTTTCTGAACAGAGAACAAAGAATAAAACAAA
TAAAACTCTAAATTTAACAGCAAATCAGAGAATAAATAGGGCAGGACATTTGTTTGCTACAAACTATGAT
CTTCTGAAGGAAAAGGAGTGTTGTAAATATCCCTTAGCAGTTTGTTGATGACAGCAGAATGAAGAGGCAT
CCTGTTACACAGTGTTGTCTGGTGTGGCTCAACCCTGCCATGGCAGTGCAACTGACCACACTTGAAAACC
AGCCGTGTCTAGTGGCACAGGGATTCTATTTCTTGGACTGGAAGCAAGTAAACAGAGACAGTTTAATATG
AAATGTGCTGTGGATAATTTAGTCTTTAGAGTTTCCACGTTTGATCTTGGTCACTCTTTTTTCCCCTAGA
CTTCAGTGTTTGCTATTTACCAGCATAACCAGGTCCATGTGGGAATAGGATGTATGCTATGGGGAGTTTA
TTTGTTTTTGAAATTTATTTATTGTTGAGTCTCGCTCTGTCGCTAGGCTGGAGTGCAGTGGCACGATCTT
GGCTCACTGCAACCTCCAACTCCCGGGTTCAAGTGATTCTCCTGCCTCAGCCTCCCAAGTAGCTGGGATT
ACAGGCATGTGCCACCATGCCCGGCTAATTTTTGTATTTTTAGTAGAGATGGGGTTTCACCATGTTGGCC
AGGATGGTCTCCATCTCCTGACCTCGTGATCTGCCCGCCTCGGCCTCCCATAGTGCTGGGATTACAGGCA
TGTGCCACCATGCCCGGCTAATTTTTGTATTTTTAGTAGAGATGGGGTTTCACCATGTTGGCCAGGATGG
TCTCCATCTCCTGACCTCGTGATCTGCCCGCCTCGGCCTCCCATAGTGCTGGGATTACAGGCGTGAGCCA
CTGCGTCCAGCTGTTTTTTTAAGAAATGTTTTTGTGCATTTTATGGTCTAATATATCCACATACCCAGTG
GATTATTACTAAGATATTTGAAGGTAGGCAGTGTCTTTGTCAAGCTTATAAATAAATATGTCTTTGGCTA
AGCCGTGATCTTATTTTTTTATTCTAGGGGGTAAAAGAGGATACCTATTTGTGTTGGAATTTGGCTGACT
GTATATGACTCCAGGTTGAGAATGAGGGTGCAGTGTCTGTTGAATGTGTCACTAATTGCAGTGTAATTAA
GAAAGTATAGCTCCTTGTCTCAGTATTTAGACCACTAGAACATTTGTTTAGAAATACCAGGGAAAATCAC
TCTGAAATTATATGCATATATAACCAATAAGAAACTCACATATTCTTTTTGAATGTAAAAAAGGAACATA
TGACTCTTTTGTCTGTGTGGGGTAGCAGAGTAGCAGTTTGGATACAGCAGAGCTGTGTGCACATTGTGAA
TTGCCTTGAAGATGTAAGGGTTGATTTCTGTCTTCCTCTCCCTCTCAGAAAATAAAATTCCCCCAAAGTA
AGGTAATAGCAACATTCTGTAAGGTTAATCTCAACAATAGTGGTCTTTTCCCAAATTCAAATTTGATGCA
AAAAATTCAGTATCTTGGTCTCTCTCTGACACTAAAAGAAGCAGACTGCAGAGGTTGACAGACTTCCAGC
AGTTCCAACAGAAGGGATACTGGATCCCTGATCCCCTTTCTCTTTCTCTCCATAAAGTCCTGTGGAAGAT
CTCATACAGCTACTTTTGTAAGTGGTAGTACTATTAGCAGCAGATGTTAAAAATATAGATTAGTCTTTTG
GCATTATTTTATTTTATTGTATGTTAGAAGCAAGCATAGAATGGCCACATTAGCACCAGTAACCACAGAC
CCCACGTTTTGAGTTGGAAAGTGAGGGAGGAGAGGTTTTGTATGGCTGGGGGCAGGAAGAGTAGGGAAGA
GGCAGGGGGCTAGTTTGGTTAGAGTGTTAAGAGGGACACATCTGTTCTTAAATTGTTAGTCCTCACTTCC
TTTGGATGTGGAGATGTTACACAGAGGATTAGTTCTTTTACAAATATCTGTAAGGAAGTAAGAGAGCAGA
GAACAAGAAACAGGTTGTGGAGCAAAAGGAATGGGCACAGTAGCTGGTGAGTAAGTGGCAGTCTTTCCAT
AGAGTAGAGAGAGAGCCAAGTTTCCATGGCTTACCCATATTGTCACAAGACCCTGTAGTGGGTAGGGTGG
AGGTCATTGTCTGAAGGTGAATCTTGCTGATTTGCAGCCTGGATTCTTGCTTTATAGGGTAATAGACTGG
AAGAACTGCTGAGCCCACATTAGCTGGAAGATAAACCATGTGCCTGCCATTTTTCCTTACATCTTTCTGT
CTAGCAGAAGCAAGTTAGTCAAGATTAAGTGATCCTTGTGGTGTGAGCTCTCGGGGCTATGAGACATTAA
AAGAAGCAGAGTGCAGAGGTTGACAGACTTCCTACAGAGGGGATATCGGATTCCTGATCCCTTTTCCTCT
AGAATCTGATGCGGGAGGGACCATGAGGAAACGGGAGGAAAGGGCTCTGAGGGACTGAAAGGGTGGTTAG
GGCTGAGCTTCTTTACCATCTCTCTGCAGCATGAGAAGGAAGATGGATTAGTCACTAACTGTCTGACAGG
AGATAGCCCCTGGGGATAGTCATGTACTGGCTTTGAGTAGATGCCAGGGATTACTAACTAATTCATGAAA
TGCTTTGTGTCATTTTGTTTGATCTTCATGATTTCCCTATGAAGTAACTGAAGCGCTATTTTACAAATGA
AGGAGCTGCAGGTTGGAGGTTTTTTAATTAATTAAATCTCATATAGCTAGTTAATGCCAGGGGTCCACAA
TTTTCTGACTCCTGTGCTCTTTCTTTTCTGCCTCATCATATTTGCATAGTGGGAAGAGAAGAAAGAGTAA
GATGGAGGGAGAGATTTTAGCAAAAGGCACAAAAGTATGATTCTATGTAATTCTGTTTTTACCAATAGGA
ATATTAAATATAAAAATGTTCATATTAACACCACAGGGATTTATCAGAGGTAAAGTCTGTTATATAGGTA
GGATGAAGGAGGTCAGTATGAGTGAGACTTTGGAACCTTCATCCCACCATTAGCCAGGTTGAAACAAAGT
TTGAAACCACTCTTTTAGGGCATTTCCTATAAATTTGTTTACTCTTTCCTTTTCCTAAGGCCCCCACTTA
AAAGTATATTTTTTCTTTCTTTTTTTTTTTTTTTTTTTTTGAGGCAGAGTTTTGCTCTTGTTGCCCAGGC
TGGAGTGCAATGGCGTCATCTCAGCTCACCACAACCTCCGCCTCCCGGGTTCAAGCAATTCTCCTGCCTC
AGCCTCCCGAGTAGGTGGGATTACAGGCATGTGCCACCACACCCGGCTAATTTTTTGTATTTTTAGTAGA
GATGGGGTTCGAGCTCCTGACCTCAGGTGATCCACCCACCTTGGCCTCCCAGAGTGCTGGGATTAAAGCC
TTAGCCACCATGCCCGGCAAAAATATATTTTTCATCAGTCTTTAAAGAACTCAGAATATAAGACATTATA
CATTTTGAGATGTTTGCCTTTTTAAAAATGAATTGTTTTTCCCATTATAAAACAGTGTGATAAGACACAG
TTGTTAGGGGTCAGTGTTGGTAAATTGACAACTAAAATCACAAAGATGTTTGATAATACCTCTCTGCCAG
GTATGGGAAGGTCGGTGGCGAGTGATCCCTCATGATGTACTACCAGACTGGCTCAAGGATAATGACTTCC
TCTTGCATGGACACCGGCCTCCTATGCCTTCTTTCCGGGCCTGTTTTAAGAGCATTTTCAGAATACACAC
AGAAACAGGCAACATTTGGACACATCTCTTAGGTATGTAATGTCAGTGATGTAATGAGCTGGTGATTCAC
TTTCTTCCTTTTTATTTTCATGTATTTGAGGGTAAGCACAGAACTTCAGAAATGTATTTGGATTTGCCAT
TTTGTTTTCTGAATTTCTAATGATGAATTTTCTGACTGGTTTACTCGTAGTTTATCCTGGTTTGCACTAT
GATTTTGTTATAACTTAAGTTATATTTCTTTCTACTAGATTTTCAGATCTTGTCCTCAAACCAGGCCAAG
TAAAAGTTGCTATACAAAATGTGGTTTCATATTGTAGTGACCACTGAGAAGAAAGTATTATTTAATAGAA
GTAATCTGATCTTTTAAAATGCATTACTTATATGCACTGCTAATTTTCAGTTTGGCCTGAAGTAGAATAT
GGACTGTAATTTTTTCTCCATAATGATTGTATATTAAAGAAAGAATACTGTAGAGGAAAGAATAGTCTTT
CCAACAAATGGTGCTGGGATAACTGAGCCATATGTAAAAGAATGATCCCGAACTCACATATGCAAAAGTG
AACCCAAAATGGATCAAAGACCCAAACATAAGAGCAAAAACTATAAAATCCTTAGGAGAAAACATATATA
AATCCTTGTAAGTTTGGATTAGGTGACTGTTTCTGAGAATTGCCACCAAAGAAAAAATAGATAAATTGGA
CTTCATCAAAATTAAAAACGTTGGTATATCAAAGTGCATTATCAAGAAAGTGAAAAGACAACCCATAGAG
AAGAGAATATTTACAAATCAGCTAGTTGGTAAGGATCTAATAAATAACTCCTACAACTCAACAATAAAGA
CAACCGATTTTAAAAATGGGTAAAACATCTGAATAGATGTTCTTCCAGAGAAGATCTAAAATGGCCAATA
AGCACATGAAAAGATGTTTGTCTTCGTTAGGCATTTAGGAAAACGCAAATCAAATGCTTTGAGAAATGCA
GTGAGAAATCTCAACACTTCACACCCACTAAGATGGCTATAATCAAGAAAATGAAAAATAACAAGTCTTG
GCAAGGATGTGGGGAAATTGGAACTTTCATACGTTGCCAGTAGGAATGTGAAATGGGGCAGTCCCCATGG
AAAAGTCTGGCAGTTCCTCAAAAAGTTACATAGAGTTACCAGAAATCAACTCCTAGTATATTACCCAAGA
TGACTGAAAACATGTTCACATAAAAACATGAACACAGAAGTTTGCAGCAACATTATTCATAATAGCTAAA
AAGTAGAAGCAACCCAGATGTCTATCGACTGGTAATGGAGAAACAAAATATGGTGTATTCATACACTGGA
GTATTATTTAGCTGCAAAAAGGGATGAAGCACTGATACATGCCACAGCATGGGTGAGCCTTGAAAACATT
TGCTAAGTGAAAGAAGCCAGACACAGAAGGTCACATGTCGTTTGATTCTACATGCATGAAATGTCCAGAA
CAGGAAAATCCATAAAGACAGTAGATTAGTGGTTGTTTAGGGCTGTAAGGAGTGGGGAATGGGGGATGAC
AGCAGATACAGTACTTCTGTTTGGGGTGATGAAAATATTTTGGAATTAGTTAGTGGTGATGGTTGTACAA
CCTTGTGAATATACTAGTTTCTGGCCTTACTCCATTTGATTTGCATTTCTTATCACACATTTGATTTCCA
TGGTGCCTCTAAGTTCCTGCCTGCTAGTATAGTGAGAGGAGAGTAGAGGGTCAAATCAAGTTTGTTGCTG
ACTTCTAATTCAAGGGTAATTTTTAATTAAAATGATAAAACAATTACTTAGCAAACGTTGAAGTTGAAAT
TTCTGTGCCTGCCTTGTGTTAGGTGGCCCCTCCCTAAGCAATAGGAACATGGCATCTTTCTGTGTAGAAC
AGTGTAAGAATTGGTTAGGAAATGGCCTGAGTAGGCATTTTGTCCTTAACTCTTGAATCTCCTGAACCAG
CTGATGAGGGCCAGAGAAACAGCATGACACTCCCTGACAGCTATTGGTAGAGGAAAGCTGTATTTCTTGG
CAGGGTTTAGGGGTATTGGAATTCATTGTAAAAACAAAAGCAAAAGCACAATGGACCTTTGAAAGCTGGA
CTATGAGGTATGTGCCAAAAACTGTATAAATGAAACTATTCTACATCTAGAGGTAGAAGTGGCTTCATTA
TATCTTCTAAGAAGTAGCAGCAGTGTCTGATTTCTTTTTAGATTATGCCCTTAGATAAGTTTTTATATTC
TGTATATTCAGTTTATGAAGCTGAAGTATTTCTGATACCAACAATGTTGGAAGTAAACCACAATTTTGGA
TAGGCTTCAGTTATTTGGAAGATTTTTCTCATTGAACTTTTGATTCAGATATTGTTGTAATGCAGAAGGA
TTGAAAACAGAATCAGTTATAATACTGTCTCTTCTAAAGGGTGATATTTTATCTATTTTCTGTGCTCTTT
TTCCTAGGTTGTGTATTCTTCCTGTGCCTGGGGATCTTTTATATGTTTCGCCCAAATATCTCCTTTGTGG
CCCCTCTGCAAGAGAAGGTGGTCTTTGGATTATTTTTCTTAGGAGCCATTCTCTGCCTTTCTTTTTCATG
GCTCTTCCACACAGTCTACTGCCACTCAGAGGGGGTCTCTCGGCTCTTCTCTAAGTAAGTATCTGTAAAG
TCCGTATTTTGGCCAATGATTTAGAGGTAGTGCGTTAGGGAAAAACATTCAGCAGAGTTGGCAGAATTCT
TAATATCATCTCATGCAAACTTTTTTTTTTTTAAAAATTAAAGAGCAACCCAGTTTGGCTCTTAGGTGTC
GTTGATGGCTTATGTCATGAGGGATTAATTGGAACCTGTAGTGGCCAGTAGTTTTTAAATTACTGCATTA
AATGGATTTTTTTTTTCTGTCATAGACTGGATTACTCTGGTATTGCTCTTCTGATTATGGGAAGTTTTGT
TCCTTGGCTTTATTATTCTTTCTACTGTAATCCACAACCTTGCTTCATCTACTTGATTGTCATCTGTGTG
CTGGGCATTGCAGCCATTATAGTCTCCCAGTGGGACATGTTTGCCACCCCTCAGTATCGGGGAGTAAGAG
CAGGTAAGAGCACGGGGAGGTTCTACATTCGACATTCATTTATTCACTAGTTAAATTCACTATTTATTAA
AGTTCTACCATTTGCCAAACATTATGCTGTGTATTTGTGATGCCAAGATGAAGAAATCATAGTTCCTATT
GTTGGGGGTTTTTTTATGTAGTAGTCGTAGTAGTTGTTGTTGTTAGTAGTGTTTATTAATTGTCTTAAAG
CATTAGGGTTGAGGGGTCATGGTGAGTATGAGGTGATATAATTTGGGACTGTTACTTACTTGAATTTATG
GCTAAACACCATCCAGAAAGCGGTAGTCTATAACTTTTGTAAACAATGATTTCTCTTACTCATTGTGGAG
TGTACTTTTTGTAGTGATTCTTTTTTTGTTGTCATTTTCTGTTTTGTTTTATATTTTTATTTTATAATGG
ATAATCTGCTTAAGGCTTCACCCCCATGCTGATTATGTTTTGTTAAAGTGGGCACCATATCTCCCTTCTG
ATGTAGGGATAAAGGGGCTTTTTATATTTAGGATAATGCCTGTTTAGCTGGAGATCATAGAGAAAGCCAC
AAGCCCCTCAGTGCCTTGGCTGAGTCTTCTTGTAGGACATGGAATTAGCAGAGATGTTCTTGTCTCCTAC
TCAGTGTCAAGCTCAGTGCCCACTGAGAATGAACAAGGAGTGTTTTTCCTTTCGTCCTTAAACCCCCAAA
ACCATTTAGTATTAAAATCTAAACCCTGGATCATCCGGGGGTACCTGGAAGGTTAGACACAGAAGTAACC
TCATGTCAGTGACCACTGTTAAAACACTCTGCCGTCCTCCTTTTTGTGTTCATATGAGCAAATGAGCATT
GGTCAGGGTCAGCTTTCAACAGTACTTGAGTAGTACAGTGGTGTTAAGCCTGCTTGAGCACATGAGATAA
GAATGTCTTTGCTGAGTACAGCTGAGCATTCTGTTATTTACATACATCAGAAGAACCAAATTCCTGAGCC
TTTGACTTAGAGAATGATAGGCAGAAACCATTAGCAGCCTTTGCTTAGCATGTAGAGGAAAAGAAAAAGT
GAATAGTGCAAAAGTCTAGCAAATCTTTCCTTAGAGGAGCAGAGGTAGGAAATAGTTTTTCTTTAGGTCT
GCTTGGTTCTTACCTGTTGTGAGAGCAGCATTGCCATTTTCCTCTTCTCTTTTAGATGTCCATTTTATTT
GTGAGTATGCATCCCATAGCTGTGCTTACATATATTGCTGTATACCAGCAGTTTATTTGGTAATGTCCAA
AGCAAGTAATAGGTTCTTTATCTGTTGAGAAAGCTTACCAAATGACAGCTTGTTTTGCAAGGGTCATTGT
TAACTCTGTGTCAAAATGTAGGAGCAGTTACTGGCAGAGGCAGGGAGCTCCTGTGTTGCTTTGGCTGATA
CACGGGTTTCAAAGTGGATTTAGATCAGTGGACTTCACATTTTTTTTTCCATCATGACATTTTCTATTAC
AGTCCTGAACACGTGTATCTATACATAAGTTCATGTATATATAAATTACAAAACTTTAACAAAATAGTAT
TTAATCCTCCACGTATGTTACAGTCTGATATTTTAAATTTTATTCTAGTTCATTTAAAAAAATGCTGGTT
GAGACTCATTTAATTGATCTTAGAACTTAGGTCATATCCAGAAGTTTAAAAAAACATTAGATGCTTCTGG
AGGCAGTAAGGCAGTAGGGCTGTGGCTGAGTGATTTGCTAGACTTTTGTACTTGATTGCTATTTAGGGGT
CTGGGAGCTTATAGTATTCTCTCCCATCCCCCACCCCCATTAGGTCATTTGTTAAAGGCATTTAACAATT
TTTAAGTGCCCAGTTATAGGTTATAGATTTAGATTAAATTTTTTCTTCTTTTTTCTTTTCTTTTCTTTTT
CTTTCTTTCTTTCTTTTTTTTTTTTTTTTTTTTTTTTTTGAGATGGAGTCTTGCTTTGTAGCCCAGGCTG
GAGTGCAGTGGTGCCATCTTGGCTCCTGACCTCAAGTGATCTGCCTGCCTCGGCCTCCCAAAGTGCTGGG
ATTACAGGTGTGAGCTGAGCCACTGCGCCCAGCCTTAGATTCCTATTAAAAATAGGTGATGCCAGGTGAT
GTGATCATTCATCGCCTCTCCACCCCAACAACGCCTGCTGCTTCAAAGAGAAGATGGGATACGTAAAATT
GCTTGTATTGTAACCTGTGTCTGTAGAAGGACAGGCTAGGACAGTTTAAATAAACTCCTGGAGCTGTCCT
TTCCGAAAACATTTAGCAACATTAATTCAAAACCCTTGAAGACTGCTGACTTACTAAGCTAAGCCTCTCT
TAGTGAGGCTCATTGCTTTTTTTTTTTTTTTTTGAGACGGAGTTTCACTCCTGTTGCCCAAGCTGGGGTG
CAATGGCGTGATCTCGGCTCACTGCAGCCTCTGCCCCCGGCTTCAGGTGATTCTCCTGCCTCAGCCTCCC
GAGTAGCTGGGATTACAGGAGTGCGCTACCACGCCTGGCTACTTTTTGGATGTTTAGTAGAAATGGGGTT
TCACCCTGTTAGCCAGGCTGGTCCCAAAGTGCTGGGATTACAGGTGTGAGCCACTGCACCCGGCTGGCTC
CTTGCATTTGTAAATCTTTAGAACATAGCTGTGTTGTCTTGTGTGACAGTGTCCCAGAGTTTGAAGAACC
TTCTTTTTCCTTTAAAACTGGAAGCTAATTTTATATGCTACTTTGAAATATTCAGTTTACAGAGTTAATG
GTGCTGTGCTGTAATACTGACTTCTGGCTCTTTGTTTCTCTTCTGCATTACTTTACTCTCTTCTTGTGAC
TCCTAGGAGTGTTTTTGGGCCTAGGCCTGAGTGGAATCATTCCTACCTTGCACTATGTCATCTCGGAGGG
GTTCCTTAAGGCCGCCACCATAGGGCAGATAGGCTGGTTGATGCTGATGGCCAGCCTCTACATCACAGGA
GCTGCCCTGTATGCTGCCCGGATCCCCGAACGCTTTTTCCCTGGCAAATGTGACATCTGGGTAAGTATGT
CGGGGTGACTGAGTGTGTAGGTATCTGCTCATGAGTTATTGGCATCCTGCAGAGTGATGCAATAGAAAAA
GTTTTAGACACAAAGTCAGGAGAGTTGTATCCTGGTCCTAACTCCCTATTGAGCTTTCTCTGGACAATAA
GTACAATAATAACTAACATTTAGTGAGCACTTCCTTGGTGCTCAGCATGGTACAGAGTGCTTTGCACGGA
TTACCTCATTACATACATTAGCCCTATGTGGTATGATAACTCTTGTTAATGTCTTCATTTTACAAAGCAG
GAGACTAAGGCTTAGGGAGGTGAGGCCACTTGTCCAAGCCATGTGGCTATTTAAGTGGTAGAACAGGAAT
TTGAATTAGGCTAGTTTTAATATCAGAATCTGTGCTCTTTTTGACAATGGCAAGTCATTTTGCCATTCTT
TGTCTCAGTTTCCTTATTTATGAAATGAGGATGATAGACCAGATTTTCACCAGAGCCCTTTTTAGGACCA
GATTTCTATCAGGGTCTTTTTTTATACCGAAATTTAGTGATTTGTAATGGGAGACTGAGTGTATGTATGT
CTGGTGGAAGAAGAGGGATTGTGAAGAAGAGGGAACTGGATTGAACTGGATTGTGGTCATTGGTAAGAGA
GGAGGAAAGAGAAGAGGGAAAAGAAAATATAAGGACTGATATAGATAGAAAAACTTATAACCAAAGAGGG
CCTGAAATATTTTCAACAAGACAACATTGGAAATACGCTTAATGTGAAATCTGACCTTTGTAATTCTGTT
CCTCAGCCTATTGCTGATTATTATAGACTAACCTGATGATCTCATCTCTTTAAAGTTTAGTTTAGCTATC
TGTAAAATGAAGATGATCTTGAACTTAAGCAATCTTTCTAGCTTCAAATTAGCTAATTTATCTTTCTGAT
TCATCATCATCATTTTAACATTGGTATGGAGGGGTTTTTGCCCTTTATCCCCTTTCTTTGGGGGAAATTA
ATCCCCTTTGGTTTCCTAGGAAACATCTCTTGGGATGTGACTTGATTTCTTCTAGTTTTTGCTTCTAAAA
TCAGTGTTTTTCTCTTGATAAATACTGCTTTGCCACTGTACTTTTTCATTTTGAGATTATAATGACAGAA
TAGTGTAATTTTTGAAGTGCAGCGAGCAATTGATTACCCTTCTTGAGATGTGGAATAGCATTCAACAGAA
CAGGTAAAATCAGAAATAATGTCACTTGCTTGTAGACCAGCTTCTGACTTGAGAAGTAAAAGGGCCTGTT
TGCTTAGTTTTTGACACATTATGTAGTTCATGAAATTTGAGAATCGTAGCACTGAAGGAAACTCTGGGGA
CCATATGTTGTGACATTCCACACACCATCCTGGAAGCCTCTTTAGTTTTTCTAACAGAGAGCAACATTTA
AATGACCATAGCTCTAGTCCTCTTGTCCATGACAAAATGTCCATCAGCAGTGAGTAGGAAAAGGACTGTA
TGTGTAATCATAGCCCATGGCTAGGTGTCAAGGGATCCTTGGGACCTTTGTGGCCTGTTAGCAAGAGAGT
CTGTGTGTCATTTGCCACTTTAGCAAGAGAGTCTGTGTGTCATTTGCCACTGGTTTCAGTGTTAAGTTTG
TAGATTTTTATGTGTAGAGCTTAGTGGCAGCACCTTGGTTTCTTTGGGAACAGCTGGCTTTCAGCTCAGC
AGAATGTGTTACACCTAATGGATGTTCCAGGATCTGTGGACGCCTTGAATTTGAGCTCTGTCACATCCAT
TCCCTGCCTTCTGCTCAGCATAAACCAGGATTAAACGATCCTAAGAATCTTTAGCAGTCTTAATAATGTA
TGGGTTTCTCTACCCCCTTCTCTTCTTTTTTCCCCTCCAGTTTCACTCTCATCAGCTGTTTCATATCTTT
GTGGTTGCTGGAGCTTTTGTTCACTTCCATGGTGTCTCAAACCTCCAGGAGTTTCGTTTCATGATCGGCG
GGGGCTGCAGTGAAGAGGATGCACTGTGATACCTACCAGTCTCCAGGGACTATGACCCTAAACCAGGGCC
TGCGGCACTTGCGGGCCTCCCTGCTGGCTACTGATGCCAGTACCAGAGGAGCCCCAAAACTTTGACAGCC
TCGTGGGCTTTGTGACGGCCCAGTGGCTCTGCGTGGTACATGACTGAGAAGAGAAAAACAAAAATAAATC
ATACCTCAAAGGATGGAGTGCATCAATTGGGAGAAAAGGAGACATAGCCCAAACCCTGGCTTATTCTTGG
GATCTACTGATTGCGGGCTCTGCAAGACCCTTGGCAAACTGGCTTCTGATCCATATCATATTTATTTGTA
GAAGATGGCGAAACAGTTTAGCTGGTGGTTCTTTCTTCTCCCTTTCTCTCTCTCTATGACAATAATACAA
ACCAATTTAAGTGAACATTTATATCCGATAAGGGGTGGGAGTGTGATTTTAAATGCTCTTTTGGGAGAAC
AAAGAAATTAATGTAAATAAGATTTCTAACTGTTTAAATAAGACTTTATATAAATGTTTAAAACATAGGG
GTAAGGGAGGGAGGGAGAATTTTTGTATAGAATGAAACATGCAAGTACCACACACTGTTTGAATTTTGCA
CAAAAAGTGACTGTAGGATCAGGTGATAGCCCCGGAATGTACAGTGTCTTGGTGCACCAAGATGCCTTCT
AAAGGCTGACATACCTTGGACCCTAATGGGGCAGAGAGTATAGCCCTAGCCCAGTGGTGACATGACCACT
CCCTTTGGGAGGCCTGAGGTAGAGGGGAGTGGTATGTGTTTTCTCAGTGGAAGCAGCACATGAGTGGGTG
ACAGGATGTTAGATAAAGGCTCTAGTTAGGGTGTCATTGTCATTTGAGAGACTGACACACTCCTAGCAGC
TGGTAAAGGGGTGCTGGAGGCCATGGAGGAGCTCTAGAAACATTAGCATGGGCTGATCTGATTACTTCCT
GGCATCCCGCTCACTTTTATGGGAAGTCTTATTAGAGGGATGGGACAGTTTTCCATATCCTTGCTGTGGA
GCTCTGGAACACTCTCTAAATTTCCCTCTATTAAAAATCACTGCCCTAACTACACTTCCTCCTTGAGGGA
ATAGAAATGGACCTTTCTCTGACATAGTTCTTGGCATGGGAGCCAGCCACAAATGAGATTCTGACGTGTC
CAGGTTTCTCCTGAGCTCATCTACATAGATTGGTAGACCCTTCCTTTGGATTAGGAAAGATGAGTTTTAC
CTCTGGTACACTGTCTTGGTAAGCCTGGATGTGACAGACACCTCGGCTCTCCTTGAATAAGAAAGCCAGC
AGAACTCTTAAAGCCAGTTGTAGTACGGAGTTGTCAGCACTCACTGAACCTCACTTTACAGGGATAAGAG
TGGTGTGGCATTTTAAATACAATGGTATGTTATTGCCAGGGAGTGAGGTACAAGACGATGGCTCATGTCA
CAGGCCTACCTGATACGGTGTCAGAGAAAGTGGTGGGGAAAGGATCTGGTTCATGGAATTCTGATCTTGG
CCCATAGGTGAACCACCAAAATAGTGCTCGAGTCTTAGGTTACTGTCATCAAAGACTTGGGATGACTCCA
TTATATCCTGGGGTTGTGGGTATTAGAACTAAATATGGAGGTCCTGAGCATGGGGACTGGCGTCCTCAGT
AGGTGTTTGGGAATATGGGAAGGGTCTCCTATTTATTCAATAGAGTTTTCTCAGTTATTTTCCTCCCTTG
CCCTTGCAATCTCCAGCAAAAGGTGGGATCTAGGAAGAAAGAATCCAGTGTAGAAGTTGAGAAGAACTTG
AACGTTTTGGTTCTGGATAAGGTCACTGTCCTAGGTGCTAGGTGGACCGAGCAAAAGACTCAGTGGATGA
ACTGGTGCAGTGCCTGACAGAATAAAGAACAGTATTAATCCCTTTGAGAAAGCATAGTCCAGCAGGACAG
TGGCCATTTGGACAGAAGCCCACTTAGTTTCTTGGGAGCAACAGCACGTATCAGAAGCCAGACTTGCTCT
TCGGTCATGCACTTTGGGATACAGCGTATAGGTGCAGCCCTGTCACAACACCAACAGAAGTAGCAGCCTC
TGGGTGCAGTCACCCACACCCCAAAGCTGGAAGGATCTGGTTCAACATAGCACAAACCCTTAGGAAAAAT
GAAATTAACATCACTGATGTGTAATCCAGTAAAATCTCCCTTTTTCGGGTGTGTATGTGGGCATGTGCCC
ATTTCTATGTGTGTGTCTACGTGCAGCTCACTACCAACAGCCTCATGTGCACTTGACCTGACAGTGCTCG
CTGAGAACTCTCACCAGGTTGGCGCCTGAATGCCTTACTCTCAGCAGTCAGAGGCTTGCTTGCTCTGTGC
AGATTTTTAATTTTCTTTTTTGGCCCTAGGCTGGTTGGGACCTCTACAGCTTCATTCTTTCACCATTAAA
TAGTGGCCTTTTTCAGTATTTTCCCTCTTCCCCTTTATAAATTATGCTAAAGCCACAAAGCACATTTTTG
GGGATCATAGAAGGTTGGGGTTCCAGAAAGGCATCTGTGTGATGGTTCCATTGATGTGGGATTTCCCTAC
TTGCTGTATTCTCAGTTTCTAATAAAAAGAACCAAATGAAATATGACCTGTCGTGTGTTTTTTTATTGCC
GTTCAGCTTACCTCCCCTTGGCTTTTGGCACTTCTCTGAAGCTCTAATTTAGACACTGTGATTTAAGAAA
TTTTTGGAAACTTCCCCTTCGGGATGCAGAAATCAAAGTGCTTATGCCCAACTCCTGTTTCCTAACTCAA
GCTTTCCTCGTAAGTTGCACCAGGTTTAGAATGGAGGGGAGAGCAGCTCTAAGGGGCAGTTCTTATTTAT
TATTGAAGTTTATAAAAATAGTCTCTAATGCCAAGGGCAATAGGCTCTACCATAAAGCCTGGGTGTGTAG
TAGGCTGTACCATCTAGGTTTGTGTAAGTGCATTCTATGATGTTTGCGTGACAACGAAATCAACTAATTA
TGAAATCACTTAATGATACATTTCTCAGAATGTATCTCTTTTGTTAAGTGAAACATTACAAAATTTATTT
TTTTGGGCACGGTGGCTCACTCCTGTAATCCCAGCACTTTGGGAGGCCAAGGCGGGCGGATCACGAGGCC
ATCTCCTACAGGCAGTGCACATTGACTAAGTTCATTCCTTGCTTTTGCTGTTGTTTGCTATTATAAATAG
GATTTTGTCTTCAATTATATCTTCTAAAAGTTCTTTTGATTGGTGAAAGCTACTGATTGCTGAATATTGG
TTTTTTACCCCGACCATTTGCTGTTTTCTCCTCCTAATCATAATTTTTTCTTAGTTGGTTTCCAGTAATA
AAGTCATGTTGTCTGAGAAAAAAAAAAATAGTCTCTAATGTAACACTTGATGCCAAGGGATGTGTTAGAG
AACTGTCGATGTCATATGATCATAATCACAACCCTCTGAGCCATTCAGGAGTATTTTCACTCTCATTTTA
TAGATGGGGAAACTGAAGCTTAGGAAGTTTAAGAATGACTCTAGTGCCAGCACTGTATTGGAGAAAAGAG
TGAAGAGAAAATTTAAGAAAACCTTTCTGTATTTGTTATTCTGATTAAACAGTGTCATATACATTGACTT
AAAGGTTTTGGGGTTCATTAACCTAGTCCCTTCTATATAAGAAGAGGATCTGGTTCGAGTTGGTGTTTGA
AATAAAAAAAAAAAAAACGGAATTCCAACCCTTTTGCCTTAGGTAAATTTAGTTTTTGGAAGGGCATTTC
CAGTATCAGGATAATATGTTCATTCTGAATTCTCATTTTTAAGTATAGATTTAACTTTAGAGTGTCCCAT
ACCTTACCTGGGCATAGACATTGGATCATTCCCTGCTTTTGACAAACAGTATTAGCTGAAGCCTACTAAT
AACCCACTTGTCTTTTCCTCGTCTCTTCCCAAAAGGCATCTGAGCCGGGCATGGTGGCTGACGCCTGTAA
TGCCAGCACTTTGGAAGGCCGAGGTGGGCGGATCACAAGGTCAGGAGTTCGAGACCACCTTGGCCAAAAT
GGTGAAACCCCGTCTCTACTATAAATACAAAAAATTAGCTGGGTGTGGTGGAGGCACCTGTAATCCAGCT
ACTTGGGAGGCTGAGGCAGGAGACTCGCTTGAACCTGGGAGGTGGAGGTTGCAGTGAGCTGAGACCATAC
CACTGCACTCCAGCCTGGGTGACAGAGCGAGACTCCATCTCCCAAAAAAAAAAAAAGTGTCTGTATGGTT
CCTGATACCCAGGTGATGTTATGTGGTTGTAGAGGAAGTAGATAAGGACAAGTTTGGATTCTGACTGTAC
AGCGTGCTAGGCTCTGGGTGCCTAGAGAACCCTCTGGGCAATGTTACTTTAAAATCATCCCAGAAACAAC
ATCTTCTTGAATATTTGGCTTCAATTGTAGTGTTTCATTTTGTCTTCCCCTCAGAGGGCATGAACCATTT
GCTAGCCAGCACTGCTCCCTACCCACCACCAACACCCTATCTAATGTACAAATCTTTTCTGCCTTCAGCT
GTTTGACTTGGTGAAACAAGGGCAGCCTGTCTCACTCATGTAGAGGCTGCTTATGATGGCATGGGGCAGG
AAGGAAGCGTCACGTGCGATGGCCTCTTGTCACCGCCATCCTACTGTCCCACCGTCCCTACCCTGCTACA
CTACCTGGAAATGAAAAGGGTAGCTTCAGATCTCAGTTGTGCCATTTGCCATGTAGCATGGGCAAGTGGC
CGTTTGCTTGTCTAGCCTCGGTGTTCTGTTAAAATGAGGCCAACGATACATCCTCTAGAGCCCACGCTGA
GATGCTGTGGTTTCTATAGATAATTTCCTCAAACTTCCCATAGTGATTGGCTGGGTTTTCTGCCAAACCC
TCGCTCCAATTCCGTCCTTTTGTGGACTTCATTTACTTTTCTGGCCTTATGATATGCCCACTTTCTTGCC
TCCCCGCCGCCCCCGAACCACAACTTCACTGAGCTTAATGAAGTGGTTGAAGCATTTTCCCTGAAAGCTC
TTCTATTAAAATACACAACATTATTATGTGGATTTAGTAGTTCACATATCCTTGGTACTGAGGAAGGAAG
ATCTGACAGCATCCTTAAGTTCCTGGTCCTCCAAAGGTGGTGTACAACATAAGATTTCAAAATTCTGCAT
TCTAATTTATCAAATGACTTAGGTTCCAAGTTCATGCGTCCGAGAGCAGGTGATGGGCTTTAGAGTGAGC
CAGGCTGCCACCTGCTGTCCTTTAGTGGTTCTGCTAAAAGTAGAAGAGGAATCGTTCTGACCTTCAAAGG
CAAAGCTTATGAGAATATTTTGTTCTCCAAGGTAGAGAAGGTTAGTTTATCAGAAATGGTTTTTCCTTCC
AATGGTCTTGAAAGGGATTCCCCTCGGCCATGCAGAGATATGGGGCCCAGAGGAAGCAGACAAAACCAGG
CCAGAAGCTGCTCCAGCTTCCTAAGCTTGGCTTTTTAAGAGTTGATAAATTTGCCTGAAAGTGCTCTACC
TGTTTGGGTGGGGATGATAACCCTTCCAAAAGATAACACTGGGTTATGGTCCTACAGAGACTGCTGTTTG
GTGTCTGTGAGTTGGTAGTTGGGGTCAAATGCAGTTGGAGGTCAGAATGTGCCTTAATGCCAAAGAATGA
ACGGCAGGTGGGCGGCCACGGCCTTGGCAGGAAGTGGCAGGGCGAGAGTTAGTGCTCACCCTGCTGTGTG
CCGCACAGGTGGTGCCTCGTGACACATGGTTGGGACACTCAGTGATGGCAGCCCAGGGGCTAGAAAGGGC
TCCTTGCTGCTTTCTTAACCCTGCTGGGCCTGTTGAACACCTTATTGTTCCTGGCATCTAAATTGTAATT
TCTTTGTCCCCACCTCCATGAAACAAGGTAATGTAATGATCGCAGTTAGCATGTATTTATTTAGCGTTTA
GTATATGCCAGGCAGTTTCAAGCATGTTACGTGAATACATCACATTCAATCTTCATACACTCTTCCGGTA
ATTTGGAGGCAATTTTCAACTCTGCTGCAGTTTCTATAACATGGGACTAATGATAGTACCCACTTTCAGG
GGGCTGTTGTGAGGATGGAATGAGCTGAAACATGTTATTTGGTACATGTAAACCTTAGCTGTTCTAGCAT
TATTCTGCCACTTAAGCTGTCTTCGAGCTGCAGTGTCTTCTGCTGTAAGGGGGCATTAATACTGCCCTCA
CAGGGTTGTTGCAAGACATGACGGTAAGAGTGACATTCTGTAACCGATAATGTAAGTGTGTTTTACAGAG
TTGGCATTTGGCCTACTCTAGAGGCAAGTTAGGGCTATTTGCTGGTAGGAAACTTGAACAGCTTTGAACT
TGAACTTTGAAGTGTGGAGGCTGAGGGTGATGGAGGAGCGGGGGGAAGCGCCCCAAAAGCCCTGCATGCT
GTCCATACCCATCACTGGGGTTTGTTAAATGGAAAGATTTGAGGCCGGCCTCTCCCACATTTCTCCATGG
CTTTGCCCTTAATGTTGCTCTCACTGTGGAGACTGTTGTCTCCTGGGATCCAGGTCTTGGGGTTTGGTCG
GGGGAGGAAGCTGCGGGGCCTTGGGACCCTGGGGGAGATGTGTGTGCGCTGGGGGTGAGTTCCCAGATAG
AGCTTCCCTCCCAGTGGACCCTGCTTAGCAAGTTGGAATGTTCCCACAAGTTCCAACCCTAAGGATTTCA
CTCCAGGGACAGAGTCAAGTAGATTCTTTTGTAAATCATTGGCCAGAAAAGAGCACAACCACTGCTCTTT
TTCACTATTTTACAGCTATTTTTGCCGTCCCCTGAATGTACATCAATTTCCATGCAGCAATTGGATGTCT
CAGCCTATTGGCTTTTCATAGATGCAAACTTTCCTTTAGGGCTTTGTAAAGAAGTTTTGGGAAGTTCTTC
AATAGCAGCAGTTTGGGGACATCACGTGTAGCCCTGTCGATATTTCCTGAATGGATTTCATTCCTTTCTC
AGTCTTGACCATTCCCTTACCCAACCCCCACCCCAACACAGTCAAAACCTTTTCGAGCAGCAGCTAGGAA
GGTGAGTGGCCTCCCACCATGAGCCTCACTTTGGGGAATCTATGCTTTAGTTGGGGCATATTCTTGCCTT
AGCCTCATCTCTGCATTTGGCCTCATTCTGCTGCTGCTTCTCAGTACCCAGAAGTCCTTGGGTTCTACCA
GAGACCACTCCGACAGAAAGGGTCAGAAACAACTGACCCTTTCTTGGCTGGGTCTGGACTCTGGAGTACA
GGAAGAACTAAATTATTTTGTCTTGGTCCAAGCTGAGCTCACGCTGGCTTCCCGAAGAGGAGACAGGAAG
GTTAGGTCAGAAGTATGCTCATGTTGAGACCTAGGGCAGATGGTACCGGGCACCATGAAGCATCTTGAAA
GTGGTGCCAGGTGATTGGTTTCCTGTTCCATCCAGCATTCTCCGGAGCCAGGGGCCACCAGCCCAGGATT
GAGAGGAGCGTTGGCCTGGGGGCGACCCAACTGCAGTTAGCTGCATCCCATGTCAGTGCTGACTTTTTGG
GATGGCTCTGGAAGGATCACCTTGCCAAAACACAGGTCAGGCTGGGTGGTGTCACCGCAGGAGTTGGGGC
AGTAGCCCCCAGGCACACACAGGCAGCAGGAGTAGGGGCGGCGAGGCTGAGGTGTCCGAGGCGCCGCCGC
AGTCCTGGGCATTCTCCTGCGAACGCAGAGCAGAGGTGACAGCGCGGCGCTCAGAGGAGGACCCTCAAAA
AGGGAGGGGCTTCCACCACTAATTTGGGCCAAAATCCTGGGGAAAGGGGAGTTTTGGAAAGCCGGGGAAG
CACTGCTACCTCTCTTAGGCCCCTACCTTTTGGGGGAAAAGCAAAGGCTGGCCCCTCAGCCTGGGAGGGG
GGCACCGGCCTTCTCTGCTTGCCCTCAGGCTTGGAGTGGCTGCGAGGTCATCCTCTCAGAATGTCCTCAA
CTCTGGGAGCATCTCTGATGGGGGTTTCTGGTCCCAGCAGACCAGACTGTATGAAGCACCCGGCTCAGGG
CCTGACACCTGGGTCTGGGTTCTGGATCCGTACCAGCCACTATGACTATTATCCACGTGACGGCCCCCTC
ACATTGCTCGATCATTCGGAACTAAGTGTAGCGGGAATGAGGACAAGATGGGAAAACTGCATTCTAATTC
ACCTAGGGCCCAAGCCCTAGGAAGCGTGGCTCTTCGTTAAAGCACCTTGGGATCCTGGTTGACTTCGTAT
TTTAATGACACAGGGGCTCTGCTGTGCTTTCACACCAGCACTTCAACAAATGTGAAGGGTTTTTCCCACC
CCACTCTCTGGACACCAACTGGGTGTCTGTCCTGAAACTCAATTCAAGTCTGACACTATTGAGAGTTAGC
ATTAGAGCTCGCAAGTTACAGGGCTCGGTCCCACAAGGTGGCCGTCACTTCAGATGCCAATCGCAAGCCC
CAGGTCTCCTGTCCTTCTGACAGACTGGCTGTAAATCTAGGGTTCCCATGAGCCTGTCTCAGGCTCCGTG
AGTTGTTAGCGTGGCTCACTCAACTCAGGAAGGTGCTTCCCTCACCATGACCCGTTTATTATAAAGGACG
CAGGTCAGGCGGCTGAGTGGAAGAGACACGTAGGCCAAGGCACGGGGTACGCAGAGCTCCCACACCCTTT
CACCAATTCGGACACTCTCCAAACACAGTGGCTCGGGGATTTTATGGAGGCCTCATTATGATCATTGATT
AAATCTTTGGCCACTGGTGATCAACTCAGTCTCCAGCCCCCTTCCTTTCCCAGAAGCCAGGGGCTTGGAG
CCAAAAGTCCCAACCCTCTAATCCTAACTTGGTGTTTCCTGTGGCCAGCTCTCATCCTGTCTAGGGCCCC
CAGCCACCAGTCATCTAATTAGCATACAAAGATACCTCTATCATTCCAAGGGTCTTAGAGGCACCTGTGA
AGGAACCAGGGACTAAGACCAAACACTATAACAAAAGATGCACCTATCACCCTAATCAGGAAATAACAAG
CGTTTTAGGAGCTTGCCAGGAATCAGGACAAAGACTAAATAAAGATCTGTTTCTTATTATATCACAGTGT
CACAGGGCATAAACGCGAATGTTTCCCCCGAGAAGCACAGGCACAGGTGTGTTCATTGGCATCCCTATAT
GCTCCTGTCTGCAGGGGCACAGACCCAGGCACAGAGGGTTTGGGGATGAAAATCCAGCCCACCCTCGATC
CGCCTCAACCCGCACCTCTGGATGGAAGGCGTGGCAGGAGTCTGGTCGCCGGCGGAGCTTCTGGGAGCGC
ATCCGGTCACATTTGACAGAGGCATTATGTGCAGAAGCCACTGTTAAGGTCAATATCTCAGGACCGGAGC
CCATTCTGCAGACTGGAAAAAGGTCTGGAAGAACCTGCTCTGGTGCCACCAGTTGCCCTGCAAGCAGCTT
TAGGGTCAGGAGGCAGCACCGGGGCCCAGGGAAGGGTTAGAGAACAAATAACGGAGCCCTGTGGAGGACA
CGGGCGGTGAAGGAGGCTGGCCCCGCTGCTCAAAATCTGAGCCCTACAGACACCTCCTACAGCCCCCGCC
CCCACCGCACACATCCCCGAGCATTGCAGGATATATTTGACTTCTGTCGCCTCCTGCAGCACTGGTGGGA
AGATGCTGCAGTCACAGGTGGGGTCTGTCACCAGGAGGAGGAGGTTACTGTTGGGAATCTGCTGCACCAC
AAATACCCTGCAGCAAAGAAAGGGAGTCGAGGATGGCTCCGTGGCGACCACGAGAAGGGCTTCTAGGGAA
GGAACTTCTGGAGACTTTAGTGTGGAGTTAGGGTGGCAGGGAAGCCCGGAACCATGAGGCAGGGCGGGTC
GGGGCAGCTAAGGGAACGGGCCTGGCAGATGGCTCAGTCCTACTGGCTTTGCTCACAAGCCTTTGCTGCT
GCATTTCCTTTCCTAGCAGACTGGAGACAGTGAGGAAGTCCAAACTGGGCCGCTTTGGAGAGTGCAGAGT
TGCTATTAATATATGCAGGGACGACAGGCGTAAATCAAGACCGTCCGGAGCAAATGGGCGGGCGAGGCAA
CACTTGCTCTGGGTGAGGGTGGCGGGGGCAGGCCCGGGCGAAGCGGCAGCGCTGGAGCTTGGCCACCAGG
TGGCGCAGTGGCCCCGCGCTCGGCGAGGCCGCCCTGCGAGGAGTGGCGCGGAGTGGGGCGGAGCCCGCCC
GCCAGGCCCACGGCCTTCCCAGCAGTCCCAGCGGCCGCCTACCGAGCGCCTCCCCTGGGATGGGCGGTCT
CACACACCCAACGTGCACACCACAGCCGTGTATACTCTCAGCCTTCACGGCTCTGTATGAGGCTGGTGTT
GTGCCCATTTTGCAGATGAGAGAATGGAGACTAGAGGAGGCGAAGTCACTTGCCCACGGTCATAAGCGGG
GGAGCGCAGAGCCAGTTTTCAATTCAGGAGTGTCTACTCTGAAGCCCTCGCTTCACTCCGGAGCCCGCTG
GCTCTATCCTCGCACCGAGCCTTTGCGACAAGTGCACAGGGAACGCTAACCCCAGCCCTCAACGCTGCAC
AGAACCCGGGGTGAAACCCGCGCAGCACGACGCGCAGGGAGTGGGGGAGAAGGGAGCCCCGATGCCAGCG
CCGCAGGTGCCGCACTGTTAAATGGTGTGCCACGGGCCCGGGGGTCTAGGGACCGCCTGGCGAGCGGCAC
GTCTGGCTGAGGCGACCCTCGAGGAGGCCGCCGCATTCCCAGCGTGGGGGTCCTCCCGGGCTTGGGTGCG
GGGGGTCAGCAGGAGAGACCCTTCCGCGCGGCCTCCCTGCCTCCCCGGGGCCAGGGCGGGTTATAACTAG
CGGCTTCCACACAGCAGGAAGCCTGACAGAGGCTGCCCGCGGGGCTCCCAGCGACCCGAGGACATCGCAC
ACTTCCGGCCTTCTCTTCTCACACTCCCTCTGCCTTCCAAGTTCTCCCCACGCAGCTCAGGCGCGGGCAC
TGGCACCGCCCTGGGCACCGCCTGAGCAGGGCTGGGGGAGACCCGCCGAAGGGCCCACTCGCCCAGAGAC
GCCGTGGGGTCACGGCTGCGGCCTCGGCGGGGTGGGCTCGACACACAGGGTCAGGCCTCGGGACCACCTC
TCCCCATCCCGGCTGTCCCGGTCCCCAGCCGTCCTGCCCGCCTCAGACCGTCCGCCTCCTGCGTGTCTCC
TCCACTGAGGACGGCGGGGGAAGACCCCGGGAGCGTGGGCTCTGCACTTAGACGCATCCCCTCCTCCCGG
CTGTGGAGCCGTTTCCCGGGGGTTCCGGGGCCCTGCCCGCACTTCCGCCTTGCCGAGGGCGGGAGGAGTG
TGGCGGGACGGGCGGCGCCGCCCTGCGGTCTTACTTCTGGCAGGGCCCGCACTCCACGATCCCGTTGGCC
TCCCGGATGGCCGGCTGGTACACGAACACGGGGTACTCCGTGTCGCAGGGCTGCAGCGGGTCCTGCTTCT
TGTGTTTGTGGGCTGCGGGCGGAGAAAGGACATCACGCCACCGAAGGGGCCTCTGGCCTGCGGTGACCTC
GCCTCGGCACTCCCAGGGCCAGAGTTCACCCCAGTGCATTTGCAGAGGGTCCCTCCTCAGGCAGTTCTCA
GGACACGCGTGGGAGGAGCCGGGCGGGGGGTGAGGGGAGGGAAGACACCCGGAGTCTCTGGAGAGGGTGG
CTTGGCACGGGGAGCGCCGCAGGGGCCCTGAGCCTCGGTGGAGGGAAAGCGCTTCCTCTGGGGAGCCTGG
CCCTCACTGAGCTGGGGGCAGTGCACCATCTCCAGGGCCCCTTCCAGCACTCAGATCCCGGGGGAGGGTC
TGAGCGGGGAGCAGAGGCAGTAGGGGCACCAAGGGGTGGCTTGGGGGTACTTAGGCCCACCACCCCTCCC
TTCACTGTGGACAGACGCAGTGCAGTTCCTGGGCAATCCTGCCCTGCTCTCCCGCACAGCCAGGCCCACA
CTCCACCCATTGAGCCTGAGCGCCTGCGGTGGGGGCAGCCGGGCAGGTGGGCTCCAGGCCTGGGGCTGCG
GAAGCCCAGAAGCCACAGCCACCCGGTGCGGGACTCCTCGGGGGCTGCGCGATTTGCCTTAGCTCCCTGG
GACAAGGACAGTGGACTCAACCAAAGGTGGAGGCGAGTGCCTCAGCCCCGCTCTGCAGGACCCGAGTCCG
TTCCCTCCCGGAGCCTCCTGAGGTGGGCACATGGCAGAGCAGGGCCCTGCCACACAGGCTGTCTGCATGG
CCCTTCTGTTCTCAGGGCCTGGCTGTGGCCCCCAGGGATAGACCTAGAGGCAGGGAGCACAGGTGTGAGC
TACCTTCCCAGGCTCATGCAGAGTCCTACAGGAGAGCATGGGTCTCCCCAGCTTCCAAATCCAGAAGCTC
CCAGCCAGAGCTGGAAACCCAGAGGGGACACTAGCTGGCGTGCAGAAAACTCTCACCGAGAGGAGCAAGC
CCACAGCGGCCAGGGCTGTGAGTTTTGTCCCCAGGGCTGGGCACAGAAGCCTGCCTGACAATGGCAGGGT
TGAGTGGCGTCCCCAGGGTCACGCAGTGGAAGGGGCTGCAGCACCTACGCCCCGGCCTGGGACTCCAAGT
CCAGTGCGCCTTCACTGGGGACACTGACATCTGTGTAGGCCAGACCAGGGGGAGGAGCTCCAGGGGACAG
GTACCCCAGAGAAGAGAAATTCTGAGCAGGCAGATTGAGGGGGATCATCTTCAACTTTTGCAAGATGCAG
CTGCTGACAGAGACAGAGGGGACAGTGTCCTGTCACTGACACCTAGTGACTGTGAACACCCCAGAGCAGC
CCCACCCGATCGCGGATGTGAGTCCAGAAGGAGTCTGGAGACACAGCCCGAACTGCCCTGAGCCCAGGGG
CAGAGGAAGCCCCAAGCCAAGGCCAGCAGAAGAGAACGAAGAGCAAGCCAGAGAGAGGACCACAGTGGAG
ATGAAGAGATGGGATAAGCTGGGGACAGCCCAGGTGGTGGGAAGTTCTGGAACCCCATGGCAAAGGTTCT
AGGAACACGGAGGAACTGAGCAGAGCCCGCTGAGGCAAGATGGCCTCCTGCAGTCATGGAGGGCCCAGGC
GCCCGGGCAGTGAGTGCTTTGAAAGGCCAGGCTCATTGCCGCCCTGCACGTGTGGCCACAGCCACCGGGC
TTTGTGTTGGGGCCTCTCATGGTTGCTGCCTCTGGGGGACTCTGTCCTCAGACACAGGGTCCCGCAGGGG
AATCATCCTGAGAGCTTCCTGGGGCCCCTGGAACCCGGAGTCCCGCTGAGGGCTGGGGTGCCGCTGTTTG
CTTCTCCAAGGGGTGGGCTGGAGGTTGCTCACTCATACTGGCTCATGGCCACCCGGCCACACCACCGGCT
TCCTCTTGGAATCTTACGTGTTCTCACCCAAGGGGAGGCTGGAGTTTCTCATTTTCCCTGGGGCCTGACC
GTCCTCATGTGGGATGAGAACTGGGCTAGACTCCCCAGTCCCCCAACCCCCAGGAATGGTACTTTAAACC
AGGAGAGCTCAGCCCTGCTTGGGGTCATTCCTGGGACAGGTCATGGAGGGTGGGTTCTTTGTAGCTCCTG
CTGCGTCCCCAACCCACCGCCAGCAGGGATGGCCTCAGCTGGGCTACTTACAGTGATGGAAGACACTTTT
GGCTGGCCGGAACATAAGCCCAGCACAGGGTGGACACGGCACAGGAAAACATGGTGGCACATGAGGGCAG
GATGTCATGGGGTGGTGATGATGGCACATGGAGTGGCGGTGTCCCAAGATGATGTCACACACAGAGCCAG
GAGTGAGGGATGTGATGAGAGAAGGCCACGCAGGGTGAGATGTGAACAACGATGCTTCAGGGTCACCTAT
CCCCACTGTCACCCACCCCACAGGGAATGGTCTCACGTTAGTGGACCAAAATGCCACTGCTCTTCAGCAC
ATGGCCCTGCAGCTCCGTGCACTCACCCTCGGCCCCTCTGTCGTACCAGGAGCCCCAGACACTCCACTCC
AGCAGGAACCTGTCAGACACAGGACTGAATCTCGGAGACCTCTGAGCCCTCCCGACTTGGGTTCCCAAGG
AGCTGGAGTATCCCTGACAGCCCCCGGCCCATCCCCTCTCCTCCAGGACACCCTCTTCCCTGCTTGAGGG
CTCTGGTTGAGCAATGAGGTCCCTCCCAGGGGCAGCATGCAGGGAAGAGGTGGCGTCCATGCCCAGGGAT
TGTGCCCTCCTTCCCCGGGGTCTGGTAGCAAGTGGGTTGTCCTGGAGACCTTGCAGCCTCCTGCTCAAGG
ACACCCTCCTAAGCCACCCTCGCATGCAGCCCTCCACCAGCCCCGTGTCTACCCCCACTCACAGCACCAG
CTCCTGCAGCAGCCACCTGGTCGCCGTCAAGAAGGCAGAAATTGGCTGGGAAGGAGAGAGTGCACACCGC
TCGCACCTAGGTCCAAGGGTGAGGGTGCCCCCCCCCCACCACCAGCACCACCCTCAGAGAGACCAGAGAG
TGGGCTGCCCTGCCACTGGGAGCAGGGGCAGCAGAGCACAGGCCAGCAGCCCAGCACCCCCCATCCCCCC
ACCTCCCACCTGCCCTGCAGAGCTGAGCTAGCTGGGAAATGCCTATTGCAGGTCAGACATCTCGGGTGGG
GTTGGACATGCCCCTCCAGGAGACGAGTCAAGCCCTCATCCTGGTGCTGCACTGCTGAGGATCCCTGGTG
ATCAAGCGGCAGTGTCAGGGGCTGAGGCTGGGGCAGGCTGCTCTCTCTGTCTCCGCCAGAGGCACTGTTG
TGTCACAACTACCAGAGCAGTCCCTCTGAAGCCCCAAAGCCAGACCCAGAGGCCTGCCTGGGAGTGGGAC
TGAGAGCAGTGGCTCTGAGCAGAAGATGGAGATGGTCAAGGTAGGGCCCTGGGGCCAGACACCCAAGGTG
GGTGAGAACAGGGACCAGTGACCTCATTCCAGGACAGGGCAGAGGACTGGCTGGCTGGCTGGCAGAGGGA
AGGGCTGGGTGACACTCACGCTGACCAGGGGCTGGGCTGCACTGTGGTGGTGACTCGAGGGTTTGCACAT
GGCCTGATAGTCATACATAGTCACTCTGAAAATCAGAAGCGGGCTGTGATGAGTCCAAAGCCACCCCCAC
CCCCTGCCCCTGCCTCAGGAGGCTTTACTAGCAGAGCATCCGTTTACCGCAATTCCCAAGGGCGCCTGAG
CTAGGACAGCAGATCAGAATACAGCTCATGCTGAAAATTACATTTCTGCACTTGGAATGGTTTAAAGACA
ATGAAGAGCAAATGGCTTTTGCCCTCGGAGTTGGACGGTTTTCCCTCTAGGGTCTGAGCTGCGGAGCCCC
TCCCAGGGATGCCCACATGCCCTTACGCCCTCCCCACATCATGCTGACGCCCTGGCTCCATAAAGGTTGC
TTTTGCAGCTCTTTGGGGGCACCCACTGTGGGTTTTGGAGGTCAGGATCCTGAGATCCGCCAGGACCACT
TAGCGTCAGCTCTCGGTTTGCTGTGTCTATTTGGACTGGGGAGGAGTGTGCCCCACTTACTGGCTGAACA
CCCCCATGCTGAGCAGCTGGGTCAGGACAGCACCATCCACCTCCCCCAGAAATCTTCCCGTCTGTGAGAG
AGGGACAGCAGAGAGAGAGGGAGGGAGAGAGATGGAGCAGGATGGAGCCTTCCGAGTCCAGCACTATTTA
TTCAGCTCAGGTCGAGGCTTTTGGTGCCAGTTGGGCTTTCCCTCTGACTGCCTCTCAGCATAATCCAGCT
GAACCCTTCCACCCACGCCCCTGTCCGGTCCTGGCTCCCTCCTCACCGTCCAGCCTGGTGCTTCCTCAAC
TGGAAGGAGTCACCCGCTAGCTGACTATGAGACCAACGTAGAGGCTGCAATGTGATTTTTTTAATGGTAC
AGAATTCCTTGGAACAATTCTGTGTGAATCACTTGCAGCAAGGTTAAAGAATGTTTTATGAAACTGGATT
TGTTTTATATGCTGTGTGTGTGTGTGTGTGTGTGTGTGTGTGTGTGTGTGTGTGTGTTGGGTCAGATATA
AAACACGTTTCTGCAGGTCAAGGTCAGAAGCAGCGCCTGTCTCATGCACGCTGGTGTCCTCATCTTCCCA
GTGTGAGAGGGCTGCTGTGGCTTTTGCTGTCCCTCCATCACCTCCCACAATCAGTGTGCCCTGTGGATCG
GTCATTTGTCCCCTCCATGACAATGACCTAACCGGATCCCTTGCCCTGTCTCCATTGCCCACCTGCCCTC
CTAACTGACCTCTCCTCTTGTCCCTGTGGCACTGCCAGAGTCATCTTACTCAAATGCAACATGGATGGGT
CACTCTTGCCTTCATTTCCAAAAAATAAAACAGGACCCTCCCTCCTGCCCCCGGCTCCCCTTTTGTTGTC
CATCAGCCAGCACTTTGTGGCTTCTGTGCCCTGTGCCAAGCAGCGGCCCCCTAGCTGGCTGAGAATGGCC
TCAGGCCCTCCACTCTGCCACCTCTCATGGCCTCTGTACCTGCCTTGGCCTCTTGAGTCTGCCATTTCCC
AGCATGTCAGGGTGGCAGAGCTTGGGCTTTGGTGTTGGATTTCGACTTTAATGTAGGCTGTCTTTAAAAA
TTGGGGATAGCGTGTGTCCGGCGTGTGGCTCCCAGTAAATACTTGAAAAATGGCAGCCATCGTTAGGAGG
AGAGGTCCGGGGTCCGGCTTGGCTGTTCTCCCTGCCGATTACCCTTCCTCTGTCCTCATGCCAAGGAACT
TCTTTCCTCACCCTCGCTGGGAAGTCTTTCTATTCTAACTTTCCCAGCCAGAACAAGGCCTTCCTCTGCA
CACCGGCAGCCTGTGGAGTCCTAGTCTCACTCTGCCTGATAAGATATTTGGTTGAAAACTGTATGAGAAC
AGGACCATGTCTTACTCATCTCTGAATTCCCCAAAGTGTCTTGGTACCTGGACATACTCAGTCAGTGTGG
AATGTTGATAGGGAAGGGAGGTGGGAACCAAGCTGATGGCCACGTCAGGTTCTTCATGGAGAGCTTTCTA
GCTGCCAGGGCCCAAAATGATGACATGTGTGATAACTACCAATAAAAAGAAATGCAAGGCTGGTACAGGA
GCTGAGGCACATGGGCCTGGCCAATCTCAAGGAAAGCCAGGCATTGTCTGGGTGCCCACCTGGGGCCTGC
CCTGGGCAGCCAAGTAGACTCCAGCAAGCCTTCTGAAGGGAACAGAGATGTATTCCAGACAAGGGCTAGG
CAGACGCACACTCCTGACACCCATGAACAACCAGAGAAGTTCTCTCTCAAATAAGAGATGATGGTG
Transcript sequence for AdipoR2 (SEQ ID No. 8):
>gi|38261972|ref|NM_024551.2| Homo sapiens adiponectin receptor 2
(ADIPOR2), mRNA
CGCGGCGGCGGCAGCGGCGGCGGCTACACCGGGCTTGGCCCCCTCCCTCCTCCGTTCCCCCCTCCTCCCC
CCTCCCCTCAGCGGTGGCTCCCAAGAAGTCCGAGACACGCGGATCAACTCACTATCCTGAAGGTCCATTC
TCCCAAGAAGAGGGGACAGAAAGACAGATCTATTTGTAAGAAAGGCTTGGGTATCCCATGAACGAGCCAA
CAGAAAACCGATTGGGGTGCAGCAGGACTCCAGAGCCAGATATAAGGCTCAGAAAAGGGCACCAACTGGA
TGGTACACGAAGAGGTGATAATGACAGCCACCAAGGAGATTTGGAGCCCATTTTAGAGGCATCTGTTCTA
TCTTCCCATCATAAAAAAAGCTCTGAGGAACATGAATACAGTGATGAAGCTCCTCAGGAAGATGAGGGCT
TTATGGGCATGTCCCCTCTCTTACAAGCCCATCATGCTATGGAAAAAATGGAAGAATTTGTTTGTAAGGT
ATGGGAAGGTCGGTGGCGAGTGATCCCTCATGATGTACTACCAGACTGGCTCAAGGATAATGACTTCCTC
TTGCATGGACACCGGCCTCCTATGCCTTCTTTCCGGGCCTGTTTTAAGAGCATTTTCAGAATACACACAG
AAACAGGCAACATTTGGACACATCTCTTAGGTTGTGTATTCTTCCTGTGCCTGGGGATCTTTTATATGTT
TCGCCCAAATATCTCCTTTGTGGCCCCTCTGCAAGAGAAGGTGGTCTTTGGATTATTTTTCTTAGGAGCC
ATTCTCTGCCTTTCTTTTTCATGGCTCTTCCACACAGTCTACTGCCACTCAGAGGGGGTCTCTCGGCTCT
TCTCTAAACTGGATTACTCTGGTATTGCTCTTCTGATTATGGGAAGTTTTGTTCCTTGGCTTTATTATTC
TTTCTACTGTAATCCACAACCTTGCTTCATCTACTTGATTGTCATCTGTGTGCTGGGCATTGCAGCCATT
ATAGTCTCCCAGTGGGACATGTTTGCCACCCCTCAGTATCGGGGAGTAAGAGCAGGAGTGTTTTTGGGCC
TAGGCCTGAGTGGAATCATTCCTACCTTGCACTATGTCATCTCGGAGGGGTTCCTTAAGGCCGCCACCAT
AGGGCAGATAGGCTGGTTGATGCTGATGGCCAGCCTCTACATCACAGGAGCTGCCCTGTATGCTGCCCGG
ATCCCCGAACGCTTTTTCCCTGGCAAATGTGACATCTGGTTTCACTCTCATCAGCTGTTTCATATCTTTG
TGGTTGCTGGAGCTTTTGTTCACTTCCATGGTGTCTCAAACCTCCAGGAGTTTCGTTTCATGATCGGCGG
GGGCTGCAGTGAAGAGGATGCACTGTGATACCTACCAGTCTCCAGGGACTATGACCCTAAACCAGGGCCT
GCGGCACTTGCGGGCCTCCCTGCTGGCTACTGATGCCAGTACCAGAGGAGCCCCAAAACTTTGACAGCCT
CGTGGGCTTTGTGACGGCCCAGGGGCTCTGCGTGGTACATGACTGAGAAGAGAAAAACAAAAATAAATCA
TACCTCAAAGGATGGAGTGCATCAATTGGGAGAAAAGGAGACATAGCCCAAACCCTGGCTTATTCTTGGG
ATCTACTGATTGCGGGCTCTGCAAGACCCTTGGCAAACTGGCTTCTGATCCATATCATATTTATTTGTAG
AAGATGGCGAAACAGTTTAGCTGGTGGTTCTTTCTTCTCCCTTTCTCTCTCTCTATGACAATAATACAAA
CCAATTTAAGTGAACATTTATATCCGATAAGGGGTGGGAGTGTGATTTTAAATGCTCTTTTGGGAGAACA
AAGAAATTAATGTAAATAAGATTTCTAACTGTTTAAATAAGACTTTATATAAATGTTTAAAACATAGGGG
TAAGGGAGGGAGGGAGAATTTTTGTATAGAATGAAACATGCAAGTACCACACACTGTTTGAATTTTGCAC
AAAAAGTGACTGTAGGATCAGGTGATAGCCCCGGAATGTACAGTGTCTTGGTGCACCAAGATGCCTTCTA
AAGGCTGACATACCTTGGACCCTAATGGGGCAGAGAGTATAGCCCTAGCCCAGTGGTGACATGACCACTC
CCTTTGGGAGGCCTGAGGTAGAGGGGAGTGGTATGTGTTTTCTCAGTGGAAGCAGCACATGAGTGGGTGA
CAGGATGTTAGATAAAGGCTCTAGTTAGGGTGTCATTGTCATTTGAGAGACTGACACACTCCTAGCAGCT
GGTAAAGGGGTGCTGGAGGCCATGGAGGAGCTCTAGAAACATTAGCATGGGCTGATCTGATTACTTCCTG
GCATCCCGCTCACTTTTATGGGAAGTCTTATTAGAGGGATGGGACAGTTTTCCATATCCTTGCTGTGGAG
CTCTGGAACACTCTCTAAATTTCCCTCTATTAAAAATCACTGCCCTAACTACACTTCCTCCTTGAGGGAA
TAGAAATGGACCTTTCTCTGACATAGTTCTTGGCATGGGAGCCAGCCACAAATGAGATTCTGACGTGTCC
AGGTTTCTCCTGAGCTCATCTACATAGATTGGTAGACCCTTCCTTTGGATTAGGAAAGATGAGTTTTACC
TCTGGTACACTGTCTTGGTAAGCCTGGATGTGACAGACACCTCGGCTCTCCTTGAATAAGAAAGCCAGCA
GAACTCTTAAAGCCAGTTGTAGTACGGAGTTGTCAGCACTCACTGAACCTCACTTTACAGGGATAAGAGT
GGTGTGGCATTTTAAATACAATGGTATGTTATTGCCAGGGAGTGAGGTACAAGACGATGGCTCATGTCAC
AGGCCTACCTGATACGGTGTCAGAGAAAGTGGTGGGGAAAGGATCTGGTTCATGGAATTCTGATCTTGGC
CCATAGGTGAACCACCAAAATAGTGCTCGAGTCTTAGGTTACTGTCATCAAAGACTTGGGATGACTCCAT
TATATCCTGGGGTTGTGGGTATTAGAACTAAATATGGAGGTCCTGAGCATGGGGACTGGCGTCCTCAGTA
GGTGTTTGGGAATATGGGAAGGGTCTCCTATTTATTCAATAGAGTTTTCTCAGTTATTTTCCTCCCTTGC
CCTTGCAATCTCCAGCAAAAGGTGGGATCTAGGAAGAAAGAATCCAGTGTAGAAGTTGAGAAGAACTTGA
ACGTTTTGGTTCTGGATAAGGTCACTGTCCTAGGTGCTAGGTGGACCGAGCAAAAGACTCAGTGGATGAA
CTGGTGCAGTGCCTGACAGAATAAAGAACAGTATTAATCCCTTTGAGAAAGCATAGTCCAGCAGGACAGT
GGCCATTTGGACAGAAGCCCACTTAGTTTCTTGGGAGCAACAGCACGTATCAGAAGCCAGACTTGCTCTT
CGGTCATGCACTTTGGGATACAGCGTATAGGTGCAGCCCTGTCACAACACCAACAGAAGTAGCAGCCTCT
GGGTGCAGTCACCCACACCCCAAAGCTGGAAGGATCTGGTTCAACATAGCACAAACCCTTAGGAAAAATG
AAATTAACATCACTGATGTGTAATCCAGTAAAATCTCCCTTTTTCGGGTGTGTATGTGGGCATGTGCCCA
TTTCTATGTGTGTGTCTACGTGCAGCTCACTACCAACAGCCTCATGTGCACTTGACCTGACAGTGCTCGC
TGAGAACTCTCACCAGGTTGGCGCCTGAATGCCTTACTCTCAGCAGTCAGAGGCTTGCTTGCTCTGTGCA
GATTTTTAATTTTCTTTTTTGGCCCTAGGCTGGTTGGGACCTCTACAGCTTCATTCTTTCACCATTAAAT
AGTGGCCTTTTTCAGTATTTTCCCTCTTCCCCTTTATAAATTATGCTAAAGCCACAAAGCACATTTTTGG
GGATCATAGAAGGTTGGGGTTCCAGAAAGGCATCTGTGTGATGGTTCCATTGATGTGGGATTTCCCTACT
TGCTGTATTCTCAGTTTCTAATAAAAAGAACCAAATGAAATATG
Protein sequence (SEQ ID No. 9):
>sp|Q86V24|ADR2_HUMAN Adiponectin receptor protein 2 OS = Homo sapiens
GN = ADIPOR2 PE = 1 SV = 1
MNEPTENRLGCSRTPEPDIRLRKGHQLDGTRRGDNDSHQGDLEPILEASVLSSHHKKSSE
EHEYSDEAPQEDEGFMGMSPLLQAHHAMEKMEEFVCKVWEGRWRVIPHDVLPDWLKDNDF
LLHGHRPPMPSFRACFKSIFRIHTETGNIWTHLLGCVFFLCLGIFYMFRPNISFVAPLQE
KVVFGLFFLGAILCLSFSWLFHTVYCHSEGVSRLFSKLDYSGIALLIMGSFVPWLYYSFY
CNPQPCFIYLIVICVLGIAAIIVSQWDMFATPQYRGVRAGVFLGLGLSGIIPTLHYVISE
GFLKAATIGQIGWLMLMASLYITGAALYAARIPERFFPGKCDIWFHSHQLFHIFVVAGAF
VHFHGVSNLQEFRFMIGGGCSEEDAL

1. (canceled)

2. (canceled)

3. A method for identifying a patient likely to develop RA, comprising:
a) determining the level of the AdipoR1 and/or AdipoR2 biomarker(s) in leukocytes in a sample from the patient;
b) comparing the determined level of said biomarker(s) with a reference level; and
c) identifying the patient based on the comparison of b).

4. A method according to claim 3, wherein patients identified as not likely to develop RA have resolving disease, including inflammation that can be treated effectively with NSAIDs.

5. A method according to claim 3, wherein the sample is a body tissue or body fluid sample.

6. A method according to claim 3, wherein the sampled leukocyte cells are PBMCs (peripheral blood mononuclear cells), PBL (peripheral blood lymphocytes), B cells or Natural Killer cell populations, e.g. NK-T-Cells.

7. A method according to claim 3, wherein changes in cell-surface protein expression of one or more of the biomarker(s) is determined, such that reduced cell-surface protein expression of AdipoR1 and/or AdipoR2 on said leukocytes is indicative of early RA.

8. A method according to claim 7, wherein a population of B cells where less than or equal to 50-70%, and preferably less than or equal to 60%, of the B-cells show either marker is indicative of the patient being likely to develop RA.

9. A method according to claim 3, wherein changes in leukocyte sub-populations expressing the protein biomarker(s) are determined, such that less than or equal to 20% NK-T-cells expressing AdipoR1 and/or AdipoR2 is indicative of that the patient is likely to develop RA.

10. (canceled)

11. A method according to claim 3, wherein a difference between AdipoR1 and AdipoR2 mRNA expression levels is indicative of RA.

12. A method according to claim 11, wherein a relative expression (REU) above 0.14 (compared to 18S as internal standard gene) for AdipoR1 contrasted with an REU below 0.1 for AdipoR2 (compared to 18S as internal standard gene) is indicative of RA.

13. A method according to claim 3, wherein the mRNA expression ratio of AdiopR2:AdipoR1 (i.e. AdipoR2 divided by AdipoR1) is determined and a score of less than or equal to 90-110%, and preferably 100%, is indicative of early RA.

14. A method according to claim 3, wherein the percentage of leukocytes displaying cell-surface protein expression of AdiopR1 multiplied by the ratio of mRNA expression of AdiopR2 over AdipoR1 is determined and a score of less or equal to a threshold score of 85 is indicative of early RA, although a score of less than 50 is particularly preferred.

15. A method according to claim 14, wherein: for B-cells very early RA patients have scores of less than or equal to a threshold score of 50; for PBL very early RA patients have scores of less than or equal to a threshold of 15; and for NK T-cells very early RA patients have scores of less than or equal to a threshold of 10.

16. A method according to claim 3, wherein the percentage of leukocytes displaying cell-surface protein expression of AdiopR2 multiplied by the ratio of mRNA expression of AdiopR2 over AdipoR1, and a score of less than or equal to a threshold of 75 although a score of less than or equal to a threshold of 10 is particularly preferred.

17. A method according to claim 16, wherein: for B-cells, very early RA patients have scores of less than or equal to a threshold of 30; for PBL, very early RA patients have scores of less than or equal to a threshold of 15; and for NK T-cells, very early RA patients have scores of less than or equal to a threshold of 8.

18. A screening method for identifying, within a population, one or more patients likely to develop RA, comprising repeating the method according to claim 3 to thereby identify said one or more than one patient likely to develop RA.

19. A method according to claim 18, further comprising stratifying the patients according to their clinical outcome or likely clinical outcome.

20. A method for the treatment or prophylaxis of RA, comprising administering, to a patient identified according to claim 3, a drug such as rituximab or adalimumab for said treatment or prophylaxis of RA.
